(19) United States
(12) Patent Application Publication
(10) Pub. No.: US 2024/0131028 A1
(43) Pub. Date: Apr. 25, 2024

(54) CHEMOVACCINATION AGAINST PLASMODIUM INFECTION WITH SELECTIVE PLASMEPSIN X INHIBITORS

(71) Applicants: Justin A. BODDEY, Parkville, Victoria (AU); David B. OLSEN, West Point, PA (US); Ryan STEEL, Parkville, Victoria (AU); John A. MCCAULEY, West Point, PA (US); Manuel DE LERA RUIZ, West Point, PA (US); Merck Sharp & Dohme LLC, Rahway, NJ (US); The Walter and Eliza Hall Institute of Medical Research, Parkville, Victoria (AU)

(72) Inventors: Justin A. Boddey, Victoria (AU); David B. Olsen, Lansdale, PA (US); Ryan Steel, Victoria (AU); John A. McCauley, Maple Glen, PA (US); Manuel De Lera Ruiz, Perkasie, PA (US)

(73) Assignees: Merck Sharp & Dohme LLC, Rahway, NJ (US); The Walter and Eliza Hall Institute of Medical Research, Parkville, Victoria, AU (AU)

(21) Appl. No.: 18/263,551

(22) PCT Filed: Feb. 2, 2022
(86) PCT No.: PCT/US2022/014814

§ 371 Date: Jul. 31, 2023

Related U.S. Application Data
(60) Provisional application No. 63/146,176, filed on Feb. 5, 2021.

(51) Int. Cl. A61K 31/513; A61K 39/015; A61K 45/06; A61P 33/06

(52) U.S. Cl. CPC A61K 31/513; A61K 39/015; A61K 45/06; A61P 33/06; A61K 2039/522

(57) ABSTRACT

The present invention relates to a method of chemovaccination against Plasmodium infection comprising administering to a patient an effective amount of a selective inhibitor of plasmepsin X, or a pharmaceutically acceptable salt thereof.

### FIELD OF THE INVENTION

The present invention relates to methods of chemovaccination against Plasmodium infection. More specifically, the present invention relates to methods of chemovaccination against Plasmodium infection comprising administering to a patient an effective amount of a selective inhibitor of plasmepsin X, or a pharmaceutically acceptable salt thereof.

### BACKGROUND OF THE INVENTION

Malaria is a life-threatening disease that afflicts more than 200 million, and kills 620,000, people annually. Malaria-causing Plasmodium sporozoites are inoculated into the host by the bite of an infected mosquito. These sporozoites rapidly home to the liver and infect a hepatocyte, initiating an obligate but clinically silent phase of infection. In the hepatocyte the parasite rapidly transforms and grows into thousands of merozoites that later egress from the liver to infect red blood cells, causing malaria. Targeting liver parasites for elimination is an attractive antimalarial strategy since liver infection precedes malaria and therefore offers humans the opportunity to develop immunity to parasites before they take hold in the blood. The strategy is ideal, since a single parasite escaping elimination at the liver stage is sufficient to cause subsequent malaria disease.

Current malaria treatment relies primarily on drugs that target the disease causing asexual blood stages (ABS) of Plasmodium parasites, the organisms responsible for human malaria. These drugs include the 4-aminoquinolines piperaquine and amodiaquine, the antifolates pyrimethamine and sulfadoxine, and the endoperoxides artemisinin and its derivatives artesunate, artemether, and dihydroartemisinin. Artemisinin, usually in combination with partner drugs, has become a mainstay in the treatment and control of malaria. However, due to the increasing threat of artemisinin-based combination therapy (ACT) drug resistance, the development of new antimalarials targeted to inhibit multiple and/or additional steps in the parasitic life cycle is an urgent priority for the malaria control field.

Therefore, a much sought after treatment for malaria is an antimalarial medicine which has a profile that includes chemovaccination. Chemovaccines typically work against the exoerythrocytic parasite forms that invade and develop in the liver and are responsible for the earliest asymptomatic stage of the infection. Such medicines could be formulated to provide long-acting prophylaxis, safeguarding individuals that are living near or traveling to areas that have parasites. Long-acting chemovaccination in endemic regions could also greatly reduce circulating parasite numbers and potentially replace a vaccine in an elimination campaign. Antonova-Koch, Y., et al., Open-source discovery of chemical leads for next-generation chemoprotective antimalarials. Science, 2018, 362(6419): p. 9446.

### SUMMARY OF THE INVENTION

As shown in the Examples described below, selective inhibitors of the Plasmodium protease plasmepsin X (“PMX”) cure Plasmodium liver infection with a unique mechanism of action. These compounds serve as a causal prophylactic treatment by preventing liver to blood transition and malaria disease, as opposed to many antimalarials that treat the disease-causing asexual blood stage parasites.

The present invention is directed to methods of chemovaccination against Plasmodium infection comprising administering to a patient an effective amount of a selective inhibitor of plasmepsin X, or a pharmaceutically acceptable salt thereof.

The present invention is also directed to the use of selective inhibitors of the Plasmodium protease plasmepsin X, or pharmaceutically acceptable salts thereof, to cure Plasmodium liver infection.

The present invention is also directed to methods of chemovaccination against Plasmodium infection comprising administering to a patient an effective amount of a selective inhibitor of plasmepsin X, or a pharmaceutically acceptable salt thereof, wherein the patient has a Plasmodium parasite infection.

The present invention is also directed to the use of a selective inhibitor of plasmepsin X, or a pharmaceutically acceptable salt thereof, for chemovaccination against Plasmodium infection or malaria in a patient who has a Plasmodium parasite infection.

The present invention is also directed to methods of chemovaccination against Plasmodium infection comprising administering to a patient an effective amount of a selective inhibitor of plasmepsin X, or a pharmaceutically acceptable salt thereof, and an effective amount of one or more additional anti-malarial agents.

The present invention is also directed to the use of a selective inhibitor of plasmepsin X, or a pharmaceutically acceptable salt thereof, and an effective amount of one or more additional anti-malarial agents, for chemovaccination against Plasmodium infection or malaria in a patient.

The present invention is also directed to methods of chemovaccination against Plasmodium infection comprising administering an effective amount of a selective inhibitor of plasmepsin X, or a pharmaceutically acceptable salt thereof, and a wild-type Plasmodium parasite to a patient, wherein the patient does not have a Plasmodium parasite infection. The present invention is also directed to methods of chemovaccination against Plasmodium infection of a patient who does not have a Plasmodium parasite infection, comprising administering an effective amount of a selective inhibitor of plasmepsin X, or a pharmaceutically acceptable salt thereof, and a wild-type Plasmodium parasite to the patient.

The present invention is also directed to methods of chemovaccination against Plasmodium infection comprising administering to a patient, wherein the patient does not have a Plasmodium parasite infection, and wherein the patient is later exposed to a wild-type Plasmodium parasite, a long-acting injectable formulation comprising an effective amount of a selective inhibitor of plasmepsin X, or a pharmaceutically acceptable salt thereof.

The present invention is also directed to methods of chemovaccination against Plasmodium infection comprising administering, an effective amount of a selective inhibitor of plasmepsin X, or a pharmaceutically acceptable salt thereof, and a genetically modified Plasmodium parasite to a patient, wherein the patient does not have a Plasmodium parasite infection.

The present invention is also directed to the use of a selective inhibitor of plasmepsin X or a pharmaceutically acceptable salt thereof, and a wild-type Plasmodium parasite, for chemovaccination against Plasmodium infection in a patient, wherein the patient does not have a Plasmodium parasite infection.

The present invention is also directed to the use of a selective inhibitor of plasmepsin X or a pharmaceutically acceptable salt thereof, for chemovaccination against Plasmodium infection or malaria in a patient, wherein the patient does not have a Plasmodium parasite infection, wherein the patient is later exposed to a Plasmodium parasite.

The present invention is also directed to the use of a selective inhibitor of plasmepsin X or a pharmaceutically acceptable salt thereof, for chemovaccination against Plasmodium infection or malaria, and a genetically modified Plasmodium parasite, in a patient, wherein the patient does not have a Plasmodium parasite infection.

The present invention is also directed to methods of chemovaccination against Plasmodium infection comprising administering an effective amount of a compound of Formula (I):

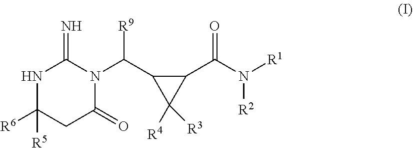

wherein R¹, R², R³, R⁴, R⁵, R⁶ and R⁹ are described below, or a pharmaceutically acceptable salt thereof to a patient.

The present invention is also directed to the methods and uses described herein, wherein the selective inhibitor of plasmepsin X is a compound of Formula (I):

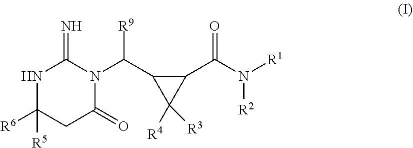

wherein R¹, R², R³, R⁴, R⁵, R⁶ and R⁹ are described below.

Also described herein are compounds capable of inducing an immune response in a patient, wherein the compound is a compound of Formula (I), (IA), (IB), (IC) or (ID).

Also described herein are compounds capable of inducing an immune response in a patient, wherein the compounds are compounds of Formula (I), (IA), (IB), (IC) or (ID), and wherein the compounds are capable of inducing an immune response to a Plasmodium parasite infection.

Also described herein are methods of inducing an immune response to a Plasmodium parasite infection, comprising administering to a patient, an effective amount of a compound, or a pharmaceutically acceptable salt thereof, wherein the compound is capable of inducing an immune response and the compound functions via selective inhibition of plasmepsin X.

Also described herein are methods of inducing an immune response to a Plasmodium parasite infection, comprising administering to a patient an effective amount of a selective inhibitor of plasmepsin X.

Also described herein are methods of inducing an immune response to a Plasmodium parasite infection, comprising administering to a patient an effective amount of a compound of Formula (I), or a pharmaceutically acceptable salt thereof.

Also described herein are methods of inducing an immune response to a Plasmodium parasite infection, comprising administering to a patient, wherein the patient has a Plasmodium parasite infection, an effective amount of a compound of Formula (I), (IA), (IB), (IC) or (ID), or a pharmaceutically acceptable salt thereof.

Also described herein are compositions capable of inducing an immune response comprising a selective inhibitor of plasmepsin X, or a pharmaceutically acceptable salt thereof; and a pharmaceutically acceptable carrier or excipient.

Also described herein are compositions capable of inducing an immune response comprising a compound of Formula (I), (IA), (IB), (IC) or (ID), or a pharmaceutically acceptable salt thereof; and a pharmaceutically acceptable carrier or excipient.

Also described herein are compositions capable of inducing an immune response comprising a compound of Formula (I), (IA), (IB), (IC) or (ID), or a pharmaceutically acceptable salt thereof; and an adjuvant.

### BRIEF DESCRIPTION OF THE DRAWINGS

FIG. 1 is a graph showing the results of mice dosed with various doses of Example 2, a selective inhibitor of plasmepsin X. As shown, mice dosed with 2×500 mg/kg of Example 2, showed chemoprophylaxis (mice challenged with sporozoites and given Example 2, never got malaria).

FIG. 2 is a graph showing the results of eight mice previously chemovaccinated with 2×100 mg/kg and 2×200 mg/kg of Example 2 which were challenged three months later with misquito bites (MB) from 10 mosquitoes infected with PbmCherryLuci sporozoites.

### DETAILED DESCRIPTION OF THE INVENTION

The present invention is directed to methods of chemovaccination against Plasmodium infection comprising administering to a patient, an effective amount of a selective inhibitor of plasmepsin X, or a pharmaceutically acceptable salt thereof. The present invention is also directed to methods of inducing an immune response to Plasmodium infection, comprising administering to a patient an effective amount of a selective inhibitor of plasmepsin X, or a pharmaceutically acceptable salt thereof.

As shown in the experiments described herein, using inhibitors of the Plasmodium protease plasmepsin X (“PMX”), Plasmodium liver infection was cured. As shown below, mice were infected with Plasmodium berghei sporozoites and treated orally with selective PMX inhibitors mid-way through liver infection. The mice were monitored in real time to see if the inhibitors cleared parasites from the liver or prevented their egress from the liver. The mice were also monitored to see if they developed a malaria-causing blood infection for 30 days post infection. If no infection was seen after 30 days, the mice were considered to have been fully protected from malaria.

From these experiments, selective PMX inhibitor compounds and doses capable of protecting mice from the malaria-causing blood infection when treated during liver infection were identified. The mechanism by which these compounds cure the malaria infected mice is not through killing liver stage parasites or completely blocking their egress from the liver; rather, the mechanism of action is very late acting in that liver-derived (exoerythrocytic) merozoites that left the liver were non-infectious to erythrocytes. In this way, the compounds serve as a causal prophylactic treatment by preventing liver to blood transition and malaria disease, as opposed to many antimalarials that treat the disease-causing asexual blood stage parasites. There are no known antimalarials either approved or in development that act with this mechanism at this late stage on liver parasites to completely prevent malaria disease.

Therefore, based on the data above and further described in the sections below, the present invention is directed to methods of chemovaccination against Plasmodium infection comprising administering to a patient an effective amount of a selective inhibitor of plasmepsin X, or a pharmaceutically acceptable salt thereof. Chemovaccination can take place in two forms, in one embodiment the selective inhibitor of plasmepsin X, or a pharmaceutically acceptable salt thereof, is administered to a patient with an existing Plasmodium infection. In this embodiment, the infection is cured, and the patient is vaccinated against future infections. In a second embodiment, a patient who does not have an existing Plasmodium infection is administered an effective amount of a selective inhibitor of plasmepsin X, or a pharmaceutically acceptable salt thereof, and is then simultaneously or sequentially administered or exposed to a Plasmodium parasite.

Additionally, based on the data described in the sections below, the present invention is directed to methods of inducing an immune response to Plasmodium infection, comprising administering to a patient an effective amount of a compound capable of inducing an immune response to Plasmodium infection. Additionally, based on the data described in the sections below, the present invention is directed to methods of inducing an immune response to Plasmodium infection, comprising administering to a patient an effective amount of a selective inhibitor of plasmepsin X, or a pharmaceutically acceptable salt thereof. Induction of an immune response can take place in two forms, in one embodiment a selective inhibitor of plasmepsin X, or a pharmaceutically acceptable salt thereof, is administered to a patient with an existing Plasmodium infection. In this embodiment, the infection is cured, and the patient is vaccinated against future infections. In a second embodiment, a patient who does not have an existing Plasmodium infection is administered an effective amount of a selective inhibitor of plasmepsin X, or a pharmaceutically acceptable salt thereof, and is then simultaneously or sequentially administered or exposed to a Plasmodium parasite.

In the methods described herein, the Plasmodium infection can be caused by Plasmodium falciparum, Plasmodium vivax, Plasmodium ovale, Plasmodium malariae or Plasmodium knowlesi.

The present invention is also directed to the use of a selective inhibitor of the Plasmodium protease plasmepsin X, or a pharmaceutically acceptable salt thereof, to cure Plasmodium liver infection.

The present invention is also directed to methods of chemovaccination against Plasmodium infection comprising administering to a patient having a Plasmodium infection, an effective amount of a selective inhibitor of plasmepsin X, or a pharmaceutically acceptable salt thereof.

The present invention is also directed to the use of a selective inhibitor of plasmepsin X or a pharmaceutically acceptable salt thereof, for chemovaccination against Plasmodium infection or malaria in a patient, wherein the patient has a Plasmodium infection.

The present invention is also directed to methods of chemovaccination against Plasmodium infection comprising administering to a patient an effective amount of a selective inhibitor of plasmepsin X, or a pharmaceutically acceptable salt thereof, and a wild-type Plasmodium parasite, wherein the patient does not have a Plasmodium parasite infection.

The present invention is also directed to methods of chemovaccination against Plasmodium infection comprising administering to a patient an effective amount of a selective inhibitor of plasmepsin X, or a pharmaceutically acceptable salt thereof, and a genetically modified Plasmodium parasite, wherein the patient does not have a Plasmodium parasite infection.

The present invention is also directed to the use of a selective inhibitor of plasmepsin X, or a pharmaceutically acceptable salt thereof and a wild-type Plasmodium parasite for chemovaccination against Plasmodium infection or malaria in a patient, wherein the patient does not have a Plasmodium infection.

The present invention is also directed to the use of a selective inhibitor of plasmepsin X, or a pharmaceutically acceptable salt thereof, for concurrent or sequential administration with a wild-type Plasmodium parasite, for chemovaccination against Plasmodium infection or malaria in a patient, wherein the patient does not have a Plasmodium infection.

The present invention is also directed to the use of a selective inhibitor of plasmepsin X, or a pharmaceutically acceptable salt thereof, and a genetically modified parasite for chemovaccination against Plasmodium infection or malaria, in a patient, wherein the patient does not have a Plasmodium infection.

The present invention is also directed to methods of chemovaccination against Plasmodium infection comprising administering to a patient an effective amount of a selective inhibitor of plasmepsin X, or a pharmaceutically acceptable salt thereof, wherein the patient does not have a Plasmodium parasite infection and wherein the patient is later exposed to a Plasmodium parasite.

The present invention is also directed to the use of a selective inhibitor of plasmepsin X or a pharmaceutically acceptable salt thereof, for chemovaccination against Plasmodium infection or malaria in a patient, wherein the patient does not have a Plasmodium infection and wherein the patient is later exposed to a wild-type Plasmodium parasite.

In certain embodiments, the exposure to a wild-type Plasmodium parasite is through a mosquito bite.

### Compounds for Chemovaccination

In certain embodiments described herein, the present invention is directed to methods of chemovaccination against Plasmodium infection comprising administering to a patient, an effective amount a compound of Formula (I). In other embodiments described herein, the present invention is directed to methods of chemovaccination against Plasmodium infection comprising administering to a patient, an effective amount of a selective inhibitor of plasmepsin X, wherein the selective inhibitor of plasmepsin X is a compound of Formula (I):

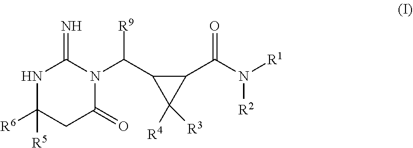

or a pharmaceutically acceptable salt thereof, wherein:
R¹ is a heterocycloalkyl, C₃-C₁₂cycloalkyl, aryl or C₁-C₆alkylaryl, wherein the heterocycloalkyl, C₃-C₁₂cycloalkyl, aryl, or C₁-C₆alkylaryl is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), N(R⁷)(R⁸) and C₁-C₆alkylN(R⁷)(R⁸);
R² is hydrogen, C₁-C₆alkylCOOH, —COOH, C₃-C₆cycloalkyl, C₁-C₆alkyl, haloC₁-C₆alkyl or C₁-C₆alkylOH;
R³ is hydrogen, halogen, —CN, —OH, C₃-C₆cycloalkyl or C₁-C₆alkyl;
R⁴ is hydrogen, halogen, —CN, —OH, C₃-C₆cycloalkyl or C₁-C₆alkyl;
R⁵ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, —COOH, C₃-C₆cycloalkyl, C₁-C₆alkyl, haloC₁-C₆alkyl or C₁-C₆alkylOH;
R⁸ is hydrogen, C₁-C₆alkylCOOH, —COOH, C₃-C₆cycloalkyl, C₁-C₆alkyl, haloC₁-C₆alkyl or C₁-C₆alkylOH; and
R⁹ is hydrogen, halogen, —CN, alkoxy, C₁-C₆alkyl, heterocycloalkyl, heteroaryl, C₃-C₁₂cycloalkyl, aryl, —COOH, oxo, —COOC₁-C₆alkyl, haloC₁-C₆alkyl, —OH, —CON(R⁷)(R⁸) and N(R⁷)(R⁸), wherein the C₁-C₆alkyl is unsubstituted or substituted with one, two or three substituents independently selected from the group consisting of halogen, —CN, —OH, alkoxy, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and —N(R⁷)(R⁸), and wherein the heterocycloalkyl, heteroaryl, C₃-C₁₂cycloalkyl and aryl are unsubstituted or substituted with one, two or three substituents independently selected from the group consisting of halogen, —CN, —OH, alkoxy, C₁-C₆alkyl, —COOH, oxo, —COOC₁-C₆alkyl, haloC₁-C₆alkyl, —CON(R⁷)(R⁸) and —N(R⁷)(R⁸).

In the embodiments described herein, R¹ is a heterocycloalkyl, C₃-C₁₂cycloalkyl, aryl, or C₁-C₆alkylaryl, wherein the heterocycloalkyl, C₃-C₁₂cycloalkyl, aryl or C₁-C₆alkylaryl is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), N(R⁷)(R⁸) and C₁-C₆alkylN(R⁷)(R⁸).

In the embodiments described herein, R¹ is a heterocycloalkyl, C₃-C₁₂cycloalkyl, or C₁-C₆alkylaryl, wherein the heterocycloalkyl, C₃-C₁₂cycloalkyl, aryl or C₁-C₆alkylaryl is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), N(R⁷)(R⁸) and C₁-C₆alkylN(R⁷)(R⁸).

In certain embodiments, R¹ is a bicyclic ring. In certain embodiments, R¹ is a bicyclic heterocycloalkyl, bicyclic C₃-C₁₂cycloalkyl or bicyclic aryl ring. In certain embodiments, R¹ is a fused bicyclic ring having an A ring fused with a B ring, wherein the A ring is a 5, 6 or 7-membered saturated ring and the B ring is a phenyl or a 5 or 6-membered heteroaryl ring. In certain embodiments, the A ring includes at least one O, N, or S. In certain embodiments, the A ring includes at least one 0.

In certain embodiments, R¹ is a heterocycloalkyl. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of bicyclic heterocycloalkyl groups include, but are not limited to,

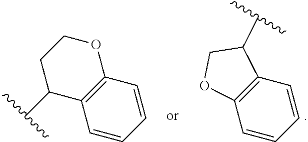 [SMILES: *C1CCOc2ccccc21 ; *C1COc2ccccc21]

In certain embodiments, R¹ is a C₃-C₁₂cycloalkyl. In certain embodiments, the cycloalkyl is a monocyclic cycloalkyl. Suitable examples of cycloalkyls include, but are not limited to, cyclopropyl, cyclobutyl, cyclopentyl and cyclohexyl. In certain embodiments, the cycloalkyl is a bicyclic cycloalkyl. Suitable examples of cycloalkyls include, but are not limited to:

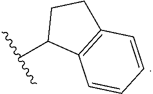 [SMILES: *C1CCc2ccccc21]

In certain embodiments, R¹ is an aryl ring. Suitable examples of aryls include, but are not limited to, monocyclic aryl groups such as, phenyl and bicyclic aryl groups such as naphthyl.

In certain embodiments, R¹ is a C₁-C₆alkylaryl ring. Suitable examples of C₁-C₆alkylaryls include, but are not limited to:

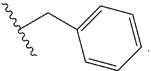 [SMILES: *Cc1ccccc1]

In certain embodiments, R¹ is a chromane or indane.

In certain embodiments, R¹ is unsubstituted. In other embodiments, R¹ is substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁵), N(R⁷)(R⁸) and C₁-C₆alkylN(R⁷)(R⁵). In certain embodiments, R¹ is substituted with 1 substituent. In certain embodiments, R¹ is substituted with 2 substituents. In certain embodiments, R¹ is substituted with 3 substituents. In certain embodiments, R¹ is substituted with 4 substituents. In certain embodiments, R¹ is substituted with 5 substituents.

In certain embodiments, R¹ is substituted with halogen. Examples of suitable halogens include, but are not limited to, chlorine, bromine, fluorine and iodine. In certain embodiments, R¹ is substituted with —CN. In certain embodiments, R¹ is substituted with —OH. In certain embodiments, R¹ is substituted with an oxo group.

In certain embodiments, R¹ is substituted with alkoxy. Suitable alkoxys include, but are not limited to, methoxy, ethoxy, n-propoxy, isopropoxy and n-butoxy. In certain embodiments, R¹ is substituted with C₁-C₆alkylOC₁-C₆alkyl. In certain embodiments, R¹ is substituted with haloalkoxy. Suitable haloalkoxys include, but are not limited to, trifluoromethoxy, fluoromethoxy and difluoromethoxy. In certain embodiments, R¹ is substituted with C₁-C₆alkylOC₁-C₆alkyl. Suitable C₁-C₆alkylOC₁-C₆alkyls include, but are not limited to, CH₂OCH₃. In certain embodiments, R¹ is substituted with C₁-C₆alkylCOOH. In certain embodiments, R¹ is substituted with —COOH. In certain embodiments, R¹ is substituted with C₁-C₆alkylCOOC₁-C₆alkyl.

In certain embodiments, R¹ is substituted with C₃-C₆cycloalkyl. Suitable examples of cycloalkyls include, but are not limited to, cyclopropyl, cyclobutyl, cyclopentyl and cyclohexyl. In certain embodiments, R¹ is substituted with cyclopropyl:

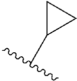 [SMILES: *C1CC1]

In certain embodiments, R¹ is substituted with spiroC₃-C₆cycloalkyl. Suitable examples of spirocycloalkyls include, but are not limited to, spirocyclopropyl, spirocyclobutyl, spirocyclopentyl and spirocyclohexyl. In certain embodiments, R¹ is substituted with spirocyclobutyl:

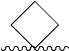

In certain embodiments, R¹ is substituted with C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹ is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is substituted with C₁-C₆alkylOH. Examples of suitable alcohols, include, but are not limited to, methanol, ethanol, propanol, butanol and iso-butanol. In certain embodiments, R¹ is substituted with —CON(R⁷)(R⁸). In certain embodiments, R¹ is substituted with N(R⁷)(R⁸). In certain embodiments, R¹ is substituted with C₁-C₆alkylN(R⁷)(R⁸), wherein R⁷ and R⁸ will be described in detail below.

In certain embodiments, R¹ is substituted with 1 to 4 substituents selected independently from the group consisting of bromine, fluorine, chlorine, methyl, ethyl, t-butyl, methoxy, —OH, —CN oxo, CH₂OCH₃, cyclopropyl, spirocyclobutyl, trifluoromethoxy and trifluoromethyl.

In certain embodiments, R¹ is

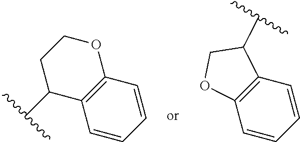 [SMILES: *C1CCOc2ccccc21 ; *C1COc2ccccc21]

or and is substituted with 1 to 4 substituents selected independently from the group consisting of bromine, fluorine, chlorine, methyl, ethyl, t-butyl, methoxy, —OH, —CN oxo, CH₂OCH₃, cyclopropyl, spirocyclobutyl, trifluoromethoxy and trifluoromethyl.

In certain embodiments, R¹ is

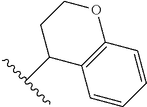 [SMILES: *C1CCOc2ccccc21]

and is substituted with 1 to 4 substituents selected independently from the group consisting of bromine, fluorine, chlorine, methyl, ethyl, t-butyl, methoxy, —OH, —CN oxo, CH₂OCH₃, cyclopropyl, spirocyclobutyl, trifluoromethoxy and trifluoromethyl.

In certain embodiments, R¹ is

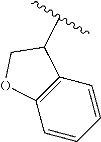 [SMILES: *C1COc2ccccc21]

and is substituted with 1 to 4 substituents selected independently from the group consisting of bromine, fluorine, chlorine, methyl, ethyl, t-butyl, methoxy, —OH, —CN oxo, CH₂OCH₃, cyclopropyl, spirocyclobutyl, trifluoromethoxy and trifluoromethyl. In certain embodiments described herein, R² is hydrogen, C₁-C₆alkylCOOH, —COOH, C₃-C₆cycloalkyl, C₁-C₆alkyl, haloC₁-C₆alkyl or C₁-C₆alkylOH. In certain embodiments, R² is hydrogen. In certain embodiments, R² is C₁-C₆alkylCOOH. In certain embodiments, R² is —COOH. In certain embodiments, R² is C₃-C₆cycloalkyl. Suitable examples of cycloalkyls include, but are not limited to, cyclopropyl, cyclobutyl, cyclopentyl and cyclohexyl. In certain embodiments, R² is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R² is C₁-C₆alkylOH. Examples of suitable alcohols, include, but are not limited to, methanol, ethanol, propanol, butanol and iso-butanol.

With regard to the compounds described herein, R³ is hydrogen, halogen, —CN, —OH, C₃-C₆cycloalkyl or C₁-C₆alkyl. In certain embodiments, R³ is hydrogen. In certain embodiments, R³ is halogen. Suitable halogens include, but are not limited to, fluorine, chlorine, bromine, or iodine. In certain embodiments, R³ is —CN. In certain embodiments, R³ is —OH.

In certain embodiments, R³ is C₃-C₆cycloalkyl. Suitable examples of cycloalkyls include, but are not limited to, cyclopropyl, cyclobutyl, cyclopentyl and cyclohexyl. In certain embodiments, R³ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R³ is hydrogen, fluorine, methyl, ethyl or

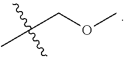 [SMILES: *COC]

In certain embodiments, R³ is taken with R⁴ to form oxetanyl.

With regard to the compounds described herein, R⁴ is hydrogen, halogen, —CN, —OH, C₃-C₆cycloalkyl or C₁-C₆alkyl. In certain embodiments, R⁴ is hydrogen. In certain embodiments, R⁴ is halogen. Suitable halogens include, but are not limited to, fluorine, chlorine, bromine, or iodine. In certain embodiments, R⁴ is —CN. In certain embodiments, R⁴ is —OH.

In certain embodiments, R⁴ is C₃-C₆cycloalkyl. Suitable examples of cycloalkyls include, but are not limited to, cyclopropyl, cyclobutyl, cyclopentyl and cyclohexyl. In certain embodiments, R⁴ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁴ is hydrogen, methyl, or fluorine. In certain embodiments, R³ and R⁴ are both hydrogen, methyl or fluorine.

In certain embodiments, R³ is hydrogen and R⁴ is hydrogen, methyl or fluorine.

With regard to the compounds described herein, R⁵ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁵), N(R⁷)(R⁸) or C₁-C₆alkylN(R⁷)(R⁸) or when taken with R⁶ forms a C₃-C₆cycloalkyl or C₃-C₆heterocycloalkyl. In certain embodiments, R⁵ is hydrogen. In certain embodiments, R⁵ is halogen. Suitable halogens include, but are not limited to, fluorine, chlorine, bromine, or iodine. In certain embodiments, R⁵ is —CN. In certain embodiments, R⁵ is —OH.

In certain embodiments, R⁵ is alkoxy. Suitable alkoxys include, but are not limited to, methoxy, ethoxy, n-propoxy, isopropoxy and n-butoxy. In certain embodiments, R⁵ is C₁-C₆alkylOC₁-C₆alkyl. In certain embodiments, R⁵ is —COOH. In certain embodiments, R⁵ is C₁-C₆alkylCOOH. In certain embodiments, R⁵ is C₃-C₆cycloalkyl. Suitable examples of cycloalkyls include, but are not limited to, cyclopropyl, cyclobutyl, cyclopentyl and cyclohexyl. In certain embodiments, R⁵ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is C₁-C₆alkylOH. Examples of suitable alcohols include, but are not limited to, methanol, ethanol, propanol, butanol and iso-butanol. In certain embodiments, R⁵ is —CON(R⁷)(R⁵). In certain embodiments, R⁵ is N(R⁷)(R⁵). In certain embodiments, R⁵ is C₁-C₆alkylN(R⁷)(R⁵). R⁷ and R⁸ will be discussed in detail below.

In certain embodiments, R⁵ is taken with R⁶ and forms a C₃-C₆cycloalkyl or C₃-C₆heterocycloalkyl. In certain embodiments, R⁵ is taken with R⁶ and forms a C₃-C₆cycloalkyl.

Suitable examples of cycloalkyls include, but are not limited to, cyclopropyl, cyclobutyl, cyclopentyl and cyclohexyl. In certain embodiments, R⁵ is taken with R⁶ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁵ is methyl, ethyl or t-butyl.

With regard to the compounds described herein, R⁶ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁵), N(R⁷)(R⁸) or C₁-C₆alkylN(R⁷)(R⁸) or when taken with R⁵ forms a C₃-C₆cycloalkyl or C₃-C₆heterocycloalkyl. In certain embodiments, R⁶ is hydrogen. In certain embodiments, R⁶ is halogen. Suitable halogens include, but are not limited to, fluorine, chlorine, bromine, or iodine. In certain embodiments, R⁶ is —CN. In certain embodiments, R⁶ is —OH.

In certain embodiments, R⁶ is alkoxy. Suitable alkoxys include, but are not limited to, methoxy, ethoxy, n-propoxy, isopropoxy and n-butoxy. In certain embodiments, R⁶ is C₁-C₆alkylOC₁-C₆alkyl. In certain embodiments, R⁶ is —COOH. In certain embodiments, R⁶ is C₁-C₆alkylCOOH. In certain embodiments, R⁶ is C₃-C₆cycloalkyl. Suitable examples of cycloalkyls include, but are not limited to, cyclopropyl, cyclobutyl, cyclopentyl and cyclohexyl. In certain embodiments, R⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁶ is C₁-C₆alkylOH. Examples of suitable alcohols include, but are not limited to, methanol, ethanol, propanol, butanol and iso-butanol. In certain embodiments, R⁶ is —CON(R⁷)(R⁵). In certain embodiments, R⁶ is N(R⁷)(R⁵). In certain embodiments, R⁶ is C₁-C₆alkylN(R⁷)(R⁵). R⁷ and R⁸ will be discussed in detail below.

In certain embodiments, R⁶ is taken with R⁵ and forms a C₃-C₆cycloalkyl or C₃-C₆heterocycloalkyl. In certain embodiments, R⁶ is taken with R⁵ and forms a C₃-C₆cycloalkyl.

Suitable examples of cycloalkyls include, but are not limited to, cyclopropyl, cyclobutyl, cyclopentyl and cyclohexyl. In certain embodiments, R⁶ is taken with R⁵ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁶ is methyl, ethyl or t-butyl.

With regard to the compounds described herein, R⁷ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁷ is hydrogen. In certain embodiments, R⁷ is C₁-C₆alkylCOOH. In certain embodiments, R⁷ is —COOH. In certain embodiments, R⁷ is C₃-C₆cycloalkyl. Suitable examples of cycloalkyls include, but are not limited to, cyclopropyl, cyclobutyl, cyclopentyl and cyclohexyl. In certain embodiments, R⁷ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is C₁-C₆alkylOH. Examples of suitable alcohols include, but are not limited to, methanol, ethanol, propanol, butanol and iso-butanol.

With regard to the compounds described herein, R⁸ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁸ is hydrogen. In certain embodiments, R⁸ is C₁-C₆alkylCOOH. In certain embodiments, R⁸ is —COOH. In certain embodiments, R⁸ is C₃-C₆cycloalkyl. Suitable examples of cycloalkyls include, but are not limited to, cyclopropyl, cyclobutyl, cyclopentyl and cyclohexyl. In certain embodiments, R⁸ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is C₁-C₆alkylOH. Examples of suitable alcohols include, but are not limited to, methanol, ethanol, propanol, butanol and iso-butanol.

With regard to the compounds described herein, R⁹ is hydrogen, halogen, —CN, —OH, alkoxy, C₁-C₆alkyl, heterocycloalkyl, heteroaryl, C₃-C₁₂cycloalkyl, aryl, —COOH, oxo, —COOC₁-C₆alkyl, haloC₁-C₆alkyl, —OH, —CON(R⁷)(R⁸) and N(R⁷)(R⁸), wherein the C₁-C₆alkyl is unsubstituted or substituted with one, two or three substituents selected independently from the group consisting of halogen, —CN, —OH, alkoxy, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and N(R⁷)(R⁸), and wherein the heterocycloalkyl, heteroaryl, C₃-C₁₂cycloalkyl and aryl are unsubstituted or substituted with one, two or three substituents independently selected from the group consisting of halogen, —CN, —OH, alkoxy, C₁-C₆alkyl, —COOH, oxo, —COOC₁-C₆alkyl, haloC₁-C₆alkyl, —CON(R⁷)(R⁸) and N(R⁷)(R⁸).

In certain embodiments, R⁹ is hydrogen. In certain embodiments, R⁹ is halogen. Suitable halogens include, but are not limited to, fluorine, chlorine, bromine, or iodine. In certain embodiments, R⁹ is —CN.

In certain embodiments, R⁹ is alkoxy. Suitable alkoxys include, but are not limited to, methoxy, ethoxy, n-propoxy, isopropoxy and n-butoxy. In certain embodiments, R⁹ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁹ is a heterocycloalkyl. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. In certain embodiments, R⁹ is a heteroaryl group. Suitable heteroaryls include, but are not limited to, pyridyl (pyridinyl), imidazolyl, triazolyl, triazinyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl. In certain embodiments, R⁹ is a pyridine.

In certain embodiments, R⁹ is a C₃-C₁₂cycloalkyl. In certain embodiments, the cycloalkyl is a monocyclic cycloalkyl. Suitable examples of cycloalkyls include, but are not limited to, cyclopropyl, cyclobutyl, cyclopentyl and cyclohexyl. In certain embodiments, the cycloalkyl is a bicyclic cycloalkyl.

In certain embodiments, R⁹ is an aryl ring. Suitable examples of aryls include, but are not limited to, monocyclic aryl groups such as phenyl and bicyclic aryl groups such as naphthyl.

In certain embodiments, R⁹ is —COOH. In certain embodiments, R⁹ is oxo. In certain embodiments, R⁹ is C₁-C₆alkylCOOC₁-C₆alkyl. In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is —CON(R⁷)(R⁸). In certain embodiments, R⁹ is N(R⁷)(R⁸). In certain embodiments, R⁹ is C₁-C₆alkylN(R⁷)(R⁸).

In certain embodiments, R⁹ is unsubstituted or substituted. In certain embodiments, when R⁹ is C₁-C₆alkyl, C₁-C₆alkyl is unsubstituted or substituted with one, two or three substituents independently selected from the group consisting of halogen, —CN, —OH, alkoxy, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and N(R⁷)(R⁸). In certain embodiments, R⁹ is

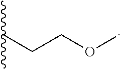

In certain embodiments, R⁹ is

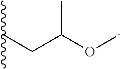

In certain embodiments, when R⁹ is heterocycloalkyl, heteroaryl, C₃-C₁₂cycloalkyl or aryl, the heterocycloalkyl, heteroaryl, C₃-C₁₂cycloalkyl or aryl the are unsubstituted or substituted with one, two or three substituents independently selected from the group consisting of halogen, —CN, —OH, alkoxy, C₁-C₆alkyl, —COOH, oxo, —COOC₁-C₆alkyl, haloC₁-C₆alkyl, —CON(R⁷)(R⁸) and N(R⁷)(R⁸). In certain embodiments, R⁹ is pyridine substituted with fluorine, chlorine or methoxy. In certain embodiments, R⁹ is phenyl substituted with —CN.

Also described herein are compounds of Formula (IA), (IB), (IC) and (ID):

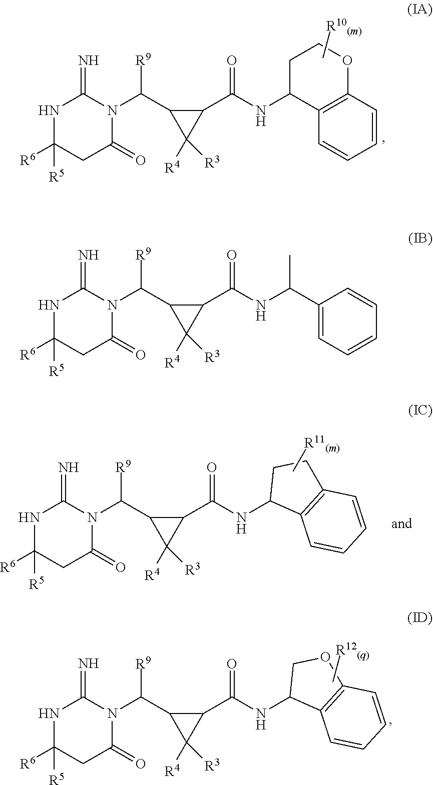

wherein R³, R⁴, R⁵, R⁶ and R⁹ are as described above;
R¹⁰ is halogen, —OH, alkoxy, haloalkoxy, C₁-C₆alkylOC₁-C₆alkyl, C₃-C₆cycloalkyl, spiroC₃-C₆cycloalkyl, C₁-C₆alkyl or haloC₁-C₆alkyl;
R¹¹ is —OH or C₁-C₆alkyl;
R¹² is C₁-C₆alkyl; and
m, n and q are independently 0, 1, 2, 3 or 4.

In each of the various embodiments of the invention, in the compounds used in the methods herein (including those in each of Formula (I), (IA), (IB), (IC) and (ID), and the various embodiments thereof) it shall be understood that each variable is to be selected independently of the others unless otherwise indicated.

In each of the various embodiments of the invention, the compounds described herein, including those in each of Formula (I), (IA), (IB), (IC) and (ID) and the various embodiments thereof, may exist in different forms of the compounds such as, for example, any solvates, hydrates, stereoisomers, and tautomers of said compounds and of any pharmaceutically acceptable salts thereof.

In certain embodiments, compounds described herein include:

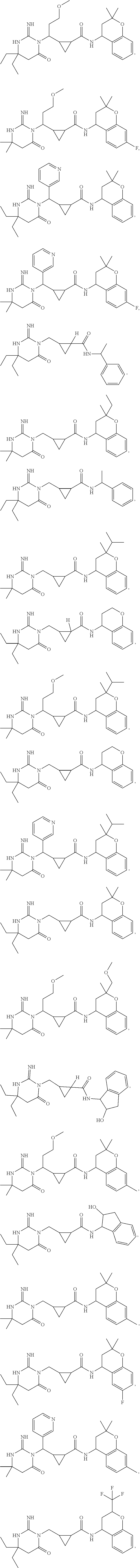

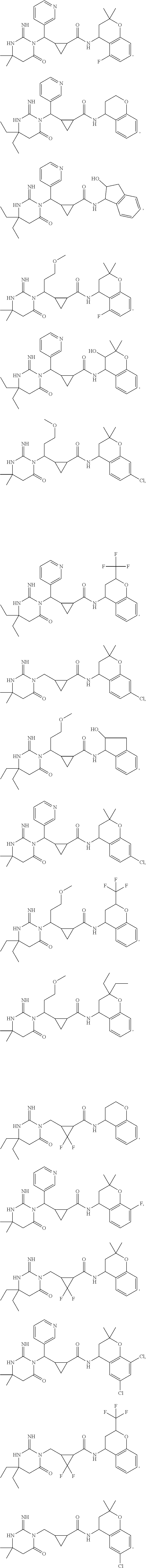

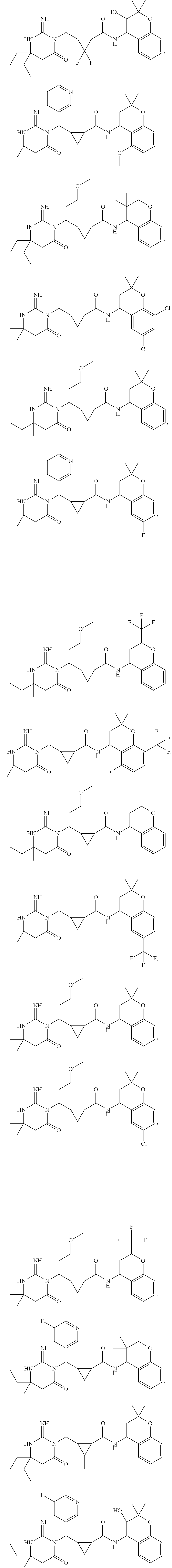

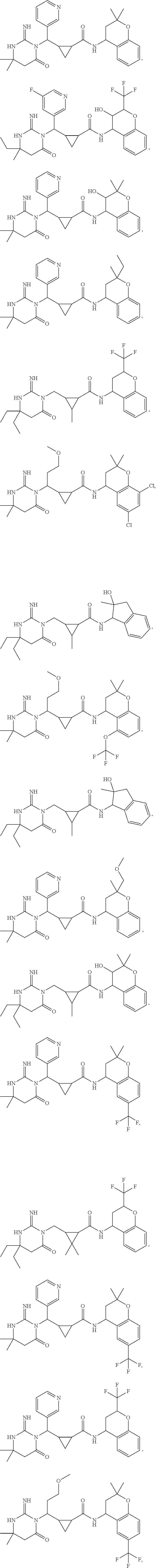

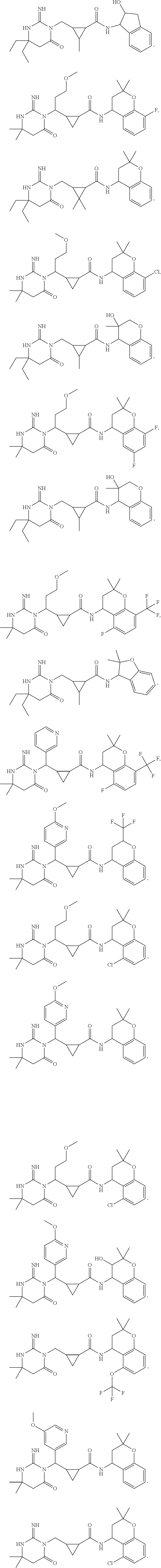

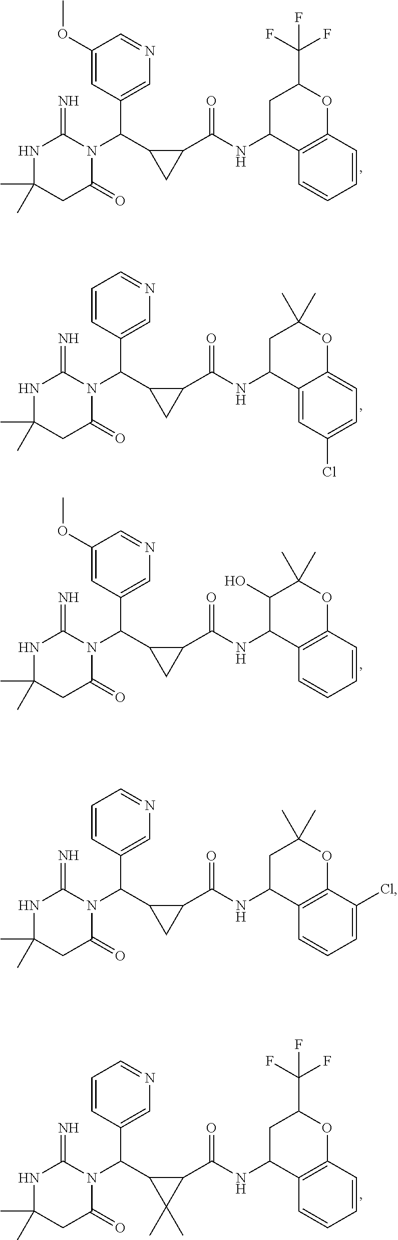 [SMILES: COc1cncc(C(C2CC2C(=O)NC2CC(C(F)(F)F)Oc3ccccc32)N2C(=N)NC(C)(C)CC2=O)c1 ; CC1(C)CC(=O)N(C(c2cccnc2)C2CC2C(=O)NC2CC(C)(C)Oc3ccc(Cl)cc32)C(=N)N1 ; COc1cncc(C(C2CC2C(=O)NC2c3ccccc3OC(C)(C)C2O)N2C(=N)NC(C)(C)CC2=O)c1 ; CC1(C)CC(=O)N(C(c2cccnc2)C2CC2C(=O)NC2CC(C)(C)Oc3c(Cl)cccc32)C(=N)N1 ; CC1(C)CC(=O)N(C(c2cccnc2)C2C(C(=O)NC3CC(C(F)(F)F)Oc4ccccc43)C2(C)C)C(=N)N1]

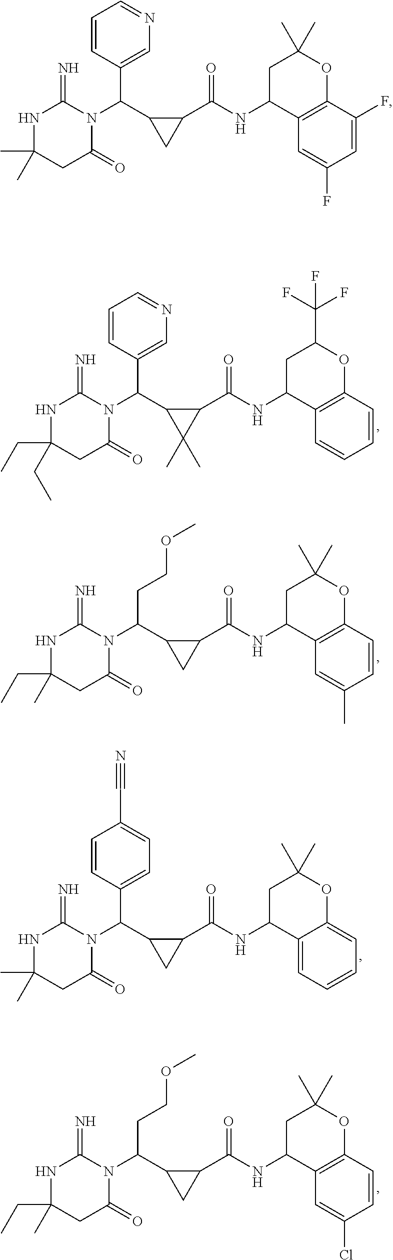 [SMILES: CC1(C)CC(=O)N(C(c2cccnc2)C2CC2C(=O)NC2CC(C)(C)Oc3c(F)cc(F)cc32)C(=N)N1 ; CCC1(CC)CC(=O)N(C(c2cccnc2)C2C(C(=O)NC3CC(C(F)(F)F)Oc4ccccc43)C2(C)C)C(=N)N1 ; CCC1(C)CC(=O)N(C(CCOC)C2CC2C(=O)NC2CC(C)(C)Oc3ccc(C)cc32)C(=N)N1 ; CC1(C)CC(=O)N(C(c2ccc(C#N)cc2)C2CC2C(=O)NC2CC(C)(C)Oc3ccccc32)C(=N)N1 ; CCC1(C)CC(=O)N(C(CCOC)C2CC2C(=O)NC2CC(C)(C)Oc3ccc(Cl)cc32)C(=N)N1]

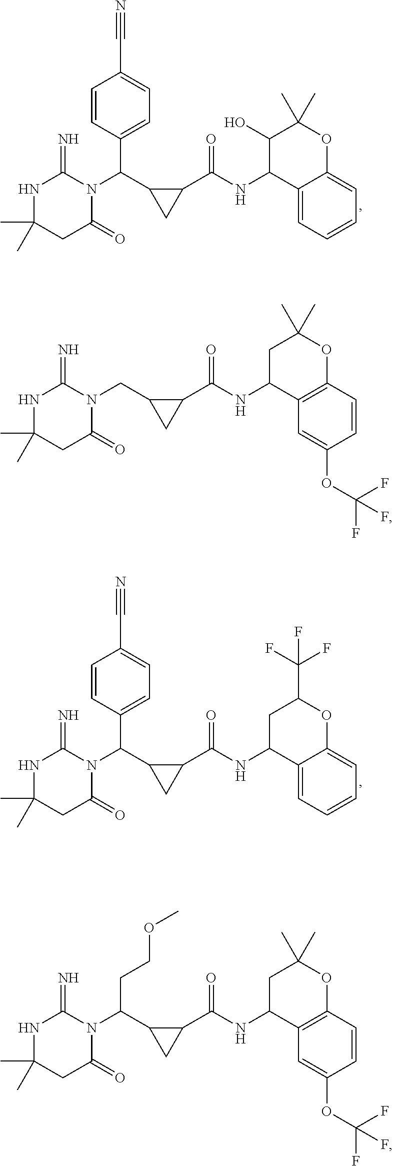 [SMILES: CC1(C)CC(=O)N(C(c2ccc(C#N)cc2)C2CC2C(=O)NC2c3ccccc3OC(C)(C)C2O)C(=N)N1 ; CC1(C)CC(=O)N(CC2CC2C(=O)NC2CC(C)(C)Oc3ccc(OC(F)(F)F)cc32)C(=N)N1 ; CC1(C)CC(=O)N(C(c2ccc(C#N)cc2)C2CC2C(=O)NC2CC(C(F)(F)F)Oc3ccccc32)C(=N)N1 ; COCCC(C1CC1C(=O)NC1CC(C)(C)Oc2ccc(OC(F)(F)F)cc21)N1C(=N)NC(C)(C)CC1=O]

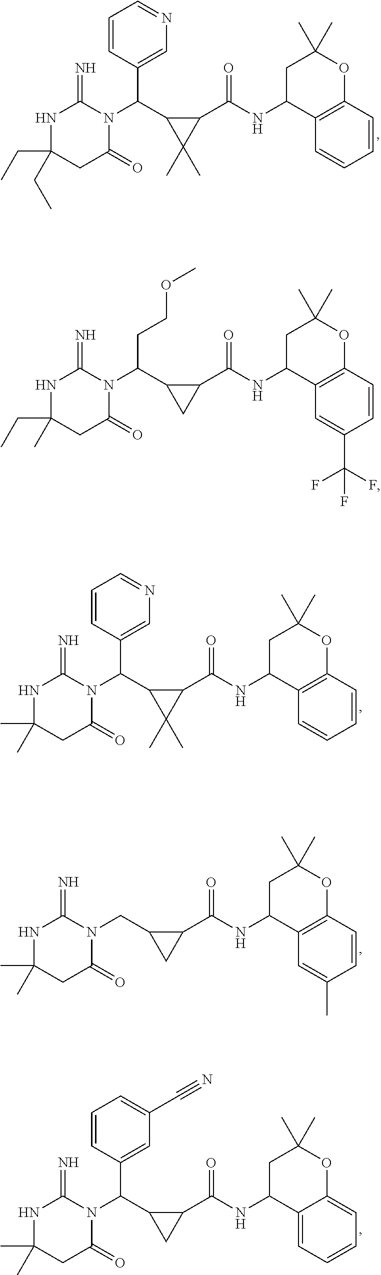 [SMILES: CCC1(CC)CC(=O)N(C(c2cccnc2)C2C(C(=O)NC3CC(C)(C)Oc4ccccc43)C2(C)C)C(=N)N1 ; CCC1(C)CC(=O)N(C(CCOC)C2CC2C(=O)NC2CC(C)(C)Oc3ccc(C(F)(F)F)cc32)C(=N)N1 ; CC1(C)CC(=O)N(C(c2cccnc2)C2C(C(=O)NC3CC(C)(C)Oc4ccccc43)C2(C)C)C(=N)N1 ; Cc1ccc2c(c1)C(NC(=O)C1CC1CN1C(=N)NC(C)(C)CC1=O)CC(C)(C)O2 ; CC1(C)CC(=O)N(C(c2cccc(C#N)c2)C2CC2C(=O)NC2CC(C)(C)Oc3ccccc32)C(=N)N1]

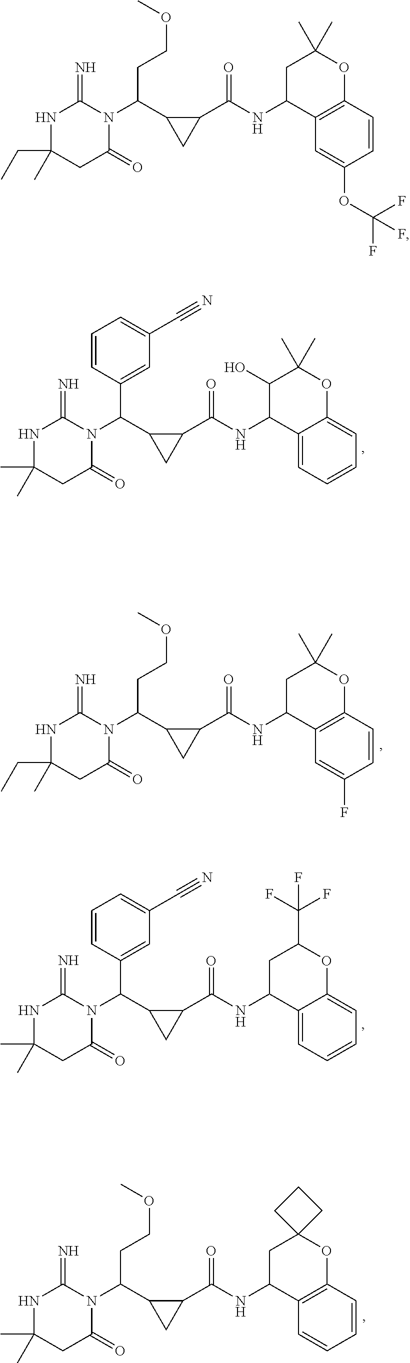 [SMILES: CCC1(C)CC(=O)N(C(CCOC)C2CC2C(=O)NC2CC(C)(C)Oc3ccc(OC(F)(F)F)cc32)C(=N)N1 ; CC1(C)CC(=O)N(C(c2cccc(C#N)c2)C2CC2C(=O)NC2c3ccccc3OC(C)(C)C2O)C(=N)N1 ; CCC1(C)CC(=O)N(C(CCOC)C2CC2C(=O)NC2CC(C)(C)Oc3ccc(F)cc32)C(=N)N1 ; CC1(C)CC(=O)N(C(c2cccc(C#N)c2)C2CC2C(=O)NC2CC(C(F)(F)F)Oc3ccccc32)C(=N)N1 ; COCCC(C1CC1C(=O)NC1CC2(CCC2)Oc2ccccc21)N1C(=N)NC(C)(C)CC1=O]

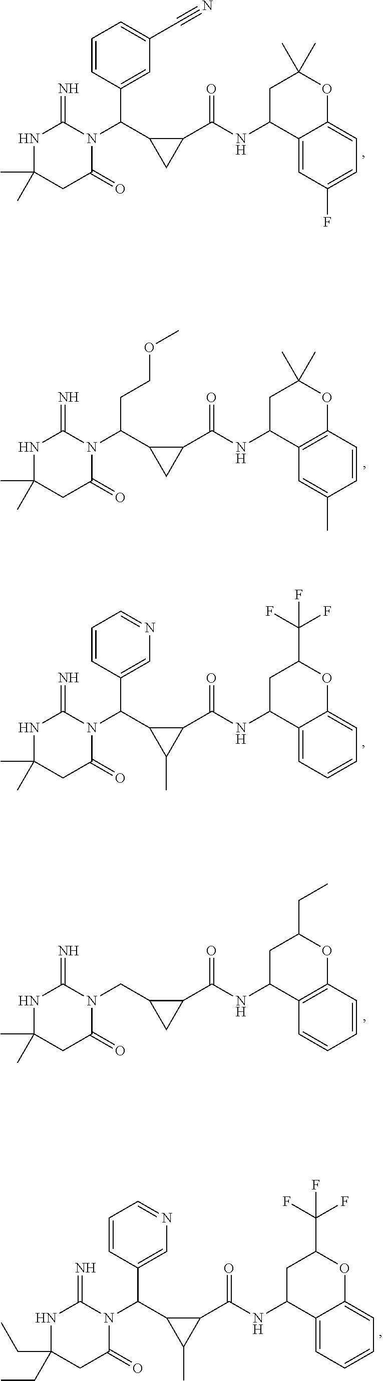 [SMILES: CC1(C)CC(=O)N(C(c2cccc(C#N)c2)C2CC2C(=O)NC2CC(C)(C)Oc3ccc(F)cc32)C(=N)N1 ; COCCC(C1CC1C(=O)NC1CC(C)(C)Oc2ccc(C)cc21)N1C(=N)NC(C)(C)CC1=O ; CC1C(C(=O)NC2CC(C(F)(F)F)Oc3ccccc32)C1C(c1cccnc1)N1C(=N)NC(C)(C)CC1=O ; CCC1CC(NC(=O)C2CC2CN2C(=N)NC(C)(C)CC2=O)c2ccccc2O1 ; CCC1(CC)CC(=O)N(C(c2cccnc2)C2C(C)C2C(=O)NC2CC(C(F)(F)F)Oc3ccccc32)C(=N)N1]

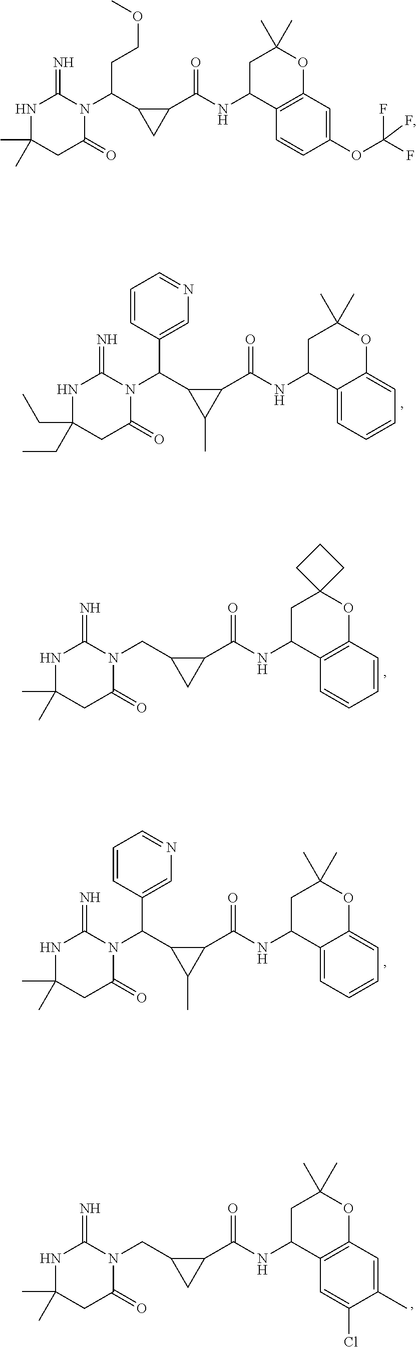

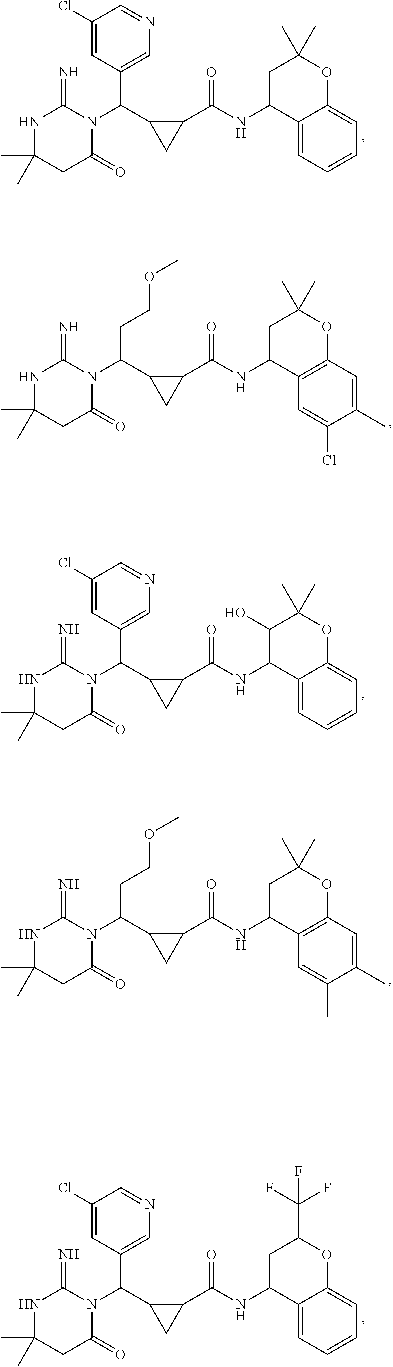

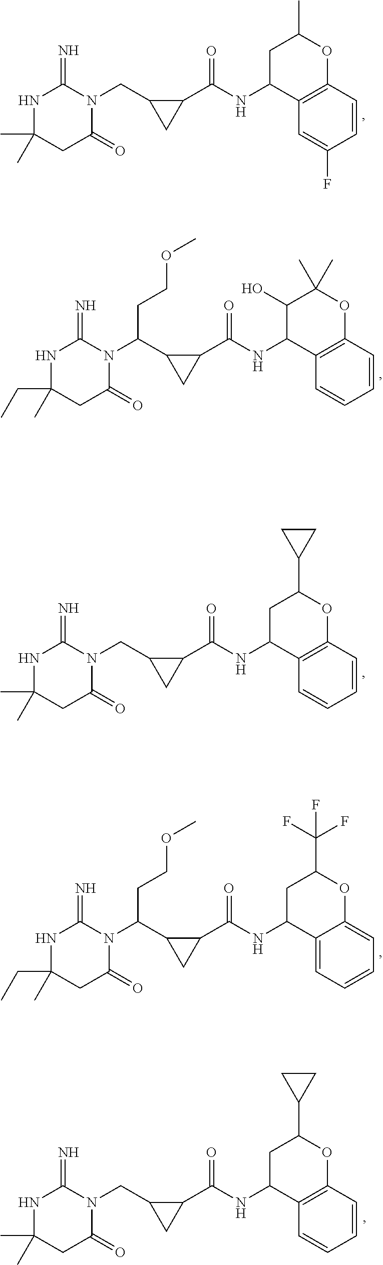 [SMILES: CC1CC(NC(=O)C2CC2CN2C(=N)NC(C)(C)CC2=O)c2cc(F)ccc2O1 ; CCC1(C)CC(=O)N(C(CCOC)C2CC2C(=O)NC2c3ccccc3OC(C)(C)C2O)C(=N)N1 ; CC1(C)CC(=O)N(CC2CC2C(=O)NC2CC(C3CC3)Oc3ccccc32)C(=N)N1 ; CCC1(C)CC(=O)N(C(CCOC)C2CC2C(=O)NC2CC(C(F)(F)F)Oc3ccccc32)C(=N)N1 ; CC1(C)CC(=O)N(CC2CC2C(=O)NC2CC(C3CC3)Oc3ccccc32)C(=N)N1]

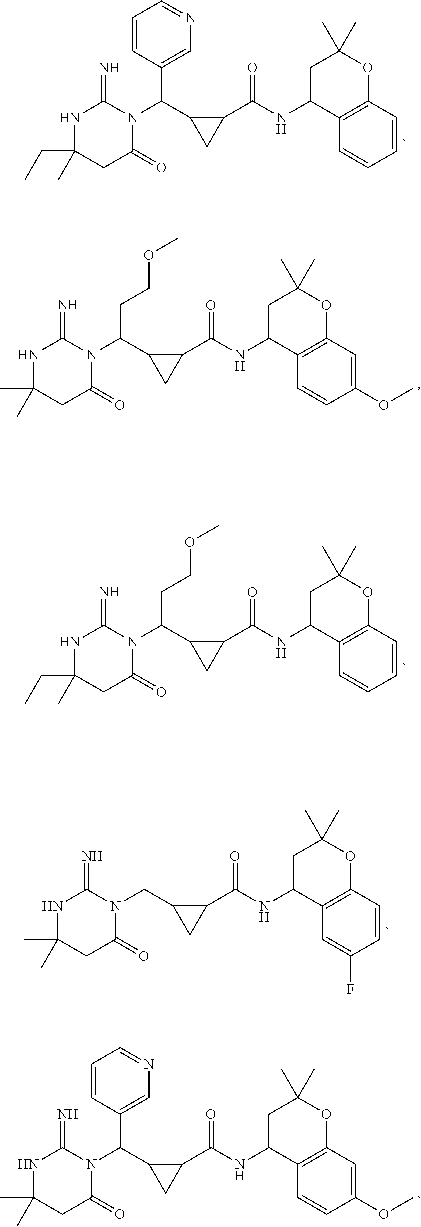

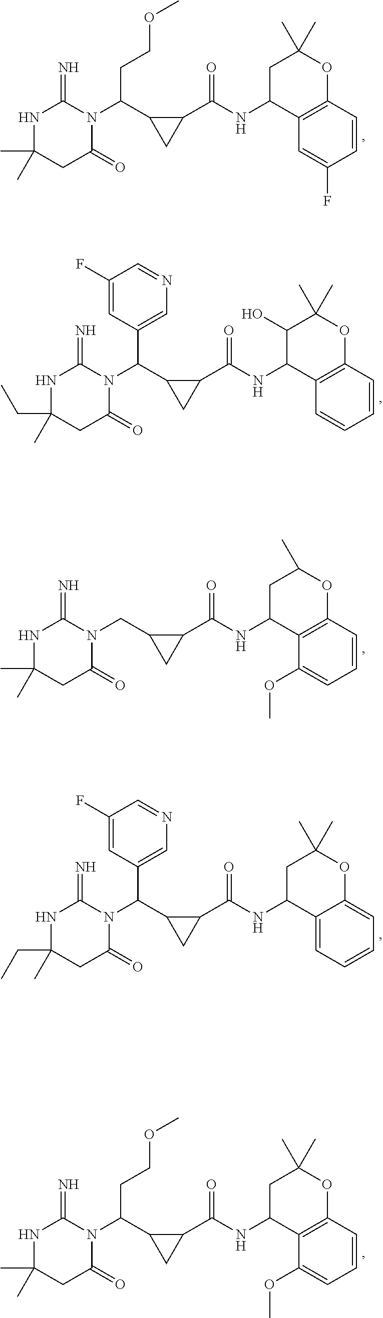 [SMILES: COCCC(C1CC1C(=O)NC1CC(C)(C)Oc2ccc(F)cc21)N1C(=N)NC(C)(C)CC1=O ; CCC1(C)CC(=O)N(C(c2cncc(F)c2)C2CC2C(=O)NC2c3ccccc3OC(C)(C)C2O)C(=N)N1 ; COc1cccc2c1C(NC(=O)C1CC1CN1C(=N)NC(C)(C)CC1=O)CC(C)O2 ; CCC1(C)CC(=O)N(C(c2cncc(F)c2)C2CC2C(=O)NC2CC(C)(C)Oc3ccccc32)C(=N)N1 ; COCCC(C1CC1C(=O)NC1CC(C)(C)Oc2cccc(OC)c21)N1C(=N)NC(C)(C)CC1=O]

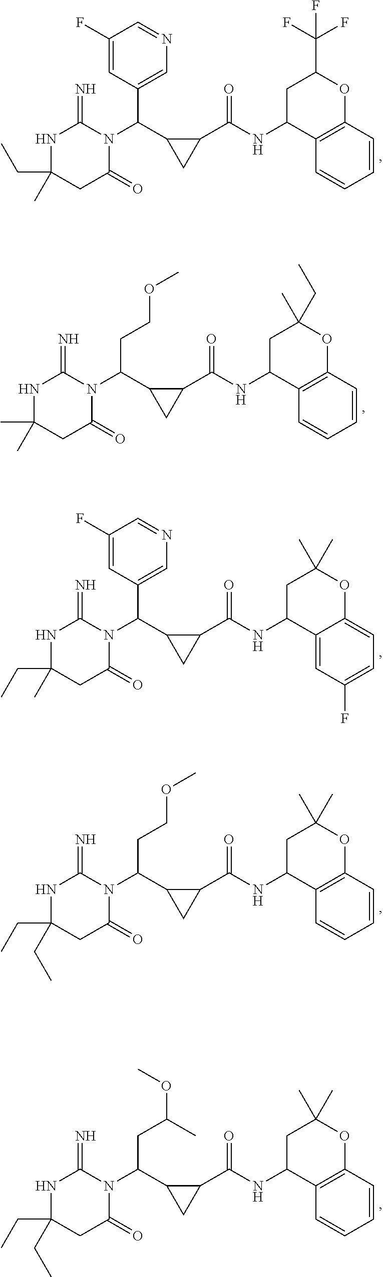 [SMILES: CCC1(C)CC(=O)N(C(c2cncc(F)c2)C2CC2C(=O)NC2CC(C(F)(F)F)Oc3ccccc32)C(=N)N1 ; CCC1(C)CC(NC(=O)C2CC2C(CCOC)N2C(=N)NC(C)(C)CC2=O)c2ccccc2O1 ; CCC1(C)CC(=O)N(C(c2cncc(F)c2)C2CC2C(=O)NC2CC(C)(C)Oc3ccc(F)cc32)C(=N)N1 ; CCC1(CC)CC(=O)N(C(CCOC)C2CC2C(=O)NC2CC(C)(C)Oc3ccccc32)C(=N)N1 ; CCC1(CC)CC(=O)N(C(CC(C)OC)C2CC2C(=O)NC2CC(C)(C)Oc3ccccc32)C(=N)N1]

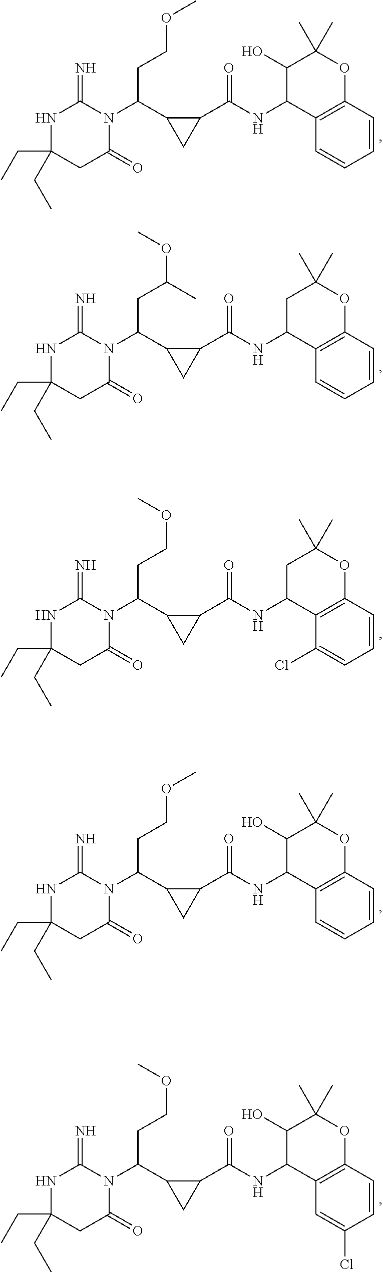 [SMILES: CCC1(CC)CC(=O)N(C(CCOC)C2CC2C(=O)NC2c3ccccc3OC(C)(C)C2O)C(=N)N1 ; CCC1(CC)CC(=O)N(C(CC(C)OC)C2CC2C(=O)NC2CC(C)(C)Oc3ccccc32)C(=N)N1 ; CCC1(CC)CC(=O)N(C(CCOC)C2CC2C(=O)NC2CC(C)(C)Oc3cccc(Cl)c32)C(=N)N1 ; CCC1(CC)CC(=O)N(C(CCOC)C2CC2C(=O)NC2c3ccccc3OC(C)(C)C2O)C(=N)N1 ; CCC1(CC)CC(=O)N(C(CCOC)C2CC2C(=O)NC2c3cc(Cl)ccc3OC(C)(C)C2O)C(=N)N1]

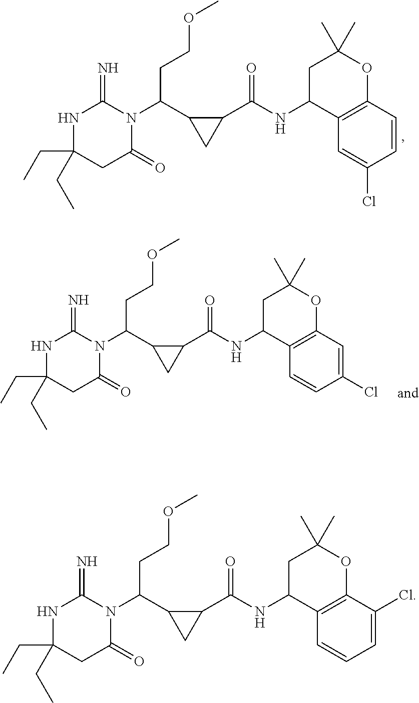 [SMILES: CCC1(CC)CC(=O)N(C(CCOC)C2CC2C(=O)NC2CC(C)(C)Oc3ccc(Cl)cc32)C(=N)N1 ; CCC1(CC)CC(=O)N(C(CCOC)C2CC2C(=O)NC2CC(C)(C)Oc3cc(Cl)ccc32)C(=N)N1 ; CCC1(CC)CC(=O)N(C(CCOC)C2CC2C(=O)NC2CC(C)(C)Oc3c(Cl)cccc32)C(=N)N1]

In certain embodiments, compounds described herein include:

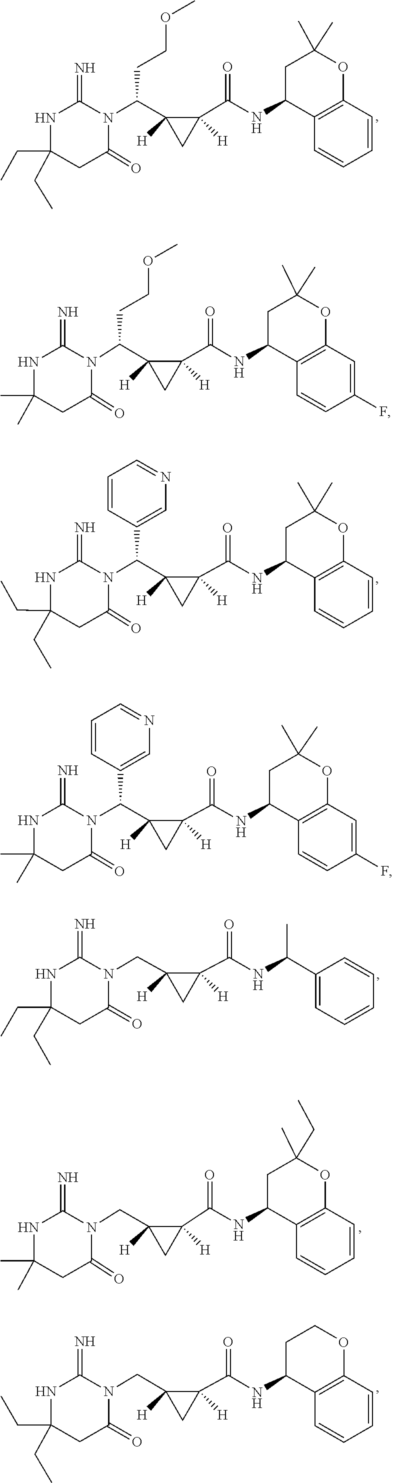

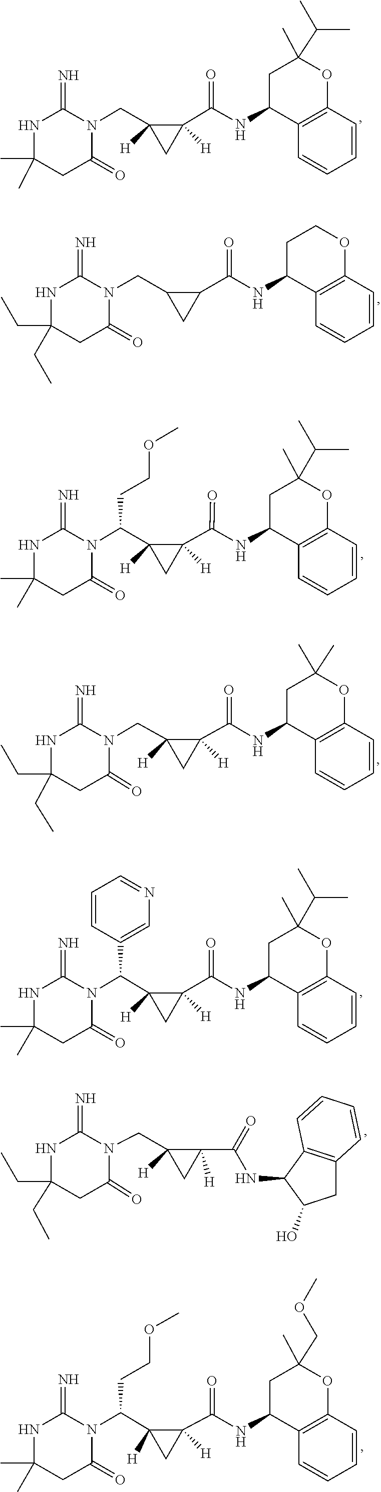

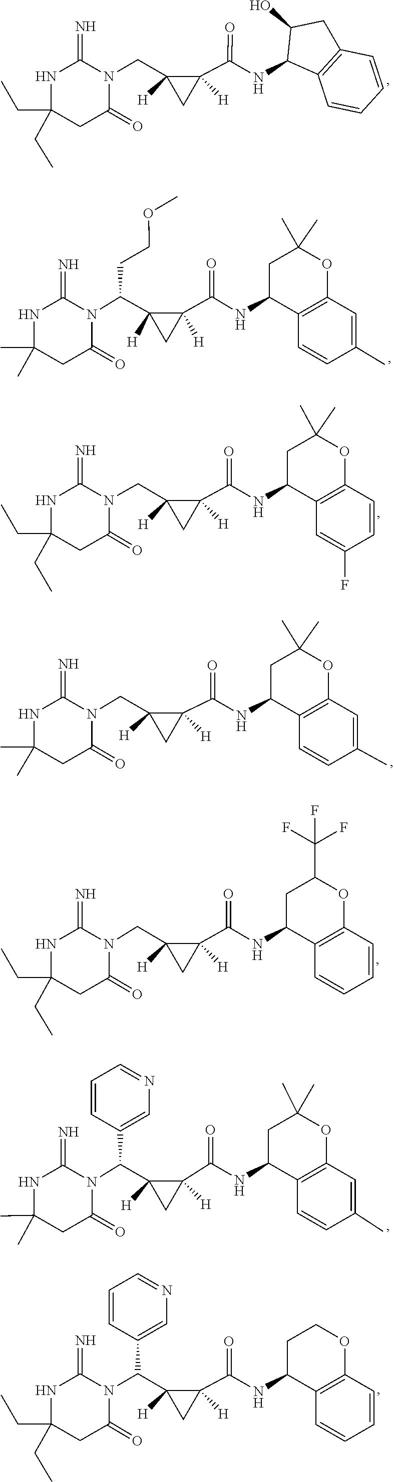

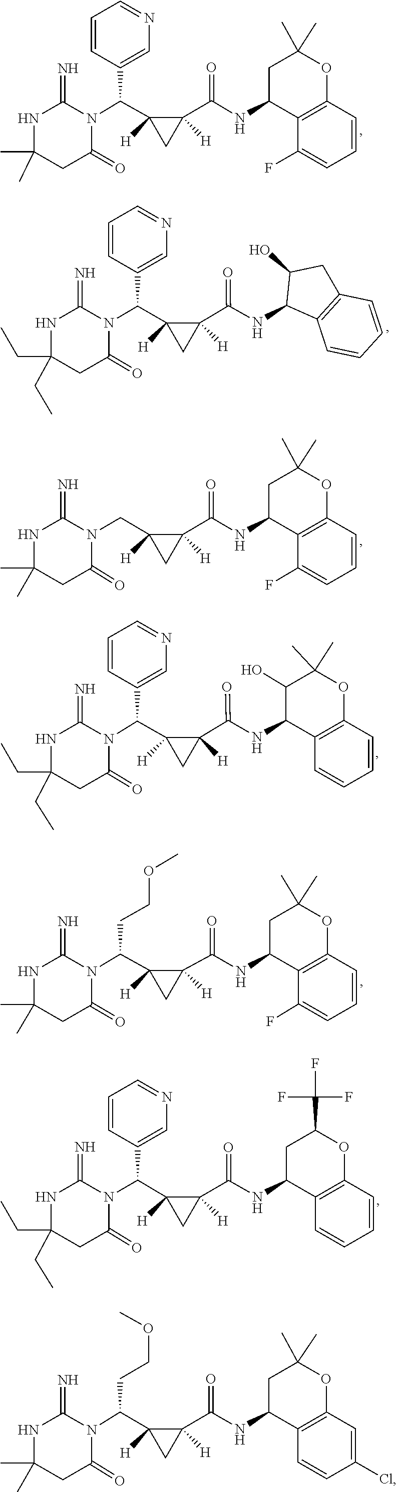

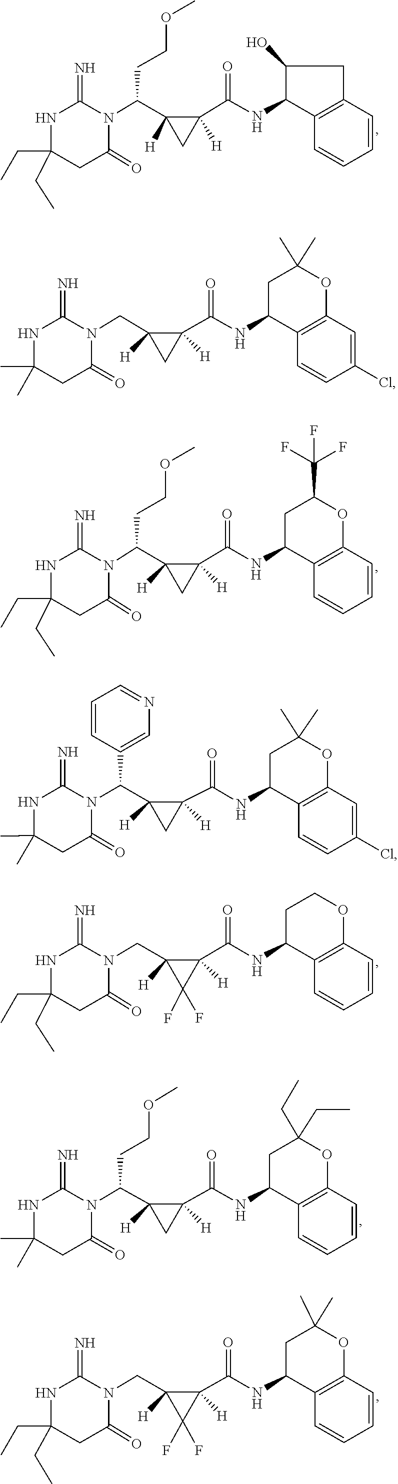

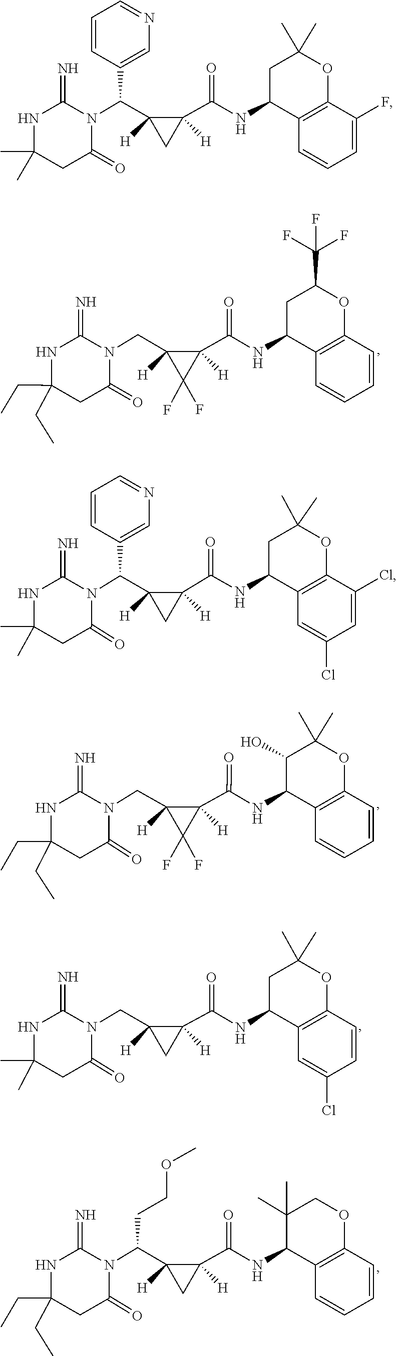

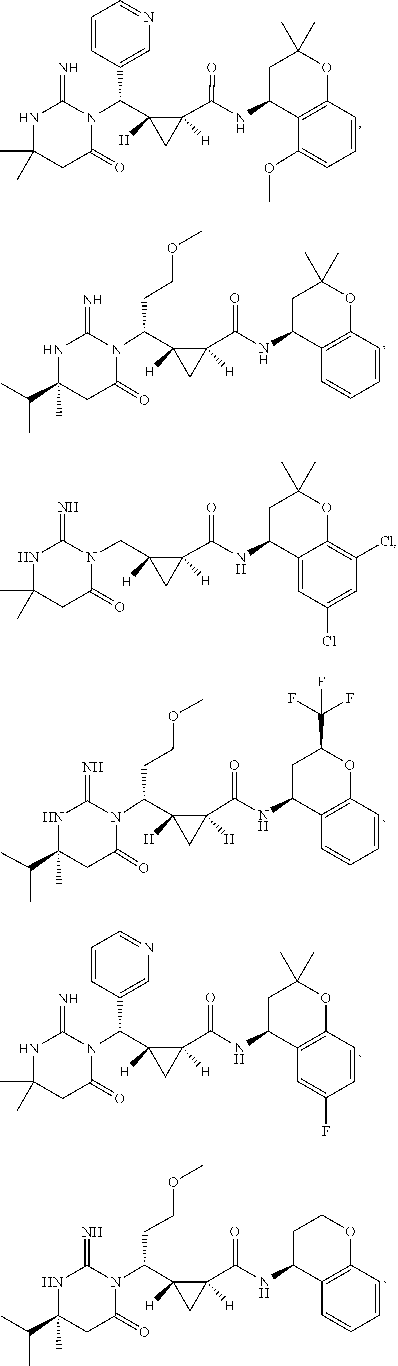

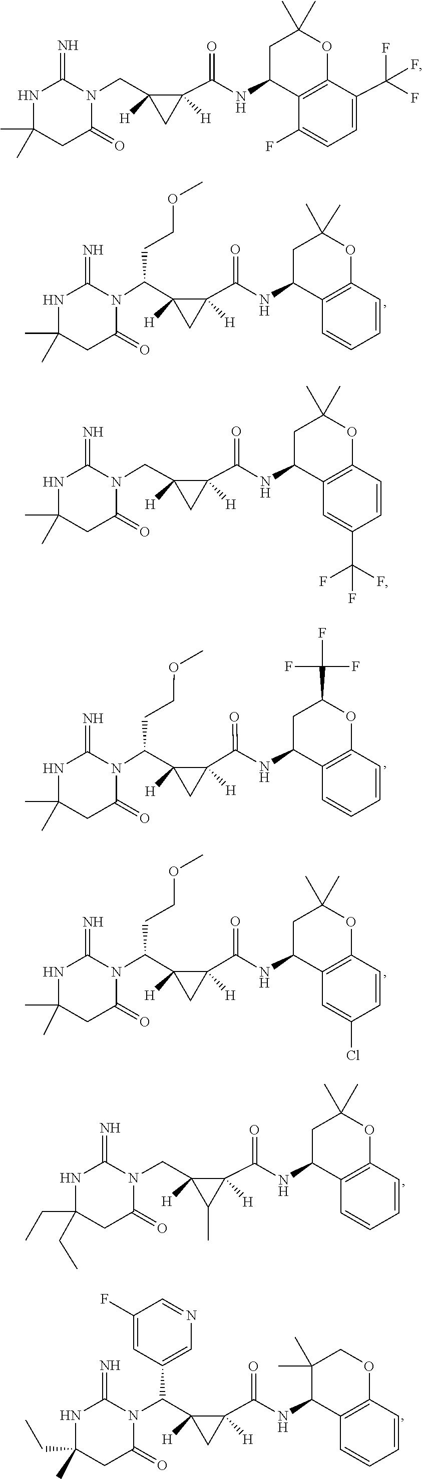

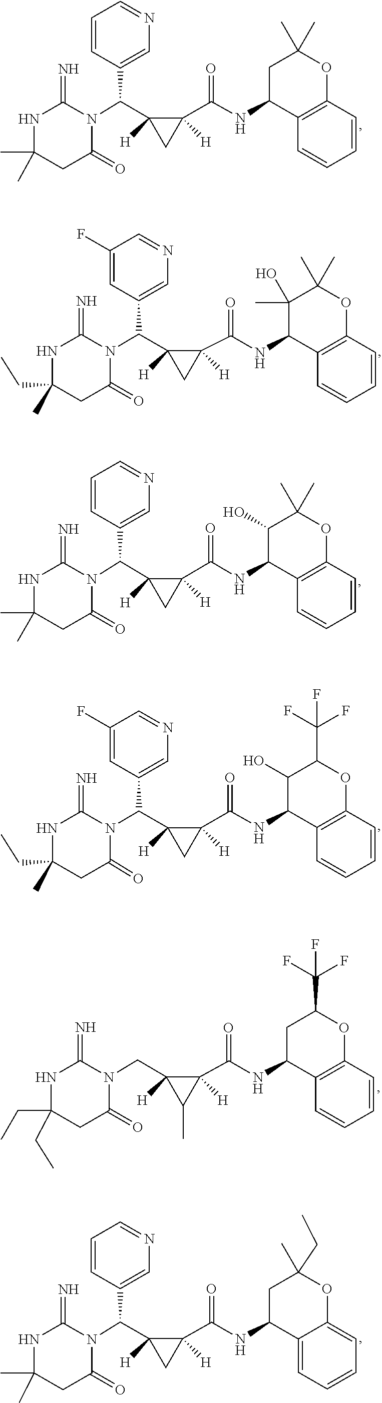

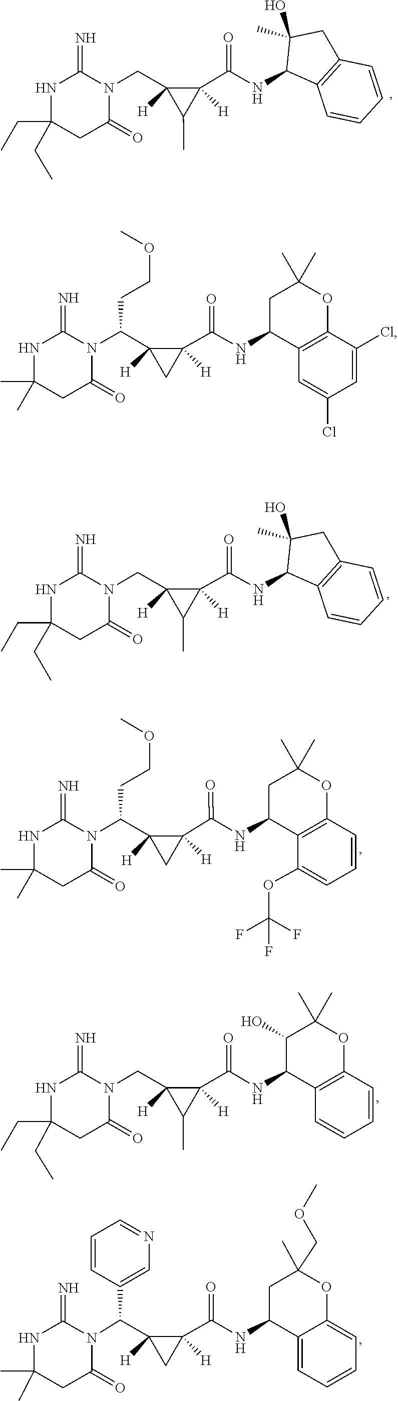 [SMILES: CCC1(CC)CC(=O)N(C[C@H]2C(C)[C@@H]2C(=O)N[C@@H]2c3ccccc3C[C@]2(C)O)C(=N)N1 ; COCC[C@H]([C@H]1C[C@@H]1C(=O)N[C@H]1CC(C)(C)Oc2c(Cl)cc(Cl)cc21)N1C(=N)NC(C)(C)CC1=O ; CCC1(CC)CC(=O)N(C[C@H]2C(C)[C@@H]2C(=O)N[C@@H]2c3ccccc3C[C@]2(C)O)C(=N)N1 ; COCC[C@H]([C@H]1C[C@@H]1C(=O)N[C@H]1CC(C)(C)Oc2cccc(OC(F)(F)F)c21)N1C(=N)NC(C)(C)CC1=O ; CCC1(CC)CC(=O)N(C[C@H]2C(C)[C@@H]2C(=O)N[C@@H]2c3ccccc3OC(C)(C)[C@H]2O)C(=N)N1 ; COCC1(C)C[C@H](NC(=O)[C@H]2C[C@@H]2[C@@H](c2cccnc2)N2C(=N)NC(C)(C)CC2=O)c2ccccc2O1]

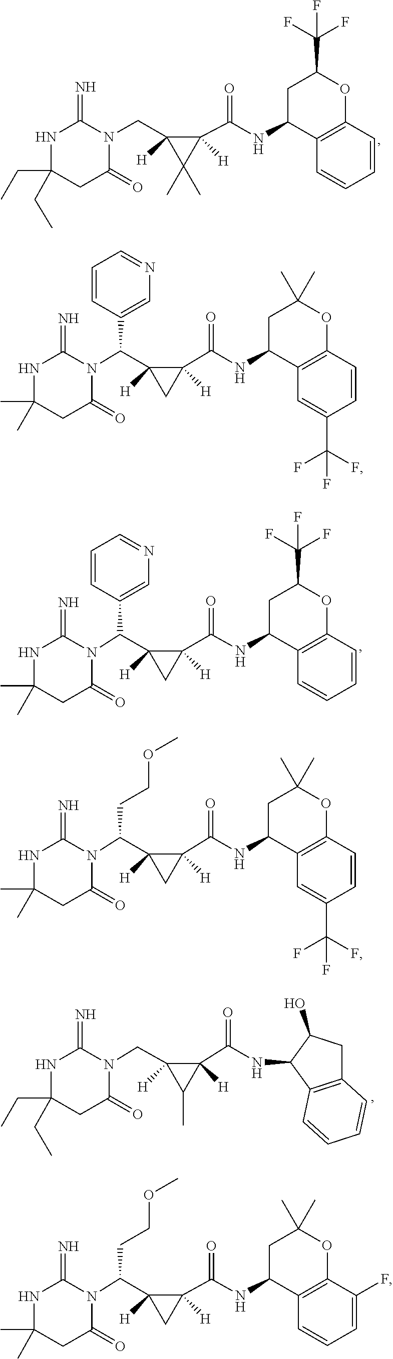

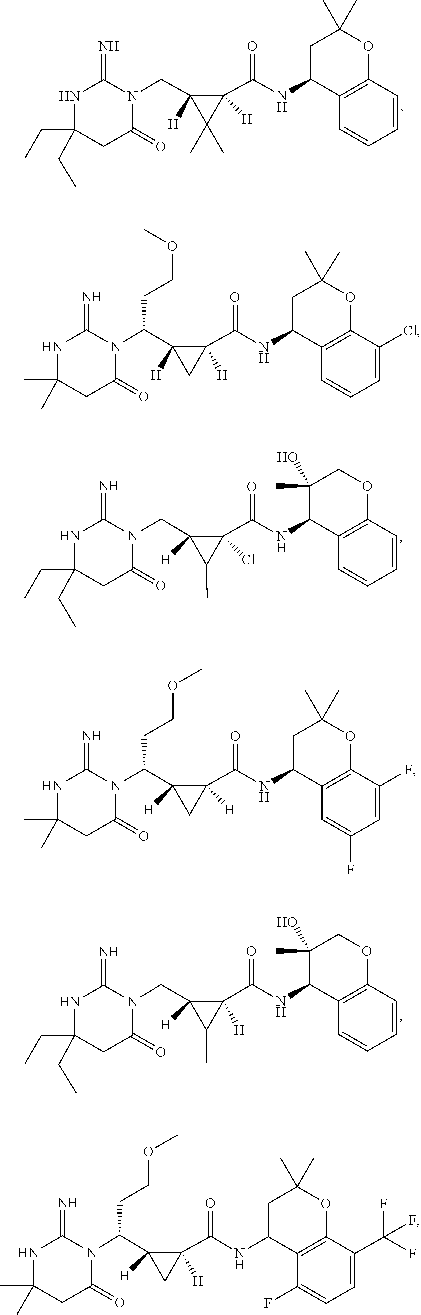 [SMILES: CCC1(CC)CC(=O)N(C[C@@H]2[C@@H](C(=O)N[C@H]3CC(C)(C)Oc4ccccc43)C2(C)C)C(=N)N1 ; COCC[C@H]([C@H]1C[C@@H]1C(=O)N[C@H]1CC(C)(C)Oc2c(Cl)cccc21)N1C(=N)NC(C)(C)CC1=O ; CCC1(CC)CC(=O)N(C[C@H]2C(C)[C@]2(Cl)C(=O)N[C@@H]2c3ccccc3OC[C@@]2(C)O)C(=N)N1 ; COCC[C@H]([C@H]1C[C@@H]1C(=O)N[C@H]1CC(C)(C)Oc2c(F)cc(F)cc21)N1C(=N)NC(C)(C)CC1=O ; CCC1(CC)CC(=O)N(C[C@H]2C(C)[C@@H]2C(=O)N[C@@H]2c3ccccc3OC[C@@]2(C)O)C(=N)N1 ; COCC[C@H]([C@H]1C[C@@H]1C(=O)NC1CC(C)(C)Oc2c(C(F)(F)F)ccc(F)c21)N1C(=N)NC(C)(C)CC1=O]

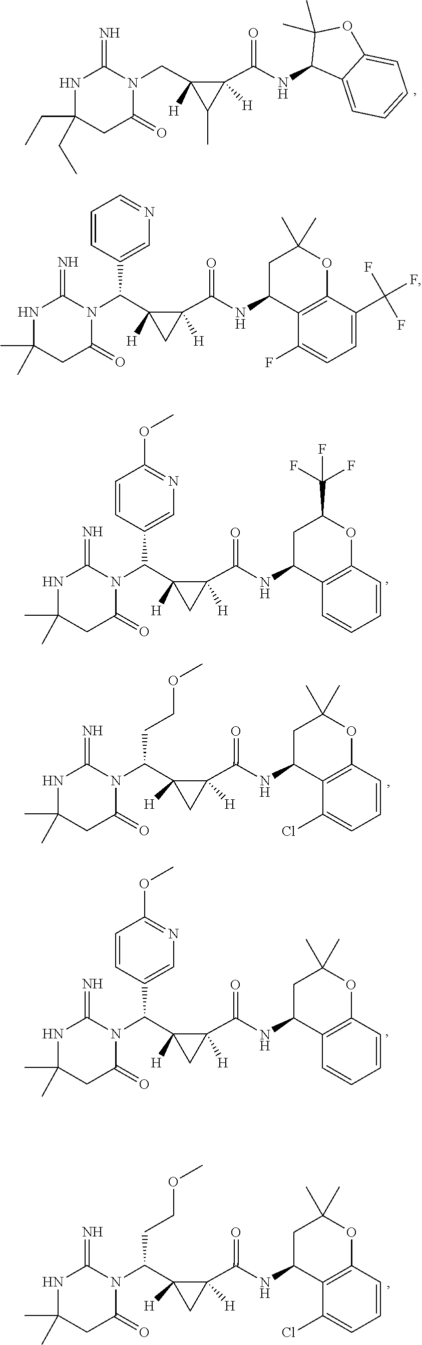

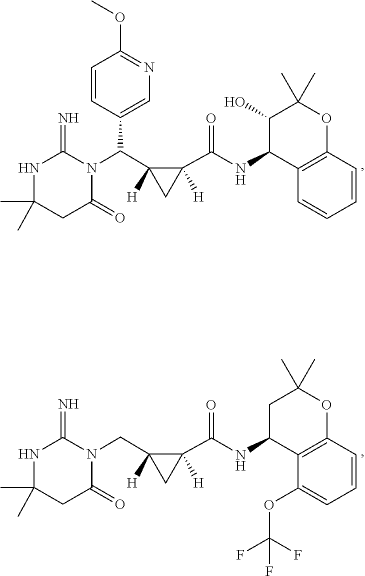

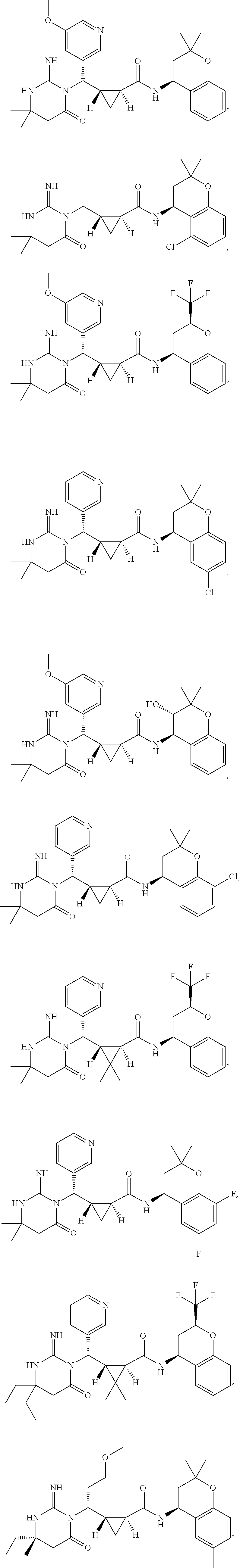

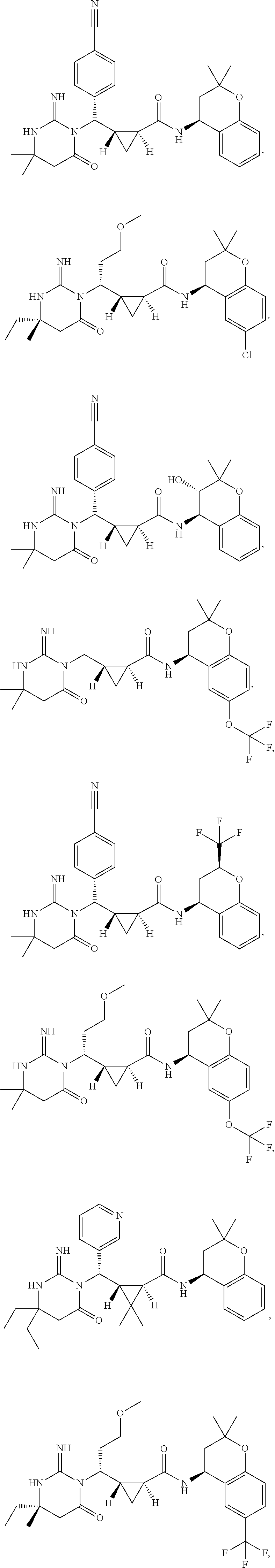

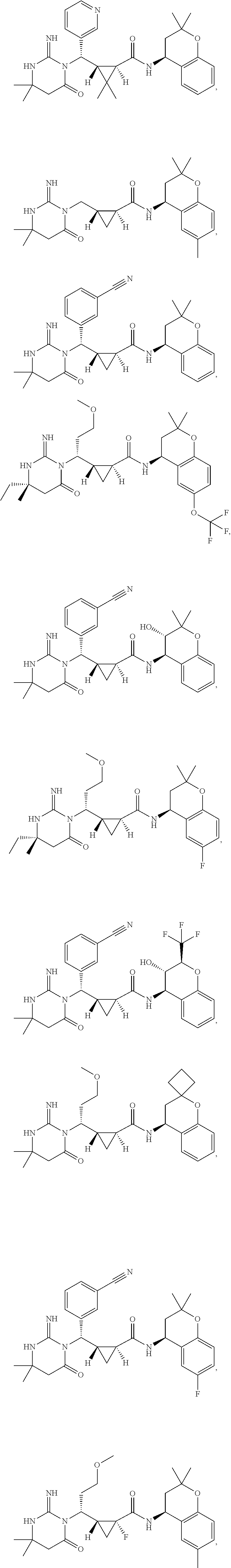

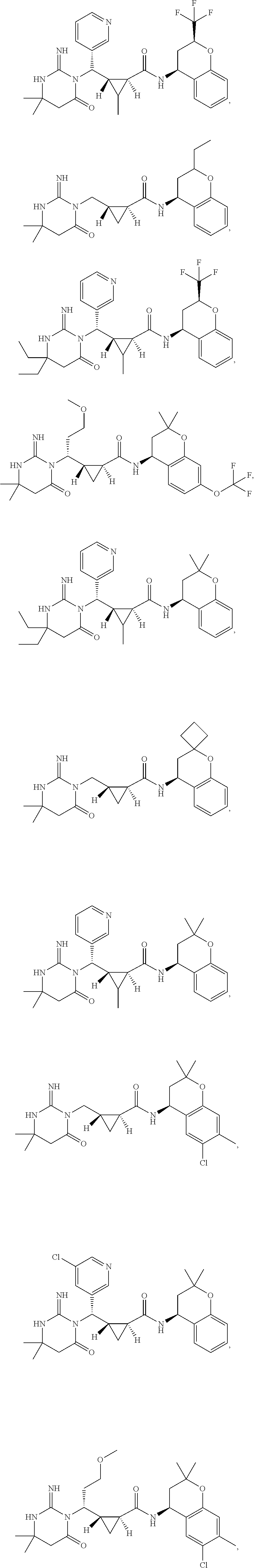

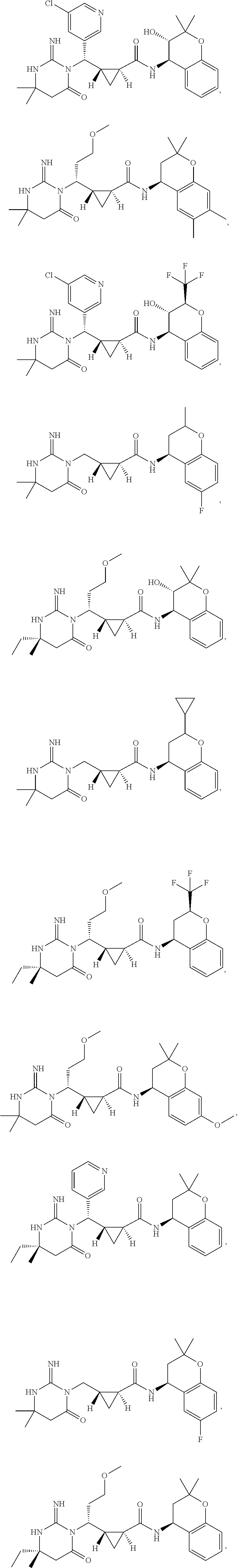

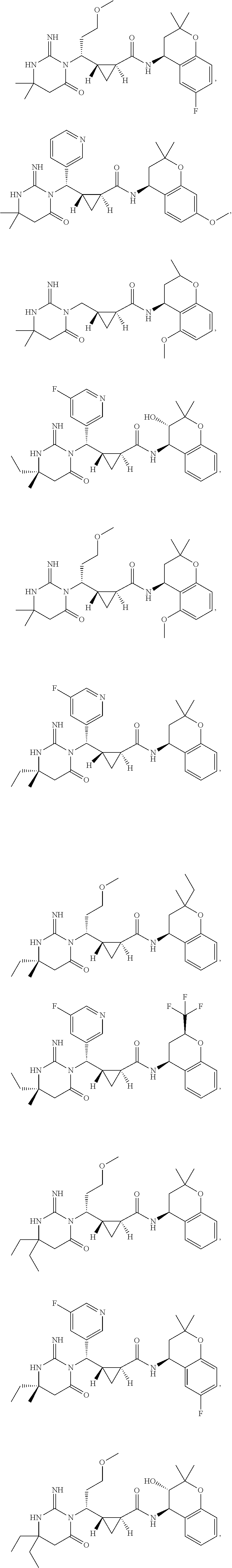

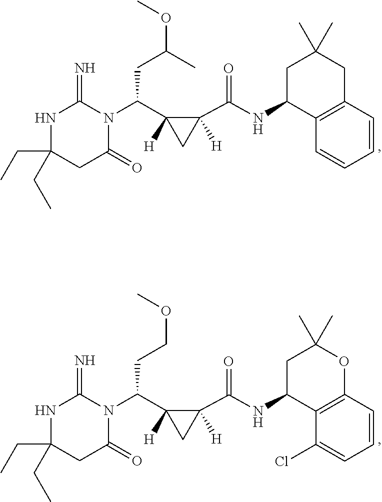 [SMILES: CCC1(CC)CC(=O)N([C@H](CC(C)OC)[C@H]2C[C@@H]2C(=O)N[C@H]2CC(C)(C)Cc3ccccc32)C(=N)N1 ; CCC1(CC)CC(=O)N([C@H](CCOC)[C@H]2C[C@@H]2C(=O)N[C@H]2CC(C)(C)Oc3cccc(Cl)c32)C(=N)N1]

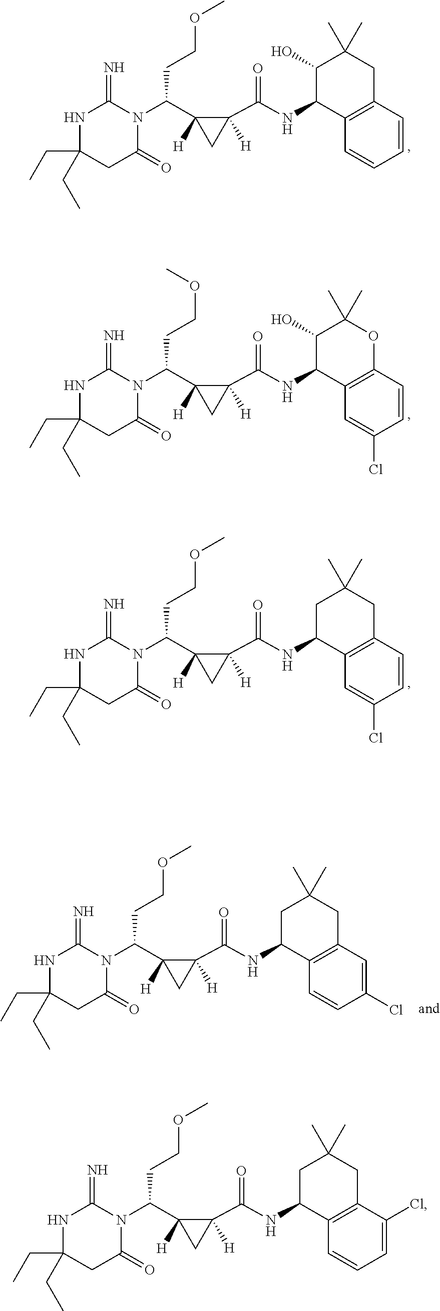 [SMILES: CCC1(CC)CC(=O)N([C@H](CCOC)[C@H]2C[C@@H]2C(=O)N[C@@H]2c3ccccc3CC(C)(C)[C@H]2O)C(=N)N1 ; CCC1(CC)CC(=O)N([C@H](CCOC)[C@H]2C[C@@H]2C(=O)N[C@@H]2c3cc(Cl)ccc3OC(C)(C)[C@H]2O)C(=N)N1 ; CCC1(CC)CC(=O)N([C@H](CCOC)[C@H]2C[C@@H]2C(=O)N[C@H]2CC(C)(C)Cc3ccc(Cl)cc32)C(=N)N1 ; CCC1(CC)CC(=O)N([C@H](CCOC)[C@H]2C[C@@H]2C(=O)N[C@H]2CC(C)(C)Cc3cc(Cl)ccc32)C(=N)N1 ; CCC1(CC)CC(=O)N([C@H](CCOC)[C@H]2C[C@@H]2C(=O)N[C@H]2CC(C)(C)Cc3c(Cl)cccc32)C(=N)N1]

In certain embodiments, compounds described herein include:

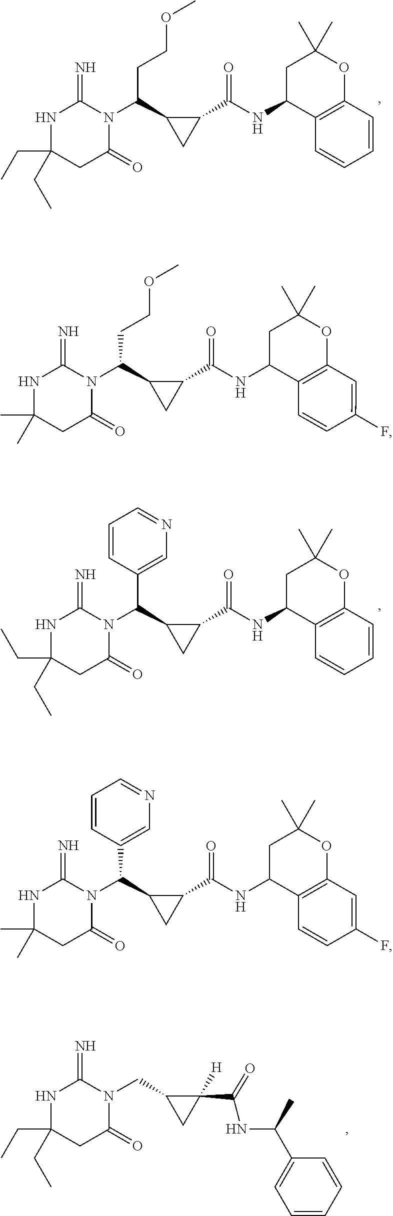 [SMILES: CCC1(CC)CC(=O)N(C(CCOC)[C@@H]2C[C@H]2C(=O)N[C@H]2CC(C)(C)Oc3ccccc32)C(=N)N1 ; COCC[C@H]([C@@H]1C[C@H]1C(=O)NC1CC(C)(C)Oc2cc(F)ccc21)N1C(=N)NC(C)(C)CC1=O ; CCC1(CC)CC(=O)N(C(c2cccnc2)[C@@H]2C[C@H]2C(=O)N[C@H]2CC(C)(C)Oc3ccccc32)C(=N)N1 ; CC1(C)CC(=O)N([C@H](c2cccnc2)[C@@H]2C[C@H]2C(=O)NC2CC(C)(C)Oc3cc(F)ccc32)C(=N)N1 ; CCC1(CC)CC(=O)N(C[C@H]2C[C@@H]2C(=O)N[C@@H](C)c2ccccc2)C(=N)N1]

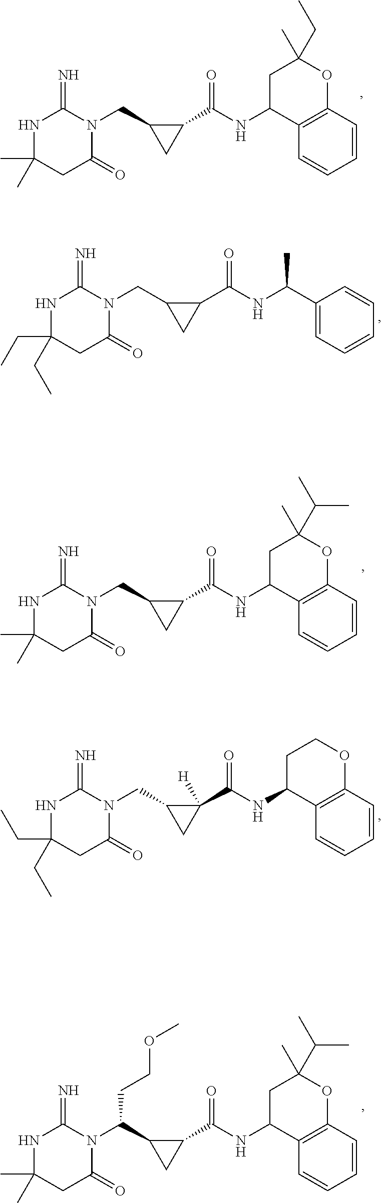 [SMILES: CCC1(C)CC(NC(=O)[C@@H]2C[C@H]2CN2C(=N)NC(C)(C)CC2=O)c2ccccc2O1 ; CCC1(CC)CC(=O)N(CC2CC2C(=O)N[C@@H](C)c2ccccc2)C(=N)N1 ; CC(C)C1(C)CC(NC(=O)[C@@H]2C[C@H]2CN2C(=N)NC(C)(C)CC2=O)c2ccccc2O1 ; CCC1(CC)CC(=O)N(C[C@H]2C[C@@H]2C(=O)N[C@H]2CCOc3ccccc32)C(=N)N1 ; COCC[C@H]([C@@H]1C[C@H]1C(=O)NC1CC(C)(C(C)C)Oc2ccccc21)N1C(=N)NC(C)(C)CC1=O]

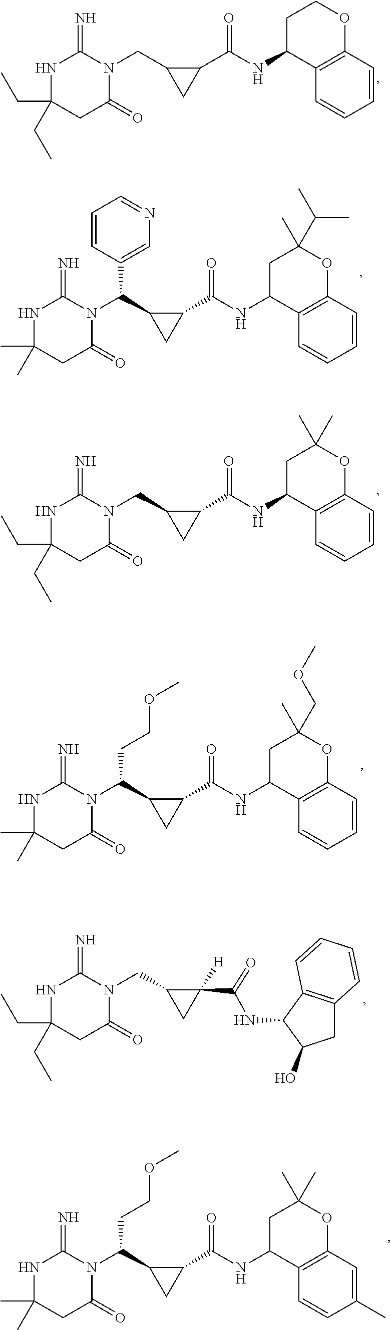

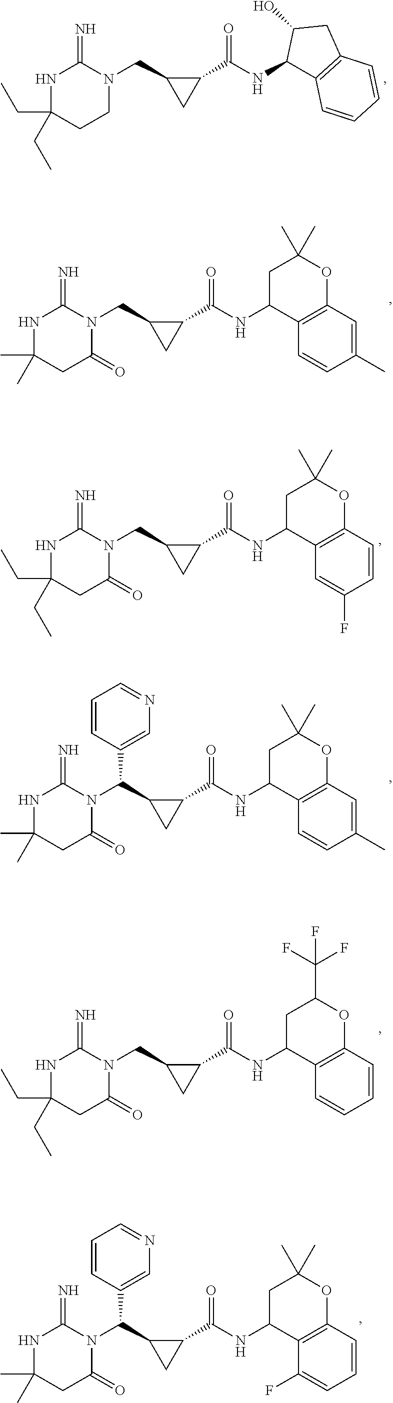

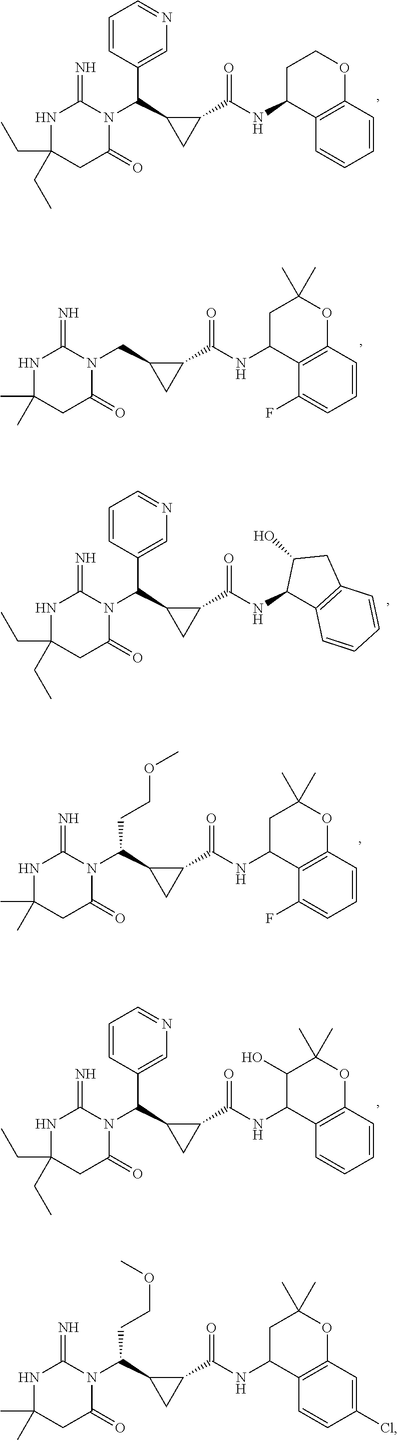 [SMILES: CCC1(CC)CC(=O)N(C(c2cccnc2)[C@@H]2C[C@H]2C(=O)N[C@H]2CCOc3ccccc32)C(=N)N1 ; CC1(C)CC(=O)N(C[C@@H]2C[C@H]2C(=O)NC2CC(C)(C)Oc3cccc(F)c32)C(=N)N1 ; CCC1(CC)CC(=O)N(C(c2cccnc2)[C@@H]2C[C@H]2C(=O)N[C@@H]2c3ccccc3C[C@H]2O)C(=N)N1 ; COCC[C@H]([C@@H]1C[C@H]1C(=O)NC1CC(C)(C)Oc2cccc(F)c21)N1C(=N)NC(C)(C)CC1=O ; CCC1(CC)CC(=O)N(C(c2cccnc2)[C@@H]2C[C@H]2C(=O)NC2c3ccccc3OC(C)(C)C2O)C(=N)N1 ; COCC[C@H]([C@@H]1C[C@H]1C(=O)NC1CC(C)(C)Oc2cc(Cl)ccc21)N1C(=N)NC(C)(C)CC1=O]

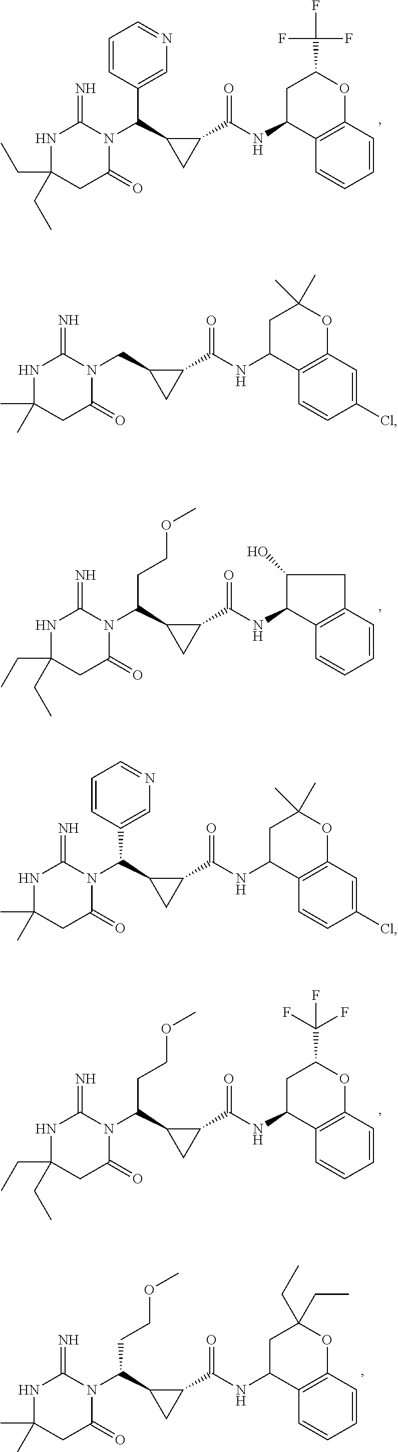 [SMILES: CCC1(CC)CC(=O)N(C(c2cccnc2)[C@@H]2C[C@H]2C(=O)N[C@H]2C[C@H](C(F)(F)F)Oc3ccccc32)C(=N)N1 ; CC1(C)CC(=O)N(C[C@@H]2C[C@H]2C(=O)NC2CC(C)(C)Oc3cc(Cl)ccc32)C(=N)N1 ; CCC1(CC)CC(=O)N(C(CCOC)[C@@H]2C[C@H]2C(=O)N[C@@H]2c3ccccc3C[C@H]2O)C(=N)N1 ; CC1(C)CC(=O)N([C@H](c2cccnc2)[C@@H]2C[C@H]2C(=O)NC2CC(C)(C)Oc3cc(Cl)ccc32)C(=N)N1 ; CCC1(CC)CC(=O)N(C(CCOC)[C@@H]2C[C@H]2C(=O)N[C@H]2C[C@H](C(F)(F)F)Oc3ccccc32)C(=N)N1 ; CCC1(CC)CC(NC(=O)[C@@H]2C[C@H]2[C@@H](CCOC)N2C(=N)NC(C)(C)CC2=O)c2ccccc2O1]

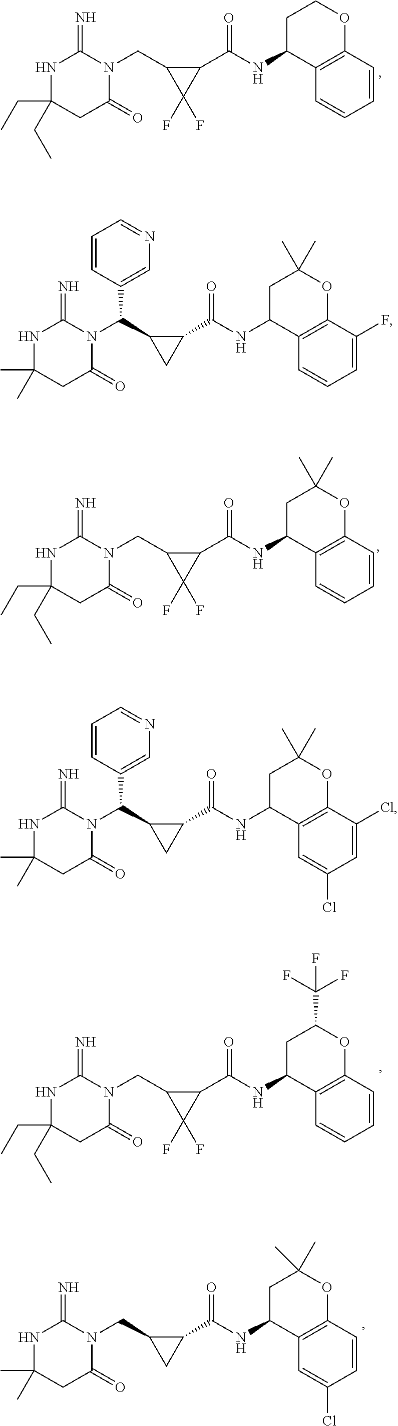 [SMILES: CCC1(CC)CC(=O)N(CC2C(C(=O)N[C@H]3CCOc4ccccc43)C2(F)F)C(=N)N1 ; CC1(C)CC(=O)N([C@H](c2cccnc2)[C@@H]2C[C@H]2C(=O)NC2CC(C)(C)Oc3c(F)cccc32)C(=N)N1 ; CCC1(CC)CC(=O)N(CC2C(C(=O)N[C@H]3CC(C)(C)Oc4ccccc43)C2(F)F)C(=N)N1 ; CC1(C)CC(=O)N([C@H](c2cccnc2)[C@@H]2C[C@H]2C(=O)NC2CC(C)(C)Oc3c(Cl)cc(Cl)cc32)C(=N)N1 ; CCC1(CC)CC(=O)N(CC2C(C(=O)N[C@H]3C[C@H](C(F)(F)F)Oc4ccccc43)C2(F)F)C(=N)N1 ; CC1(C)CC(=O)N(C[C@@H]2C[C@H]2C(=O)N[C@H]2CC(C)(C)Oc3ccc(Cl)cc32)C(=N)N1]

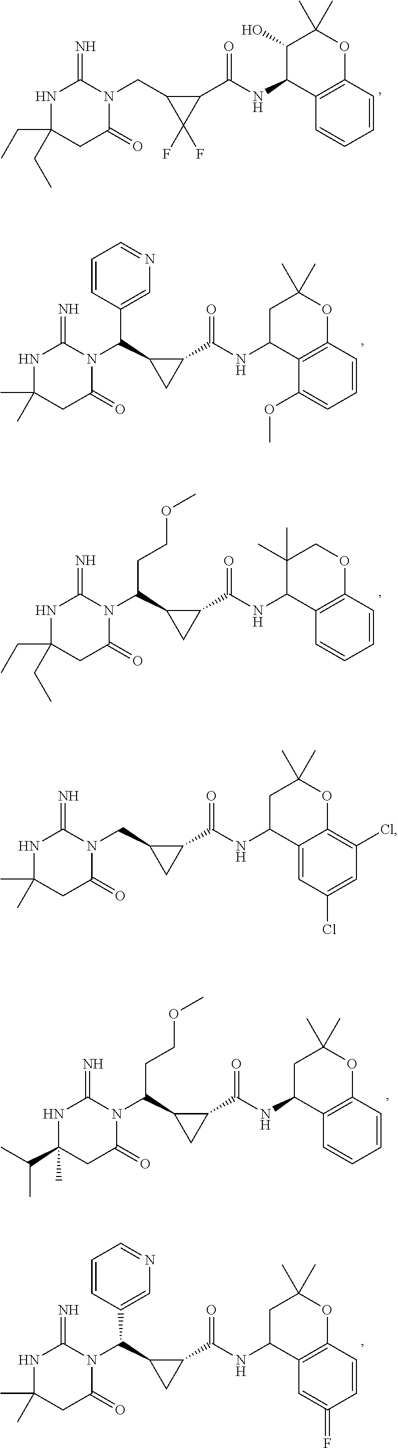

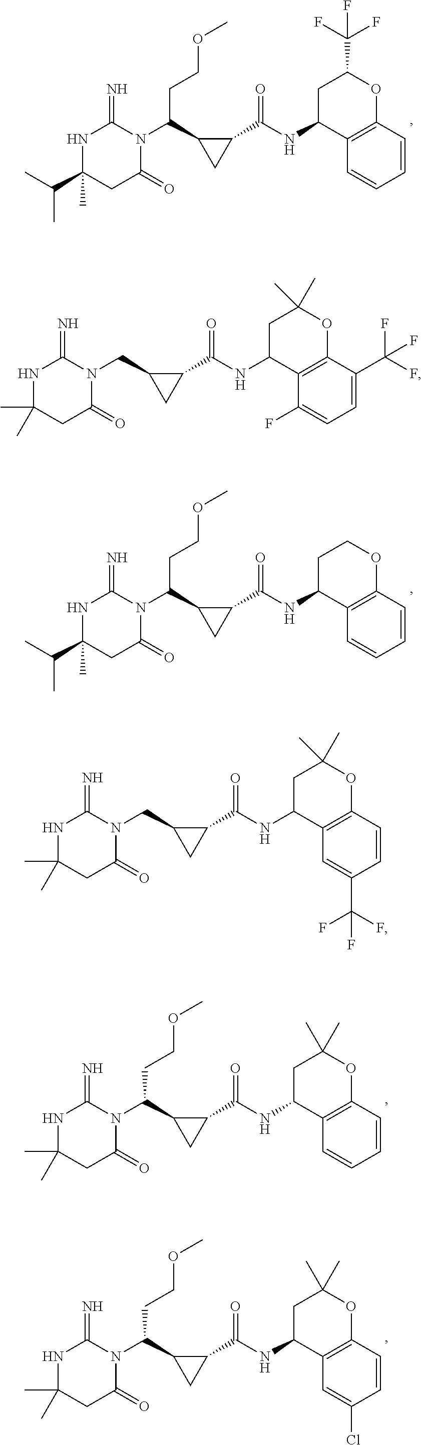 [SMILES: COCCC([C@@H]1C[C@H]1C(=O)N[C@H]1C[C@H](C(F)(F)F)Oc2ccccc21)N1C(=N)N[C@](C)(C(C)C)CC1=O ; CC1(C)CC(=O)N(C[C@@H]2C[C@H]2C(=O)NC2CC(C)(C)Oc3c(C(F)(F)F)ccc(F)c32)C(=N)N1 ; COCCC([C@@H]1C[C@H]1C(=O)N[C@H]1CCOc2ccccc21)N1C(=N)N[C@](C)(C(C)C)CC1=O ; CC1(C)CC(=O)N(C[C@@H]2C[C@H]2C(=O)NC2CC(C)(C)Oc3ccc(C(F)(F)F)cc32)C(=N)N1 ; COCC[C@H]([C@@H]1C[C@H]1C(=O)N[C@@H]1CC(C)(C)Oc2ccccc21)N1C(=N)NC(C)(C)CC1=O ; COCC[C@H]([C@@H]1C[C@H]1C(=O)N[C@H]1CC(C)(C)Oc2ccc(Cl)cc21)N1C(=N)NC(C)(C)CC1=O]

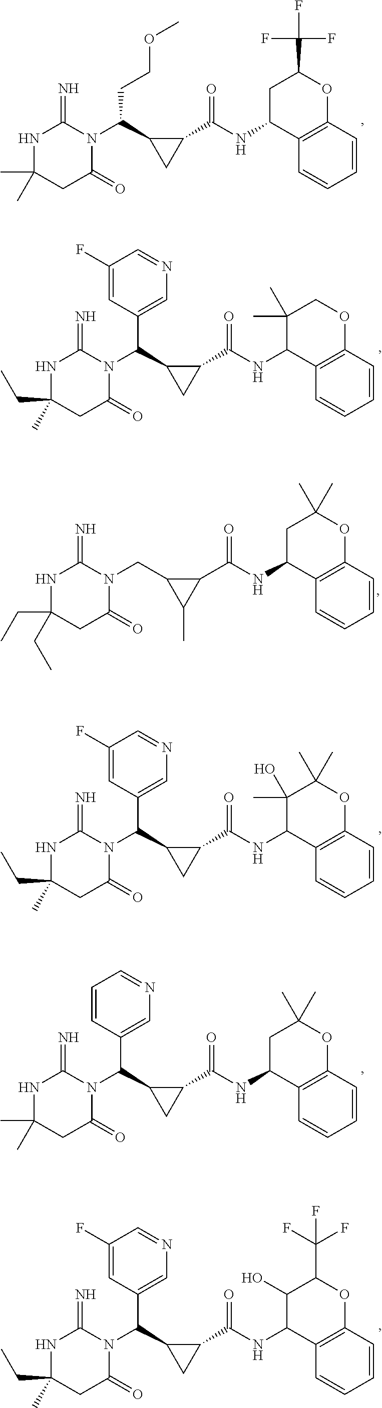 [SMILES: COCC[C@H]([C@@H]1C[C@H]1C(=O)N[C@@H]1C[C@@H](C(F)(F)F)Oc2ccccc21)N1C(=N)NC(C)(C)CC1=O ; CC[C@]1(C)CC(=O)N(C(c2cncc(F)c2)[C@@H]2C[C@H]2C(=O)NC2c3ccccc3OCC2(C)C)C(=N)N1 ; CCC1(CC)CC(=O)N(CC2C(C)C2C(=O)N[C@H]2CC(C)(C)Oc3ccccc32)C(=N)N1 ; CC[C@]1(C)CC(=O)N(C(c2cncc(F)c2)[C@@H]2C[C@H]2C(=O)NC2c3ccccc3OC(C)(C)C2(C)O)C(=N)N1 ; CC1(C)CC(=O)N(C(c2cccnc2)[C@@H]2C[C@H]2C(=O)N[C@H]2CC(C)(C)Oc3ccccc32)C(=N)N1 ; CC[C@]1(C)CC(=O)N(C(c2cncc(F)c2)[C@@H]2C[C@H]2C(=O)NC2c3ccccc3OC(C(F)(F)F)C2O)C(=N)N1]

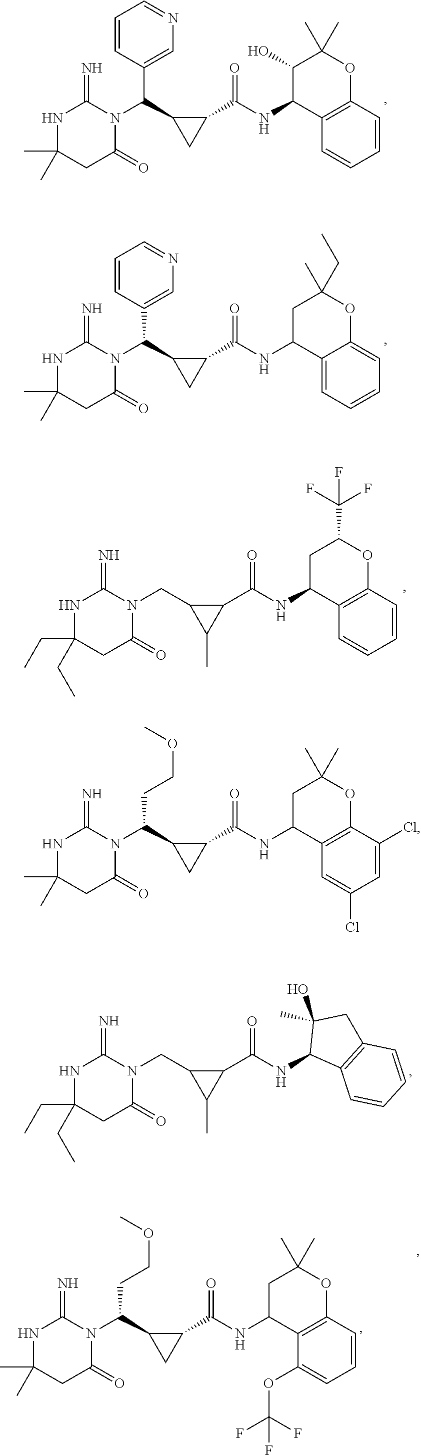 [SMILES: CC1(C)CC(=O)N(C(c2cccnc2)[C@@H]2C[C@H]2C(=O)N[C@@H]2c3ccccc3OC(C)(C)[C@H]2O)C(=N)N1 ; CCC1(C)CC(NC(=O)[C@@H]2C[C@H]2[C@@H](c2cccnc2)N2C(=N)NC(C)(C)CC2=O)c2ccccc2O1 ; CCC1(CC)CC(=O)N(CC2C(C)C2C(=O)N[C@H]2C[C@H](C(F)(F)F)Oc3ccccc32)C(=N)N1 ; COCC[C@H]([C@@H]1C[C@H]1C(=O)NC1CC(C)(C)Oc2c(Cl)cc(Cl)cc21)N1C(=N)NC(C)(C)CC1=O ; CCC1(CC)CC(=O)N(CC2C(C)C2C(=O)N[C@@H]2c3ccccc3C[C@]2(C)O)C(=N)N1 ; COCC[C@H]([C@@H]1C[C@H]1C(=O)NC1CC(C)(C)Oc2cccc(OC(F)(F)F)c21)N1C(=N)NC(C)(C)CC1=O]

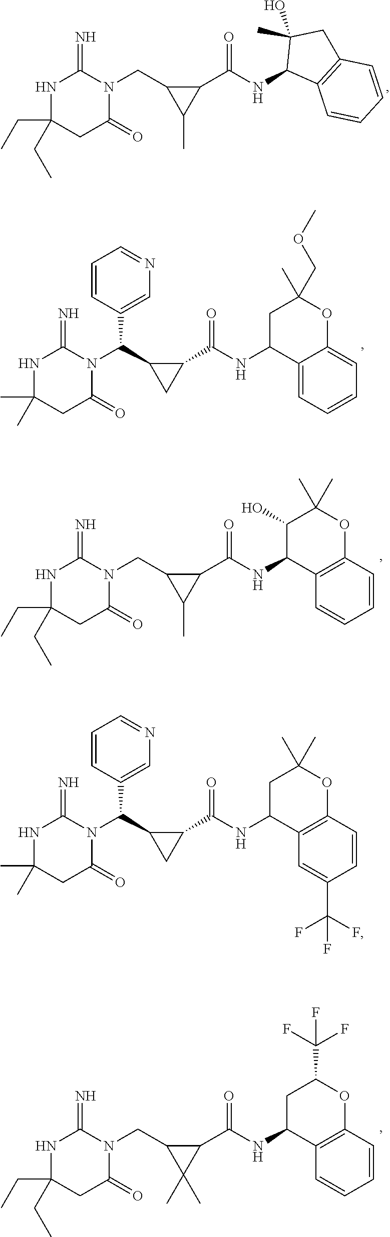 [SMILES: CCC1(CC)CC(=O)N(CC2C(C)C2C(=O)N[C@@H]2c3ccccc3C[C@@]2(C)O)C(=N)N1 ; COCC1(C)CC(NC(=O)[C@@H]2C[C@H]2[C@@H](c2cccnc2)N2C(=N)NC(C)(C)CC2=O)c2ccccc2O1 ; CCC1(CC)CC(=O)N(CC2C(C)C2C(=O)N[C@@H]2c3ccccc3OC(C)(C)[C@H]2O)C(=N)N1 ; CC1(C)CC(=O)N([C@H](c2cccnc2)[C@@H]2C[C@H]2C(=O)NC2CC(C)(C)Oc3ccc(C(F)(F)F)cc32)C(=N)N1 ; CCC1(CC)CC(=O)N(CC2C(C(=O)N[C@H]3C[C@H](C(F)(F)F)Oc4ccccc43)C2(C)C)C(=N)N1]

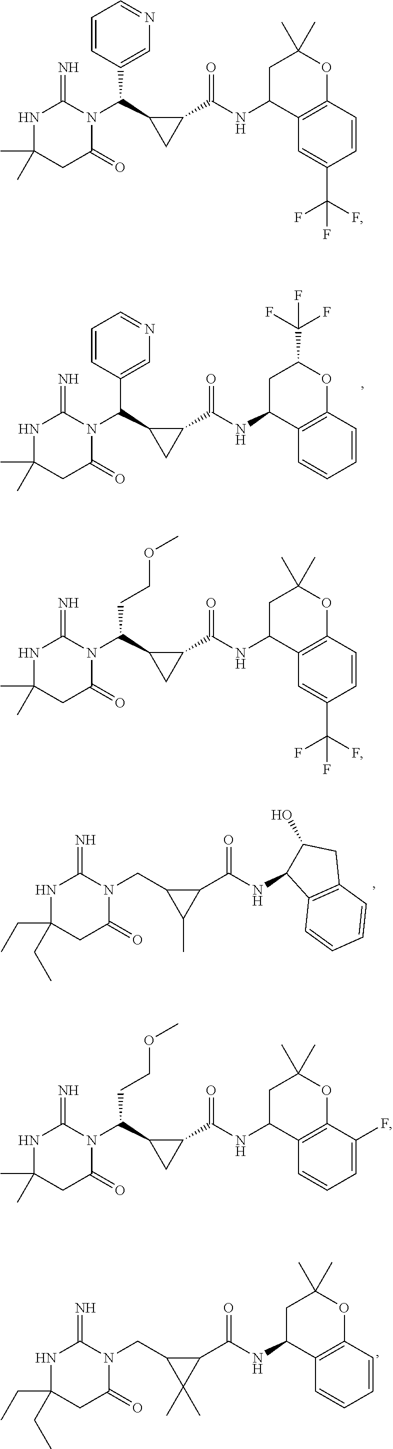 [SMILES: CC1(C)CC(=O)N([C@H](c2cccnc2)[C@@H]2C[C@H]2C(=O)NC2CC(C)(C)Oc3ccc(C(F)(F)F)cc32)C(=N)N1 ; CC1(C)CC(=O)N(C(c2cccnc2)[C@@H]2C[C@H]2C(=O)N[C@H]2C[C@H](C(F)(F)F)Oc3ccccc32)C(=N)N1 ; COCC[C@H]([C@@H]1C[C@H]1C(=O)NC1CC(C)(C)Oc2ccc(C(F)(F)F)cc21)N1C(=N)NC(C)(C)CC1=O ; CCC1(CC)CC(=O)N(CC2C(C)C2C(=O)N[C@@H]2c3ccccc3C[C@H]2O)C(=N)N1 ; COCC[C@H]([C@@H]1C[C@H]1C(=O)NC1CC(C)(C)Oc2c(F)cccc21)N1C(=N)NC(C)(C)CC1=O ; CCC1(CC)CC(=O)N(CC2C(C(=O)N[C@H]3CC(C)(C)Oc4ccccc43)C2(C)C)C(=N)N1]

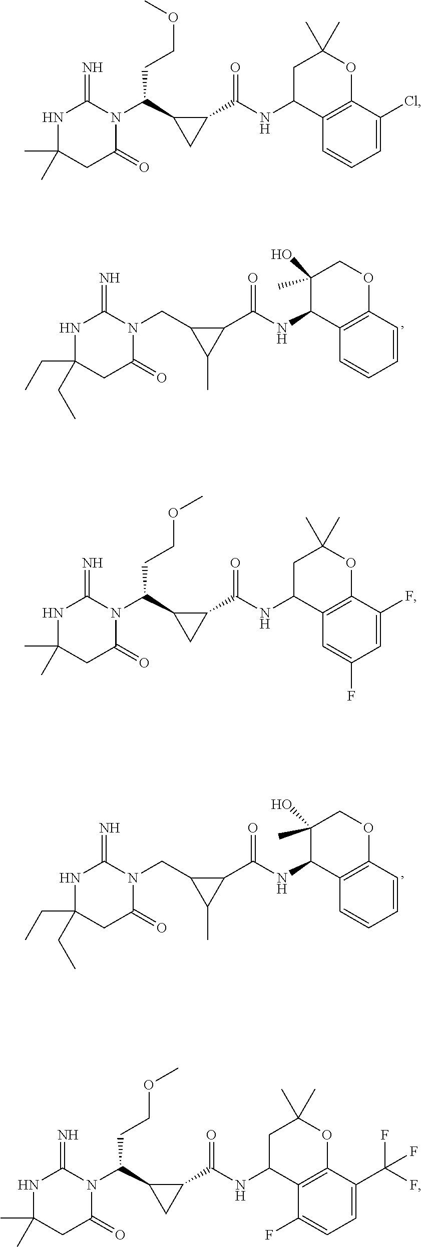 [SMILES: COCC[C@H]([C@@H]1C[C@H]1C(=O)NC1CC(C)(C)Oc2c(Cl)cccc21)N1C(=N)NC(C)(C)CC1=O ; CCC1(CC)CC(=O)N(CC2C(C)C2C(=O)N[C@@H]2c3ccccc3OC[C@]2(C)O)C(=N)N1 ; COCC[C@H]([C@@H]1C[C@H]1C(=O)NC1CC(C)(C)Oc2c(F)cc(F)cc21)N1C(=N)NC(C)(C)CC1=O ; CCC1(CC)CC(=O)N(CC2C(C)C2C(=O)N[C@@H]2c3ccccc3OC[C@@]2(C)O)C(=N)N1 ; COCC[C@H]([C@@H]1C[C@H]1C(=O)NC1CC(C)(C)Oc2c(C(F)(F)F)ccc(F)c21)N1C(=N)NC(C)(C)CC1=O]

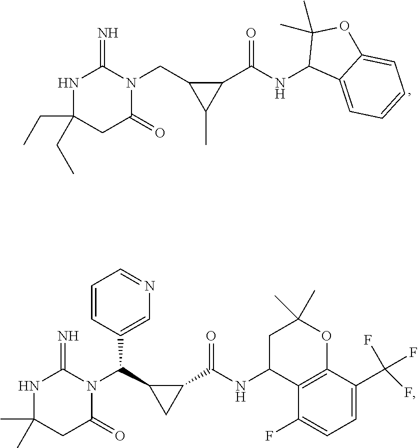 [SMILES: CCC1(CC)CC(=O)N(CC2C(C)C2C(=O)NC2c3ccccc3OC2(C)C)C(=N)N1 ; CC1(C)CC(=O)N([C@H](c2cccnc2)[C@@H]2C[C@H]2C(=O)NC2CC(C)(C)Oc3c(C(F)(F)F)ccc(F)c32)C(=N)N1]

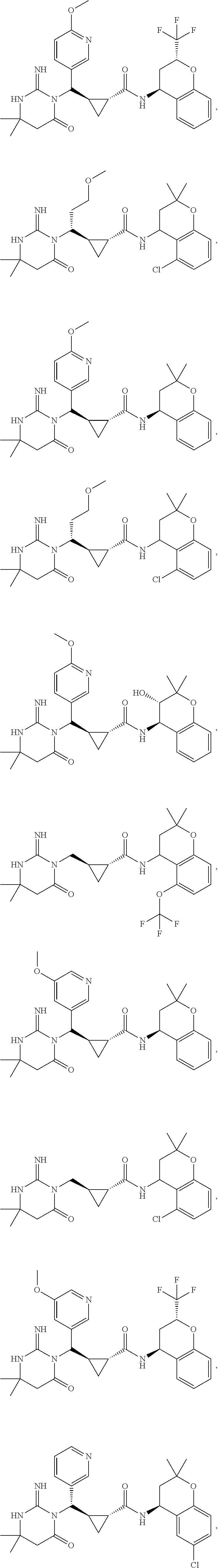

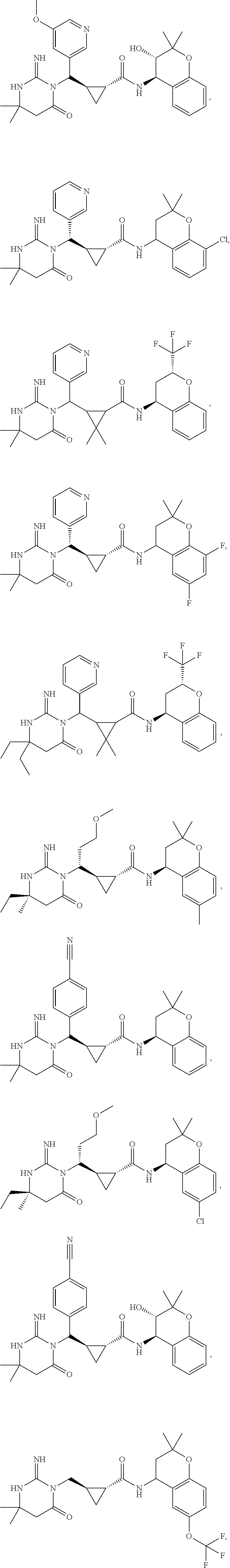

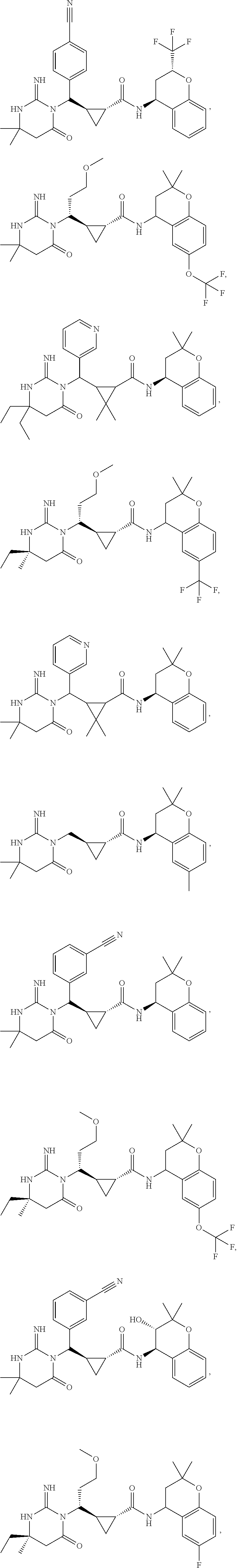

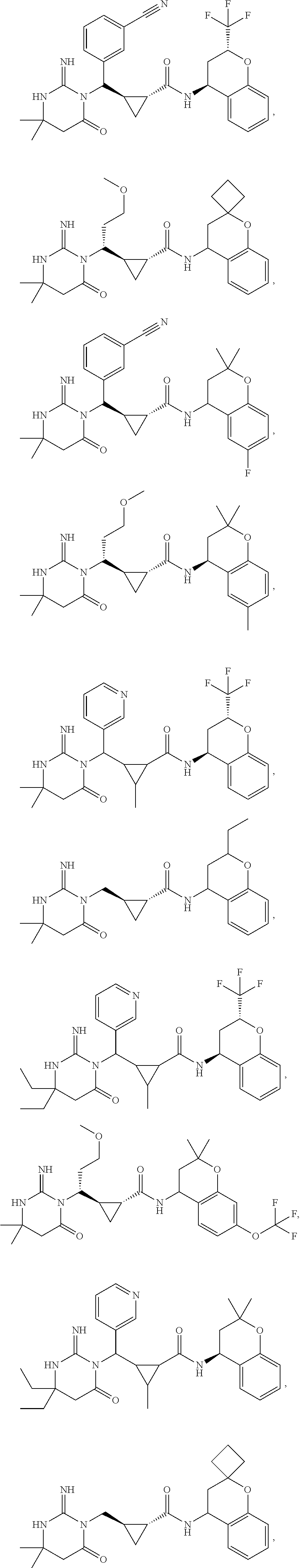

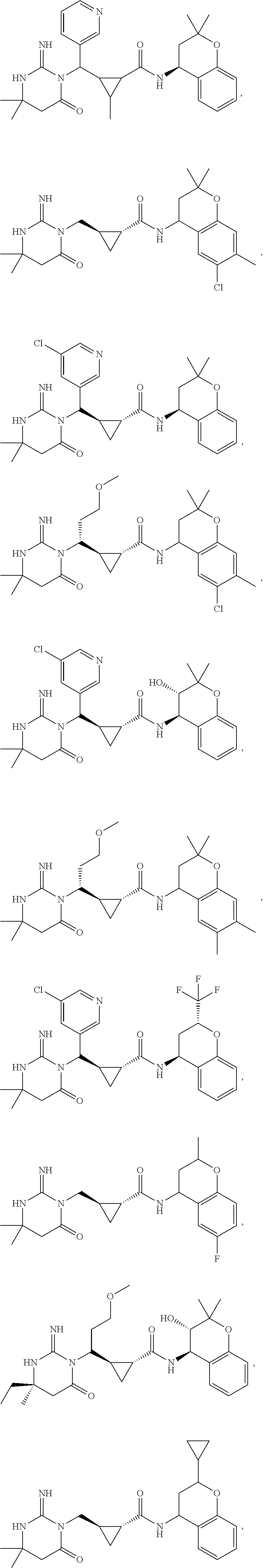

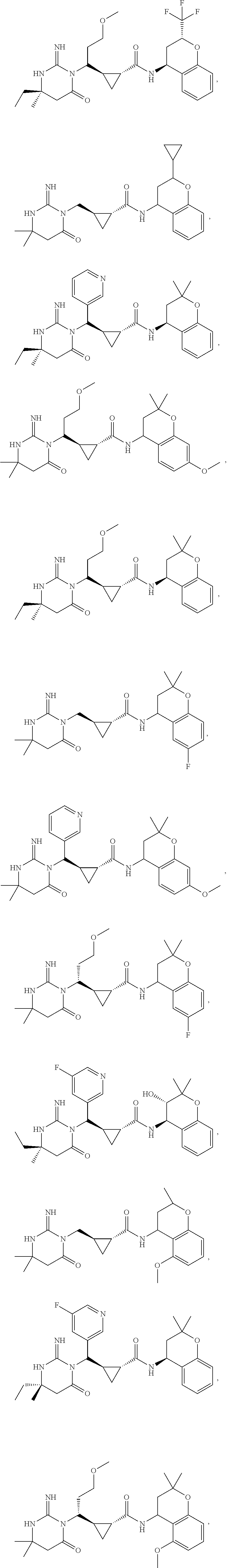

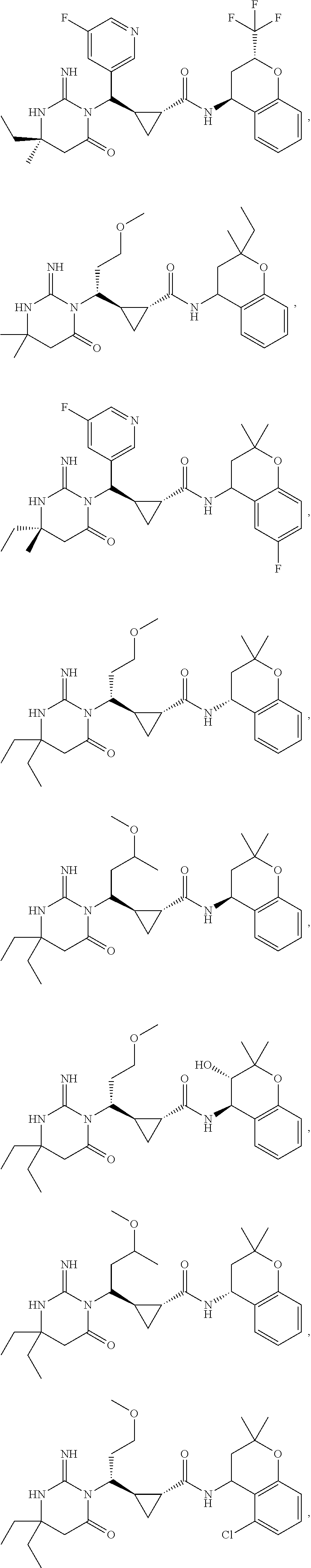

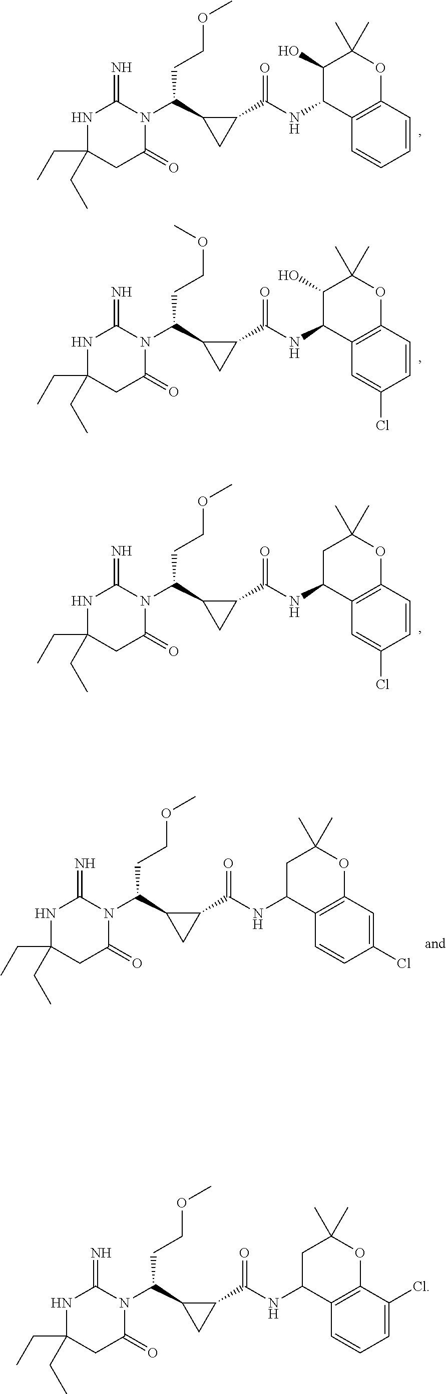 [SMILES: CCC1(CC)CC(=O)N([C@H](CCOC)[C@@H]2C[C@H]2C(=O)N[C@H]2c3ccccc3OC(C)(C)[C@@H]2O)C(=N)N1 ; CCC1(CC)CC(=O)N([C@H](CCOC)[C@@H]2C[C@H]2C(=O)N[C@@H]2c3cc(Cl)ccc3OC(C)(C)[C@H]2O)C(=N)N1 ; CCC1(CC)CC(=O)N([C@H](CCOC)[C@@H]2C[C@H]2C(=O)N[C@H]2CC(C)(C)Oc3ccc(Cl)cc32)C(=N)N1 ; CCC1(CC)CC(=O)N([C@H](CCOC)[C@@H]2C[C@H]2C(=O)NC2CC(C)(C)Oc3cc(Cl)ccc32)C(=N)N1 ; CCC1(CC)CC(=O)N([C@H](CCOC)[C@@H]2C[C@H]2C(=O)NC2CC(C)(C)Oc3c(Cl)cccc32)C(=N)N1]

The compounds described and exemplified herein, can also be described using IUPAC nomenclature:
[1-[1-[(1R,2R)-2-[[(4S)-2,2-dimethylchroman-4-yl]carbamoyl]cyclopropyl]-3-methoxy-propyl]-4,4-diethyl-6-oxo-hexahydropyrimidin-2-ylidene]ammonium;2,2,2-trifluoroacetate;
[1-[1-[(1R,2R)-2-[[(4R)-2,2-dimethylchroman-4-yl]carbamoyl]cyclopropyl]-3-methoxy-propyl]-4,4-diethyl-6-oxo-hexahydropyrimidin-2-ylidene]ammonium;2,2,2-trifluoroacetate;
[1-[[(1R,2R)-2-[[(4S)-2,2-dimethylchroman-4-yl]carbamoyl]cyclopropyl]-pyridin-1-ium-3-yl-methyl]-4,4-diethyl-6-oxo-hexahydropyrimidin-2-ylidene]ammonium;2,2,2-trifluoroacetate;
[4,4-diethyl-6-oxo-1-[[(1S,2S)-2-[[(1S)-1-phenylethyl]carbamoyl]cyclopropyl]methyl]hexahydropyrimidin-2-ylidene]ammonium;2,2,2-trifluoroacetate;
[4,4-diethyl-6-oxo-1-[[2-[[(1R)-1-phenylethyl]carbamoyl]cyclopropyl]methyl]hexahydropyrimidin-2-ylidene]ammonium;2,2,2-trifluoroacetate;
[1-[[(1S,2S)-2-[[(4S)-chroman-4-yl]carbamoyl]cyclopropyl]methyl]-4,4-diethyl-6-oxo-hexahydropyrimidin-2-ylidene]ammonium;2,2,2-trifluoroacetate;
[1-[[(1S,2S)-2-[[(4R)-chroman-4-yl]carbamoyl]cyclopropyl]methyl]-4,4-diethyl-6-oxo-hexahydropyrimidin-2-ylidene]ammonium;2,2,2-trifluoroacetate;
[1-[[2-[[(4S)-chroman-4-yl]carbamoyl]cyclopropyl]methyl]-4,4-diethyl-6-oxo-hexahydropyrimidin-2-ylidene]ammonium;2,2,2-trifluoroacetate;
[1-[[(1R,2R)-2-[[(4S)-2,2-dimethylchroman-4-yl]carbamoyl]cyclopropyl]methyl]-4,4-diethyl-6-oxo-hexahydropyrimidin-2-ylidene]ammonium;2,2,2-trifluoroacetate;
[4,4-diethyl-1-[[(1S,2S)-2-[[(1R,2R)-2-hydroxyindan-1-yl]carbamoyl]cyclopropyl]methyl]-6-oxo-hexahydropyrimidin-2-ylidene]ammonium;2,2,2-trifluoroacetate;
[4,4-diethyl-1-[[(1R,2R)-2-[(6-fluoro-2,2-dimethyl-chroman-4-yl)carbamoyl]cyclopropyl]methyl]-6-oxo-hexahydropyrimidin-2-ylidene]ammonium;2,2,2-trifluoroacetate;
[4,4-diethyl-6-oxo-1-[[(1R,2R)-2-[[2-(trifluoromethyl)chroman-4-yl]carbamoyl]cyclopropyl]methyl]hexahydropyrimidin-2-ylidene]ammonium;2,2,2-trifluoroacetate;
(1R,2R)—N-[(4S)-chroman-4-yl]-2-[(4,4-diethyl-2-imino-6-oxo-hexahydropyrimidin-1-yl)-(3-pyridyl)methyl]cyclopropanecarboxamide;
(1R,2R)-2-[(4,4-diethyl-2-imino-6-oxo-hexahydropyrimidin-1-yl)-(3-pyridyl)methyl]-N-[(1R,2R)-2-hydroxyindan-1-yl]cyclopropanecarboxamide;
[4,4-diethyl-1-[[(1R,2R)-2-[(3-hydroxy-2,2-dimethyl-chroman-4-yl)carbamoyl]cyclopropyl]-pyridin-1-ium-3-yl-methyl]-6-oxo-hexahydropyrimidin-2-ylidene]ammonium;2,2,2-trifluoroacetate;
[4,4-diethyl-6-oxo-1-[pyridin-1-ium-3-yl-[(1R,2R)-2-[[(2R,4S)-2-(trifluoromethyl)chroman-4-yl]carbamoyl]cyclopropyl]methyl]hexahydropyrimidin-2-ylidene]ammonium;2,2,2-trifluoroacetate;
[4,4-diethyl-1-[1-[(1R,2R)-2-[[(1R,2R)-2-hydroxyindan-1-yl]carbamoyl]cyclopropyl]-3-methoxy-propyl]-6-oxo-hexahydropyrimidin-2-ylidene]ammonium;2,2,2-trifluoroacetate;
[4,4-diethyl-1-[3-methoxy-1-[(1R,2R)-2-[[(2R,4S)-2-(trifluoromethyl)chroman-4-yl]carbamoyl]cyclopropyl]propyl]-6-oxo-hexahydropyrimidin-2-ylidene]ammonium;2,2,2-trifluoroacetate;
[1-[[3-[[(4S)-chroman-4-yl]carbamoyl]-2,2-difluoro-cyclopropyl]methyl]-4,4-diethyl-6-oxo-hexahydropyrimidin-2-ylidene]ammonium;2,2,2-trifluoroacetate;
[1-[[3-[[(4R)-chroman-4-yl]carbamoyl]-2,2-difluoro-cyclopropyl]methyl]-4,4-diethyl-6-oxo-hexahydropyrimidin-2-ylidene]ammonium;2,2,2-trifluoroacetate;
[1-[[3-[[(4S)-2,2-dimethylchroman-4-yl]carbamoyl]-2,2-difluoro-cyclopropyl]methyl]-4,4-diethyl-6-oxo-hexahydropyrimidin-2-ylidene]ammonium;2,2,2-trifluoroacetate;
[1-[[3-[[(4R)-2,2-dimethylchroman-4-yl]carbamoyl]-2,2-difluoro-cyclopropyl]methyl]-4,4-diethyl-6-oxo-hexahydropyrimidin-2-ylidene]ammonium;2,2,2-trifluoroacetate;
[1-[[2,2-difluoro-3-[[(2R,4S)-2-(trifluoromethyl)chroman-4-yl]carbamoyl]cyclopropyl]methyl]-4,4-diethyl-6-oxo-hexahydropyrimidin-2-ylidene]ammonium;2,2,2-trifluoroacetate;
[1-[[2,2-difluoro-3-[[(2R,4R)-2-(trifluoromethyl)chroman-4-yl]carbamoyl]cyclopropyl]methyl]-4,4-diethyl-6-oxo-hexahydropyrimidin-2-ylidene]ammonium;2,2,2-trifluoroacetate;
[1-[[2,2-difluoro-3-[[(3S,4R)-3-hydroxy-2,2-dimethyl-chroman-4-yl]carbamoyl]cyclopropyl]methyl]-4,4-diethyl-6-oxo-hexahydropyrimidin-2-ylidene]ammonium;2,2,2-trifluoroacetate;
[1-[[2,2-difluoro-3-[[(3S,4S)-3-hydroxy-2,2-dimethyl-chroman-4-yl]carbamoyl]cyclopropyl]methyl]-4,4-diethyl-6-oxo-hexahydropyrimidin-2-ylidene]ammonium;2,2,2-trifluoroacetate;
[1-[1-[(1R,2R)-2-[(3,3-dimethylchroman-4-yl)carbamoyl]cyclopropyl]-3-methoxy-propyl]-4,4-diethyl-6-oxo-hexahydropyrimidin-2-ylidene]ammonium;2,2,2-trifluoroacetate;
[(4S)-1-[1-[(1R,2R)-2-[[(4S)-2,2-dimethylchroman-4-yl]carbamoyl]cyclopropyl]-3-methoxy-propyl]-4-isopropyl-4-methyl-6-oxo-hexahydropyrimidin-2-ylidene]ammonium;2,2,2-trifluoroacetate;
[(4R)-4-isopropyl-1-[3-methoxy-1-[(1R,2R)-2-[[(2R,4S)-2-(trifluoromethyl)chroman-4-yl]carbamoyl]cyclopropyl]propyl]-4-methyl-6-oxo-hexahydropyrimidin-2-ylidene]ammonium;2,2,2-trifluoroacetate;
[(4S)-4-isopropyl-1-[3-methoxy-1-[(1R,2R)-2-[[(2R,4S)-2-(trifluoromethyl)chroman-4-yl]carbamoyl]cyclopropyl]propyl]-4-methyl-6-oxo-hexahydropyrimidin-2-ylidene]ammonium;2,2,2-trifluoroacetate;
[(4R)-1-[1-[(1R,2R)-2-[[(4S)-chroman-4-yl]carbamoyl]cyclopropyl]-3-methoxy-propyl]-4-isopropyl-4-methyl-6-oxo-hexahydropyrimidin-2-ylidene]ammonium;2,2,2-trifluoroacetate;
[1-[(1R)-1-[(1R,2R)-2-[[(4R)-2,2-dimethylchroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-[(1R,2R)-2-[[(4S)-2,2-dimethylchroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-3-methoxy-1-[(1R,2R)-2-[[(2S,4R)-2-(trifluoromethyl)chroman-4-yl]carbamoyl]cyclopropyl]propyl]-4,4-dimethyl-6-oxo-hexahydropyrimidin-2-ylidene]ammonium;2,2,2-trifluoroacetate;
[1-[3-methoxy-1-[(1R,2R)-2-[[(2R,4S)-2-(trifluoromethyl)chroman-4-yl]carbamoyl]cyclopropyl]propyl]-4,4-dimethyl-6-oxo-hexahydropyrimidin-2-ylidene]ammonium;2,2,2-trifluoroacetate;
[1-[3-methoxy-1-[(1R,2R)-2-[[(2R,4R)-2-(trifluoromethyl)chroman-4-yl]carbamoyl]cyclopropyl]propyl]-4,4-dimethyl-6-oxo-hexahydropyrimidin-2-ylidene]ammonium;2,2,2-trifluoroacetate;
[1-[[2-[[(4S)-2,2-dimethylchroman-4-yl]carbamoyl]-3-methyl-cyclopropyl]methyl]-4,4-diethyl-6-oxo-hexahydropyrimidin-2-ylidene]ammonium;2,2,2-trifluoroacetate;
[1-[[2-[[(4R)-2,2-dimethylchroman-4-yl]carbamoyl]-3-methyl-cyclopropyl]methyl]-4,4-diethyl-6-oxo-hexahydropyrimidin-2-ylidene]ammonium;2,2,2-trifluoroacetate;
[1-[[(1R,2R)-2-[[(4S)-2,2-dimethylchroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[[(3S,4R)-3-hydroxy-2,2-dimethyl-chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[4,4-diethyl-1-[[2-methyl-3-[[(2R,4S)-2-(trifluoromethyl)chroman-4-yl]carbamoyl]cyclopropyl]methyl]-6-oxo-hexahydropyrimidin-2-ylidene]ammonium;2,2,2-trifluoroacetate;
[4,4-diethyl-1-[[2-methyl-3-[[(2R,4R)-2-(trifluoromethyl)chroman-4-yl]carbamoyl]cyclopropyl]methyl]-6-oxo-hexahydropyrimidin-2-ylidene]ammonium;2,2,2-trifluoroacetate;
2-[(4,4-diethyl-2-imino-6-oxo-hexahydropyrimidin-1-yl)methyl]-N-[(1R,2S)-2-hydroxy-2-methyl-indan-1-yl]-3-methyl-cyclopropanecarboxamide;
2-[(4,4-diethyl-2-imino-6-oxo-hexahydropyrimidin-1-yl)methyl]-N-[(1R,2R)-2-hydroxy-2-methyl-indan-1-yl]-3-methyl-cyclopropanecarboxamide;
2-[(4,4-diethyl-2-imino-6-oxo-hexahydropyrimidin-1-yl)methyl]-N-[(1S,2S)-2-hydroxy-2-methyl-indan-1-yl]-3-methyl-cyclopropanecarboxamide;
[4,4-diethyl-1-[[2-[[(3S,4R)-3-hydroxy-2,2-dimethyl-chroman-4-yl]carbamoyl]-3-methyl-cyclopropyl]methyl]-6-oxo-hexahydropyrimidin-2-ylidene]ammonium;2,2,2-trifluoroacetate;
[4,4-diethyl-1-[[2-[[(3R,4R)-3-hydroxy-2,2-dimethyl-chroman-4-yl]carbamoyl]-3-methyl-cyclopropyl]methyl]-6-oxo-hexahydropyrimidin-2-ylidene]ammonium;2,2,2-trifluoroacetate;
[1-[[2,2-dimethyl-3-[[(2R,4S)-2-(trifluoromethyl)chroman-4-yl]carbamoyl]cyclopropyl]methyl]-4,4-diethyl-6-oxo-hexahydropyrimidin-2-ylidene]ammonium;2,2,2-trifluoroacetate;
3-[(4,4-diethyl-2-imino-6-oxo-hexahydropyrimidin-1-yl)methyl]-2,2-dimethyl-N-[(2R,4S)-2-(trifluoromethyl)chroman-4-yl]cyclopropanecarboxamide;
[4,4-dimethyl-6-oxo-1-[pyridin-1-ium-3-yl-[(1R,2R)-2-[[(2R,4S)-2-(trifluoromethyl)chroman-4-yl]carbamoyl]cyclopropyl]methyl]hexahydropyrimidin-2-ylidene]ammonium;2,2,2-trifluoroacetate;
[4,4-diethyl-1-[[2-[[(1R,2R)-2-hydroxyindan-1-yl]carbamoyl]-3-methyl-cyclopropyl]methyl]-6-oxo-hexahydropyrimidin-2-ylidene]ammonium;2,2,2-trifluoroacetate;
3-[(4,4-diethyl-2-imino-6-oxo-hexahydropyrimidin-1-yl)methyl]-N-[(4S)-2,2-dimethylchroman-4-yl]-2,2-dimethyl-cyclopropanecarboxamide;
2-[(4,4-diethyl-2-imino-6-oxo-hexahydropyrimidin-1-yl)methyl]-N-[(3R,4R)-3-hydroxy-3-methyl-chroman-4-yl]-3-methyl-cyclopropanecarboxamide;
2-[(4,4-diethyl-2-imino-6-oxo-hexahydropyrimidin-1-yl)methyl]-N-[(3S,4R)-3-hydroxy-3-methyl-chroman-4-yl]-3-methyl-cyclopropanecarboxamide;
[1-[[(2S)-[(2,2-dimethyl-3H-benzofuran-3-yl)carbamoyl]-3-methyl-cyclopropyl]methyl]-4,4-diethyl-6-oxo-hexahydropyrimidin-2-ylidene]ammonium;2,2,2-trifluoroacetate;
[1-[[(2R)-[(2,2-dimethyl-3H-benzofuran-3-yl)carbamoyl]-3-methyl-cyclopropyl]methyl]-4,4-diethyl-6-oxo-hexahydropyrimidin-2-ylidene]ammonium;2,2,2-trifluoroacetate;
[1-[(6-methoxy-3-pyridyl)-[(1R,2R)-2-[[(2R,4S)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[[(4S)-2,2-dimethylchroman-4-yl]carbamoyl]cyclopropyl]-(6-methoxy-3-pyridyl)methyl]-4,4-dimethyl-6-oxo-hexahydropyrimidin-2-ylidene]ammonium;2,2,2-trifluoroacetate;
[1-[[(1R,2R)-2-[[(3S,4R)-3-hydroxy-2,2-dimethyl-chroman-4-yl]carbamoyl]cyclopropyl]-(6-methoxy-3-pyridyl)methyl]-4,4-dimethyl-6-oxo-hexahydropyrimidin-2-ylidene]ammonium;2,2,2-trifluoroacetate;
[1-[[(1R,2R)-2-[[(4R)-2,2-dimethylchroman-4-yl]carbamoyl]cyclopropyl]-(5-methoxy-3-pyridyl)methyl]-4,4-dimethyl-6-oxo-hexahydropyrimidin-2-ylidene]ammonium;2,2,2-trifluoroacetate;
[1-[(5-methoxy-3-pyridyl)-[(1R,2R)-2-[[(2R,4S)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[[(3S,4R)-3-hydroxy-2,2-dimethyl-chroman-4-yl]carbamoyl]cyclopropyl]-(5-methoxy-3-pyridyl)methyl]-4,4-dimethyl-6-oxo-hexahydropyrimidin-2-ylidene]ammonium;2,2,2-trifluoroacetate;
[1-[[2,2-dimethyl-3-[[(2R,4S)-2-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[2,2-dimethyl-3-[[(2R,4R)-2-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[2,2-dimethyl-3-[[(2S,4S)-2-(trifluoromethyl)chroman-4-yl]carbamoyl]cyclopropyl]-pyridin-1-ium-3-yl-methyl]-4,4-diethyl-6-oxo-hexahydropyrimidin-2-ylidene]ammonium;2,2,2-trifluoroacetate;
[1-[[2,2-dimethyl-3-[[(2S,4R)-2-(trifluoromethyl)chroman-4-yl]carbamoyl]cyclopropyl]-pyridin-1-ium-3-yl-methyl]-4,4-diethyl-6-oxo-hexahydropyrimidin-2-ylidene]ammonium;2,2,2-trifluoroacetate;
[1-[(4-cyanophenyl)-[(1R,2R)-2-[[(4S)-2,2-dimethylchroman-4-yl]carbamoyl]cyclopropyl]methyl]-4,4-dimethyl-6-oxo-hexahydropyrimidin-2-ylidene]ammonium;2,2,2-trifluoroacetate;
[1-[(4-cyanophenyl)-[(1R,2R)-2-[[(4R)-2,2-dimethylchroman-4-yl]carbamoyl]cyclopropyl]methyl]-4,4-dimethyl-6-oxo-hexahydropyrimidin-2-ylidene]ammonium;2,2,2-trifluoroacetate;
[1-[(4-cyanophenyl)-[(1R,2R)-2-[[(3S,4R)-3-hydroxy-2,2-dimethyl-chroman-4-yl]carbamoyl]cyclopropyl]methyl]-4,4-dimethyl-6-oxo-hexahydropyrimidin-2-ylidene]ammonium;2,2,2-trifluoroacetate;
[1-[(4-cyanophenyl)-[(1R,2R)-2-[[(2R,4S)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[(4-cyanophenyl)-[(1R,2R)-2-[[(2R,4R)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[[3-[[(4S)-2,2-dimethylchroman-4-yl]carbamoyl]-2,2-dimethyl-cyclopropyl]-pyridin-1-ium-3-yl-methyl]-4,4-diethyl-6-oxo-hexahydropyrimidin-2-ylidene]ammonium;2,2,2-trifluoroacetate;
[1-[[3-[[(4R)-2,2-dimethylchroman-4-yl]carbamoyl]-2,2-dimethyl-cyclopropyl]-pyridin-1-ium-3-yl-methyl]-4,4-diethyl-6-oxo-hexahydropyrimidin-2-ylidene]ammonium;2,2,2-trifluoroacetate;
[1-[(3-cyanophenyl)-[(1R,2R)-2-[[(4S)-2,2-dimethylchroman-4-yl]carbamoyl]cyclopropyl]methyl]-4,4-dimethyl-6-oxo-hexahydropyrimidin-2-ylidene]ammonium;2,2,2-trifluoroacetate;
[1-[(3-cyanophenyl)-[(1R,2R)-2-[[(3S,4R)-3-hydroxy-2,2-dimethyl-chroman-4-yl]carbamoyl]cyclopropyl]methyl]-4,4-dimethyl-6-oxo-hexahydropyrimidin-2-ylidene]ammonium;2,2,2-trifluoroacetate;
[1-[(3-cyanophenyl)-[(1R,2R)-2-[[(2R,4S)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[(3-cyanophenyl)-[(1R,2R)-2-[(6-fluoro-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[4,4-dimethyl-1-[[2-methyl-3-[[(2R,4S)-2-(trifluoromethyl)chroman-4-yl]carbamoyl]cyclopropyl]-pyridin-1-ium-3-yl-methyl]-6-oxo-hexahydropyrimidin-2-ylidene]ammonium;2,2,2-trifluoroacetate;
[4,4-diethyl-1-[[2-methyl-3-[[(2R,4S)-2-(trifluoromethyl)chroman-4-yl]carbamoyl]cyclopropyl]-pyridin-1-ium-3-yl-methyl]-6-oxo-hexahydropyrimidin-2-ylidene]ammonium;2,2,2-trifluoroacetate;
[4,4-diethyl-1-[[2-methyl-3-[[(2R,4S)-2-(trifluoromethyl)chroman-4-yl]carbamoyl]cyclopropyl]-pyridin-1-ium-3-yl-methyl]-6-oxo-hexahydropyrimidin-2-ylidene]ammonium;2,2,2-trifluoroacetate;
[1-[[2-[[(4S)-2,2-dimethylchroman-4-yl]carbamoyl]-3-methyl-cyclopropyl]-pyridin-1-ium-3-yl-methyl]-4,4-diethyl-6-oxo-hexahydropyrimidin-2-ylidene]ammonium;2,2,2-trifluoroacetate;
[1-[[2-[[(4R)-2,2-dimethylchroman-4-yl]carbamoyl]-3-methyl-cyclopropyl]-pyridin-1-ium-3-yl-methyl]-4,4-diethyl-6-oxo-hexahydropyrimidin-2-ylidene]ammonium;2,2,2-trifluoroacetate;
[1-[(5-chloro-3-pyridyl)-[(1R,2R)-2-[[(4S)-2,2-dimethylchroman-4-yl]carbamoyl]cyclopropyl]methyl]-4,4-dimethyl-6-oxo-hexahydropyrimidin-2-ylidene]ammonium;2,2,2-trifluoroacetate;
[1-[(5-chloro-3-pyridyl)-[(1R,2R)-2-[[(4R)-2,2-dimethylchroman-4-yl]carbamoyl]cyclopropyl]methyl]-4,4-dimethyl-6-oxo-hexahydropyrimidin-2-ylidene]ammonium;2,2,2-trifluoroacetate;
[1-[(5-chloro-3-pyridyl)-[(1R,2R)-2-[[(3S,4R)-3-hydroxy-2,2-dimethyl-chroman-4-yl]carbamoyl]cyclopropyl]methyl]-4,4-dimethyl-6-oxo-hexahydropyrimidin-2-ylidene]ammonium;2,2,2-trifluoroacetate;
[1-[(5-chloro-3-pyridyl)-[(1R,2R)-2-[[(2R,4S)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[(5-chloro-3-pyridyl)-[(1R,2R)-2-[[(2R,4R)-2-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[(4R)-4-ethyl-1-[1-[(1R,2R)-2-[[(3S,4R)-3-hydroxy-2,2-dimethyl-chroman-4-yl]carbamoyl]cyclopropyl]-3-methoxy-propyl]-4-methyl-6-oxo-hexahydropyrimidin-2-ylidene]ammonium;2,2,2-trifluoroacetate;
[(4R)-4-ethyl-1-[3-methoxy-1-[(1R,2R)-2-[[(2R,4S)-2-(trifluoromethyl)chroman-4-yl]carbamoyl]cyclopropyl]propyl]-4-methyl-6-oxo-hexahydropyrimidin-2-ylidene]ammonium;2,2,2-trifluoroacetate;
[(4R)-4-ethyl-1-[3-methoxy-1-[(1R,2R)-2-[[(2R,4S)-2-(trifluoromethyl)chroman-4-yl]carbamoyl]cyclopropyl]propyl]-4-methyl-6-oxo-hexahydropyrimidin-2-ylidene]ammonium;2,2,2-trifluoroacetate;
[(4R)-1-[[(1R,2R)-2-[[(4S)-2,2-dimethylchroman-4-yl]carbamoyl]cyclopropyl]-pyridin-1-ium-3-yl-methyl]-4-ethyl-4-methyl-6-oxo-hexahydropyrimidin-2-ylidene]ammonium;2,2,2-trifluoroacetate;
[(4R)-1-[1-[(1R,2R)-2-[[(4R)-2,2-dimethylchroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[(4S)-1-[1-[(1R,2R)-2-[[(4S)-2,2-dimethylchroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[1-[[(1R,2R)-2-[(7-methoxy-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[(4R)-4-ethyl-1-[(5-fluoro-3-pyridyl)-[(1R,2R)-2-[[(3S,4R)-3-hydroxy-2,2-dimethyl-chroman-4-yl]carbamoyl]cyclopropyl]methyl]-4-methyl-6-oxo-hexahydropyrimidin-2-ylidene]ammonium;2,2,2-trifluoroacetate;
[(4S)-1-[[(1R,2R)-2-[[(4S)-2,2-dimethylchroman-4-yl]carbamoyl]cyclopropyl]-(5-fluoro-3-pyridyl)methyl]-4-ethyl-4-methyl-6-oxo-hexahydropyrimidin-2-ylidene]ammonium;2,2,2-trifluoroacetate;
[(4R)-1-[[(1R,2R)-2-[[(4R)-2,2-dimethylchroman-4-yl]carbamoyl]cyclopropyl]-(5-fluoro-3-pyridyl)methyl]-4-ethyl-4-methyl-6-oxo-hexahydropyrimidin-2-ylidene]ammonium;2,2,2-trifluoroacetate;
[(4R)-4-ethyl-1-[(5-fluoro-3-pyridyl)-[(1R,2R)-2-[[(2R,4S)-2-(trifluoromethyl)chroman-4-yl]carbamoyl]cyclopropyl]methyl]-4-methyl-6-oxo-hexahydropyrimidin-2-ylidene]ammonium;2,2,2-trifluoroacetate;
[(4S)-4-ethyl-1-[[(1R,2R)-2-[(6-fluoro-2,2-dimethyl-chroman-4-yl)carbamoyl]cyclopropyl]-(5-fluoro-3-pyridyl)methyl]-4-methyl-6-oxo-hexahydropyrimidin-2-ylidene]ammonium;2,2,2-trifluoroacetate;
[(4R)-4-ethyl-1-[[(1R,2R)-2-[(6-fluoro-2,2-dimethyl-chroman-4-yl)carbamoyl]cyclopropyl]-(5-fluoro-3-pyridyl)methyl]-4-methyl-6-oxo-hexahydropyrimidin-2-ylidene]ammonium;2,2,2-trifluoroacetate;
[1-[3-methoxy-1-[(1R,2R)-2-[(7-methoxy-2,2-dimethyl-chroman-4-yl)carbamoyl]cyclopropyl]propyl]-4,4-dimethyl-6-oxo-hexahydropyrimidin-2-ylidene]ammonium;2,2,2-trifluoroacetate;
[1-[[(1R,2R)-2-[(6-fluoro-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[(5-methoxy-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(1R)-3-methoxy-1-[(1R,2R)-2-[(5-methoxy-2,2-dimethyl-chroman-4-yl)carbamoyl]cyclopropyl]propyl]-4,4-dimethyl-6-oxo-hexahydropyrimidin-2-ylidene]ammonium;2,2,2-trifluoroacetate;
[1-[(1S)-3-methoxy-1-[(1R,2R)-2-[(5-methoxy-2,2-dimethyl-chroman-4-yl)carbamoyl]cyclopropyl]propyl]-4,4-dimethyl-6-oxo-hexahydropyrimidin-2-ylidene]ammonium;2,2,2-trifluoroacetate;
[1-[(1R)-1-[(1R,2R)-2-[(2-ethyl-2-methyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(2-ethyl-2-methyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(7-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(7-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[(7-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(7-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[(2-ethyl-2-methyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[(2-isopropyl-2-methyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[(2-isopropyl-2-methyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(1R)-1-[(1R,2R)-2-[(2-isopropyl-2-methyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(2-isopropyl-2-methyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[(2-isopropyl-2-methyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(2-isopropyl-2-methyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(1R)-3-methoxy-1-[(1R,2R)-2-[[2-(methoxymethyl)-2-methyl-chroman-4-yl]carbamoyl]cyclopropyl]propyl]-4,4-dimethyl-6-oxo-hexahydropyrimidin-2-ylidene]ammonium;2,2,2-trifluoroacetate;
[1-[(1S)-3-methoxy-1-[(1R,2R)-2-[[2-(methoxymethyl)-2-methyl-chroman-4-yl]carbamoyl]cyclopropyl]propyl]-4,4-dimethyl-6-oxo-hexahydropyrimidin-2-ylidene]ammonium;2,2,2-trifluoroacetate;
[1-[(1R)-3-methoxy-1-[(1R,2R)-2-[(2,2,7-trimethylchroman-4-yl)carbamoyl]cyclopropyl]propyl]-4,4-dimethyl-6-oxo-hexahydropyrimidin-2-ylidene]ammonium;2,2,2-trifluoroacetate;
[4,4-dimethyl-6-oxo-1-[[(1R,2R)-2-[(2,2,7-trimethylchroman-4-yl)carbamoyl]cyclopropyl]methyl]hexahydropyrimidin-2-ylidene]ammonium;2,2,2-trifluoroacetate;
[4,4-dimethyl-6-oxo-1-[(S)-pyridin-1-ium-3-yl-[(1R,2R)-2-[(2,2,7-trimethylchroman-4-yl)carbamoyl]cyclopropyl]methyl]hexahydropyrimidin-2-ylidene]ammonium;2,2,2-trifluoroacetate;
[4,4-dimethyl-6-oxo-1-[(R)-pyridin-1-ium-3-yl-[(1R,2R)-2-[(2,2,7-trimethylchroman-4-yl)carbamoyl]cyclopropyl]methyl]hexahydropyrimidin-2-ylidene]ammonium;2,2,2-trifluoroacetate;
[1-[(S)-[(1R,2R)-2-[(5-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(5-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[(5-fluoro-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(1R)-1-[(1R,2R)-2-[(5-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(5-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(7-chloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[[(1R,2R)-2-[(7-chloro-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(S)-[(1R,2R)-2-[(7-chloro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(7-chloro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(1R)-1-[(1R,2R)-2-[(2,2-diethylchroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(2,2-diethylchroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[(8-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(8-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(S)-[(1R,2R)-2-[(6,8-dichloro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(6,8-dichloro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[[(4S)-6-chloro-2,2-dimethyl-chroman-4-yl]carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[(5-methoxy-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[(5-methoxy-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[(6,8-dichloro-2,2-dimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(S)-[(1R,2R)-2-[(6-fluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[[(1R,2R)-2-[[5-fluoro-2,2-dimethyl-8-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[[2,2-dimethyl-6-(trifluoromethyl)chroman-4-yl]carbamoyl]cyclopropyl]methyl]-4,4-dimethyl-6-oxo-hexahydropyrimidin-2-ylidene]ammonium;2,2,2-trifluoroacetate;
[1-[(1R)-1-[(1R,2R)-2-[[(4S)-6-chloro-2,2-dimethyl-chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[(4R)-1-[[(1R,2R)-2-[(3,3-dimethylchroman-4-yl)carbamoyl]cyclopropyl]-(5-fluoro-3-pyridyl)methyl]-4-ethyl-4-methyl-6-oxo-hexahydropyrimidin-2-ylidene]ammonium;2,2,2-trifluoroacetate;
[(4S)-1-[[(1R,2R)-2-[(3,3-dimethylchroman-4-yl)carbamoyl]cyclopropyl]-(5-fluoro-3-pyridyl)methyl]-4-ethyl-4-methyl-6-oxo-hexahydropyrimidin-2-ylidene]ammonium;2,2,2-trifluoroacetate;
[(4R)-4-ethyl-1-[(5-fluoro-3-pyridyl)-[(1R,2R)-2-[(3-hydroxy-2,2,3-trimethyl-chroman-4-yl)carbamoyl]cyclopropyl]methyl]-4-methyl-6-oxo-hexahydropyrimidin-2-ylidene]ammonium;2,2,2-trifluoroacetate;
[(4S)-4-ethyl-1-[(5-fluoro-3-pyridyl)-[(1R,2R)-2-[(3-hydroxy-2,2,3-trimethyl-chroman-4-yl)carbamoyl]cyclopropyl]methyl]-4-methyl-6-oxo-hexahydropyrimidin-2-ylidene]ammonium;2,2,2-trifluoroacetate;
[(4R)-4-ethyl-1-[(5-fluoro-3-pyridyl)-[(1R,2R)-2-[[3-hydroxy-2-(trifluoromethyl)chroman-4-yl]carbamoyl]cyclopropyl]methyl]-4-methyl-6-oxo-hexahydropyrimidin-2-ylidene]ammonium;2,2,2-trifluoroacetate;
[(4S)-4-ethyl-1-[(5-fluoro-3-pyridyl)-[(1R,2R)-2-[[3-hydroxy-2-(trifluoromethyl)chroman-4-yl]carbamoyl]cyclopropyl]methyl]-4-methyl-6-oxo-hexahydropyrimidin-2-ylidene]ammonium;2,2,2-trifluoroacetate;
[1-[(S)-[(1R,2R)-2-[(2-ethyl-2-methyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(2-ethyl-2-methyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(1R)-1-[(1R,2R)-2-[(6,8-dichloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(6,8-dichloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[[2,2-dimethyl-5-(trifluoromethoxy)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[[2,2-dimethyl-5-(trifluoromethoxy)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[[2-(methoxymethyl)-2-methyl-chroman-4-yl]carbamoyl]cyclopropyl]-(3-pyridyl)methyl]-4,4-dimethyl-6-oxo-hexahydropyrimidin-2-ylidene]ammonium;2,2,2-trifluoroacetate;
[1-[(R)-[(1R,2R)-2-[[2-(methoxymethyl)-2-methyl-chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(S)-[(1R,2R)-2-[[2-(methoxymethyl)-2-methyl-chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(S)-[(1R,2R)-2-[[2-(methoxymethyl)-2-methyl-chroman-4-yl]carbamoyl]cyclopropyl]-(3-pyridyl)methyl]-4,4-dimethyl-6-oxo-hexahydropyrimidin-2-ylidene]ammonium;2,2,2-trifluoroacetate;
[1-[(S)-[(1R,2R)-2-[[2,2-dimethyl-6-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[[2,2-dimethyl-6-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(1R)-1-[(1R,2R)-2-[[2,2-dimethyl-6-(trifluoromethyl)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[[2,2-dimethyl-6-(trifluoromethyl)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(8-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(8-fluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(8-chloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(8-chloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(6,8-difluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[(6,8-difluoro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[[5-fluoro-2,2-dimethyl-8-(trifluoromethyl)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[[5-fluoro-2,2-dimethyl-8-(trifluoromethyl)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[[5-fluoro-2,2-dimethyl-8-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[[5-fluoro-2,2-dimethyl-8-(trifluoromethyl)chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(1R)-1-[(1R,2R)-2-[(5-chloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(5-chloro-2,2-dimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(S)-[(1R,2R)-2-[(5-chloro-2,2-dimethyl-chroman-4-yl)carbamoyl]cyclopropyl]-(3-pyridyl)methyl]-4,4-dimethyl-6-oxo-hexahydropyrimidin-2-ylidene]ammonium;2,2,2-trifluoroacetate;
[1-[(R)-[(1R,2R)-2-[(5-chloro-2,2-dimethyl-chroman-4-yl)carbamoyl]cyclopropyl]-(3-pyridyl)methyl]-4,4-dimethyl-6-oxo-hexahydropyrimidin-2-ylidene]ammonium;2,2,2-trifluoroacetate;
[1-[[(1R,2R)-2-[[2,2-dimethyl-5-(trifluoromethoxy)chroman-4-yl]carbamoyl]cyclopropyl]methyl]-4,4-dimethyl-6-oxo-hexahydropyrimidin-2-ylidene]ammonium;2,2,2-trifluoroacetate;
(1R,2R)—N-(5-chloro-2,2-dimethyl-chroman-4-yl)-2-[(2-imino-4,4-dimethyl-6-oxo-hexahydropyrimidin-1-yl)methyl]cyclopropanecarboxamide;
[1-[(S)-[(1R,2R)-2-[[(4S)-6-chloro-2,2-dimethyl-chroman-4-yl]carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(8-chloro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(S)-[(1R,2R)-2-[(8-chloro-2,2-dimethyl-chroman-4-yl)carbamoyl]cyclopropyl]-(3-pyridyl)methyl]-4,4-dimethyl-6-oxo-hexahydropyrimidin-2-ylidene]ammonium;2,2,2-trifluoroacetate;
[1-[(S)-[(1R,2R)-2-[(6,8-difluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[1-[(R)-[(1R,2R)-2-[(6,8-difluoro-2,2-dimethyl-chroman-4-yl)carbamoyl]cyclopropyl]-pyridin-1-ium-3-yl-methyl]-4,4-dimethyl-6-oxo-hexahydropyrimidin-2-ylidene]ammonium;2,2,2-trifluoroacetate;
[(4R)-4-ethyl-1-[(1R)-3-methoxy-1-[(1R,2R)-2-[[(4S)-2,2,6-trimethylchroman-4-yl]carbamoyl]cyclopropyl]propyl]-4-methyl-6-oxo-hexahydropyrimidin-2-ylidene]ammonium;2,2,2-trifluoroacetate;
[(4R)-1-[(1R)-1-[(1R,2R)-2-[[(4S)-6-chloro-2,2-dimethyl-chroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[1-[[(1R,2R)-2-[[2,2-dimethyl-6-(trifluoromethoxy)chroman-4-yl]carbamoyl]cyclopropyl]methyl]-4,4-dimethyl-6-oxo-hexahydropyrimidin-2-ylidene]ammonium;2,2,2-trifluoroacetate;
[1-[(1R)-1-[(1R,2R)-2-[[2,2-dimethyl-6-(trifluoromethoxy)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[(4R)-1-[(1R)-1-[(1R,2R)-2-[[2,2-dimethyl-6-(trifluoromethyl)chroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[4,4-dimethyl-6-oxo-1-[[(1R,2R)-2-[[(4S)-2,2,6-trimethylchroman-4-yl]carbamoyl]cyclopropyl]methyl]hexahydropyrimidin-2-ylidene]ammonium;2,2,2-trifluoroacetate;
[(4R)-1-[(1R)-1-[(1R,2R)-2-[[2,2-dimethyl-6-(trifluoromethoxy)chroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[(4S)-1-[(1R)-1-[(1R,2R)-2-[[2,2-dimethyl-6-(trifluoromethoxy)chroman-4-yl]carbamoyl]cyclopropyl]-3-methoxy-propyl]-4-ethyl-4-methyl-6-oxo-hexahydropyrimidin-2-ylidene]ammonium;2,2,2-trifluoroacetate;
[(4R)-4-ethyl-1-[(1R)-1-[(1R,2R)-2-[(6-fluoro-2,2-dimethyl-chroman-4-yl)carbamoyl]cyclopropyl]-3-methoxy-propyl]-4-methyl-6-oxo-hexahydropyrimidin-2-ylidene]ammonium;2,2,2-trifluoroacetate;
[(4S)-4-ethyl-1-[(1R)-1-[(1R,2R)-2-[(6-fluoro-2,2-dimethyl-chroman-4-yl)carbamoyl]cyclopropyl]-3-methoxy-propyl]-4-methyl-6-oxo-hexahydropyrimidin-2-ylidene]ammonium;2,2,2-trifluoroacetate;
[1-[(1R)-3-methoxy-1-[(1R,2R)-2-(spiro[chromane-2,1′-cyclobutane]-4-ylcarbamoyl)cyclopropyl]propyl]-4,4-dimethyl-6-oxo-hexahydropyrimidin-2-ylidene]ammonium;2,2,2-trifluoroacetate;
[1-[(1S)-3-methoxy-1-[(1R,2R)-2-(spiro[chromane-2,1′-cyclobutane]-4-ylcarbamoyl)cyclopropyl]propyl]-4,4-dimethyl-6-oxo-hexahydropyrimidin-2-ylidene]ammonium;2,2,2-trifluoroacetate;
[1-[(1R)-3-methoxy-1-[(1R,2R)-2-[[(4S)-2,2,6-trimethylchroman-4-yl]carbamoyl]cyclopropyl]propyl]-4,4-dimethyl-6-oxo-hexahydropyrimidin-2-ylidene]ammonium;2,2,2-trifluoroacetate;
[1-[[(1R,2R)-2-[(2-ethylchroman-4-yl)carbamoyl]cyclopropyl]methyl]-4,4-dimethyl-6-oxo-hexahydropyrimidin-2-ylidene]ammonium;2,2,2-trifluoroacetate;
[1-[(1R)-1-[(1R,2R)-2-[[2,2-dimethyl-7-(trifluoromethoxy)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[1-[(1S)-1-[(1R,2R)-2-[[2,2-dimethyl-7-(trifluoromethoxy)chroman-4-yl]carbamoyl]cyclopropyl]-3-methoxy-propyl]-4,4-dimethyl-6-oxo-hexahydropyrimidin-2-ylidene]ammonium;2,2,2-trifluoroacetate;
[4,4-dimethyl-6-oxo-1-[[(1R,2R)-2-(spiro[chromane-2,1′-cyclobutane]-4-ylcarbamoyl)cyclopropyl]methyl]hexahydropyrimidin-2-ylidene]ammonium;2,2,2-trifluoroacetate;
[1-[[(1R,2R)-2-[(6-chloro-2,2,7-trimethyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[(1S)-1-[(1R,2R)-2-[(6-chloro-2,2,7-trimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-1-[(1R,2R)-2-[(6-chloro-2,2,7-trimethyl-chroman-4-yl)carbamoyl]cyclopropyl]-3-methoxy-propyl]-4,4-dimethyl-6-oxo-hexahydropyrimidin-2-ylidene]ammonium;2,2,2-trifluoroacetate;
[1-[(1R)-3-methoxy-1-[(1R,2R)-2-[(2,2,6,7-tetramethylchroman-4-yl)carbamoyl]cyclopropyl]propyl]-4,4-dimethyl-6-oxo-hexahydropyrimidin-2-ylidene]ammonium;2,2,2-trifluoroacetate;
[1-[(1S)-3-methoxy-1-[(1R,2R)-2-[(2,2,6,7-tetramethylchroman-4-yl)carbamoyl]cyclopropyl]propyl]-4,4-dimethyl-6-oxo-hexahydropyrimidin-2-ylidene]ammonium;2,2,2-trifluoroacetate;
[1-[[(1R,2R)-2-[(6-fluoro-2-methyl-chroman-4-yl)carbamoyl]cyclopropyl]methyl]-4,4-dimethyl-6-oxo-hexahydropyrimidin-2-ylidene]ammonium;2,2,2-trifluoroacetate;
[1-[[(1R,2R)-2-[(2-cyclopropylchroman-4-yl)carbamoyl]cyclopropyl]methyl]-4,4-dimethyl-6-oxo-hexahydropyrimidin-2-ylidene]ammonium;2,2,2-trifluoroacetate;
(1R,2R)-2-[(1R)-1-(4,4-diethyl-2-imino-6-oxo-hexahydropyrimidin-1-yl)-3-methoxy-propyl]-N-[(4R)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4S)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4S)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4S)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4S)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4R)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4R)-2,2-dimethylchroman-4-yl]cyclopropanecarboxamide;
(1R,2R)-2-[1-(4,4-diethyl-2-imino-6-oxo-hexahydropyrimidin-1-yl)-3-methoxy-butyl]-N-[(4R)-2,2-dimethylchroman-4-yl]cyclopropanecarboxamide;
(1R,2R)-2-[(1R)-1-(4,4-diethyl-2-imino-6-oxo-hexahydropyrimidin-1-yl)-3-methoxy-propyl]-N-[(3R,4S)-3-hydroxy-2,2-dimethyl-chroman-4-yl]cyclopropanecarboxamide;
(1R,2R)-2-[(1R)-1-(4,4-diethyl-2-imino-6-oxo-hexahydropyrimidin-1-yl)-3-methoxy-propyl]-N-[(3S,4R)-3-hydroxy-2,2-dimethyl-chroman-4-yl]cyclopropanecarboxamide;
(1R,2R)—N-[(4S)-6-chloro-2,2-dimethyl-chroman-4-yl]-2-[(1R)-1-(4,4-diethyl-2-imino-6-oxo-hexahydropyrimidin-1-yl)-3-methoxy-propyl]cyclopropanecarboxamide;
(1R,2R)—N-(7-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide;
(1R,2R)—N-(7-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide;
(1R,2R)—N-(8-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide;
(1R,2R)—N-(8-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide;
(1R,2R)—N-(5-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide;
(1R,2R)—N-(5-chloro-2,2-dimethyl-chroman-4-yl)-2-[(1R)-1-(4,4-diethyl-2-imino-6-oxo-hexahydropyrimidin-1-yl)-3-methoxy-propyl]cyclopropanecarboxamide; and
(1R,2R)—N-[(3S,4R)-6-chloro-3-hydroxy-2,2-dimethyl-chroman-4-yl]-2-[(1R)-1-(4,4-diethyl-2-imino-6-oxo-hexahydropyrimidin-1-yl)-3-methoxy-propyl]cyclopropanecarboxamide.

The present invention is also directed to methods of chemovaccination against Plasmodium infection comprising administering to a patient an effective amount of a compound of Formula (I), or a pharmaceutically acceptable salt thereof. In certain embodiments, the compounds of Formula (I), or pharmaceutically acceptable salts thereof, are administered in the form of a pharmaceutical composition, further comprising a pharmaceutically acceptable carrier or excipient.

In certain embodiments described herein, the present invention is directed to methods of chemovaccination against malaria comprising administering to a patient, an effective amount of a selective inhibitor of plasmepsin X, wherein the selective inhibitor of plasmepsin X is a compound of Formula (I), described herein. In some embodiments, the compounds of Formula (I), or pharmaceutically acceptable salts thereof, are administered with a pharmaceutically acceptable carrier, as a pharmaceutical composition. Also provided herein are various embodiments of these methods, as described, infra.

The invention also relates to the use of a compound of Formula (I), (IA), (IB), (IC), or (ID) or a pharmaceutically acceptable salt thereof for selectively inhibiting plasmepsin X activity, for chemovaccination against a Plasmodium infection, or for chemovaccination against malaria. The invention further relates to the use of a compound of Formula (I), (IA), (IB), (IC), or (ID) or a pharmaceutically acceptable salt thereof in the manufacture of a medicament for selectively inhibiting plasmepsin X activity, for chemovaccination against a Plasmodium infection, or for chemovaccination against malaria. The compounds of Formula (I), (IA), (IB), (IC), or (ID) or pharmaceutically acceptable salts thereof described in any of the embodiments of the invention herein are useful for any of the uses above.

Accordingly, another embodiment provides methods for chemovaccination against malaria or for chemovaccination against Plasmodium infection, comprising administration of combinations comprising an amount of at least one compound of Formula (I), (IA), (IB), (IC), or (ID), or a pharmaceutically acceptable salt, solvate, ester or prodrug thereof, and treating malaria by administering an effective amount of one or more additional agents described below. In certain embodiments, described herein are methods for chemovaccination against and treatment of malaria or for chemovaccination against Plasmodium infection and treatment of Plasmodium infection, comprising administration of combinations comprising an amount of at least one compound of Formula (I), (IA), (IB), (IC), or (ID), or a pharmaceutically acceptable salt, solvate, ester or prodrug thereof, and an effective amount of one or more additional anti-malarial agents. In certain embodiments, described herein are methods for chemovaccination against malaria by inhibition of plasmepsin X and treating malaria via at least one other mechanism, comprising administration of combinations comprising an amount of at least one compound of Formula (I), (IA), (IB), (IC), or (ID), or a pharmaceutically acceptable salt, solvate, ester or prodrug thereof, and an effective amount of one or more additional anti-malarial agents, wherein the additional anti-malarial agents act through a different mechanism than inhibiting plasmepsin X.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names and chemical structures may be used interchangeably to describe that same structure. These definitions apply regardless of whether a term is used by itself or in combination with other terms, unless otherwise indicated. Hence the definition of “alkyl” applies to “alkyl” as well as the “alkyl” portion of “hydroxyalkyl”, “haloalkyl”, arylalkyl-, alkylaryl-, “alkoxy” etc.

It shall be understood that, in the various embodiments of the invention described herein, any variable not explicitly defined in the context of the embodiment is as defined in Formula (I).

In the various embodiments described herein, each variable is selected independently of the others unless otherwise indicated.

“Chemovaccination” means induction of adaptive immune responses to Plasmodium infection during anti-viral drug administration.

“Drug resistant” means, in connection with a Plasmodium parasite strain, a Plasmodium species which is no longer susceptible to at least one previously effective drug; which has developed the ability to withstand attack by at least one previously effective drug. A drug resistant strain may relay that ability to withstand to its progeny. Said resistance may be due to random genetic mutations in the bacterial cell that alters its sensitivity to a single drug or to different drugs.

“Patient” includes both human and non-human animals. Non-human animals include those research animals and companion animals such as mice, rats, primates, monkeys, chimpanzees, great apes, dogs, and house cats. In some embodiments of the methods described herein, a patient is a human.

As used herein “patient” means any patient with a liver stage Plasmodium infection, e.g. of Plasmodium falciparum or Plasmodium vivax. Alternatively, a “patient” could mean a patient without Plasmodium parasite infection, that is administered a Plasmodium parasite inoculum, such as a wild-type Plasmodium parasite or a genetically modified Plasmodium parasite and a selective inhibitor of plasmepsin X.

“Pharmaceutical composition” (or “pharmaceutically acceptable composition”) means a composition suitable for administration to a patient. Such compositions may contain the neat compound (or compounds) of the invention or mixtures thereof, or salts, solvates, prodrugs, isomers, or tautomers thereof, and one or more pharmaceutically acceptable carriers or diluents. The term “pharmaceutical composition” is also intended to encompass both the bulk composition and individual dosage units comprised of one or more (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

“Halogen” and “halo” mean fluorine, chlorine, bromine, or iodine. Preferred are fluorine, chlorine and bromine.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl and t-butyl.

“Haloalkyl” means an alkyl as defined above wherein one or more hydrogen atoms on the alkyl is replaced by a halo group defined above.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl. “Monocyclic aryl” means phenyl.

“Cycloalkyl” means a non-aromatic mono- or multicyclic ring system comprising about 3 to about 12 carbon atoms, preferably about 3 to about 10 carbon atoms. Preferred cycloalkyl rings contain about 5 to about 10 ring atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Multicyclic cycloalkyls refers to multicyclic, including bicyclic, rings that include a non-aromatic ring. Non-limiting examples of suitable multicyclic cycloalkyls include 1-decalinyl, norbornyl, adamantyl and the like. In certain embodiments, a non-aromatic ring is fused to an aromatic ring. Further non-limiting examples of cycloalkyl include the following:

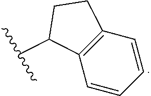 [SMILES: *C1CCc2ccccc21]

“Heterocycloalkyl” (or “heterocyclyl”) means a non-aromatic, saturated or partially saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

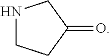

As used herein, the term “monocyclic heterocycloalkyl” refers monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. A non-limiting example of a monocyclic heterocycloalkyl group include the moiety:

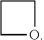

Non-limiting examples of multicyclic heterocycloalkyl groups include, bicyclic heterocycloalkyl groups. Specific examples include, but are not limited to,

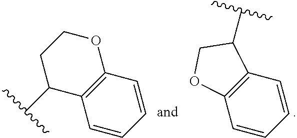 [SMILES: *C1CCOc2ccccc21 ; *C1COc2ccccc21]

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

When a variable appears more than once in a group, e.g., R⁸ in —N(R⁸)₂, or a variable appears more than once in a structure presented herein, the variables can be the same or different.

A solid line [symbol] , as a bond generally indicates a mixture of, or either of, the possible isomers, e.g., containing (R)- and (S)-stereochemistry. For example:

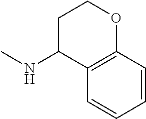

means containing either one of or both

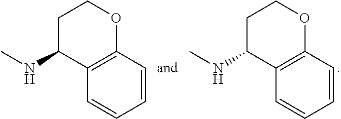

The wavy line [symbol] , as used herein shown crossing a line representing a chemical bond, indicates a point of attachment to the rest of the compound. Lines drawn into the ring systems, such as, for example

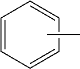

indicates that the indicated line (bond) may be attached to any of the substitutable ring atoms.

“Oxo” is defined as an oxygen atom that is double bonded to a ring carbon in a cycloalkyl, cycloalkenyl, heterocycloalkyl, heterocycloalkenyl, or another ring described herein, e.g.,

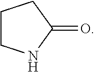

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

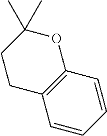

represents

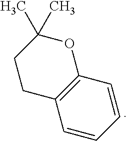

In another embodiment, the compounds useful in the methods of the invention, and/or compositions comprising them useful in said methods, are present in isolated and/or purified form. The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound (or a tautomer or stereoisomer thereof, or pharmaceutically acceptable salt or solvate of said compound, said stereoisomer, or said tautomer) after being obtained from a purification process or processes described herein or well known to the skilled artisan (e.g., chromatography, recrystallization and the like), in sufficient purity to be suitable for in vivo or medicinal use and/or characterizable by standard analytical techniques described herein or well known to the skilled artisan.

It shall be understood that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Another embodiment provides prodrugs and/or solvates of the compounds of the invention. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound or a pharmaceutically acceptable salt thereof, useful in the methods of the invention contains a carboxylic acid functional group, a prodrug can comprise an ester formed by the replacement of the hydrogen atom of the acid group with a group such as, for example, (C₁-C₅)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound used in the methods of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxy carbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound used in the methods of the invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds used in the methods of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of the invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds used in the methods of the invention may optionally be converted to a solvate. Preparation of solvates is generally known. Thus, for example M. Caira et al, J. Pharmaceutical Sci., 1993, 3, 601-611, describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition used in the methods of the present invention effective in inhibiting the above-noted diseases or enzyme activity and thus producing the desired therapeutic, ameliorative, inhibitory or preventative effect.

“Selective inhibitor” means an inhibitor that inhibits a target at least >95 fold better than another target. For example, certain compounds described herein are plasmepsin X inhibitors meaning such compounds inhibit plasmepsin X at least >95 fold better than plasmepsin IX. In certain embodiments, certain compounds described herein, are plasmepsin X inhibitors which inhibit plasmepsin X at least 200-300 fold better than plasmepsin IX.

Another embodiment provides pharmaceutically acceptable salts of the compounds to be used in the methods of the invention. Thus, reference to a compound used in the methods of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds used in the methods of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quartemized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Another embodiment provides pharmaceutically acceptable esters of the compounds used in the methods of the invention. Such esters include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁-20 alcohol or reactive derivative thereof, or by a 2,3-di (C₆₋₂₄)acyl glycerol.

As mentioned herein, another embodiment provides tautomers of the compounds of the invention to be used in the methods herein, and salts, solvates, esters and prodrugs of said tautomers. It shall be understood that all tautomeric forms of such compounds are within the scope of the compounds used in the methods of the invention. For example, all keto-enol and imine-enamine forms of the compounds, when present, are included in the invention.

The compounds used in the methods of the invention may contain asymmetric or chiral centers, and, therefore, exist in different stereoisomeric forms. It is intended that all stereoisomeric forms of the compounds used in the methods of the invention as well as mixtures thereof, including racemic mixtures, form part of the present invention. In addition, the present invention embraces use of all geometric and positional isomers. For example, if a compound used in the methods of the invention incorporates a double bond or a fused ring, both the cis- and trans-forms, as well as mixtures, are embraced within the scope of the invention.

Another embodiment provides for diastereomeric mixtures and individual enantiomers of the compounds used in the methods of the invention. Diastereomeric mixtures can be separated into their individual diastereomers based on their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds used in the methods of the invention may be atropisomers (e.g., substituted biaryls) and are considered as part of this invention. Enantiomers can also be separated by use of chiral HPLC column.

All stereoisomers (for example, geometric isomers, optical isomers and the like) of the compounds used in the methods of the invention (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated as embodiments within the scope of this invention, as are positional isomers (such as, for example, 4-pyridyl and 3-pyridyl). (For example, if a compound of the invention incorporates a double bond or a fused ring, both the cis- and trans-forms, as well as mixtures, are embraced within the scope of the invention. Also, for example, all keto-enol and imine-enamine forms of the compounds are included in the methods of the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

Another embodiment provides isotopically-labelled compounds to be used in the methods the invention. Such compounds are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the invention can generally be prepared by following procedures analogous to those disclosed in the Schemes and/or in the Examples hereinbelow, by substituting an appropriate isotopically labelled reagent for a non-isotopically labelled reagent.

In the compounds used in the methods of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (H) and deuterium (H). The presence of deuterium in the compounds of the invention is indicated by “D”. Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the schemes and examples herein using appropriate isotopically-enriched reagents and/or intermediates.

Polymorphic forms of the compounds used in the methods of the invention, and of the salts, solvates, esters and prodrugs of the compounds of the invention, are intended to be included in the present invention.

As defined herein, an “adjuvant” is a substance that serves to enhance the immunogenicity of an immunogenic composition of the invention. An immune adjuvant may enhance an immune response to an antigen that is weakly immunogenic when administered alone. Thus, adjuvants are often given to boost the immune response and are well known to the skilled artisan. Suitable adjuvants to enhance effectiveness of the composition include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either micro fluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA; (3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein); (4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetrade-canoylaminojethyl 2-Deoxy-4-O-phosphono-3-O—[(R)-3-tetradecanoyloxytetradecanoyl]-2-[(R)-3-tetradecanoyloxy-tetradecanoylamino]-b-D-glucopyranoside, which is also known as 529 (formerly known as RC529), which is formulated as an aqueous form or as a stable emulsion (5) synthetic polynucleotides such as oligonucleotides containing CpG motif(s) (U.S. Pat. No. 6,207,646); and (6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and (7) complement, such as a trimer of complement component C3d.

### Dosage and Administration

Another embodiment provides suitable dosages and dosage forms of the compounds used in the methods of the invention. Suitable doses for administering compounds used in the methods of the invention to patients may readily be determined by those skilled in the art, e.g., by an attending physician, pharmacist, or other skilled worker, and may vary according to patient health, age, weight, frequency of administration, use with other active ingredients, and/or indication for which the compounds are administered. Doses may range from about 0.001 to 500 mg/kg of body weight/day of the compound of the invention. In one embodiment, the dosage is from about 0.01 to about 25 mg/kg of body weight/day of a compound of the invention, or a pharmaceutically acceptable salt or solvate of said compound. In another embodiment, the quantity of active compound in a unit dose of preparation may be varied or adjusted from about 1 mg to about 100 mg, in specific embodiments from about 1 mg to about 50 mg, in specific embodiments from about 1 mg to about 25 mg, according to the particular application. In another embodiment, a typical recommended daily dosage regimen for oral administration can range from about 1 mg/day to about 500 mg/day, in specific embodiments 1 mg/day to 200 mg/day, in two to four divided doses.

As discussed above, the amount and frequency of administration of the compounds of the invention and/or the pharmaceutically acceptable salts thereof will be regulated according to the judgment of the attending clinician considering such factors as age, condition and size of the patient as well as severity of the symptoms being treated.

Liquid form preparations include solutions, suspensions and emulsions. As an example, may be the addition of the compounds described herein and water or water-propylene glycol solutions for parenteral injection or the addition of sweeteners and opacifiers for oral solutions, suspensions and emulsions. Liquid form preparations may also include solutions for intranasal administration. Liquid preparations can also include an adjuvant.

Aerosol preparations suitable for inhalation may include solutions and solids in powder form, which may be in combination with a pharmaceutically acceptable carrier, such as an inert compressed gas, e.g. nitrogen.

In certain embodiments described herein, the preparation can be a long-acting injectable formulation. In certain embodiments of the methods described herein, a plasmepsin X inhibitor is formulated as a long-acting injectable. In certain embodiments of the methods described herein, a compound of Formula (I), (IA), (IB), (IC), or (ID), or a pharmaceutically acceptable salt thereof is formulated as a long-acting injectable.

In certain embodiments, the present invention is also directed to methods of chemovaccination against Plasmodium infection comprising administering to a patient, wherein the patient does not have a Plasmodium parasite infection, a long-acting injectable formulation comprising an effective amount of a selective inhibitor of plasmepsin X, or a pharmaceutically acceptable salt thereof, wherein the patient is eventually exposed to a Plasmodium parasite. In certain embodiments, the exposure to the Plasmodium parasite is through a mosquito bit.

Also included are solid form preparations that are intended to be converted, shortly before use, to liquid form preparations for either oral or parenteral administration. Such liquid forms include solutions, suspensions and emulsions.

Another embodiment provides for use of compositions comprising a compound of Formula (I), (IA), (IB), (IC), or (ID), or a pharmaceutically acceptable salt thereof, formulated for transdermal delivery. The transdermal compositions can take the form of creams, lotions, aerosols and/or emulsions and can be included in a transdermal patch of the matrix or reservoir type as are conventional in the art for this purpose.

Another embodiment provides for use of compositions comprising a compound of Formula (I), (IA), (IB), (IC), or (ID), or a pharmaceutically acceptable salt thereof, formulated for subcutaneous delivery. Another embodiment provides for use of compositions suitable for oral delivery. In some embodiments, it may be advantageous for the pharmaceutical preparation comprising one or more compounds of Formula (I), (IA), (IB), (IC), or (ID), or a pharmaceutically acceptable salt thereof to be prepared in a unit dosage form. In such forms, the preparation is subdivided into suitably sized unit doses containing appropriate quantities of the active component, e.g., an effective amount to achieve the desired purpose. Each of the foregoing alternatives is considered as included in the various embodiments of the invention.

When used in combination with one or more additional therapeutic agents (“combination therapy”), the compounds used in the methods of this invention, i.e. the compounds of Formula (I), (IA), (IB), (IC), or (ID), may be administered together or sequentially. When administered sequentially, compounds of the invention may be administered before or after the one or more additional therapeutic agents, as determined by those skilled in the art or patient preference.

If formulated as a fixed dose, such combination products employ the compounds of Formula (I), (IA), (IB), (IC), or (ID), or a pharmaceutically acceptable salt thereof, within the dosage range described herein and the other pharmaceutically active agent or treatment within its dosage range.

### Combination Therapy

Another embodiment provides for possible methods for chemovaccination using pharmaceutically acceptable compositions comprising a compound of the invention, either as the neat chemical or optionally further comprising additional ingredients. Such compositions are contemplated for preparation and use alone or in combination therapy. For preparing pharmaceutical compositions from the compounds of the invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pennsylvania.

Non-limiting examples of additional drugs and active agents useful in combination therapies for chemovaccination against malaria, include the following: Coartem® (Novartis International AG, Basel, Switzerland; artemether+lumefantrine), Eurartesim® (Sigma-Tau Pharmaceuticals, Inc., Rome, Italy; dihydroartemisinin-piperaquine), Pyramax® (Shin Poong Pharmaceutical Co., Ltd., Seoul, Korea; pyronaridine-artesunate), ASAQ Winthrop@ (Sanofi SA (Gentilly, France)/DNDi (Geneva, Switzerland); artesunate+amodiaquine), ASMQ (Cipla Limited (Mumbai, India)/DNDi, artesunate+mefloquine), SPAQ-CO™ (Guilin Pharmaceutical Co., Ltd. (Shanghai), amodiaquine+sulfadoxine, pyrimethamine), Artesun® (Guilin Pharmaceutical, artesunate), artemether, artesunate, dihydroartemisinin, lumefantrine, amodiaquine, mefloquine, piperaquine, quinine, chloroquine, atovaquone and proguanil and sulfadoxine-pyrimethamine, Tafenoquine (Glaxosmithkline), OZ439/PQP (Sanofi), OZ439/FQ (Sanofi), KAE609 (Novartis), KAF156 (Novartis), DSM265 (NIH/Takeda), and MK-4815 (Merck & Co., Inc., Powles et al., AntimicrobialAgents and Chemotherapy 56(5): 2414-2419(2012)). Selection of such additional active ingredients will be according to the diseases or disorders present for which treatment is desired, as determined by the attending physician or other health care provider.

Thus, the invention also provides methods of using a compound of Formula (I), (IA), (IB), (IC), or (ID), or a pharmaceutically acceptable salt thereof, to selectively inhibit plasmepsin X, and for chemovaccination against Plasmodium infection or chemovaccination against malaria wherein the method further comprises administering to a subject, one or more additional anti-malarial agents. In some embodiments, the one or more additional anti-malarial agents are selected from the group consisting of: artemether, lumefantrine, dihydroartemisinin, piperaquine, pyronaridine, artesunate, amodiaquine, mefloquine, sulfadoxine, pyrimethamine, lumefantrine, quinine, chloroquine, atovaquone, and proguanil.

### Processes of Making Plasmepsin X Inhibitors

ACN=acetonitrile=MeCN
AcOEt=ethylacetate
DCM=dichloromethane
DIAD=Diisopropyl azodicarboxylate
DIEA=N, N-Diisopropylethylamine, or Hunig's base
DMF=N,N-Dimethylformamide
DMP=dimethyl phthalate
EDC=EDCI=1-ethyl-3-(3-dimethylaminopropyl)carbodiimide
EtOAc=ethyl acetate
EtOH=ethanol
h=hours
H₂=hydrogen
HPLC=High Performance Liquid Chromatography
HOBt=Hydroxybenzotriazole
LCMS=Liquid chromatography-mass spectrometry
LHMDS=LiHMDS=lithium bis(trimethylsilyl)amide
LiAlH₄=lithium aluminum hydride
min=minutes
Me=methyl
MeOH═CH₃OH=methanol
N₂=nitrogen
NaBH₄=sodium borohydride
NaH=sodium hydride
Na₂SO₄=sodium sulfate
NH₄Cl=Ammonium chloride
Pd(dppf)Cl₂=[1,1′-Bis(diphenylphosphino)ferrocene]palladium(II) dichloride
SFC=Supercritical Fluid Chromatography
SiO₂=Silicon dioxide
TBAF=Tetra-n-butylammonium fluoride
TFA=trifluoroacetic acid
THF=tetrahydrofuran
TMS=Trimethylsilyl
CDCl₃=heavy chloroform
CD₃OD=heavy methanol
1 Standard atmosphere [atm]=101325 pascal [Pa]=14.6959488 psi

The meanings of the abbreviations in the nuclear magnetic resonance spectra are shown below: s=singlet, d=doublet, dd=double doublet, dt=double triplet, ddd=double double doublet, sept=septet, t=triplet, m=multiplet, br=broad, brs=broad singlet, q=quartet, J=coupling constant and Hz=hertz.

Several methods for preparing the compounds of this disclosure are described in the following Schemes and Examples. Starting materials and intermediates were purchased commercially from common catalog sources or were made using known procedures, or as otherwise illustrated. Some frequently applied routes to the compounds of Formula I are described in in the Schemes that follow. In some cases, the order of carrying out the reaction steps in the schemes may be varied to facilitate the reaction or to avoid unwanted reaction products. An asterisk (*) may be used in a chemical structure drawing that indicates the location of a chiral center.

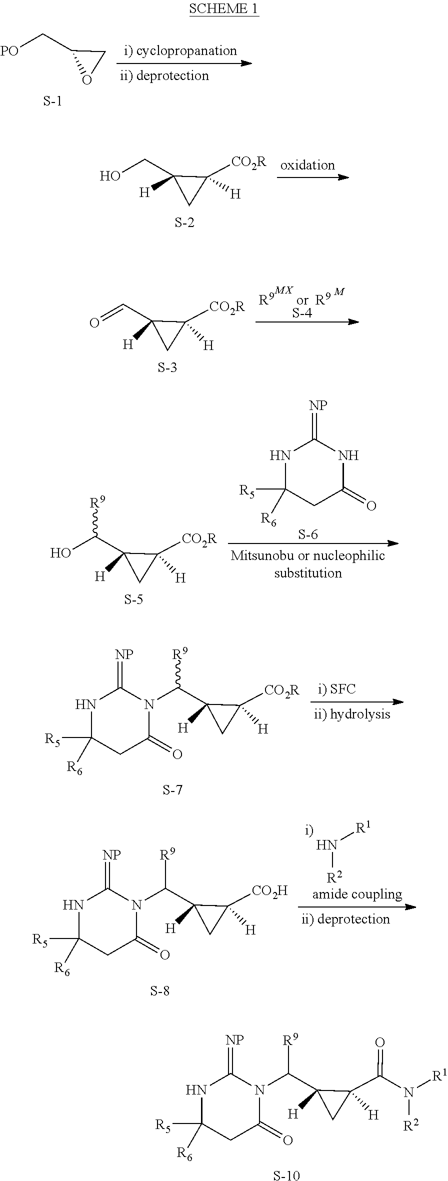

Intermediate compounds of Formula S-2 are prepared from chiral epoxides S-1, in which P is an alcohol protecting group, after cyclopropanation conditions such as (EtO)₂POCH₂CO₂R/NaH followed by alcohol deprotection. Alcohol oxidation in S-2 can be performed using oxidants such as Dess-Martin periodinane to yield aldehydes S-3. Treatment of S-3 with an organometallic reagent S-4, in which M is a metal and X is a halogen, such as organozinc (M=Zn) and organomagnesium (M=Mg) species gives secondary alcohol intermediates S-5. Iminopyrimidones or iminohydantions S-6 are introduced under Mitsunobu conditions to give intermediates S-7. Alternatively, alcohol in intermediates S-5 could be transformed into a leaving group such as a mesylate, tosylate, triflate or halogen which can be displaced with intermediates S-6 to give intermediates S-7. At this step diastereomeric mixtures can be separated by SFC and the resulting chiral esters subjected to acid or base catalyzed hydrolysis or hydrogenation to give chiral acid intermediates S-8. Finally, S-8 intermediates are coupled with amines S-7 to provide final products of Formula S-10 after iminopyrimidone deprotection using acids as TFA or Zn.

Reactions sensitive to moisture or air were performed inside a glove-box or under nitrogen or argon using anhydrous solvents and reagents. The progress of reactions was determined by either analytical thin layer chromatography (TLC) usually performed with E. Merck pre-coated TLC plates, silica gel 60F-254, layer thickness 0.25 mm or liquid chromatography-mass spectrometry (LC/MS).

Typically, the analytical LC-MS system used consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative HPLC purifications were usually performed using either a mass spectrometry directed system or a non-mass guided system. Usually they were performed on a Waters Chromatography Workstation configured with LC-MS System consisting of: Waters ZQ™ single quad MS system with Electrospray Ionization, Waters 2525 Gradient Pump, Waters 2767 Injecto/Collector, Waters 996 PDA Detector, the MS Conditions of: 150-750 amu, Positive Electrospray, Collection Triggered by MS, and a Waters SUNFIRE® C-18 5-micron, 30 mm (id)×100 mm column. The mobile phases consisted of mixtures of acetonitrile (10-100%) in water containing 0.1% TFA. Flow rates were maintained at 50 mL/min, the injection volume was 1800 μL, and the UV detection range was 210-400 nm. An alternate preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C-18 5-micron, 50 mm (id)×250 mm column or a Waters XBridge™ C-18 5-micron OBD™, 30 mm (id)×250 mm column. The mobile phases consisted of mixtures of acetonitrile (0-75%) in water containing 5 mmol (NH₄)HCO₃. Flow rates were maintained at 50 mL/min for the Waters Xbridge™ column and 90 mL/min for the Phenomenex Gemini column. The injection volume ranged from 1000-8000 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage. Concentration of solutions was carried out on a rotary evaporator under reduced pressure. Flash chromatography was usually performed using either a Biotage® Flash Chromatography apparatus (Dyax Corp.), an ISCO CombiFlash® Rf apparatus, or an ISCO CombiFlash® Companion XL on silica gel (32-63 μM, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was most commonly performed on one of CHIRALPAK® AS, CHIRALPAK® AD, CHIRALCEL® OD, CHIRALCEL® IA, or CHIRALCEL® OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of CHIRALPAK AS, of CHIRALPAK AD, CHIRALCEL® OD, CHIRALCEL® IA, CHIRALCEL® OJ columns (20×250 mm) (Daicel Chemical Industries, Ltd.) with desired isocratic solvent systems identified on chiral analytical chromatography or by supercritical fluid (SFC) conditions. It is understood that a chiral center in a compound may exist in the “S” or “R” stereo-configuration, or as a mixture of both. Within a molecule, each bond drawn as a straight line from a chiral center includes both the (R) and (S) stereoisomers as well as mixtures thereof.

### Intermediate 1

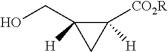

### (1S,2S)-ethyl 2-(hydroxymethyl)cyclopropanecarboxylate

### Step 1:

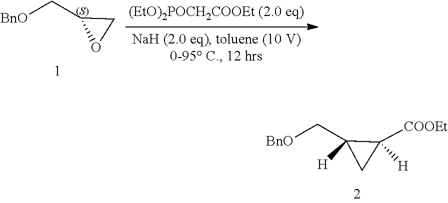

Ethyl 2-diethoxyphosphorylacetate (546 g, 2.44 mol, 2.0 eq) was added dropwise to a suspension of NaH (97.4 g, 2.44 mol, 60% purity, 2.0 eq) in toluene (2.0 L) at 5° C. The resulting mixture was stirred for 30 min at 5° C. Then (S)-2-((benzyloxy)methyl)oxirane (compound 1) (200 g, 1.22 mol, 1.0 eq) was added to the mixture at 5° C. and the resulting mixture was stirred for 12 h at 95° C. Then, the mixture was poured into sat. NH₄Cl (30 L) at 0° C. under N2, and the aqueous phase was extracted with EtOAc (4 L×2). The combined organic layer was washed with brine (3.0 L) and dried with Na₂SO₄, filtered, and the solvent evaporated in vacuo to give a residue that was purified by silica gel chromatography (SiO₂, petroleum ether:EtOAc=10:0 to 0:1) to give compound 2.

H-NMR: (400 MHz, DMSO): δ: 7.26-7.37 (m, 5H), 4.47 (s, 2H), 4.02-4.08 (m, 2H), 3.44-3.48 (m, 1H), 3.28-3.31 (m, 1H), 1.55-1.59 (m, 2H), 1.16-1.20 (m, 3H), 1.03-1.04 (m, 1H), 0.88-0.89 (m, 1H).

Step 2:

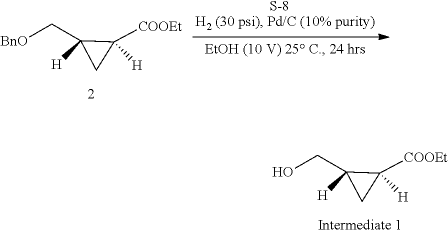

A solution of compound 2 (100 g, 362.80 mmol, 1 eq) in EtOH (1000 mL) was added into a hydrogenation bottle previously charged with Pd/C (20 g, 10% purity) at 25° C. The mixture was purged with H2 three times and stirred under H2 (30 Psi) at 25° C. for 24 h. Then the mixture was filtered and the volatiles removed in vacuo to give a residue that was purified by silica gel chromatography (SiO₂, Petroleum ether:EtOAc=10:1 to 0:1) to give Intermediate 1.

H-NMR: (400 MHz, CDCl₃): δ: 4.10-4.17 (m, 2H), 3.61-3.64 (m, 1H), 3.48-3.51 (m, 1H), 1.57-1.78 (m, 3H), 1.21-1.28 (m, 4H), 0.86-0.88 (m, 1H).

### Example 1

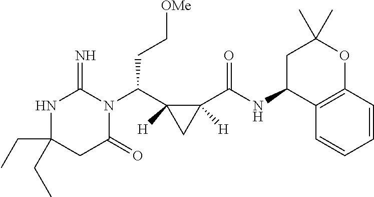 [SMILES: CCC1(CC)CC(=O)N([C@H](CCOC)[C@H]2C[C@@H]2C(=O)N[C@H]2CC(C)(C)Oc3ccccc32)C(=N)N1]

### Step 1:

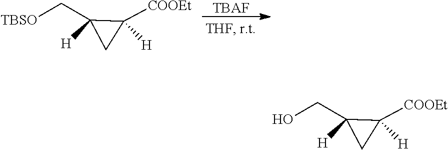

TBAF (155 mL, 155 mmol) was added to a solution of (1S,2S)-ethyl 2-(((tert-butyldimethylsilyl)oxy)methyl)cyclopropanecarboxylate (20 g, 77 mmol) in THF (150 mL) and the mixture was stirred at 26° C. for 2 hours. The mixture was diluted with water (300 mL), extracted with EtOAc (400 mL×3). The organic layers were washed with brine (500 mL), dried over anhydrous Na₂SO₄, filtered and concentrated. The residue was purified by column chromatography (Petroleum ether/EtOAc=100:1-10:1) to afford product (1S,2S)-ethyl 2-(hydroxymethyl)cyclopropanecarboxylate.

¹H NMR (400 MHz, chloroform-d) δ=4.22-4.05 (m, 2H), 3.64 (td, J=5.9, 11.6 Hz, 1H), 3.55-3.42 (m, 1H), 1.80-1.68 (m, 1H), 1.57 (td, J=4.4, 8.5 Hz, 1H), 1.49 (t, J=5.7 Hz, 1H), 1.32-1.18 (m, 4H), 0.87 (ddd, J=4.4, 6.2, 8.4 Hz, 1H)

### Step 2:

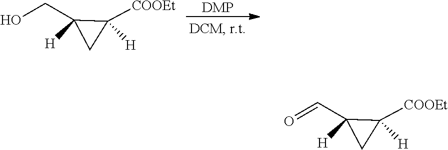

DMP (46.3 g, 109 mmol) was added to a solution of (1S,2S)-ethyl 2-(hydroxymethyl)cyclopropanecarboxylate (10.5 g, 72.8 mmol) in DCM (400 mL). The mixture was stirred at 25° C. for 10 h under N2 atmosphere. The mixture was filtered, then concentrated and purified by flash column (Petroleum ether/EtOAc=100:0 to 10:1) to afford product (1S,2S)-ethyl 2-formylcyclopropanecarboxylate.

¹H NMR (500 MHz, chloroform-d) δ=9.31 (s, 1H), 4.15-4.22 (m, 2H), 2.39-2.49 (m, 1H), 2.06-2.26 (m, 1H), 1.60-1.62 (m, 1H), 1.50-1.51 (m, 1H), 1.27-1.30 (m, 3H)

### Step 3:

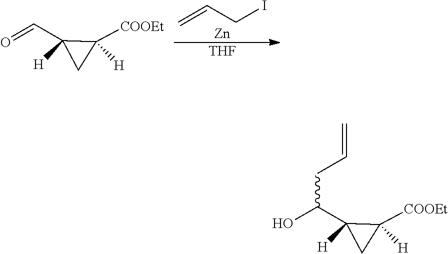

Zinc (4.60 g, 70.3 mmol) was added to a solution of (1S,2S)-ethyl 2-formylcyclopropanecarboxylate (5 g, 35.2 mmol) and 3-iodoprop-1-ene (8.86 g, 52.8 mmol) in THF (80 mL) under N2. The mixture was stirred at 25° C. for 12 h. The mixture was filtered, and the filtrate was concentrated. The residue was purified by column chromatography (Petroleum ether/EtOAc=30:1-5:1) to afford product (1S,2S)-ethyl 2-(1-hydroxybut-3-en-1-yl)cyclopropanecarboxylate.

¹H NMR (500 MHz, chloroform-d) δ=5.76-5.94 (m, 1H), 5.08-5.23 (m, 2H), 4.07-4.23 (m, 2H), 3.15-3.40 (m, 1H), 2.26-2.49 (m, 2H), 1.58-1.67 (m, 2H), 1.26-1.28 (m, 3H), 1.25-1.26 (m, 1H), 0.84-1.03 (m, 1H)

### Step 4:

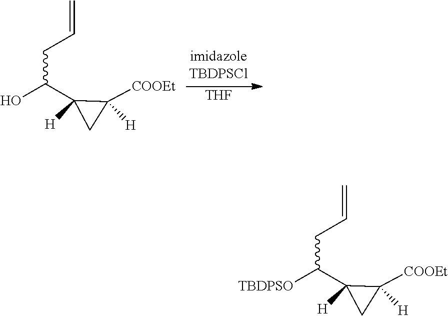

Tertbutylchlorodiphenylsilane (5.97 g, 21.71 mmol) and imidazole (2.217 g, 32.6 mmol) were added to a solution of (1S,2S)-ethyl 2-(1-hydroxybut-3-en-1-yl)cyclopropanecarboxylate (2 g, 10.86 mmol) in DCM (70 mL), and the mixture was stirred at 25° C. for 12 hours under N2 atmosphere. The mixture was diluted with water (50 mL), extracted with DCM (50 mL×3). The organic layers were washed with brine (50 mL), dried over anhydrous Na₂SO₄, filtered and concentrated. The residue was purified by column chromatography (Petroleum ether/EtOAc=100:1-50:1) to afford product (1S,2S)-ethyl 2-(1-((tert-butyldiphenylsilyl)oxy)but-3-en-1-yl)cyclopropanecarboxylate.

¹H NMR (400 MHz, chloroform-d) δ 7.62-7.79 (m, 4H), 7.31-7.50 (m, 6H), 5.69-5.94 (m, 1H), 4.90-5.07 (m, 2H), 3.86-4.15 (m, 2H), 3.16-3.38 (m, 1H), 2.31 (s, 2H), 1.57-1.69 (m, 1H), 1.32-1.42 (m, 1H), 1.14-1.29 (m, 4H), 1.03-1.05 (m, 10H), 0.81-0.99 (m, 1H), 0.47-0.71 (m, 1H)

### Step 5:

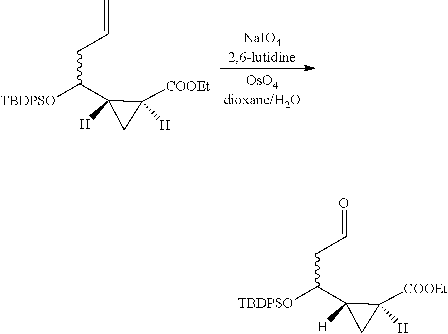

2,6-dimethylpyridine (2.370 mL, 20.35 mmol), osmium tetroxide (0.160 mL, 0.509 mmol) and sodium periodate (8.70 g, 40.7 mmol) were added to a solution of (1S,2S)-ethyl 2-(1-((tert-butyldiphenylsilyl)oxy)but-3-en-1-yl)cyclopropanecarboxylate (4.3 g, 10.17 mmol) in 1,4-dioxane (90 mL) and water (30 mL). The mixture was stirred at 25° C. for 2 h. The mixture was quenched with water (80 mL), then extracted with EtOAc (100 mL×3). The organic layers were washed with brine (100 mL), dried over Na₂SO₄, filtered and concentrated in vacuo to give (1S,2S)-ethyl 2-(1-((tert-butyldiphenylsilyl)oxy)-3-oxopropyl)cyclopropanecarboxylate, which was used for the next step directly.

¹H NMR (500 MHz, chloroform-d) δ 9.75-9.83 (m, 1H), 7.68-7.74 (m, 3H), 7.65 (dd, J=1.5, 8.0 Hz, 1H), 7.41-7.48 (m, 3H), 7.38-7.41 (m, 3H), 4.06-4.10 (m, 1H), 3.80-4.01 (m, 1H), 3.55-3.77 (m, 1H), 2.56-2.77 (m, 2H), 1.63-1.77 (m, 1H), 1.17-1.42 (m, 4H), 1.02 (d, J=16.0 Hz, 10H), 0.46-0.64 (m, 1H)

### Step 6:

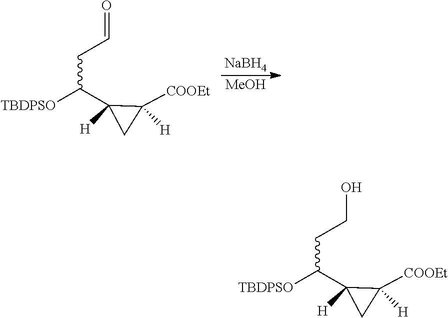

NaBH₄ (0.766 g, 20.25 mmol) was added to solution of (1S,2S)-ethyl 2-(1-((tert-butyldiphenylsilyl)oxy)-3-oxopropyl)cyclopropanecarboxylate (4.3 g, 0.00 mmol) in MeOH (50 mL). The mixture was stirred at −40° C. for 10 min. The mixture was then diluted with water (60 mL) and extracted with EtOAc (80 mL×3). The organic layers were washed with brine (80 mL), dried over anhydrous Na₂SO₄, filtered and concentrated. The residue was purified by column chromatography (Petroleum ether/EtOAc=20:1) to afford product (1S,2S)-ethyl 2-(1-((tert-butyldiphenylsilyl)oxy)-3-hydroxypropyl)cyclopropanecarboxylate.

H NMR (500 MHz, chloroform-d) δ 7.67-7.75 (m, 4H), 7.37-7.48 (m, 6H), 4.07 (q, J=7.0 Hz, 1H), 3.85-3.99 (m, 1H), 3.69-3.85 (m, 2H), 3.25-3.42 (m, 1H), 1.78-1.94 (m, 2H), 1.61-1.74 (m, 2H), 1.29-1.32 (m, 0.5H), 1.14-1.24 (m, 3H), 1.08-1.11 (m, 0.5H), 1.04 (d, J=15.0 Hz, 10H), 0.35-0.58 (m, 1H)

### Step 7:

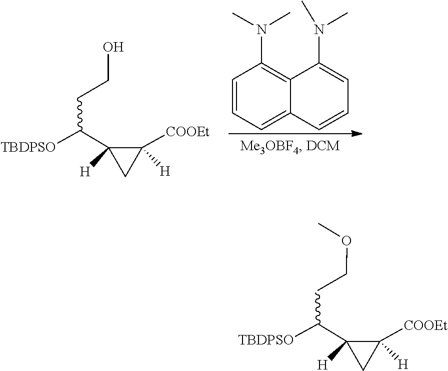

Trimethyloxonium tetrafluoroborate (1.352 g, 9.14 mmol) and 1,8-bis(dimethylamino)naphthalene (2.61 g, 12.19 mmol) was added to a solution of (1S,2S)-ethyl 2-(1-((tert-butyldiphenylsilyl)oxy)-3-hydroxypropyl)cyclopropanecarboxylate (1.3 g, 3.05 mmol) in DCM (50 mL). The mixture was stirred at 25° C. for 13 hrs. The mixture was diluted with water (30 mL) and extracted with DCM (30 mL×3). The organic layers were washed with brine (40 mL), dried over anhydrous Na₂SO₄, filtered and concentrated. The residue was purified by Prep.TLC (Petroleum ether/EtOAc=20:1) to afford product (1S,2S)-ethyl 2-(1-((tert-butyldiphenylsilyl)oxy)-3-methoxypropyl)cyclopropanecarboxylate.

¹H NMR (500 MHz, chloroform-d) δ 7.62-7.82 (m, 4H), 7.31-7.51 (m, 6H), 4.04-4.13 (m, 1H), 3.76-4.00 (m, 1H), 3.42-3.57 (m, 2H), 3.20-3.34 (m, 4H), 1.73-2.01 (m, 2H), 1.60-1.67 (m, 0.5H), 1.31-1.34 (m, 0.5H), 1.12-1.25 (m, 3H), 0.99-1.03 (m, 10H), 0.79-0.91 (m, 1H), 0.58-0.59 (m, 0.5H), 0.36-0.40 (m, 0.5H)

### Step 8:

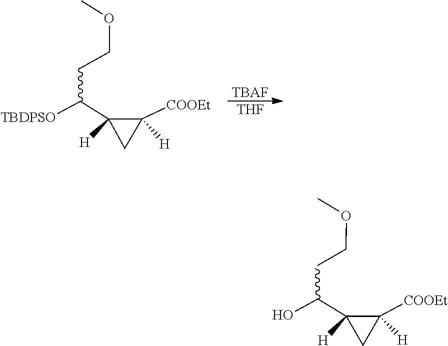

TBAF (6.99 mL, 6.99 mmol) was added to a solution of (1S,2S)-ethyl 2-(1-((tert-butyldiphenylsilyl)oxy)-3-methoxypropyl)cyclopropanecarboxylate (1.54 g, 3.49 mmol) in THF (20 mL). The mixture was stirred at 26° C. for 2 hours. The mixture was then diluted with water (20 mL) and extracted with EtOAc (20 mL×3). The organic layers were washed with brine (30 mL), dried over anhydrous Na₂SO₄, filtered and concentrated. The residue was purified by column chromatography (Petroleum ether/EtOAc=20:1-2:1) to afford product (1S,2S)-ethyl 2-(1-hydroxy-3-methoxypropyl)cyclopropanecarboxylate.

¹H NMR (500 MHz, chloroform-d) δ 4.08-4.18 (m, 2H), 3.67 (td, J=5.0, 9.5 Hz, 1H), 3.53-3.62 (m, 1H), 3.40-3.53 (m, 1H), 3.36 (d, J=2.0 Hz, 3H), 2.90 (d, J=2.0 Hz, 1H), 1.81-1.91 (m, 2H), 1.58-1.73 (m, 2H), 1.27 (dt, J=2.5, 7.0 Hz, 3H), 1.17 (qd, J=4.5, 16.5 Hz, 1H), 0.86-1.05 (m, 1H)

### Step 9:

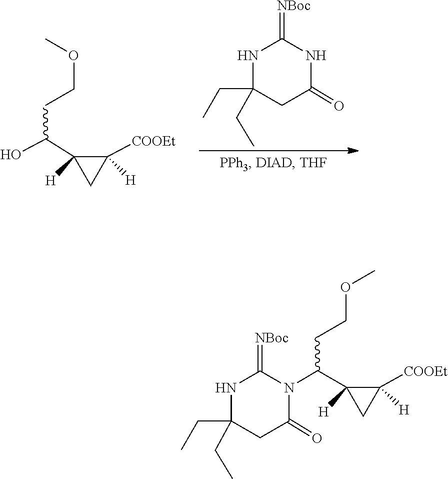

(E)-diisopropyl diazene-1,2-dicarboxylate (0.916 mL, 4.65 mmol) was added dropwise, at 0° C., under N2 atmosphere to a solution of (E)-tert-butyl (4,4-diethyl-6-oxotetrahydropyrimidin-2(1H)-ylidene)carbamate (626 mg, 2.324 mmol), (1S,2S)-ethyl 2-(1-hydroxy-3-methoxypropyl)cyclopropanecarboxylate (470 mg, 2.324 mmol) and triphenylphosphine (1219 mg, 4.65 mmol) in THF (15 mL). The mixture was stirred at 25° C. for 12 h. The mixture was then concentrated in vacuo. The residue was purified by column chromatography (Petroleum ether:EtOAc=15:1-5:1) to afford product (1S,2S)-ethyl 2-(1-((E)-2-((tert-butoxycarbonyl)imino)-4,4-diethyl-6-oxotetrahydropyrimidin-1(2H)-yl)-3-methoxypropyl)cyclopropanecarboxylate. MS (ESI) m/z 454.3 (M+H⁺)

### Step 10:

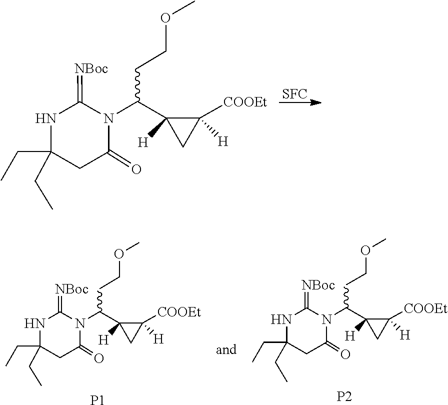

(1S,2S)-ethyl 2-(1-(2-((tert-butoxycarbonyl)imino)-4,4-diethyl-6-oxotetrahydropyrimidin-1(2H)-yl)-3-methoxypropyl)cyclopropanecarboxylate (800 mg, 1.429 mmol) was separated by SFC (Instrument SFC-16 Method Column Phenomenex-Cellulose-2 (250 mm*30 mm, 5 um) Condition 0.1% NH₃H₂O EtOH Begin B 15% End B 15% Gradient Time(min) 100% B Hold Time(min) FlowRate(mL/min) 60 Injections 150) to afford product (1S,2S)-ethyl 2-((R)-1-(2-((tert-butoxycarbonyl)imino)-4,4-diethyl-6-oxotetrahydropyrimidin-1(2H)-yl)-3-methoxypropyl)cyclopropanecarboxylate (Peak 1, Rt=2.072) and (1S,2S)-ethyl 2-((S)-1-(2-((tert-butoxycarbonyl)imino)-4,4-diethyl-6-oxotetrahydropyrimidin-1(2H)-yl)-3-methoxypropyl)cyclopropanecarboxylate (Peak 2, Rt=2.356) both as oils.

MS (ESI) m/z 454.3 (M+H⁺).

Peak 1: ¹H NMR (500 MHz, chloroform-d) δ=9.60-10.17 (m, 1H), 4.36-4.52 (m, 1H), 3.97-4.14 (m, 2H), 3.32-3.53 (m, 2H), 3.28 (s, 3H), 2.50-2.63 (m, 3H), 2.30-2.45 (m, 1H), 2.01-2.19 (m, 1H), 1.60-1.66 (m, 5H), 1.39-1.52 (m, 9H), 1.19-1.21 (m, 4H), 0.87-1.03 (m, 7H)

Peak 2: ¹H NMR (500 MHz, chloroform-d) δ=9.72-10.07 (m, 1H), 4.45 (s, 1H), 4.12-4.13 (m, 2H), 3.39-3.42 (m, 2H), 3.33 (s, 3H), 2.56 (s, 2H), 2.23-2.42 (m, 2H), 1.62 (s, 5H), 1.46-1.52 (m, 9H), 1.24-1.27 (m, 4H), 0.86-1.04 (m, 7H)

### Step 11:

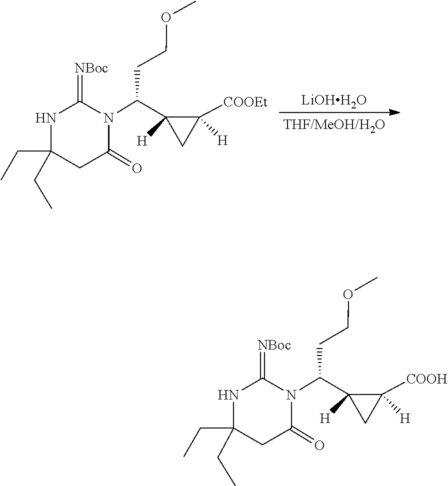

Lithium hydroxide monohydrate (486 mg, 11.57 mmol) was added to a solution of (1S,2S)-ethyl 2-((R)-1-((E)-2-((tert-butoxycarbonyl)imino)-4,4-diethyl-6-oxotetrahydropyrimidin-1(2H)-yl)-3-methoxypropyl)cyclopropanecarboxylate (350 mg, 0.772 mmol) in MeOH (6 mL), THF (6 mL) and water (1.2 mL). The mixture was stirred at 30° C. for 4 hours. EtOAc (4 mL) was added to the mixture and the mixture was neutralized to pH 5-6 with citric acid. The mixture was then quenched with water (10 mL), and extracted with EtOAc (15 mL×3). The organic layers were washed with brine (15 mL), dried over Na₂SO₄, filtered, and concentrated in vacuo to afford crude product (1S,2S)-2-((R)-1-((E)-2-((tert-butoxycarbonyl)imino)-4,4-diethyl-6-oxotetrahydropyrimidin-1(2H)-yl)-3-methoxypropyl)cyclopropanecarboxylic acid, which was used for next step directly.

MS (ESI) m/z 426.2 (M+H⁺).

### Step 12:

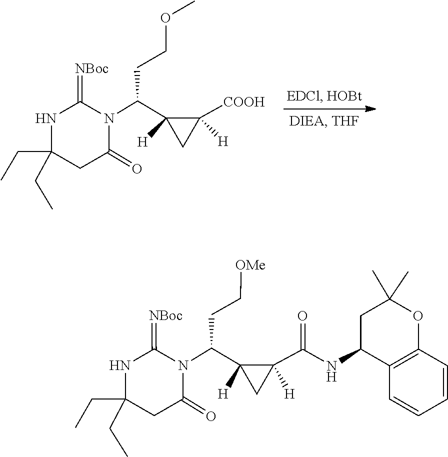

DIEA (0.205 mL, 1.175 mmol) was added to a solution of (1S,2S)-2-((R)-1-(2-((tert-butoxycarbonyl)imino)-4,4-diethyl-6-oxotetrahydropyrimidin-1(2H)-yl)-3-methoxypropyl)cyclopropanecarboxylic acid (100 mg, 0.00 mmol), (S)-2,2-dimethylchroman-4-amine hydrochloride (60.3 mg, 0.282 mmol), HOBt (72.0 mg, 0.470 mmol) and EDCI (180 mg, 0.940 mmol) in THF (6 mL). The mixture was stirred at 25° C. for 2 hours. The mixture was quenched with water (10 mL), and extracted with EtOAc (10 mL×3). The organic layers were washed with brine (15 mL), dried over Na₂SO₄, filtered, and concentrated in vacuo to afford crude product, then purified by Prep-TLC (Petroleum ether/EtOAc=1:2) to give the product tert-butyl (1-((S)-1-((1R,2S)-2-(((S)-2,2-dimethylchroman-4-yl)carbamoyl)cyclopropyl)-3-methoxypropyl)-4,4-diethyl-6-oxotetrahydropyrimidin-2(1H)-ylidene)carbamate.

MS (ESI) m/z 585.4 (M+H⁺).

### Step 13:

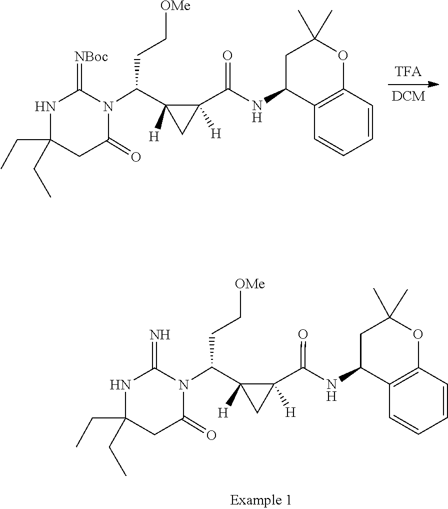

TFA (4 mL) at 22° C. was added to a solution of tert-butyl (1-((R)-1-((1S,2S)-2-(((S)-2,2-dimethylchroman-4-yl)carbamoyl)cyclopropyl)-3-methoxypropyl)-4,4-diethyl-6-oxotetrahydropyrimidin-2(1H)-ylidene)carbamate (100 mg, 0.171 mmol) in DCM (12 mL). The reaction was stirred for 1 h. The mixture was concentrated in vacuo, purified by HPLC (Boston Green ODS 150*30 5u, Condition water(0.1% TFA)-ACN, Begin B 33, End B 63, Gradient Time(min) 10, 100% B Hold Time(min) 2, FlowRate(mL/min) 25, Injections 3), then lyophilized to give the product (1S,2S)-2-((R)-1-(4,4-diethyl-2-imino-6-oxotetrahydropyrimidin-1(2H)-yl)-3-methoxypropyl)-N—((S)-2,2-dimethylchroman-4-yl)cyclopropanecarboxamide. MS (ESI) m/z 485.4 (M+H⁺).

Example 1: ¹H NMR (500 MHz, METHANOL-d4) δ=8.50-8.23 (m, 1H), 7.15-7.14 (m, 1H), 7.08 (d, J=7.5 Hz, 1H), 6.88 (dt, J=1.0, 7.5 Hz, 1H), 6.74 (dd, J=1.0, 8.0 Hz, 1H), 5.37-5.18 (m, 1H), 3.58-3.44 (m, 2H), 3.39 (s, 1H), 3.35 (s, 3H), 2.89 (d, J=16.0 Hz, 1H), 2.70 (d, J=16.0 Hz, 1H), 2.51 (s, 2H), 2.58-2.40 (m, 1H), 2.22 (m, 1H), 2.14-2.02 (m, 1H), 1.88-1.50 (m, 6H), 1.44 (s, 3H), 1.35 (m, 1H), 1.32-1.25 (m, 3H), 0.99-0.71 (m, 6H).

### Example 2

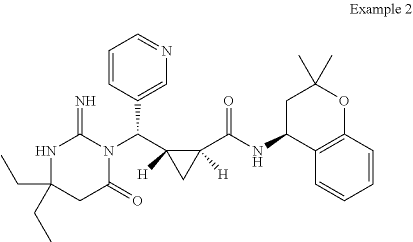 [SMILES: CCC1(CC)CC(=O)N([C@H](c2cccnc2)[C@H]2C[C@@H]2C(=O)N[C@H]2CC(C)(C)Oc3ccccc32)C(=N)N1]

### Step 1:

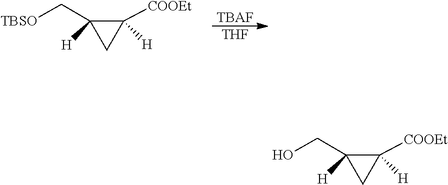

TBAF (232 mL, 232 mmol) was added to a solution of (1S,2S)-ethyl 2-(((tert-butyldimethylsilyl)oxy)methyl)cyclopropanecarboxylate (30 g, 116 mmol) in THF (150 mL). The mixture was stirred at 26° C. for 2 hours. The mixture was diluted with water (300 mL), and extracted with EtOAc (400 mL×3). The organic layers were washed with brine (500 mL), dried over anhydrous Na₂SO₄, filtered and concentrated. The residue was purified by column chromatography (Petroleum ether/EtOAc=100:1-10:1) to afford product (1S,2S)-ethyl 2-(hydroxymethyl)cyclopropanecarboxylate.

¹H NMR (500 MHz, chloroform-d) δ=4.19-4.07 (m, 2H), 3.63 (td, J₁=5.0, J₂=11.0 Hz, 1H), 3.54-3.43 (m, 1H), 1.72-1.73 (m, 1H), 1.56-1.58 (m, 2H), 1.31-1.25 (m, 3H), 1.22-1.23 (m, 1H), 0.92-0.82 (m, 1H).

### Step 2:

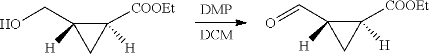 [SMILES: CCOC(=O)[C@H]1C[C@@H]1CO ; CCOC(=O)[C@H]1C[C@@H]1C=O]

DMP (70.6 g, 166 mmol) was added to a solution of (1S,2S)-ethyl 2-(hydroxymethyl)cyclopropanecarboxylate (16 g, 111 mmol) in DCM (600 mL). The mixture was stirred under nitrogen at 20° C. for 10 h. The mixture was filtered, then concentrated and purified by flash column (Petroleum ether/EtOAc=100:0 to 10:1) to afford product (1S,2S)-ethyl 2-formylcyclopropanecarboxylate.

¹H NMR (500 MHz, chloroform-d) δ=9.31 (d, J=4.0 Hz, 1H), 4.27-4.15 (m, 2H), 2.41-2.44 (m, 1H), 2.32-2.22 (m, 1H), 1.61-1.62 (m, 1H), 1.57-1.49 (m, 1H), 1.30-1.27 (m, 3H)

### Step 3:

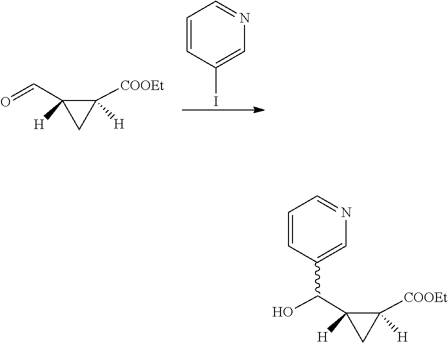 [SMILES: CCOC(=O)[C@H]1C[C@@H]1C=O ; Ic1cccnc1 ; CCOC(=O)[C@H]1C[C@@H]1C(O)c1cccnc1]

Isopropylmagnesium lithium chloride (30.3 mL, 39.4 mmol) was added dropwise to a solution of 3-iodopyridine (8.08 g, 39.4 mmol) and zinc(II) chloride (1.970 mL, 1.970 mmol) in THF (50 mL) at 0° C. under N2 atmosphere. Then the mixture was stirred at 15° C. for 20 min. Then the mixture was cooled to −20° C. (1S,2S)-ethyl 2-formylcyclopropanecarboxylate (4 g, 19.70 mmol) in THF (20 mL) was added to the mixture dropwise. The mixture was stirred at −20° C. for 1 h. The mixture was quenched with sat. a.q. NH₄Cl (40 mL), and extracted with EtOAc (50 mL×3). The combined organic layers were washed with brine (50 mL), dried over Na₂SO₄, filtered, concentrated in vacuo. The residue was purified by flash column (Petroleum ether/EtOAc=10:1 to 1:1) to afford (1S,2S)-ethyl 2-(hydroxy(pyridin-3-yl)methyl)cyclopropanecarboxylate. MS (ESI) m/z 222.1 (M+H⁺).

¹H NMR (500 MHz, chloroform-d) δ 8.57 (s, 1H), 8.51 (td, J₁=1.5 Hz, J₂=5.0 Hz, 1H), 7.84-7.72 (m, 1H), 7.31 (td, J₁=4.0 Hz, J₂=8.0 Hz, 1H), 4.52 (d, J=6.0 Hz, 1H), 4.29 (d, J=7.0 Hz, 1H), 4.16-4.03 (m, 2H), 1.89-1.76 (m, 2H), 1.29-1.22 (m, 4H), 1.19-1.11 (m, 1H), 1.06-0.96 (m, 1H)

### Step 4:

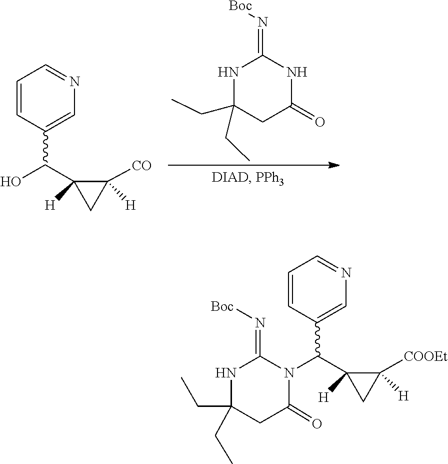

(E)-diisopropyl diazene-1,2-dicarboxylate (1.247 mL, 6.33 mmol) was added dropwise to a solution of (E)-tert-butyl (4,4-diethyl-6-oxotetrahydropyrimidin-2(1H)-ylidene)carbamate (0.852 g, 3.16 mmol), (1S,2S)-ethyl 2-(hydroxy(pyridin-3-yl)methyl)cyclopropanecarboxylate (0.7 g, 3.16 mmol) and triphenylphosphine (1.660 g, 6.33 mmol) in THF (20 mL) at 0° C. under N2 atmosphere. Then the mixture was stirred at 23° C. for 12 h. Water (20 mL) was added to the mixture, and extracted with EtOAc (15 mL×3). The combined organic layers were washed with brine (15 mL), dried over Na₂SO₄, filtered, concentrated in vacuo. The residue was purified by column chromatography (Petroleum ether:EtOAc=10:1-5:1), then purified by Prep-TLC (Petroleum ether:EtOAc=3:2) to afford (1S,2S)-ethyl 2-(((E)-2-((tert-butoxycarbonyl)imino)-4,4-diethyl-6-oxotetrahydropyrimidin-1(2H)-yl)(pyridin-3-yl)methyl)cyclopropanecarboxylate. MS (ESI) m/z 473.3 (M+H⁺).

¹H NMR (500 MHz, METHANOL-d4) δ=8.55 (d, J=2.0 Hz, 1H), 8.45-8.37 (m, 1H), 7.83 (d, J=8.5 Hz, 1H), 7.46-7.34 (m, 1H), 5.66-5.51 (m, 1H), 4.23-4.12 (m, 2H), 2.69 (s, 2H), 1.78-1.58 (m, 5H), 1.55-1.41 (m, 10H), 1.32-1.16 (m, 7H), 1.13-1.04 (m, 1H), 1.01-0.85 (m, 7H)

### Step 5:

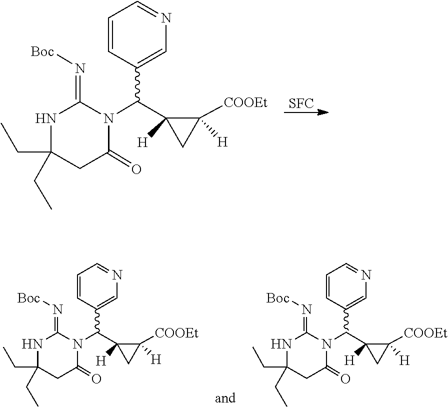 [SMILES: CCOC(=O)[C@H]1C[C@@H]1C(c1cccnc1)N1C(=O)CC(CC)(CC)N/C1=N\C(=O)OC(C)(C)C ; "SFC" ; CCOC(=O)[C@H]1C[C@@H]1C(c1cccnc1)N1C(=O)CC(CC)(CC)N/C1=N\C(=O)OC(C)(C)C ; CCOC(=O)[C@H]1C[C@@H]1C(c1cccnc1)N1C(=O)CC(CC)(CC)N/C1=N\C(=O)OC(C)(C)C]

(1S,2S)-ethyl 2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylate (440 mg, 0.931 mmol) was separated by SFC (Instrument SFC 13 Method Column DAICEL CHIRALPAK IC(250 mm*30 mm, 5 um) Condition 0.1% NH₃H₂O EtOH Begin B 35% End B 35% Gradient Time(min) 100% B Hold Time(min) FlowRate(mL/min) 60 Injections 120) to afford product (1S,2S)-ethyl 2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylate (Peak 1, Rt=3.403) and (1S,2S)-ethyl 2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylate (Peak 2, Rt=4.649).

MS (ESI) m/z 473.3 (M+H⁺)

### Step 6:

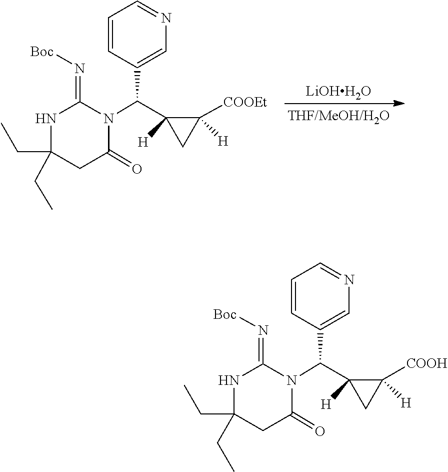 [SMILES: CCOC(=O)[C@H]1C[C@@H]1[C@@H](c1cccnc1)N1C(=O)CC(CC)(CC)N/C1=N\C(=O)OC(C)(C)C ; "LiOH·H2O" ; CCC1(CC)CC(=O)N([C@H](c2cccnc2)[C@H]2C[C@@H]2C(=O)O)/C(=N/C(=O)OC(C)(C)C)N1]

Lithium hydroxide monohydrate (124 mg, 2.96 mmol) was added to a solution of (1S,2S)-ethyl 2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylate (140 mg, 0.296 mmol) in MeOH (4 mL), THF (4 mL) and water (0.4 mL). The mixture was stirred at 25° C. for 2 hours. EtOAc (4 mL) was added to the mixture and the mixture was neutralized to pH 5-6 with citric acid. The mixture was quenched with water (10 mL), and extracted with EtOAc (15 mL×3). The organic layers were washed with brine (15 mL), dried over Na₂SO₄, filtered, and concentrated in vacuo to afford crude product (1S,2S)-2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylic acid, which was used for next step directly. MS (ESI) m/z 445.2 (M+H⁺)

### Step 7:

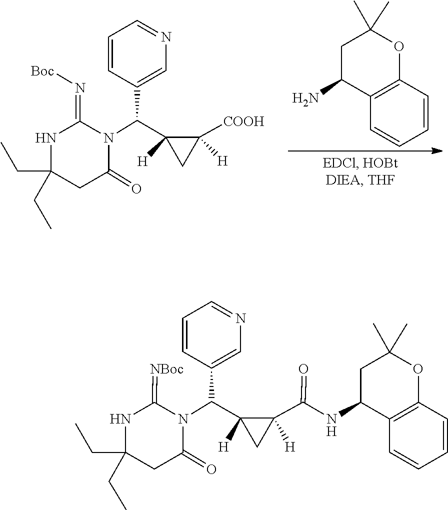

DIEA (0.196 mL, 1.125 mmol) was added to a solution of (1S,2S)-2-((S)-((E)-2-((tert-butoxycarbonyl)imino)-4,4-diethyl-6-oxotetrahydropyrimidin-1(2H)-yl)(pyridin-3-yl)methyl)cyclopropanecarboxylic acid (100 mg, crude), (S)-2,2-dimethylchroman-4-amine (61.7 mg, 0.225 mmol), HOBt (68.9 mg, 0.450 mmol), EDCI (86 mg, 0.450 mmol) in THF (8 mL). The mixture was stirred at 22° C. for 12 hours. The mixture was quenched with water (15 mL), and extracted with EtOAc (15 mL×3). The organic layers were washed with brine (15 mL), dried over Na₂SO₄, filtered, and concentrated in vacuo to afford crude product, then purified by Prep-TLC (Petroleum ether/EtOAc=1:2) to give the product tert-butyl (1-((S)-((1S,2S)-2-(((S)-2,2-dimethylchroman-4-yl)carbamoyl)cyclopropyl)(pyridin-3-yl)methyl)-4,4-diethyl-6-oxotetrahydropyrimidin-2(1H)-ylidene)carbamate. MS (ESI) m/z 604.3 (M+H⁺)

### Step 8:

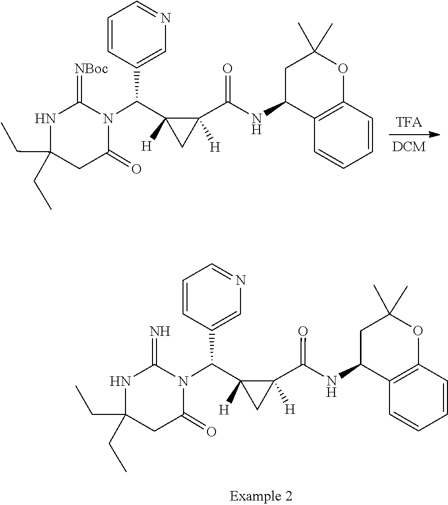

TFA (2 mL) was added to a solution of tert-butyl (1-((S)-((1S,2S)-2-(((S)-2,2-dimethylchroman-4-yl)carbamoyl)cyclopropyl)(pyridin-3-yl)methyl)-4,4-diethyl-6-oxotetrahydropyrimidin-2(1H)-ylidene)carbamate (90 mg, 0.149 mmol) in DCM (6 mL). The reaction was stirred at 23° C. for 1 h. The mixture was concentrated in vacuo, then purified by HPLC (Instrument Method Column YMC-Actus Pro C18 150*30 5u Condition water(0.1% TFA)-ACN Begin B 24 End B 54 Gradient Time(min) 11 100% B Hold Time(min) 1.1 FlowRate(mL/min) 40 Injections 3) to give the product (1S,2S)-2-((S)-(4,4-diethyl-2-imino-6-oxotetrahydropyrimidin-1(2H)-yl)(pyridin-3-yl)methyl)-N—((S)-2,2-dimethylchroman-4-yl)cyclopropanecarboxamide. MS (ESI) m/z 504.2 (M+H⁺).

¹H NMR (500 MHz, METHANOL-d4) δ 8.85 (s, 1H), 8.66 (s, 1H), 8.58 (d, J=8.5 Hz, 1H), 8.29 (d, J=8.0 Hz, 1H), 7.74 (s, 1H), 7.17-7.08 (m, 2H), 6.89-6.82 (m, 1H), 6.74 (d, J=8.0 Hz, 1H), 5.25-5.18 (m, 1H), 4.95 (d, J=3.0 Hz, 1H), 2.98-2.90 (m, 1H), 2.87-2.66 (m, 2H), 2.09-2.11 (m, 1H), 1.85-1.92 (m, 1H), 1.82 (t, J=12.5 Hz, 1H), 1.75-1.66 (m, 3H), 1.65-1.56 (m, 2H), 1.43 (s, 3H), 1.29 (s, 3H), 1.21 (s, 1H), 0.92 (t, J=7.5 Hz, 3H), 0.83 (t, J=7.5 Hz, 3H)

The compounds in Table 1 and 2 were made in accordance with the general schemes and Example 1 and 2 described above.

TABLE 1

| | | LC/MS |
|---|---|---|
| Ex. | Structure | (M + 1)+ |
| 1A | 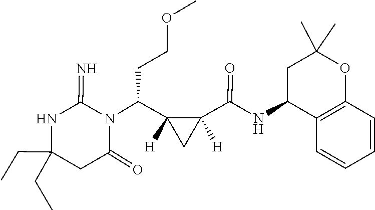 [SMILES: CCC1(CC)CC(=O)N([C@H](CCOC)[C@H]2C[C@@H]2C(=O)N[C@H]2CC(C)(C)Oc3ccccc32)C(=N)N1] | 485.3 |
| 1B | 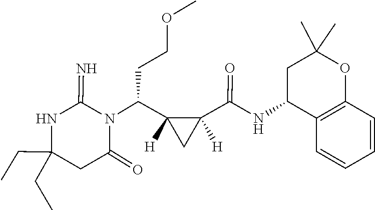 [SMILES: CCC1(CC)CC(=O)N([C@H](CCOC)[C@H]2C[C@@H]2C(=O)N[C@@H]2CC(C)(C)Oc3ccccc32)C(=N)N1] | 485.4 |
| 2 | 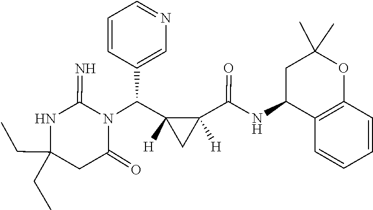 [SMILES: CCC1(CC)CC(=O)N([C@H](c2cccnc2)[C@H]2C[C@@H]2C(=O)N[C@H]2CC(C)(C)Oc3ccccc32)C(=N)N1] | 504.3 |
| 3A | 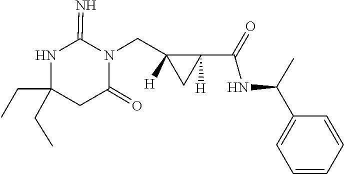 [SMILES: CCC1(CC)CC(=O)N(C[C@H]2C[C@@H]2C(=O)N[C@@H](C)c2ccccc2)C(=N)N1] | 371.1 |
| 3B | 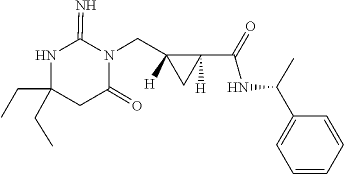 [SMILES: CCC1(CC)CC(=O)N(C[C@H]2C[C@@H]2C(=O)N[C@H](C)c2ccccc2)C(=N)N1] | 371.3 |
| 4A | 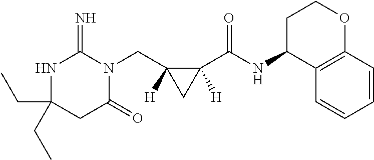 [SMILES: CCC1(CC)CC(=O)N(C[C@H]2C[C@@H]2C(=O)N[C@H]2CCOc3ccccc32)C(=N)N1] | 399.2 |
| 4B | 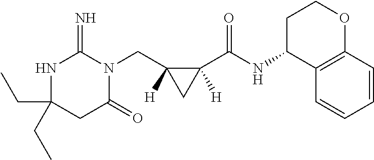 [SMILES: CCC1(CC)CC(=O)N(C[C@H]2C[C@@H]2C(=O)N[C@@H]2CCOc3ccccc32)C(=N)N1] | 399.2 |
| 4C | 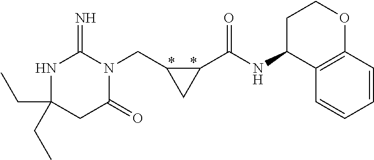 [SMILES: CCC1(CC)CC(=O)N(CC2CC2C(=O)N[C@H]2CCOc3ccccc32)C(=N)N1] | 399.3 |
| 4D | 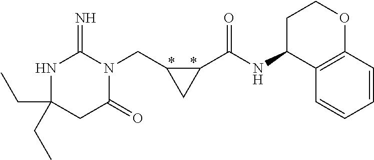 [SMILES: CCC1(CC)CC(=O)N(CC2CC2C(=O)N[C@H]2CCOc3ccccc32)C(=N)N1] | 399.2 |
| 5 | 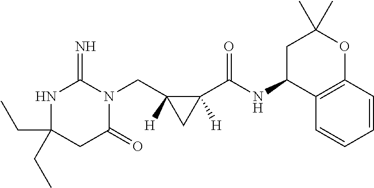 [SMILES: CCC1(CC)CC(=O)N(C[C@H]2C[C@@H]2C(=O)N[C@H]2CC(C)(C)Oc3ccccc32)C(=N)N1] | 427.3 |
| 6A | 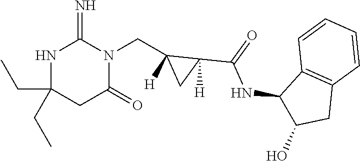 [SMILES: CCC1(CC)CC(=O)N(C[C@H]2C[C@@H]2C(=O)N[C@H]2c3ccccc3C[C@@H]2O)C(=N)N1] | 399.1 |
| 6B | 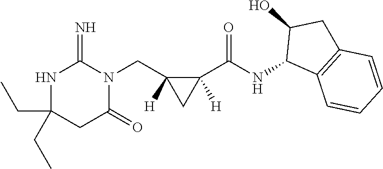 [SMILES: CCC1(CC)CC(=O)N(C[C@H]2C[C@@H]2C(=O)N[C@H]2c3ccccc3C[C@@H]2O)C(=N)N1] | 399.2 |
| 7A | 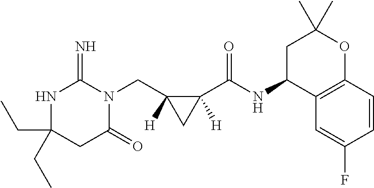 [SMILES: CCC1(CC)CC(=O)N(C[C@H]2C[C@@H]2C(=O)N[C@H]2CC(C)(C)Oc3ccc(F)cc32)C(=N)N1] | 445.3 |
| 7B | 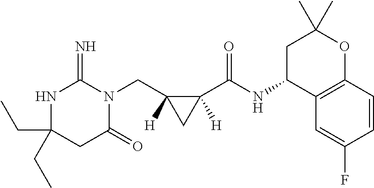 [SMILES: CCC1(CC)CC(=O)N(C[C@H]2C[C@@H]2C(=O)N[C@@H]2CC(C)(C)Oc3ccc(F)cc32)C(=N)N1] | 445.3 |
| 8A | 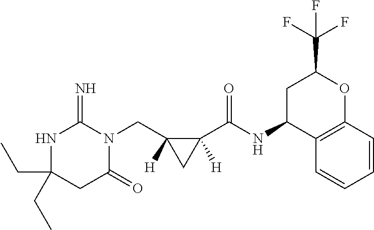 [SMILES: CCC1(CC)CC(=O)N(C[C@H]2C[C@@H]2C(=O)N[C@H]2C[C@@H](C(F)(F)F)Oc3ccccc32)C(=N)N1] | 467.3 |
| 8B | 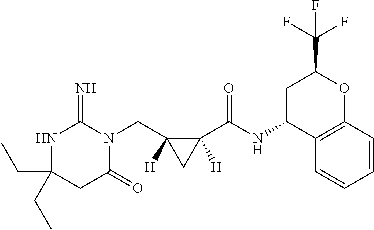 [SMILES: CCC1(CC)CC(=O)N(C[C@H]2C[C@@H]2C(=O)N[C@@H]2C[C@@H](C(F)(F)F)Oc3ccccc32)C(=N)N1] | 467.3 |
| 9 | 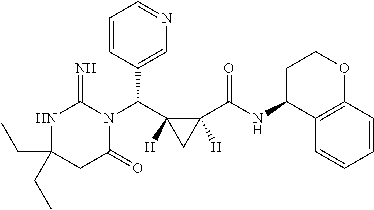 [SMILES: CCC1(CC)CC(=O)N([C@H](c2cccnc2)[C@H]2C[C@@H]2C(=O)N[C@H]2CCOc3ccccc32)C(=N)N1] | 476.3 |
| 10 | 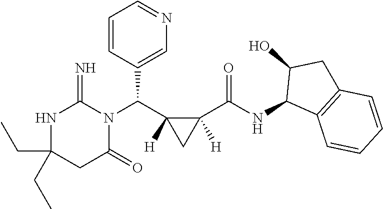 [SMILES: CCC1(CC)CC(=O)N([C@H](c2cccnc2)[C@H]2C[C@@H]2C(=O)N[C@@H]2c3ccccc3C[C@@H]2O)C(=N)N1] | 476.3 |
| 11A | 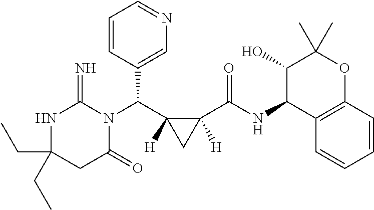 [SMILES: CCC1(CC)CC(=O)N([C@H](c2cccnc2)[C@H]2C[C@@H]2C(=O)N[C@@H]2c3ccccc3OC(C)(C)[C@H]2O)C(=N)N1] | 520.3 |
| 11B | 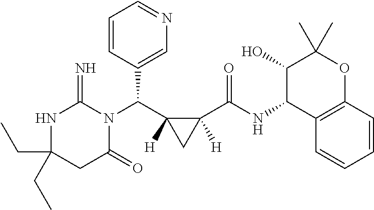 [SMILES: CCC1(CC)CC(=O)N([C@H](c2cccnc2)[C@H]2C[C@@H]2C(=O)N[C@H]2c3ccccc3OC(C)(C)[C@H]2O)C(=N)N1] | 520.3 |
| 12 | 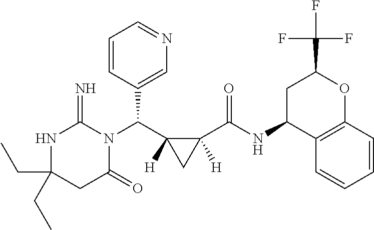 [SMILES: CCC1(CC)CC(=O)N([C@H](c2cccnc2)[C@H]2C[C@@H]2C(=O)N[C@H]2C[C@@H](C(F)(F)F)Oc3ccccc32)C(=N)N1] | 544.3 |
| 13 | 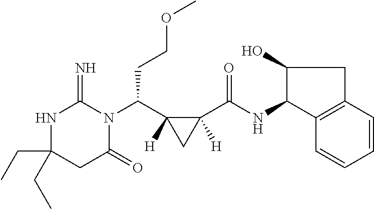 [SMILES: CCC1(CC)CC(=O)N([C@H](CCOC)[C@H]2C[C@@H]2C(=O)N[C@@H]2c3ccccc3C[C@@H]2O)C(=N)N1] | 457.4 |
| 14 | 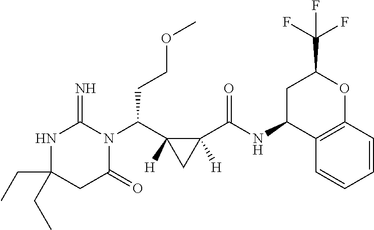 [SMILES: CCC1(CC)CC(=O)N([C@H](CCOC)[C@H]2C[C@@H]2C(=O)N[C@H]2C[C@@H](C(F)(F)F)Oc3ccccc32)C(=N)N1] | 525.3 |
| 15A | 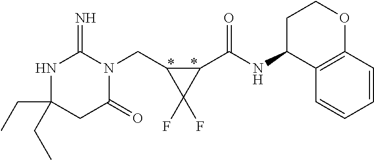 [SMILES: CCC1(CC)CC(=O)N(CC2C(C(=O)N[C@H]3CCOc4ccccc43)C2(F)F)C(=N)N1] | 435.2 |
| 15B | 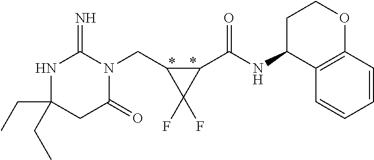 [SMILES: CCC1(CC)CC(=O)N(CC2C(C(=O)N[C@H]3CCOc4ccccc43)C2(F)F)C(=N)N1] | 435.2 |
| 16A | 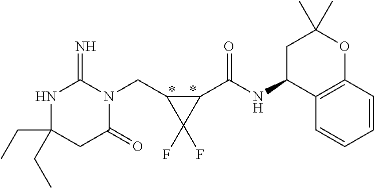 [SMILES: CCC1(CC)CC(=O)N(CC2C(C(=O)N[C@H]3CC(C)(C)Oc4ccccc43)C2(F)F)C(=N)N1] | 463.3 |
| 16B | 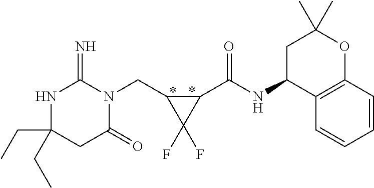 [SMILES: CCC1(CC)CC(=O)N(CC2C(C(=O)N[C@H]3CC(C)(C)Oc4ccccc43)C2(F)F)C(=N)N1] | 463.3 |
| 17A | 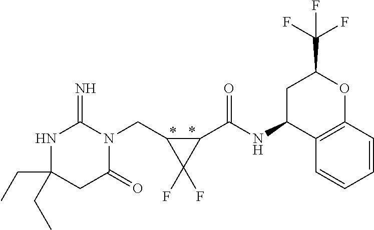 [SMILES: CCC1(CC)CC(=O)N(CC2C(C(=O)N[C@H]3C[C@@H](C(F)(F)F)Oc4ccccc43)C2(F)F)C(=N)N1] | 503.2 |
| 17B | 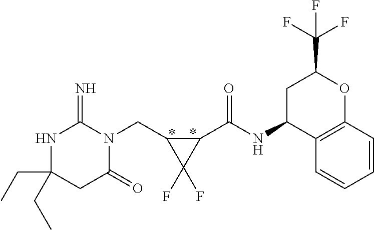 [SMILES: CCC1(CC)CC(=O)N(CC2C(C(=O)N[C@H]3C[C@@H](C(F)(F)F)Oc4ccccc43)C2(F)F)C(=N)N1] | 503.2 |
| 18A | 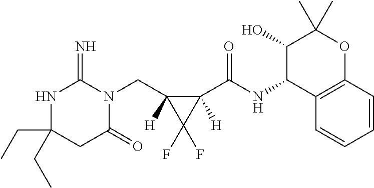 [SMILES: CCC1(CC)CC(=O)N(C[C@@H]2[C@@H](C(=O)N[C@H]3c4ccccc4OC(C)(C)[C@H]3O)C2(F)F)C(=N)N1] | 479.2 |
| 18B | 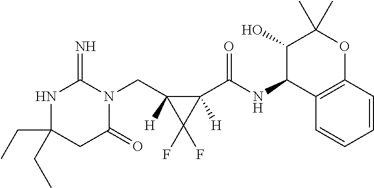 [SMILES: CCC1(CC)CC(=O)N(C[C@@H]2[C@@H](C(=O)N[C@@H]3c4ccccc4OC(C)(C)[C@H]3O)C2(F)F)C(=N)N1] | 479.3 |
| 19A | 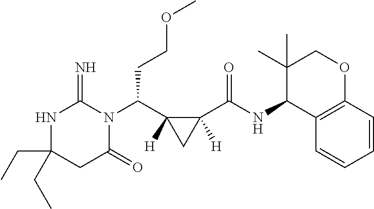 [SMILES: CCC1(CC)CC(=O)N([C@H](CCOC)[C@H]2C[C@@H]2C(=O)N[C@@H]2c3ccccc3OCC2(C)C)C(=N)N1] | 485.3 |
| 19B | 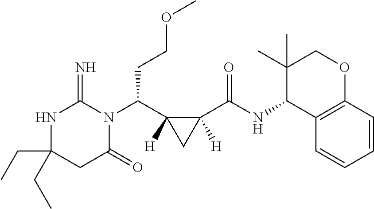 [SMILES: CCC1(CC)CC(=O)N([C@H](CCOC)[C@H]2C[C@@H]2C(=O)N[C@H]2c3ccccc3OCC2(C)C)C(=N)N1] | 485.4 |
| 20 | 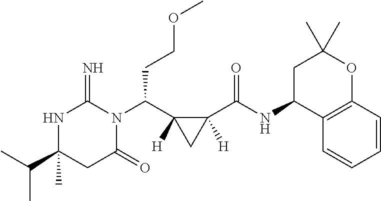 [SMILES: COCC[C@H]([C@H]1C[C@@H]1C(=O)N[C@H]1CC(C)(C)Oc2ccccc21)N1C(=N)N[C@](C)(C(C)C)CC1=O] | 485.3 |
| 21A | 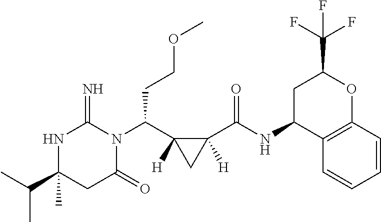 [SMILES: COCC[C@H]([C@H]1C[C@@H]1C(=O)N[C@H]1C[C@@H](C(F)(F)F)Oc2ccccc21)N1C(=N)N[C@](C)(C(C)C)CC1=O] | 525.2 |
| 21B | 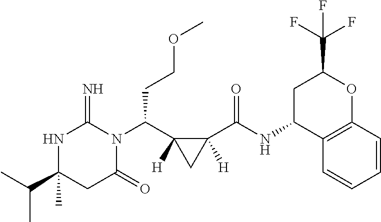 [SMILES: COCC[C@H]([C@H]1C[C@@H]1C(=O)N[C@@H]1C[C@@H](C(F)(F)F)Oc2ccccc21)N1C(=N)N[C@](C)(C(C)C)CC1=O] | 525.2 |
| 22 | 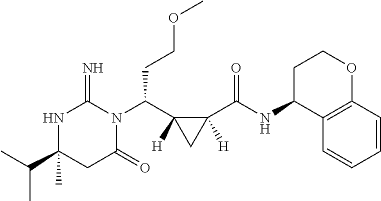 [SMILES: COCC[C@H]([C@H]1C[C@@H]1C(=O)N[C@H]1CCOc2ccccc21)N1C(=N)N[C@](C)(C(C)C)CC1=O] | 457.2 |
| 23A | 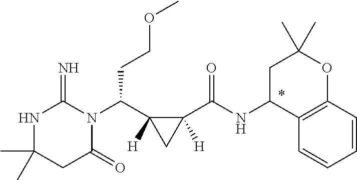 [SMILES: COCC[C@H]([C@H]1C[C@@H]1C(=O)NC1CC(C)(C)Oc2ccccc21)N1C(=N)NC(C)(C)CC1=O] | 457.3 |
| 23B | 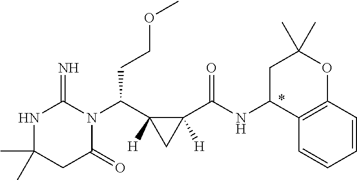 [SMILES: COCC[C@H]([C@H]1C[C@@H]1C(=O)NC1CC(C)(C)Oc2ccccc21)N1C(=N)NC(C)(C)CC1=O] | 457.3 |
| 24A | 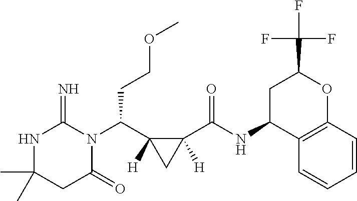 [SMILES: COCC[C@H]([C@H]1C[C@@H]1C(=O)N[C@H]1C[C@@H](C(F)(F)F)Oc2ccccc21)N1C(=N)NC(C)(C)CC1=O] | 497.2 |
| 24B | 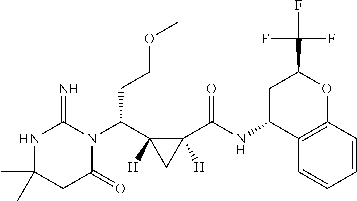 [SMILES: COCC[C@H]([C@H]1C[C@@H]1C(=O)N[C@@H]1C[C@@H](C(F)(F)F)Oc2ccccc21)N1C(=N)NC(C)(C)CC1=O] | 497.2 |
| 24C | 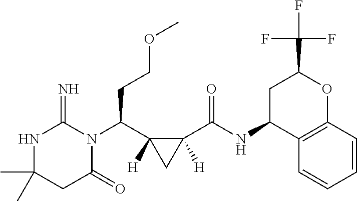 [SMILES: COCC[C@@H]([C@H]1C[C@@H]1C(=O)N[C@H]1C[C@@H](C(F)(F)F)Oc2ccccc21)N1C(=N)NC(C)(C)CC1=O] | 497.1 |
| 25A | 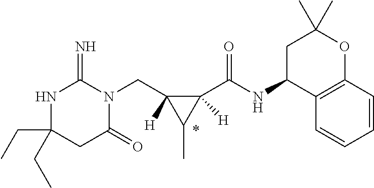 [SMILES: CCC1(CC)CC(=O)N(C[C@H]2C(C)[C@@H]2C(=O)N[C@H]2CC(C)(C)Oc3ccccc32)C(=N)N1] | 441.3 |
| 25B | 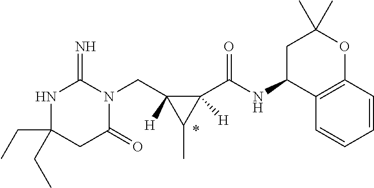 [SMILES: CCC1(CC)CC(=O)N(C[C@H]2C(C)[C@@H]2C(=O)N[C@H]2CC(C)(C)Oc3ccccc32)C(=N)N1] | 441.3 |
| 26 | 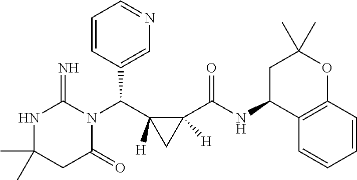 [SMILES: CC1(C)CC(=O)N([C@H](c2cccnc2)[C@H]2C[C@@H]2C(=O)N[C@H]2CC(C)(C)Oc3ccccc32)C(=N)N1] | 476.3 |
| 27 | 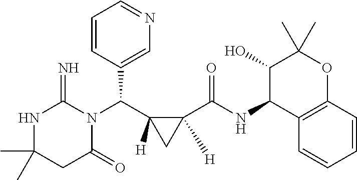 [SMILES: CC1(C)CC(=O)N([C@H](c2cccnc2)[C@H]2C[C@@H]2C(=O)N[C@@H]2c3ccccc3OC(C)(C)[C@H]2O)C(=N)N1] | 492.3 |
| 28A | 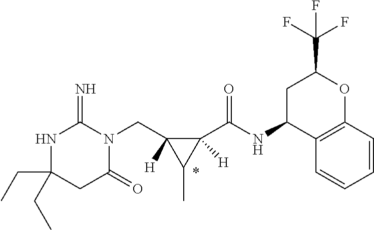 [SMILES: CCC1(CC)CC(=O)N(C[C@H]2C(C)[C@@H]2C(=O)N[C@H]2C[C@@H](C(F)(F)F)Oc3ccccc32)C(=N)N1] | 481.3 |
| 28B | 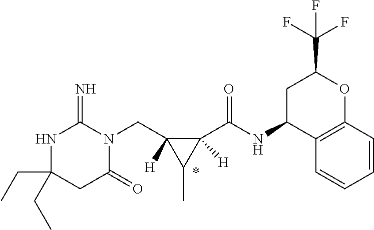 [SMILES: CCC1(CC)CC(=O)N(C[C@H]2C(C)[C@@H]2C(=O)N[C@H]2C[C@@H](C(F)(F)F)Oc3ccccc32)C(=N)N1] | 481.3 |
| 29A | 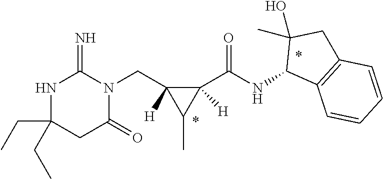 [SMILES: CCC1(CC)CC(=O)N(C[C@H]2C(C)[C@@H]2C(=O)N[C@H]2c3ccccc3CC2(C)O)C(=N)N1] | 427.3 |
| 29B | 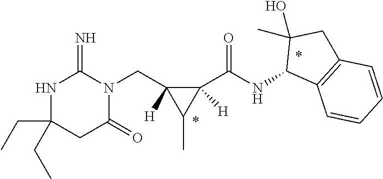 [SMILES: CCC1(CC)CC(=O)N(C[C@H]2C(C)[C@@H]2C(=O)N[C@H]2c3ccccc3CC2(C)O)C(=N)N1] | 427.3 |
| 29C | 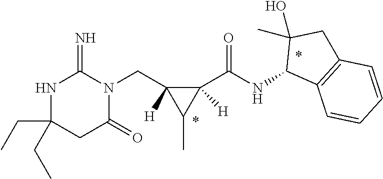 [SMILES: CCC1(CC)CC(=O)N(C[C@H]2C(C)[C@@H]2C(=O)N[C@H]2c3ccccc3CC2(C)O)C(=N)N1] | 427.3 |
| 30A | 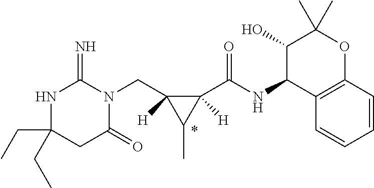 [SMILES: CCC1(CC)CC(=O)N(C[C@H]2C(C)[C@@H]2C(=O)N[C@@H]2c3ccccc3OC(C)(C)[C@H]2O)C(=N)N1] | 457.3 |
| 30B | 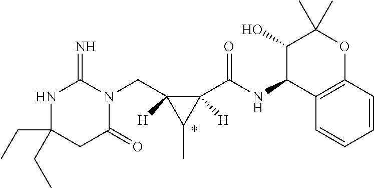 [SMILES: CCC1(CC)CC(=O)N(C[C@H]2C(C)[C@@H]2C(=O)N[C@@H]2c3ccccc3OC(C)(C)[C@H]2O)C(=N)N1] | 457.3 |
| 31A | 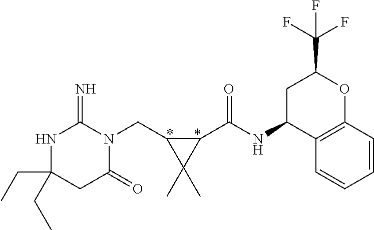 [SMILES: CCC1(CC)CC(=O)N(CC2C(C(=O)N[C@H]3C[C@@H](C(F)(F)F)Oc4ccccc43)C2(C)C)C(=N)N1] | 495.3 |
| 31B | 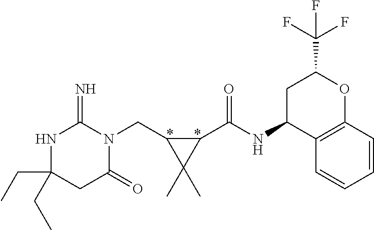 [SMILES: CCC1(CC)CC(=O)N(CC2C(C(=O)N[C@H]3C[C@H](C(F)(F)F)Oc4ccccc43)C2(C)C)C(=N)N1] | 495.2 |
| 32 | 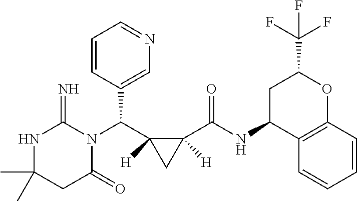 [SMILES: CC1(C)CC(=O)N([C@H](c2cccnc2)[C@H]2C[C@@H]2C(=O)N[C@H]2C[C@H](C(F)(F)F)Oc3ccccc32)C(=N)N1] | 516.2 |
| 33 | 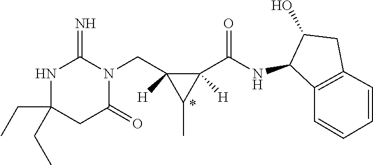 [SMILES: CCC1(CC)CC(=O)N(C[C@H]2C(C)[C@@H]2C(=O)N[C@@H]2c3ccccc3C[C@H]2O)C(=N)N1] | 413.3 |
| 34 | 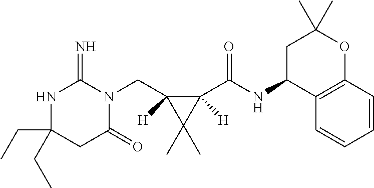 [SMILES: CCC1(CC)CC(=O)N(C[C@@H]2[C@@H](C(=O)N[C@H]3CC(C)(C)Oc4ccccc43)C2(C)C)C(=N)N1] | 455.3 |
| 35A | 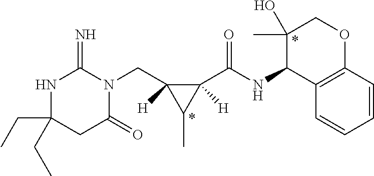 [SMILES: CCC1(CC)CC(=O)N(C[C@H]2C(C)[C@@H]2C(=O)N[C@@H]2c3ccccc3OCC2(C)O)C(=N)N1] | 443.3 |
| 35B | 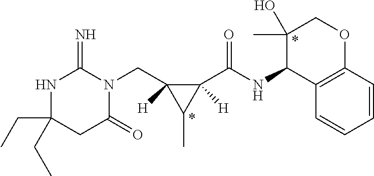 [SMILES: CCC1(CC)CC(=O)N(C[C@H]2C(C)[C@@H]2C(=O)N[C@@H]2c3ccccc3OCC2(C)O)C(=N)N1] | 443.1 |
| 36A | 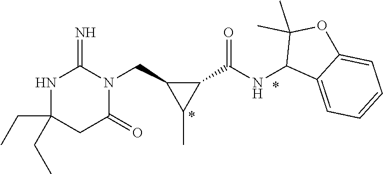 [SMILES: CCC1(CC)CC(=O)N(C[C@@H]2C(C)[C@H]2C(=O)NC2c3ccccc3OC2(C)C)C(=N)N1] | 427.3 |
| 36B | 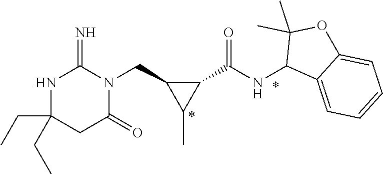 [SMILES: CCC1(CC)CC(=O)N(C[C@@H]2C(C)[C@H]2C(=O)NC2c3ccccc3OC2(C)C)C(=N)N1] | 427.3 |
| 37 | 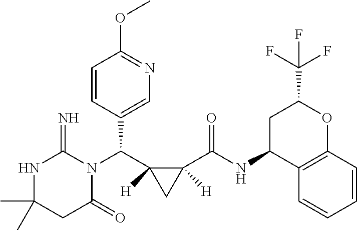 [SMILES: COc1ccc([C@H]([C@H]2C[C@@H]2C(=O)N[C@H]2C[C@H](C(F)(F)F)Oc3ccccc32)N2C(=N)NC(C)(C)CC2=O)cn1] | 546.2 |
| 38 | 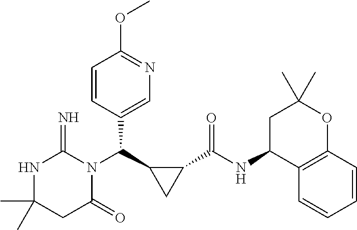 [SMILES: COc1ccc([C@H]([C@@H]2C[C@H]2C(=O)N[C@H]2CC(C)(C)Oc3ccccc32)N2C(=N)NC(C)(C)CC2=O)cn1] | 506.3 |
| 39 | 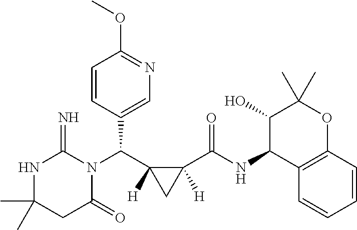 [SMILES: COc1ccc([C@H]([C@H]2C[C@@H]2C(=O)N[C@@H]2c3ccccc3OC(C)(C)[C@H]2O)N2C(=N)NC(C)(C)CC2=O)cn1] | 522.3 |
| 40 | 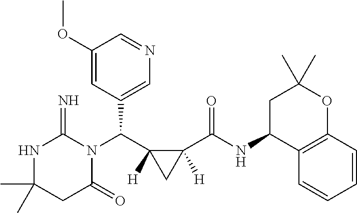 [SMILES: COc1cncc([C@H]([C@H]2C[C@@H]2C(=O)N[C@H]2CC(C)(C)Oc3ccccc32)N2C(=N)NC(C)(C)CC2=O)c1] | 506.2 |
| 41 | 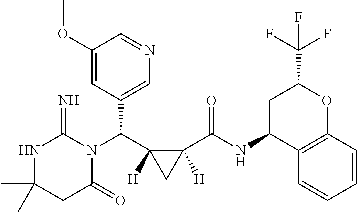 [SMILES: COc1cncc([C@H]([C@H]2C[C@@H]2C(=O)N[C@H]2C[C@H](C(F)(F)F)Oc3ccccc32)N2C(=N)NC(C)(C)CC2=O)c1] | 546.1 |
| 42 | 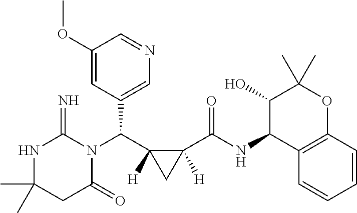 [SMILES: COc1cncc([C@H]([C@H]2C[C@@H]2C(=O)N[C@@H]2c3ccccc3OC(C)(C)[C@H]2O)N2C(=N)NC(C)(C)CC2=O)c1] | 522.2 |
| 43A | 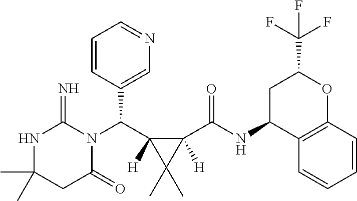 [SMILES: CC1(C)CC(=O)N([C@H](c2cccnc2)[C@@H]2[C@@H](C(=O)N[C@H]3C[C@H](C(F)(F)F)Oc4ccccc43)C2(C)C)C(=N)N1] | 544.2 |
| 43B | 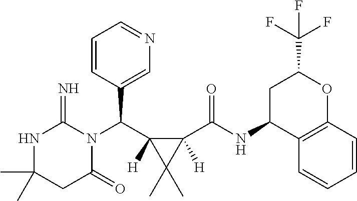 [SMILES: CC1(C)CC(=O)N([C@@H](c2cccnc2)[C@@H]2[C@@H](C(=O)N[C@H]3C[C@H](C(F)(F)F)Oc4ccccc43)C2(C)C)C(=N)N1] | 544.2 |
| 44A | 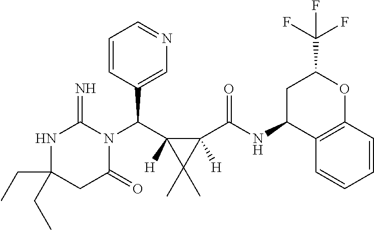 [SMILES: CCC1(CC)CC(=O)N([C@@H](c2cccnc2)[C@@H]2[C@@H](C(=O)N[C@H]3C[C@H](C(F)(F)F)Oc4ccccc43)C2(C)C)C(=N)N1] | 572.2 |
| 44B | 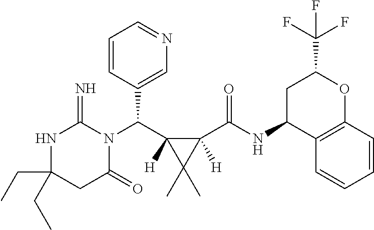 [SMILES: CCC1(CC)CC(=O)N([C@H](c2cccnc2)[C@@H]2[C@@H](C(=O)N[C@H]3C[C@H](C(F)(F)F)Oc4ccccc43)C2(C)C)C(=N)N1] | 572.3 |
| 45A | 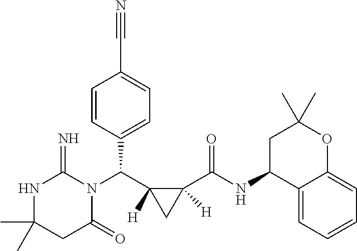 [SMILES: CC1(C)CC(=O)N([C@H](c2ccc(C#N)cc2)[C@H]2C[C@@H]2C(=O)N[C@H]2CC(C)(C)Oc3ccccc32)C(=N)N1] | 500.2 |
| 45B | 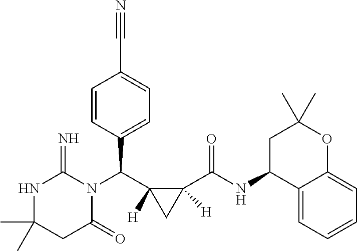 [SMILES: CC1(C)CC(=O)N([C@@H](c2ccc(C#N)cc2)[C@H]2C[C@@H]2C(=O)N[C@H]2CC(C)(C)Oc3ccccc32)C(=N)N1] | 500.2 |
| 46 | 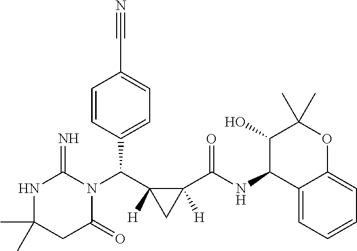 [SMILES: CC1(C)CC(=O)N([C@H](c2ccc(C#N)cc2)[C@H]2C[C@@H]2C(=O)N[C@@H]2c3ccccc3OC(C)(C)[C@H]2O)C(=N)N1] | 516.2 |
| 47A | 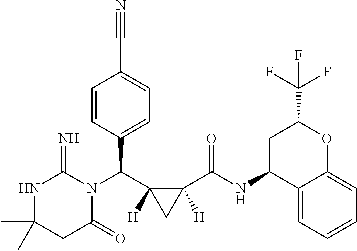 [SMILES: CC1(C)CC(=O)N([C@@H](c2ccc(C#N)cc2)[C@H]2C[C@@H]2C(=O)N[C@H]2C[C@H](C(F)(F)F)Oc3ccccc32)C(=N)N1] | 540.2 |
| 47B | 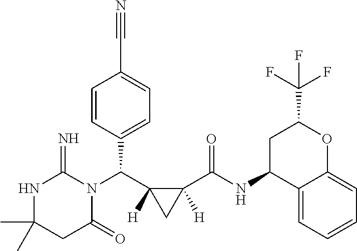 [SMILES: CC1(C)CC(=O)N([C@H](c2ccc(C#N)cc2)[C@H]2C[C@@H]2C(=O)N[C@H]2C[C@H](C(F)(F)F)Oc3ccccc32)C(=N)N1] | 540.1 |
| 48A | 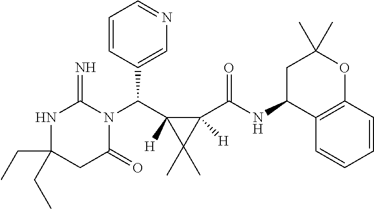 [SMILES: CCC1(CC)CC(=O)N([C@H](c2cccnc2)[C@@H]2[C@@H](C(=O)N[C@H]3CC(C)(C)Oc4ccccc43)C2(C)C)C(=N)N1] | 532.3 |
| 48B | 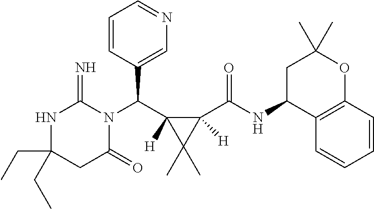 [SMILES: CCC1(CC)CC(=O)N([C@@H](c2cccnc2)[C@@H]2[C@@H](C(=O)N[C@H]3CC(C)(C)Oc4ccccc43)C2(C)C)C(=N)N1] | 532.3 |
| 49A | 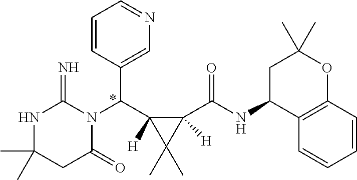 [SMILES: CC1(C)CC(=O)N(C(c2cccnc2)[C@@H]2[C@@H](C(=O)N[C@H]3CC(C)(C)Oc4ccccc43)C2(C)C)C(=N)N1] | 504.3 |
| 49B | 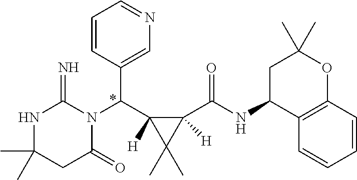 [SMILES: CC1(C)CC(=O)N(C(c2cccnc2)[C@@H]2[C@@H](C(=O)N[C@H]3CC(C)(C)Oc4ccccc43)C2(C)C)C(=N)N1] | 504.2 |
| 50 | 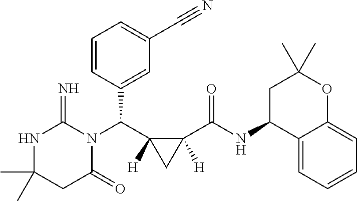 [SMILES: CC1(C)CC(=O)N([C@H](c2cccc(C#N)c2)[C@H]2C[C@@H]2C(=O)N[C@H]2CC(C)(C)Oc3ccccc32)C(=N)N1] | 500.2 |
| 51 | 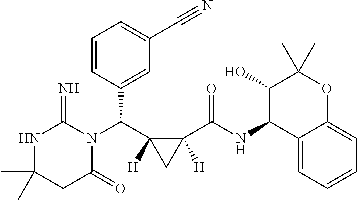 [SMILES: CC1(C)CC(=O)N([C@H](c2cccc(C#N)c2)[C@H]2C[C@@H]2C(=O)N[C@@H]2c3ccccc3OC(C)(C)[C@H]2O)C(=N)N1] | 516.2 |
| 52 | 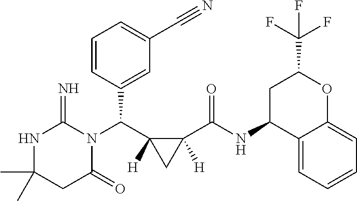 [SMILES: CC1(C)CC(=O)N([C@H](c2cccc(C#N)c2)[C@H]2C[C@@H]2C(=O)N[C@H]2C[C@H](C(F)(F)F)Oc3ccccc32)C(=N)N1] | 540.2 |
| 53A | 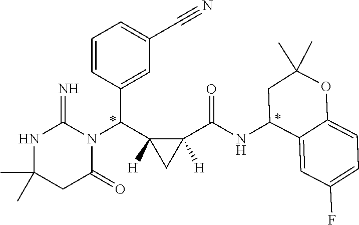 [SMILES: CC1(C)CC(=O)N(C(c2cccc(C#N)c2)[C@H]2C[C@@H]2C(=O)NC2CC(C)(C)Oc3ccc(F)cc32)C(=N)N1] | 518.2 |
| 53B | 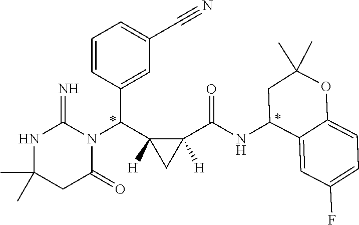 [SMILES: CC1(C)CC(=O)N(C(c2cccc(C#N)c2)[C@H]2C[C@@H]2C(=O)NC2CC(C)(C)Oc3ccc(F)cc32)C(=N)N1] | 518.3 |
| 54A | 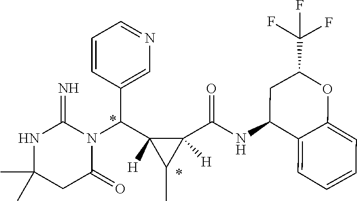 [SMILES: CC1[C@H](C(c2cccnc2)N2C(=N)NC(C)(C)CC2=O)[C@H]1C(=O)N[C@H]1C[C@H](C(F)(F)F)Oc2ccccc21] | 530.2 |
| 54B | 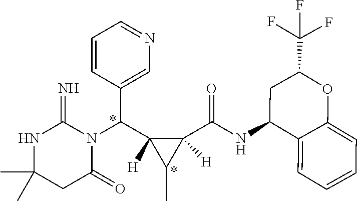 [SMILES: CC1[C@H](C(c2cccnc2)N2C(=N)NC(C)(C)CC2=O)[C@H]1C(=O)N[C@H]1C[C@H](C(F)(F)F)Oc2ccccc21] | 530 |
| 55A | 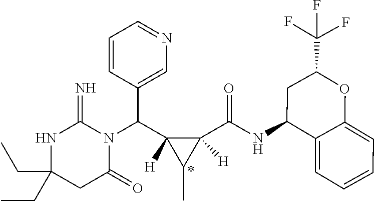 [SMILES: CCC1(CC)CC(=O)N(C(c2cccnc2)[C@H]2C(C)[C@@H]2C(=O)N[C@H]2C[C@H](C(F)(F)F)Oc3ccccc32)C(=N)N1] | 558.3 |
| 55B | 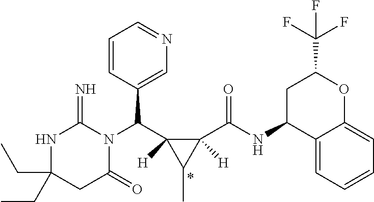 | 558 |
| 56A | 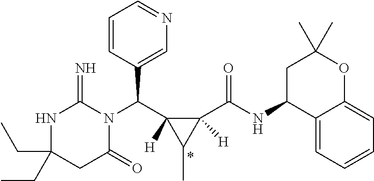 [SMILES: CCC1(CC)CC(=O)N([C@@H](c2cccnc2)[C@H]2C(C)[C@@H]2C(=O)N[C@H]2CC(C)(C)Oc3ccccc32)C(=N)N1] | 518.1 |
| 56B | 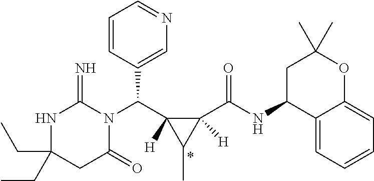 [SMILES: CCC1(CC)CC(=O)N([C@H](c2cccnc2)[C@H]2C(C)[C@@H]2C(=O)N[C@H]2CC(C)(C)Oc3ccccc32)C(=N)N1] | 518.1 |
| 57A | 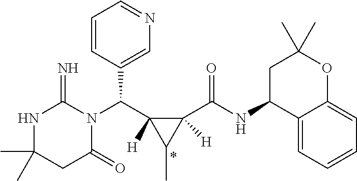 [SMILES: CC1[C@H]([C@@H](c2cccnc2)N2C(=N)NC(C)(C)CC2=O)[C@H]1C(=O)N[C@H]1CC(C)(C)Oc2ccccc21] | 490.3 |
| 57B | 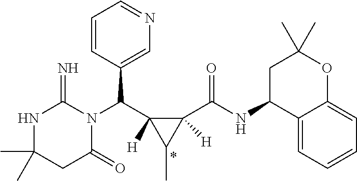 | 490.1 |
| 58A | 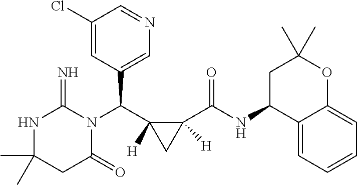 [SMILES: CC1(C)CC(=O)N([C@@H](c2cncc(Cl)c2)[C@H]2C[C@@H]2C(=O)N[C@H]2CC(C)(C)Oc3ccccc32)C(=N)N1] | 510.2 |
| 58B | 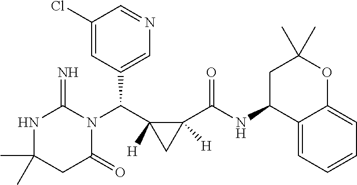 [SMILES: CC1(C)CC(=O)N([C@H](c2cncc(Cl)c2)[C@H]2C[C@@H]2C(=O)N[C@H]2CC(C)(C)Oc3ccccc32)C(=N)N1] | 510.2 |
| 59 | 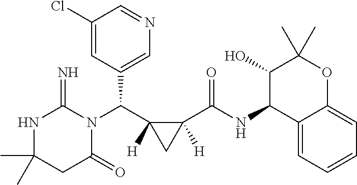 [SMILES: CC1(C)CC(=O)N([C@H](c2cncc(Cl)c2)[C@H]2C[C@@H]2C(=O)N[C@@H]2c3ccccc3OC(C)(C)[C@H]2O)C(=N)N1] | 526.2 |
| 60A | 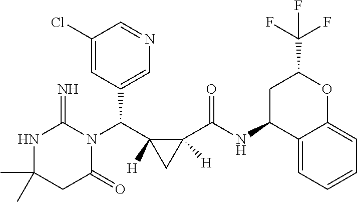 [SMILES: CC1(C)CC(=O)N([C@H](c2cncc(Cl)c2)[C@H]2C[C@@H]2C(=O)N[C@H]2C[C@H](C(F)(F)F)Oc3ccccc32)C(=N)N1] | 550.2 |
| 60B | 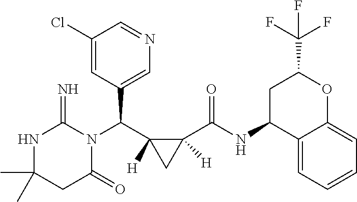 [SMILES: CC1(C)CC(=O)N([C@@H](c2cncc(Cl)c2)[C@H]2C[C@@H]2C(=O)N[C@H]2C[C@H](C(F)(F)F)Oc3ccccc32)C(=N)N1] | 550.2 |
| 61 | 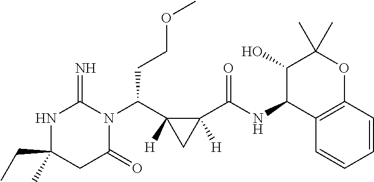 [SMILES: CC[C@]1(C)CC(=O)N([C@H](CCOC)[C@H]2C[C@@H]2C(=O)N[C@@H]2c3ccccc3OC(C)(C)[C@H]2O)C(=N)N1] | 487.3 |
| 62A | 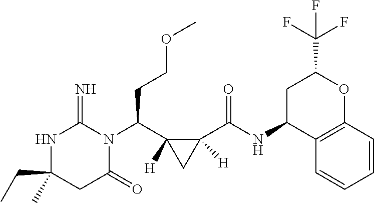 [SMILES: CC[C@]1(C)CC(=O)N([C@@H](CCOC)[C@H]2C[C@@H]2C(=O)N[C@H]2C[C@H](C(F)(F)F)Oc3ccccc32)C(=N)N1] | 511.3 |
| 62B | 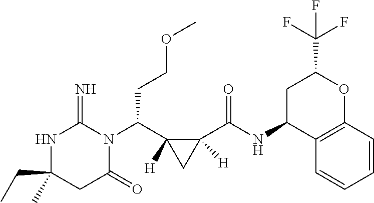 [SMILES: CC[C@]1(C)CC(=O)N([C@H](CCOC)[C@H]2C[C@@H]2C(=O)N[C@H]2C[C@H](C(F)(F)F)Oc3ccccc32)C(=N)N1] | 511.3 |
| 63 | 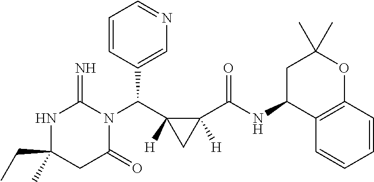 [SMILES: CC[C@]1(C)CC(=O)N([C@H](c2cccnc2)[C@H]2C[C@@H]2C(=O)N[C@H]2CC(C)(C)Oc3ccccc32)C(=N)N1] | 490.3 |
| 64A | 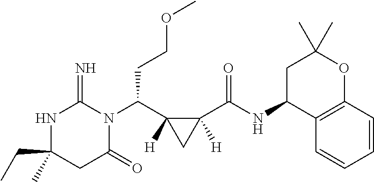 [SMILES: CC[C@]1(C)CC(=O)N([C@H](CCOC)[C@H]2C[C@@H]2C(=O)N[C@H]2CC(C)(C)Oc3ccccc32)C(=N)N1] | 471.3 |
| 64B | 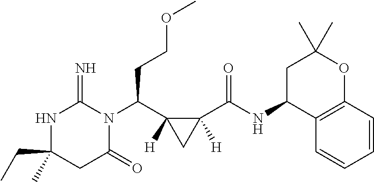 [SMILES: CC[C@]1(C)CC(=O)N([C@@H](CCOC)[C@H]2C[C@@H]2C(=O)N[C@H]2CC(C)(C)Oc3ccccc32)C(=N)N1] | 471.3 |
| 65A | 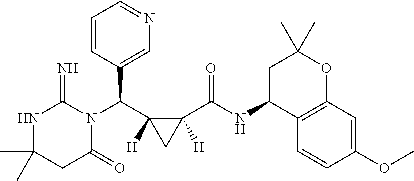 | 506.3 |
| 65B | 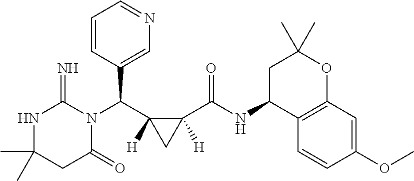 | 506.3 |
| 66 | 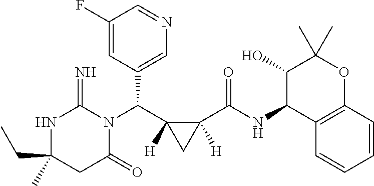 [SMILES: CC[C@]1(C)CC(=O)N([C@H](c2cncc(F)c2)[C@H]2C[C@@H]2C(=O)N[C@@H]2c3ccccc3OC(C)(C)[C@H]2O)C(=N)N1] | 524.3 |
| 67A | 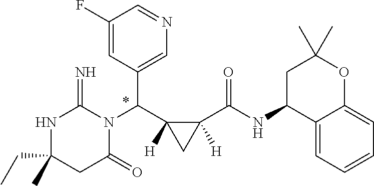 [SMILES: CC[C@@]1(C)CC(=O)N(C(c2cncc(F)c2)[C@H]2C[C@@H]2C(=O)N[C@H]2CC(C)(C)Oc3ccccc32)C(=N)N1] | 508.3 |
| 67B | 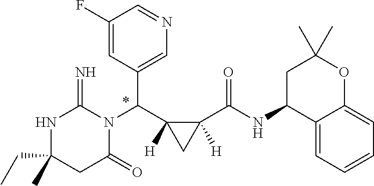 [SMILES: CC[C@@]1(C)CC(=O)N(C(c2cncc(F)c2)[C@H]2C[C@@H]2C(=O)N[C@H]2CC(C)(C)Oc3ccccc32)C(=N)N1] | 508.3 |
| 68 | 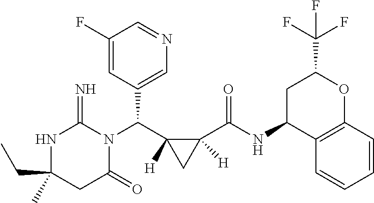 [SMILES: CC[C@]1(C)CC(=O)N([C@H](c2cncc(F)c2)[C@H]2C[C@@H]2C(=O)N[C@H]2C[C@H](C(F)(F)F)Oc3ccccc32)C(=N)N1] | 548.2 |
| 69A | 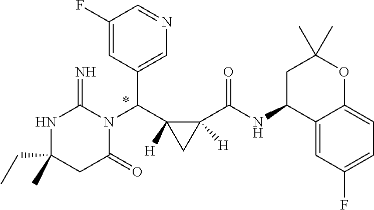 [SMILES: CC[C@@]1(C)CC(=O)N(C(c2cncc(F)c2)[C@H]2C[C@@H]2C(=O)N[C@H]2CC(C)(C)Oc3ccc(F)cc32)C(=N)N1] | 526.2 |
| 69B | 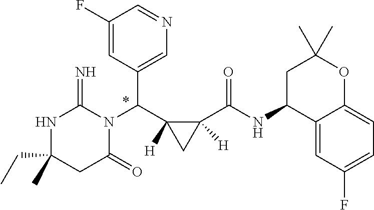 [SMILES: CC[C@@]1(C)CC(=O)N(C(c2cncc(F)c2)[C@H]2C[C@@H]2C(=O)N[C@H]2CC(C)(C)Oc3ccc(F)cc32)C(=N)N1] | 526.3 |
| 69C | 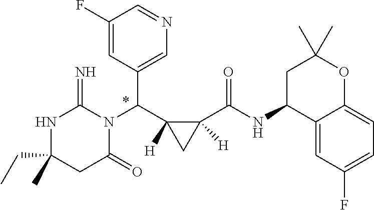 [SMILES: CC[C@@]1(C)CC(=O)N(C(c2cncc(F)c2)[C@H]2C[C@@H]2C(=O)N[C@H]2CC(C)(C)Oc3ccc(F)cc32)C(=N)N1] | 526.3 |
| 69D | 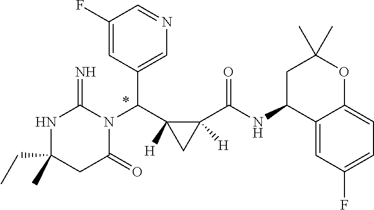 [SMILES: CC[C@@]1(C)CC(=O)N(C(c2cncc(F)c2)[C@H]2C[C@@H]2C(=O)N[C@H]2CC(C)(C)Oc3ccc(F)cc32)C(=N)N1] | 526.2 |
| 69E | 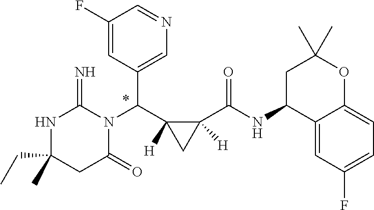 [SMILES: CC[C@@]1(C)CC(=O)N(C(c2cncc(F)c2)[C@H]2C[C@@H]2C(=O)N[C@H]2CC(C)(C)Oc3ccc(F)cc32)C(=N)N1] | 526.2 |
| 70A | 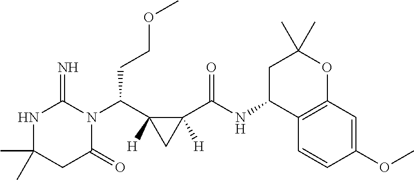 | 487.3 |
| 70B | 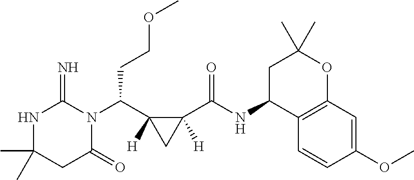 | 487.3 |
| 71 | 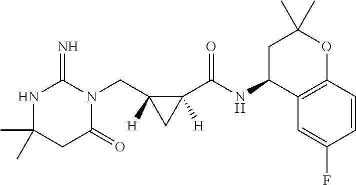 [SMILES: CC1(C)CC(=O)N(C[C@H]2C[C@@H]2C(=O)N[C@H]2CC(C)(C)Oc3ccc(F)cc32)C(=N)N1] | 417.2 |
| 72A | 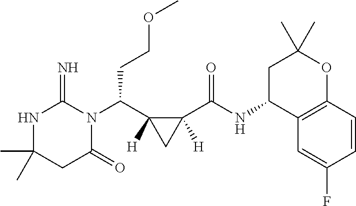 [SMILES: COCC[C@H]([C@H]1C[C@@H]1C(=O)N[C@@H]1CC(C)(C)Oc2ccc(F)cc21)N1C(=N)NC(C)(C)CC1=O] | 475.3 |
| 72B | 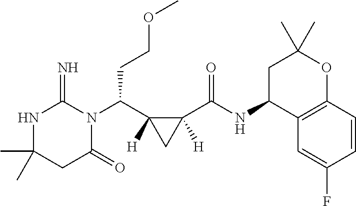 [SMILES: COCC[C@H]([C@H]1C[C@@H]1C(=O)N[C@H]1CC(C)(C)Oc2ccc(F)cc21)N1C(=N)NC(C)(C)CC1=O] | 475.3 |
| 73 | 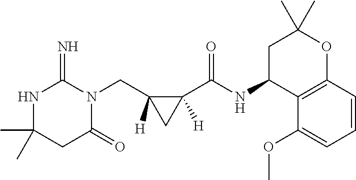 [SMILES: COc1cccc2c1[C@@H](NC(=O)[C@H]1C[C@@H]1CN1C(=N)NC(C)(C)CC1=O)CC(C)(C)O2] | 429.3 |
| 74A | 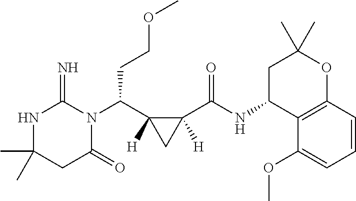 [SMILES: COCC[C@H]([C@H]1C[C@@H]1C(=O)N[C@@H]1CC(C)(C)Oc2cccc(OC)c21)N1C(=N)NC(C)(C)CC1=O] | 487.3 |
| 74B | 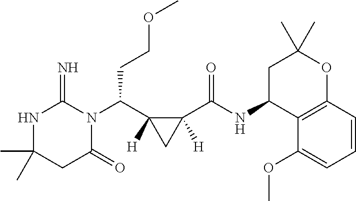 [SMILES: COCC[C@H]([C@H]1C[C@@H]1C(=O)N[C@H]1CC(C)(C)Oc2cccc(OC)c21)N1C(=N)NC(C)(C)CC1=O] | 487.3 |
| 75A | 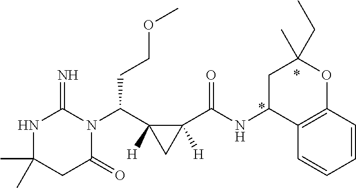 [SMILES: CCC1(C)CC(NC(=O)[C@H]2C[C@@H]2[C@@H](CCOC)N2C(=N)NC(C)(C)CC2=O)c2ccccc2O1] | 471.3 |
| 75B | 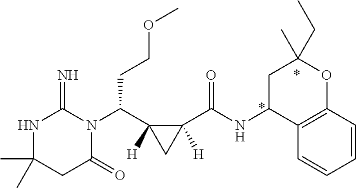 [SMILES: CCC1(C)CC(NC(=O)[C@H]2C[C@@H]2[C@@H](CCOC)N2C(=N)NC(C)(C)CC2=O)c2ccccc2O1] | 471.3 |
| 75C | 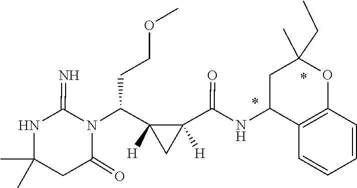 [SMILES: CCC1(C)CC(NC(=O)[C@H]2C[C@@H]2[C@@H](CCOC)N2C(=N)NC(C)(C)CC2=O)c2ccccc2O1] | 471.3 |
| 75D | 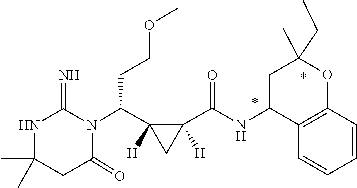 [SMILES: CCC1(C)CC(NC(=O)[C@H]2C[C@@H]2[C@@H](CCOC)N2C(=N)NC(C)(C)CC2=O)c2ccccc2O1] | 471.3 |
| 76A | 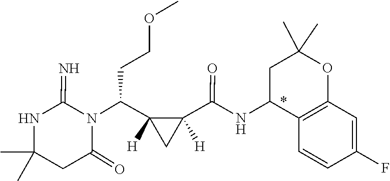 [SMILES: COCC[C@H]([C@H]1C[C@@H]1C(=O)NC1CC(C)(C)Oc2cc(F)ccc21)N1C(=N)NC(C)(C)CC1=O] | 475.3 |
| 76B | 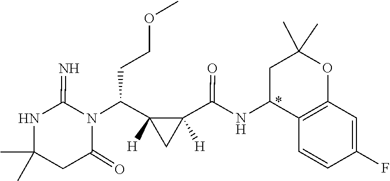 [SMILES: COCC[C@H]([C@H]1C[C@@H]1C(=O)NC1CC(C)(C)Oc2cc(F)ccc21)N1C(=N)NC(C)(C)CC1=O] | 475.3 |
| 77A | 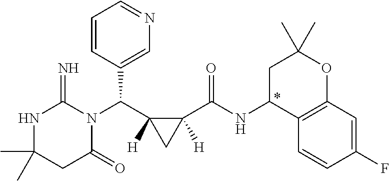 [SMILES: CC1(C)CC(=O)N([C@H](c2cccnc2)[C@H]2C[C@@H]2C(=O)NC2CC(C)(C)Oc3cc(F)ccc32)C(=N)N1] | 494.3 |
| 77B | 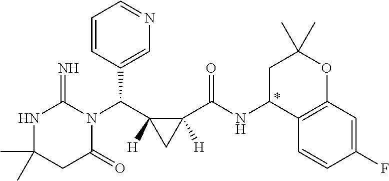 [SMILES: CC1(C)CC(=O)N([C@H](c2cccnc2)[C@H]2C[C@@H]2C(=O)NC2CC(C)(C)Oc3cc(F)ccc32)C(=N)N1] | 494.3 |
| 78 | 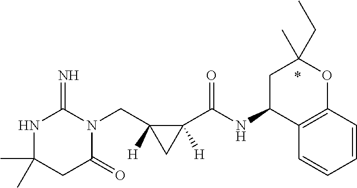 [SMILES: CCC1(C)C[C@H](NC(=O)[C@H]2C[C@@H]2CN2C(=N)NC(C)(C)CC2=O)c2ccccc2O1] | 413.3 |
| 79A | 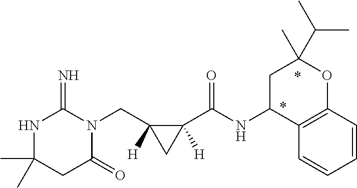 [SMILES: CC(C)C1(C)CC(NC(=O)[C@H]2C[C@@H]2CN2C(=N)NC(C)(C)CC2=O)c2ccccc2O1] | 427.3 |
| 79B | 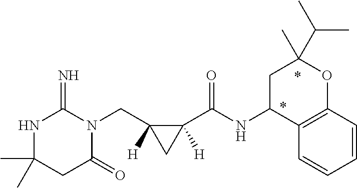 [SMILES: CC(C)C1(C)CC(NC(=O)[C@H]2C[C@@H]2CN2C(=N)NC(C)(C)CC2=O)c2ccccc2O1] | 427.3 |
| 79C | 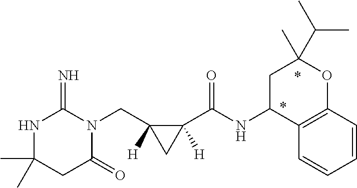 [SMILES: CC(C)C1(C)CC(NC(=O)[C@H]2C[C@@H]2CN2C(=N)NC(C)(C)CC2=O)c2ccccc2O1] | 427.3 |
| 80A | 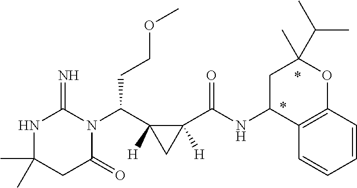 [SMILES: COCC[C@H]([C@H]1C[C@@H]1C(=O)NC1CC(C)(C(C)C)Oc2ccccc21)N1C(=N)NC(C)(C)CC1=O] | 485.4 |
| 80B | 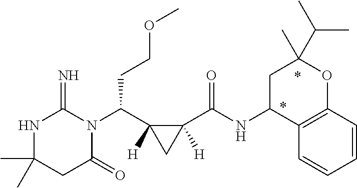 [SMILES: COCC[C@H]([C@H]1C[C@@H]1C(=O)NC1CC(C)(C(C)C)Oc2ccccc21)N1C(=N)NC(C)(C)CC1=O] | 485.3 |
| 80C | 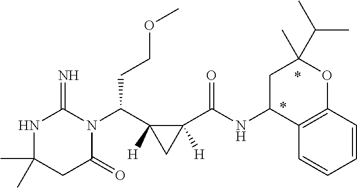 [SMILES: COCC[C@H]([C@H]1C[C@@H]1C(=O)NC1CC(C)(C(C)C)Oc2ccccc21)N1C(=N)NC(C)(C)CC1=O] | 485.3 |
| 80D | 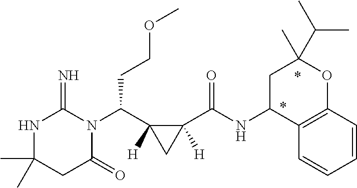 [SMILES: COCC[C@H]([C@H]1C[C@@H]1C(=O)NC1CC(C)(C(C)C)Oc2ccccc21)N1C(=N)NC(C)(C)CC1=O] | 485.3 |
| 81A | 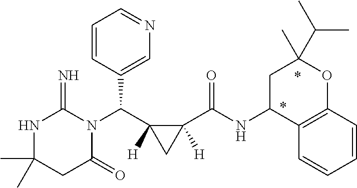 [SMILES: CC(C)C1(C)CC(NC(=O)[C@H]2C[C@@H]2[C@@H](c2cccnc2)N2C(=N)NC(C)(C)CC2=O)c2ccccc2O1] | 504.4 |
| 81B | 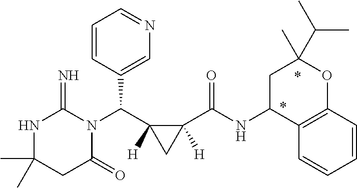 [SMILES: CC(C)C1(C)CC(NC(=O)[C@H]2C[C@@H]2[C@@H](c2cccnc2)N2C(=N)NC(C)(C)CC2=O)c2ccccc2O1] | 504.3 |
| 81C | 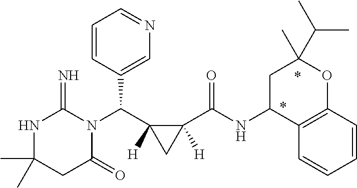 [SMILES: CC(C)C1(C)CC(NC(=O)[C@H]2C[C@@H]2[C@@H](c2cccnc2)N2C(=N)NC(C)(C)CC2=O)c2ccccc2O1] | 504.4 |
| 81D | 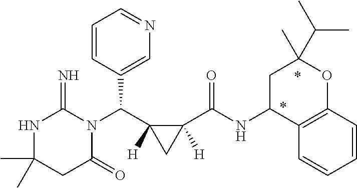 [SMILES: CC(C)C1(C)CC(NC(=O)[C@H]2C[C@@H]2[C@@H](c2cccnc2)N2C(=N)NC(C)(C)CC2=O)c2ccccc2O1] | 504.4 |
| 82A | 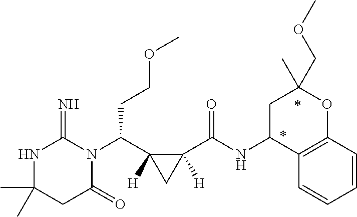 [SMILES: COCC[C@H]([C@H]1C[C@@H]1C(=O)NC1CC(C)(COC)Oc2ccccc21)N1C(=N)NC(C)(C)CC1=O] | 487.3 |
| 82B | 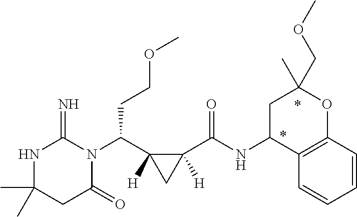 [SMILES: COCC[C@H]([C@H]1C[C@@H]1C(=O)NC1CC(C)(COC)Oc2ccccc21)N1C(=N)NC(C)(C)CC1=O] | 487.3 |
| 82C | 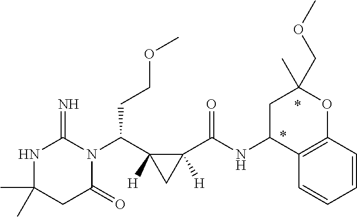 [SMILES: COCC[C@H]([C@H]1C[C@@H]1C(=O)NC1CC(C)(COC)Oc2ccccc21)N1C(=N)NC(C)(C)CC1=O] | 487.3 |
| 82D | 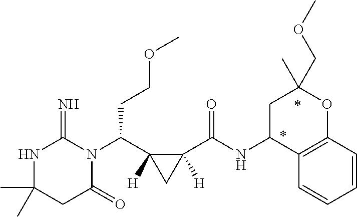 [SMILES: COCC[C@H]([C@H]1C[C@@H]1C(=O)NC1CC(C)(COC)Oc2ccccc21)N1C(=N)NC(C)(C)CC1=O] | 487.3 |
| 83A | 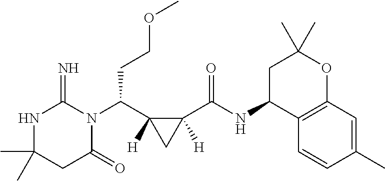 | 471.3 |
| 83B | 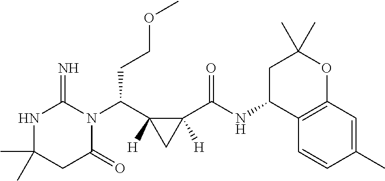 | 471.3 |
| 84 | 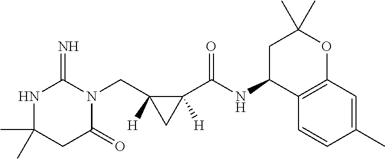 | 413.2 |
| 85A | 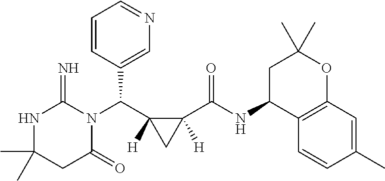 | 490.3 |
| 85B | 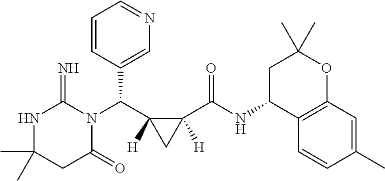 | 490.3 |
| 86A | 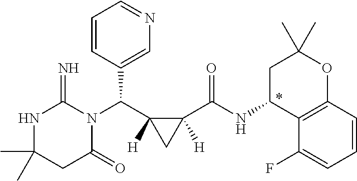 [SMILES: CC1(C)CC(=O)N([C@H](c2cccnc2)[C@H]2C[C@@H]2C(=O)N[C@@H]2CC(C)(C)Oc3cccc(F)c32)C(=N)N1] | 494.3 |
| 86B | 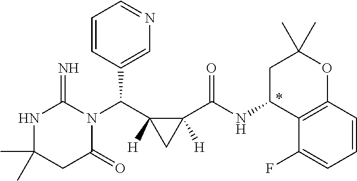 [SMILES: CC1(C)CC(=O)N([C@H](c2cccnc2)[C@H]2C[C@@H]2C(=O)N[C@@H]2CC(C)(C)Oc3cccc(F)c32)C(=N)N1] | 494.3 |
| 87 | 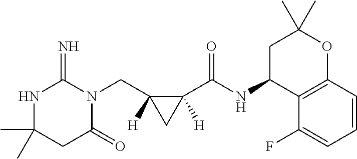 [SMILES: CC1(C)CC(=O)N(C[C@H]2C[C@@H]2C(=O)N[C@H]2CC(C)(C)Oc3cccc(F)c32)C(=N)N1] | 417.3 |
| 88A | 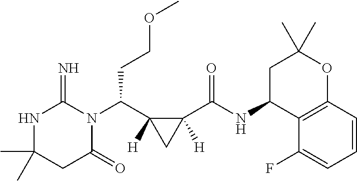 [SMILES: COCC[C@H]([C@H]1C[C@@H]1C(=O)N[C@H]1CC(C)(C)Oc2cccc(F)c21)N1C(=N)NC(C)(C)CC1=O] | 475.3 |
| 88B | 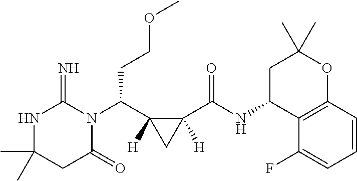 [SMILES: COCC[C@H]([C@H]1C[C@@H]1C(=O)N[C@@H]1CC(C)(C)Oc2cccc(F)c21)N1C(=N)NC(C)(C)CC1=O] | 475.3 |
| 89A | 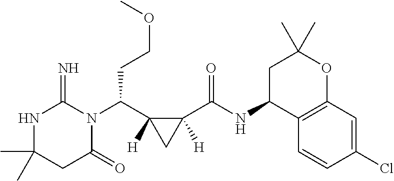 [SMILES: COCC[C@H]([C@H]1C[C@@H]1C(=O)N[C@H]1CC(C)(C)Oc2cc(Cl)ccc21)N1C(=N)NC(C)(C)CC1=O] | 491.3 |
| 89B | 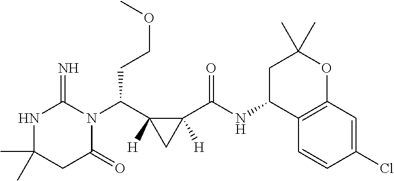 [SMILES: COCC[C@H]([C@H]1C[C@@H]1C(=O)N[C@@H]1CC(C)(C)Oc2cc(Cl)ccc21)N1C(=N)NC(C)(C)CC1=O] | 491.3 |
| 90 | 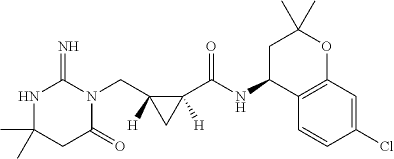 [SMILES: CC1(C)CC(=O)N(C[C@H]2C[C@@H]2C(=O)N[C@H]2CC(C)(C)Oc3cc(Cl)ccc32)C(=N)N1] | 433.2 |
| 91A | 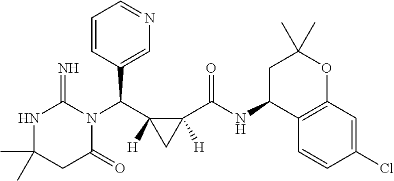 [SMILES: CC1(C)CC(=O)N([C@@H](c2cccnc2)[C@H]2C[C@@H]2C(=O)N[C@H]2CC(C)(C)Oc3cc(Cl)ccc32)C(=N)N1] | 510.3 |
| 91B | 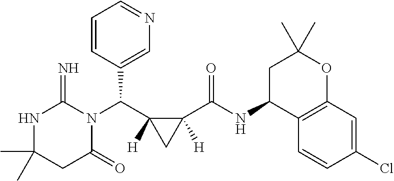 [SMILES: CC1(C)CC(=O)N([C@H](c2cccnc2)[C@H]2C[C@@H]2C(=O)N[C@H]2CC(C)(C)Oc3cc(Cl)ccc32)C(=N)N1] | 510.3 |
| 92A | 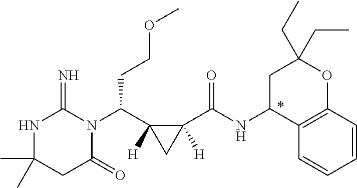 [SMILES: CCC1(CC)CC(NC(=O)[C@H]2C[C@@H]2[C@@H](CCOC)N2C(=N)NC(C)(C)CC2=O)c2ccccc2O1] | 485.3 |
| 92B | 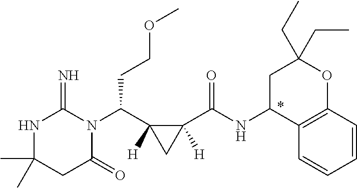 [SMILES: CCC1(CC)CC(NC(=O)[C@H]2C[C@@H]2[C@@H](CCOC)N2C(=N)NC(C)(C)CC2=O)c2ccccc2O1] | 485.4 |
| 93A | 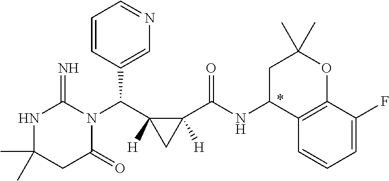 [SMILES: CC1(C)CC(=O)N([C@H](c2cccnc2)[C@H]2C[C@@H]2C(=O)NC2CC(C)(C)Oc3c(F)cccc32)C(=N)N1] | 494.3 |
| 93B | 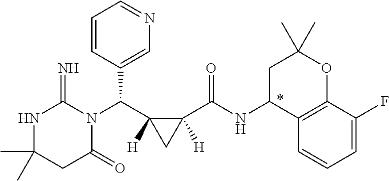 [SMILES: CC1(C)CC(=O)N([C@H](c2cccnc2)[C@H]2C[C@@H]2C(=O)NC2CC(C)(C)Oc3c(F)cccc32)C(=N)N1] | 494.3 |
| 94A | 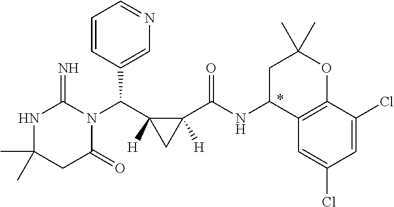 [SMILES: CC1(C)CC(=O)N([C@H](c2cccnc2)[C@H]2C[C@@H]2C(=O)NC2CC(C)(C)Oc3c(Cl)cc(Cl)cc32)C(=N)N1] | 544.2, 546.2 |
| 94B | 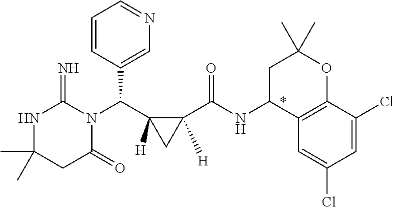 [SMILES: CC1(C)CC(=O)N([C@H](c2cccnc2)[C@H]2C[C@@H]2C(=O)NC2CC(C)(C)Oc3c(Cl)cc(Cl)cc32)C(=N)N1] | 544.2, 546.2 |
| 95 | 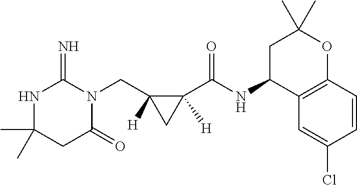 [SMILES: CC1(C)CC(=O)N(C[C@H]2C[C@@H]2C(=O)N[C@H]2CC(C)(C)Oc3ccc(Cl)cc32)C(=N)N1] | 433.2 |
| 96A | 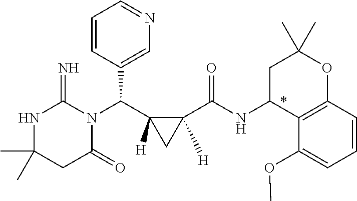 [SMILES: COc1cccc2c1C(NC(=O)[C@H]1C[C@@H]1[C@@H](c1cccnc1)N1C(=N)NC(C)(C)CC1=O)CC(C)(C)O2] | 506.3 |
| 96B | 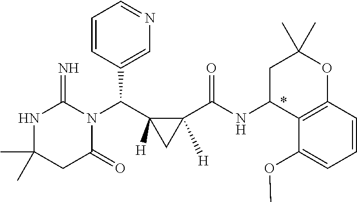 [SMILES: COc1cccc2c1C(NC(=O)[C@H]1C[C@@H]1[C@@H](c1cccnc1)N1C(=N)NC(C)(C)CC1=O)CC(C)(C)O2] | 506.3 |
| 97A | 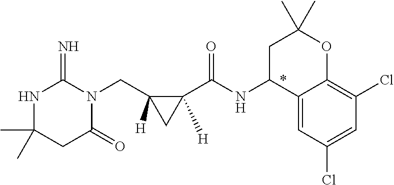 [SMILES: CC1(C)CC(=O)N(C[C@H]2C[C@@H]2C(=O)NC2CC(C)(C)Oc3c(Cl)cc(Cl)cc32)C(=N)N1] | 467.1, 469.2 |
| 97B | 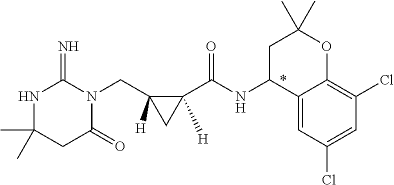 [SMILES: CC1(C)CC(=O)N(C[C@H]2C[C@@H]2C(=O)NC2CC(C)(C)Oc3c(Cl)cc(Cl)cc32)C(=N)N1] | 467.1, 469.2 |
| 98A | 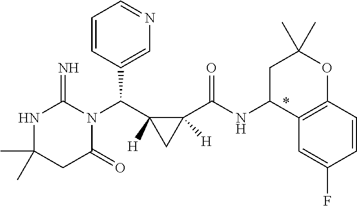 [SMILES: CC1(C)CC(=O)N([C@H](c2cccnc2)[C@H]2C[C@@H]2C(=O)NC2CC(C)(C)Oc3ccc(F)cc32)C(=N)N1] | 494.3 |
| 98B | 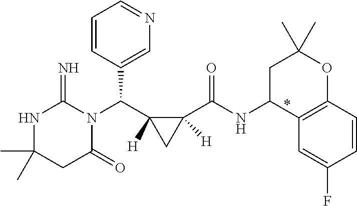 [SMILES: CC1(C)CC(=O)N([C@H](c2cccnc2)[C@H]2C[C@@H]2C(=O)NC2CC(C)(C)Oc3ccc(F)cc32)C(=N)N1] | 494.3 |
| 99 | 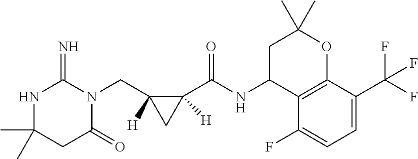 [SMILES: CC1(C)CC(=O)N(C[C@H]2C[C@@H]2C(=O)NC2CC(C)(C)Oc3c(C(F)(F)F)ccc(F)c32)C(=N)N1] | 485.2 |
| 100 | 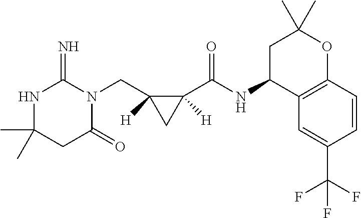 [SMILES: CC1(C)CC(=O)N(C[C@H]2C[C@@H]2C(=O)N[C@H]2CC(C)(C)Oc3ccc(C(F)(F)F)cc32)C(=N)N1] | 467.2 |
| 101 | 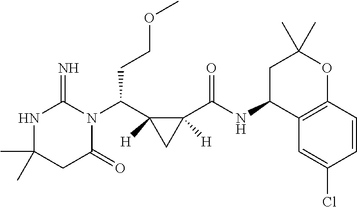 [SMILES: COCC[C@H]([C@H]1C[C@@H]1C(=O)N[C@H]1CC(C)(C)Oc2ccc(Cl)cc21)N1C(=N)NC(C)(C)CC1=O] | 491.3 |
| 102A | 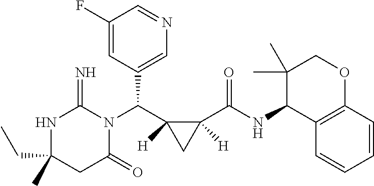 [SMILES: CC[C@@]1(C)CC(=O)N([C@H](c2cncc(F)c2)[C@H]2C[C@@H]2C(=O)N[C@@H]2c3ccccc3OCC2(C)C)C(=N)N1] | 508.2 |
| 102B | 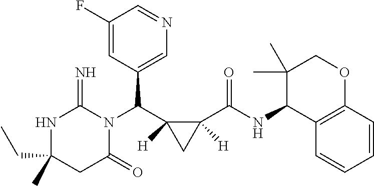 | 508.2 |
| 103A | 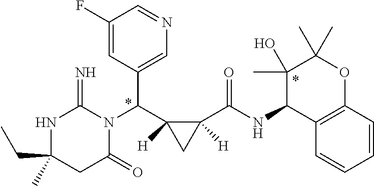 [SMILES: CC[C@]1(C)CC(=O)N(C(c2cncc(F)c2)[C@H]2C[C@@H]2C(=O)N[C@@H]2c3ccccc3OC(C)(C)C2(C)O)C(=N)N1] | 538.2 |
| 103B | 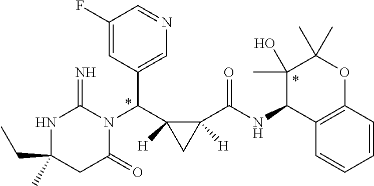 [SMILES: CC[C@]1(C)CC(=O)N(C(c2cncc(F)c2)[C@H]2C[C@@H]2C(=O)N[C@@H]2c3ccccc3OC(C)(C)C2(C)O)C(=N)N1] | 538.3 |
| 104A | 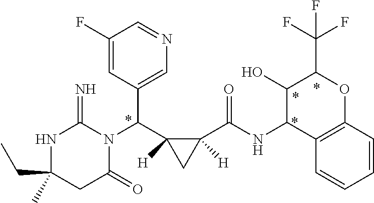 [SMILES: CC[C@]1(C)CC(=O)N(C(c2cncc(F)c2)[C@H]2C[C@@H]2C(=O)NC2c3ccccc3OC(C(F)(F)F)C2O)C(=N)N1] | 564.2 |
| 104B | 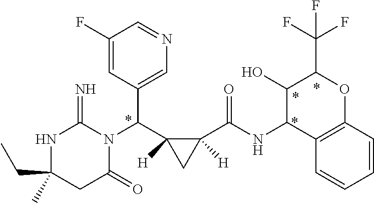 [SMILES: CC[C@]1(C)CC(=O)N(C(c2cncc(F)c2)[C@H]2C[C@@H]2C(=O)NC2c3ccccc3OC(C(F)(F)F)C2O)C(=N)N1] | 564.2 |
| 105A | 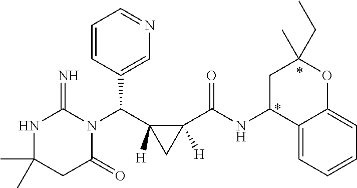 [SMILES: CCC1(C)CC(NC(=O)[C@H]2C[C@@H]2[C@@H](c2cccnc2)N2C(=N)NC(C)(C)CC2=O)c2ccccc2O1] | 490.3 |
| 105B | 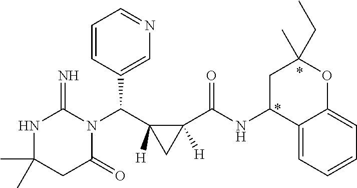 [SMILES: CCC1(C)CC(NC(=O)[C@H]2C[C@@H]2[C@@H](c2cccnc2)N2C(=N)NC(C)(C)CC2=O)c2ccccc2O1] | 490.3 |
| 105C | 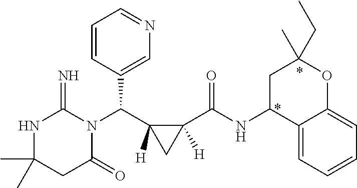 [SMILES: CCC1(C)CC(NC(=O)[C@H]2C[C@@H]2[C@@H](c2cccnc2)N2C(=N)NC(C)(C)CC2=O)c2ccccc2O1] | 490.3 |
| 105D | 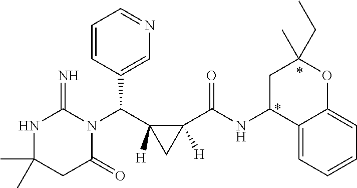 [SMILES: CCC1(C)CC(NC(=O)[C@H]2C[C@@H]2[C@@H](c2cccnc2)N2C(=N)NC(C)(C)CC2=O)c2ccccc2O1] | 490.3 |
| 106A | 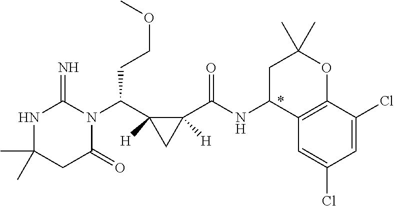 [SMILES: COCC[C@H]([C@H]1C[C@@H]1C(=O)NC1CC(C)(C)Oc2c(Cl)cc(Cl)cc21)N1C(=N)NC(C)(C)CC1=O] | 525.2, 527.2 |
| 106B | 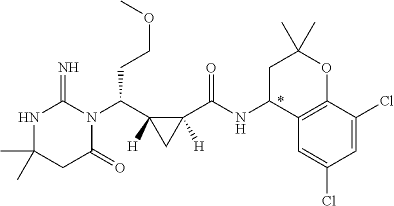 [SMILES: COCC[C@H]([C@H]1C[C@@H]1C(=O)NC1CC(C)(C)Oc2c(Cl)cc(Cl)cc21)N1C(=N)NC(C)(C)CC1=O] | 525.2, 527.2 |
| 107A | 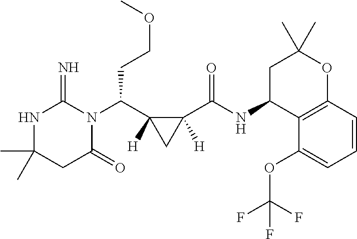 [SMILES: COCC[C@H]([C@H]1C[C@@H]1C(=O)N[C@H]1CC(C)(C)Oc2cccc(OC(F)(F)F)c21)N1C(=N)NC(C)(C)CC1=O] | 541.3 |
| 107B | 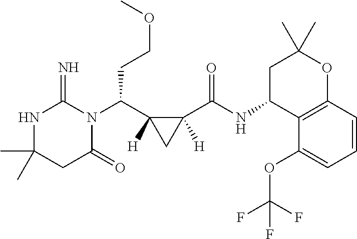 [SMILES: COCC[C@H]([C@H]1C[C@@H]1C(=O)N[C@@H]1CC(C)(C)Oc2cccc(OC(F)(F)F)c21)N1C(=N)NC(C)(C)CC1=O] | 541.3 |
| 108A | 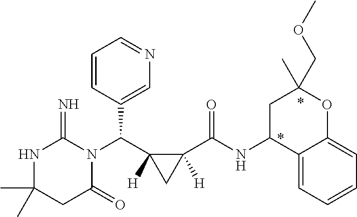 [SMILES: COCC1(C)CC(NC(=O)[C@H]2C[C@@H]2[C@@H](c2cccnc2)N2C(=N)NC(C)(C)CC2=O)c2ccccc2O1] | 506.3 |
| 108B | 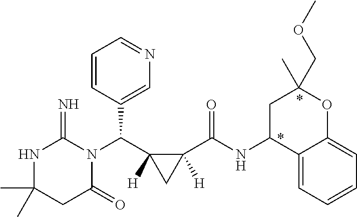 [SMILES: COCC1(C)CC(NC(=O)[C@H]2C[C@@H]2[C@@H](c2cccnc2)N2C(=N)NC(C)(C)CC2=O)c2ccccc2O1] | 506.2 |
| 108C | 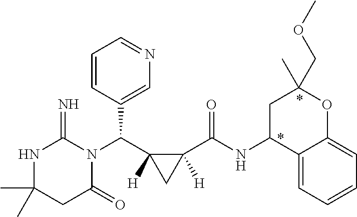 [SMILES: COCC1(C)CC(NC(=O)[C@H]2C[C@@H]2[C@@H](c2cccnc2)N2C(=N)NC(C)(C)CC2=O)c2ccccc2O1] | 506.3 |
| 108D | 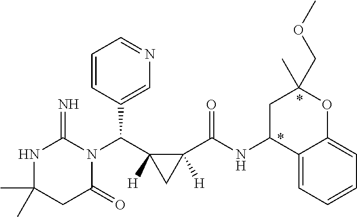 [SMILES: COCC1(C)CC(NC(=O)[C@H]2C[C@@H]2[C@@H](c2cccnc2)N2C(=N)NC(C)(C)CC2=O)c2ccccc2O1] | 506.3 |
| 109A | 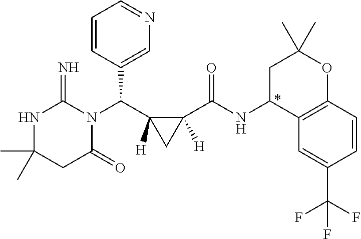 [SMILES: CC1(C)CC(=O)N([C@H](c2cccnc2)[C@H]2C[C@@H]2C(=O)NC2CC(C)(C)Oc3ccc(C(F)(F)F)cc32)C(=N)N1] | 544.3 |
| 109B | 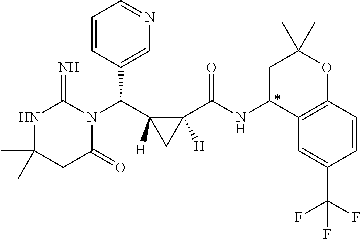 [SMILES: CC1(C)CC(=O)N([C@H](c2cccnc2)[C@H]2C[C@@H]2C(=O)NC2CC(C)(C)Oc3ccc(C(F)(F)F)cc32)C(=N)N1] | 544.3 |
| 110A | 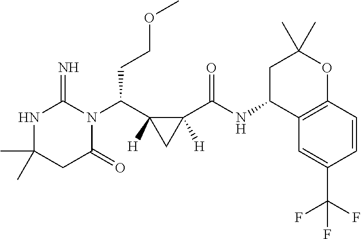 [SMILES: COCC[C@H]([C@H]1C[C@@H]1C(=O)N[C@@H]1CC(C)(C)Oc2ccc(C(F)(F)F)cc21)N1C(=N)NC(C)(C)CC1=O] | 525.3 |
| 110B | 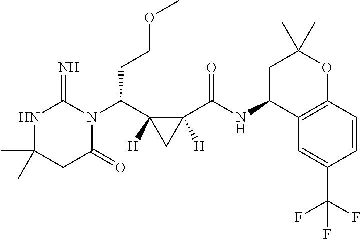 [SMILES: COCC[C@H]([C@H]1C[C@@H]1C(=O)N[C@H]1CC(C)(C)Oc2ccc(C(F)(F)F)cc21)N1C(=N)NC(C)(C)CC1=O] | 525.3 |
| 111A | 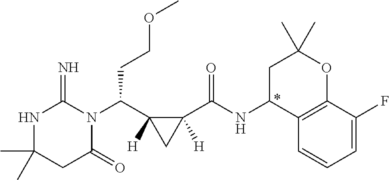 [SMILES: COCC[C@H]([C@H]1C[C@@H]1C(=O)NC1CC(C)(C)Oc2c(F)cccc21)N1C(=N)NC(C)(C)CC1=O] | 475.3 |
| 111B | 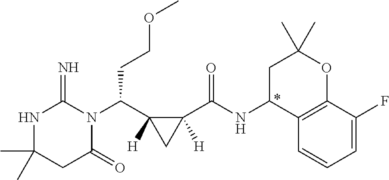 [SMILES: COCC[C@H]([C@H]1C[C@@H]1C(=O)NC1CC(C)(C)Oc2c(F)cccc21)N1C(=N)NC(C)(C)CC1=O] | 475.3 |
| 112A | 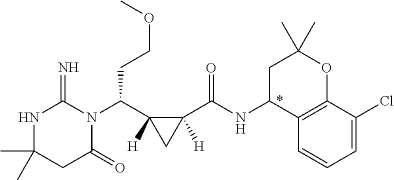 [SMILES: COCC[C@H]([C@H]1C[C@@H]1C(=O)NC1CC(C)(C)Oc2c(Cl)cccc21)N1C(=N)NC(C)(C)CC1=O] | 491.3 |
| 112B | 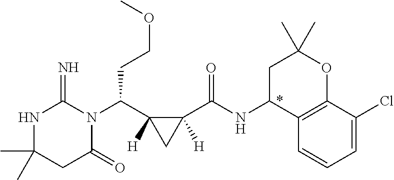 [SMILES: COCC[C@H]([C@H]1C[C@@H]1C(=O)NC1CC(C)(C)Oc2c(Cl)cccc21)N1C(=N)NC(C)(C)CC1=O] | 491.3 |
| 113A | 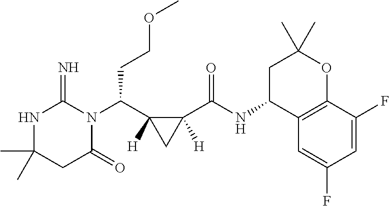 [SMILES: COCC[C@H]([C@H]1C[C@@H]1C(=O)N[C@@H]1CC(C)(C)Oc2c(F)cc(F)cc21)N1C(=N)NC(C)(C)CC1=O] | 493.3 |
| 113B | 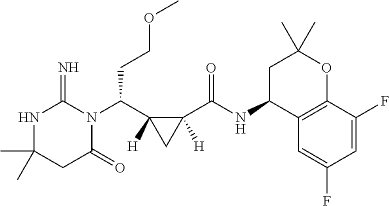 [SMILES: COCC[C@H]([C@H]1C[C@@H]1C(=O)N[C@H]1CC(C)(C)Oc2c(F)cc(F)cc21)N1C(=N)NC(C)(C)CC1=O] | 493.3 |
| 114A | 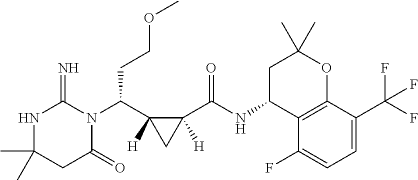 [SMILES: COCC[C@H]([C@H]1C[C@@H]1C(=O)N[C@@H]1CC(C)(C)Oc2c(C(F)(F)F)ccc(F)c21)N1C(=N)NC(C)(C)CC1=O] | 543.3 |
| 114B | 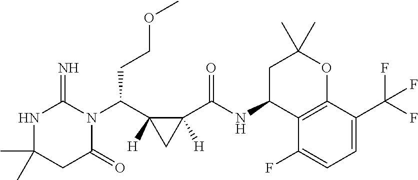 [SMILES: COCC[C@H]([C@H]1C[C@@H]1C(=O)N[C@H]1CC(C)(C)Oc2c(C(F)(F)F)ccc(F)c21)N1C(=N)NC(C)(C)CC1=O] | 543.2 |
| 115A | 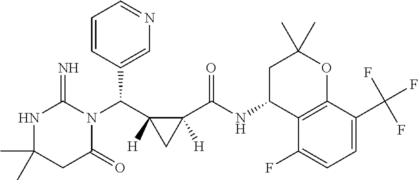 [SMILES: CC1(C)CC(=O)N([C@H](c2cccnc2)[C@H]2C[C@@H]2C(=O)N[C@@H]2CC(C)(C)Oc3c(C(F)(F)F)ccc(F)c32)C(=N)N1] | 562.2 |
| 115B | 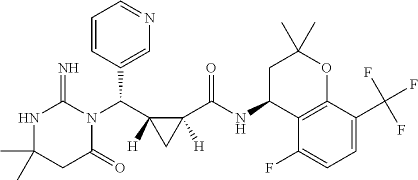 [SMILES: CC1(C)CC(=O)N([C@H](c2cccnc2)[C@H]2C[C@@H]2C(=O)N[C@H]2CC(C)(C)Oc3c(C(F)(F)F)ccc(F)c32)C(=N)N1] | 562.2 |
| 116A | 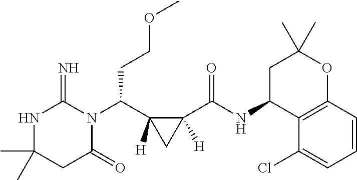 [SMILES: COCC[C@H]([C@H]1C[C@@H]1C(=O)N[C@H]1CC(C)(C)Oc2cccc(Cl)c21)N1C(=N)NC(C)(C)CC1=O] | 491.2 |
| 116B | 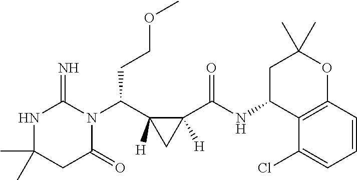 [SMILES: COCC[C@H]([C@H]1C[C@@H]1C(=O)N[C@@H]1CC(C)(C)Oc2cccc(Cl)c21)N1C(=N)NC(C)(C)CC1=O] | 491.3 |
| 117A | 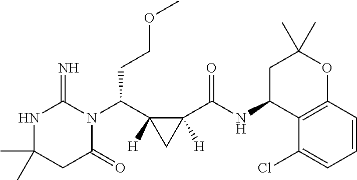 [SMILES: COCC[C@H]([C@H]1C[C@@H]1C(=O)N[C@H]1CC(C)(C)Oc2cccc(Cl)c21)N1C(=N)NC(C)(C)CC1=O] | 510.2 |
| 117B | 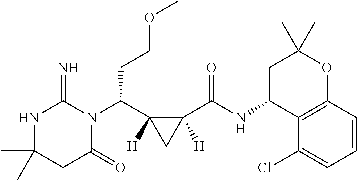 [SMILES: COCC[C@H]([C@H]1C[C@@H]1C(=O)N[C@@H]1CC(C)(C)Oc2cccc(Cl)c21)N1C(=N)NC(C)(C)CC1=O] | 510.3 |
| 118 | 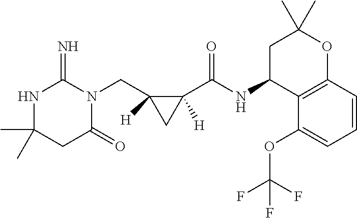 [SMILES: CC1(C)CC(=O)N(C[C@H]2C[C@@H]2C(=O)N[C@H]2CC(C)(C)Oc3cccc(OC(F)(F)F)c32)C(=N)N1] | 483.2 |
| 119 | 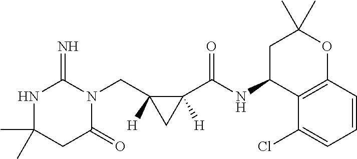 [SMILES: CC1(C)CC(=O)N(C[C@H]2C[C@@H]2C(=O)N[C@H]2CC(C)(C)Oc3cccc(Cl)c32)C(=N)N1] | 433.2 |
| 120 | 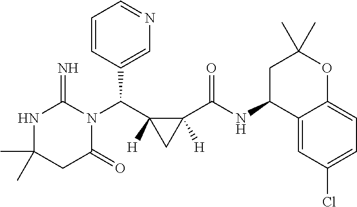 [SMILES: CC1(C)CC(=O)N([C@H](c2cccnc2)[C@H]2C[C@@H]2C(=O)N[C@H]2CC(C)(C)Oc3ccc(Cl)cc32)C(=N)N1] | 510.3 |
| 121A | 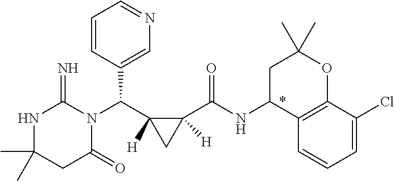 [SMILES: CC1(C)CC(=O)N([C@H](c2cccnc2)[C@H]2C[C@@H]2C(=O)NC2CC(C)(C)Oc3c(Cl)cccc32)C(=N)N1] | 510.2 |
| 121B | 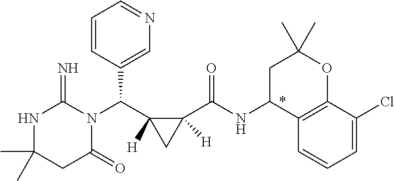 [SMILES: CC1(C)CC(=O)N([C@H](c2cccnc2)[C@H]2C[C@@H]2C(=O)NC2CC(C)(C)Oc3c(Cl)cccc32)C(=N)N1] | 510.3 |
| 122A | 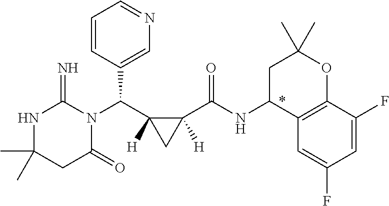 [SMILES: CC1(C)CC(=O)N([C@H](c2cccnc2)[C@H]2C[C@@H]2C(=O)NC2CC(C)(C)Oc3c(F)cc(F)cc32)C(=N)N1] | 512.2 |
| 122B | 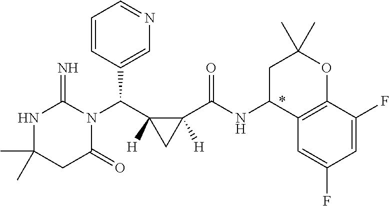 [SMILES: CC1(C)CC(=O)N([C@H](c2cccnc2)[C@H]2C[C@@H]2C(=O)NC2CC(C)(C)Oc3c(F)cc(F)cc32)C(=N)N1] | 512.2 |
| 123 | 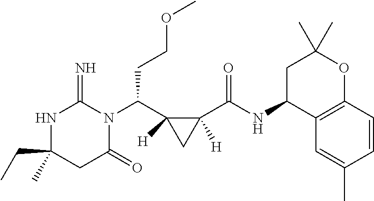 [SMILES: CC[C@]1(C)CC(=O)N([C@H](CCOC)[C@H]2C[C@@H]2C(=O)N[C@H]2CC(C)(C)Oc3ccc(C)cc32)C(=N)N1] | 485.3 |
| 124 | 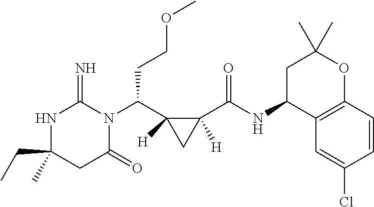 [SMILES: CC[C@]1(C)CC(=O)N([C@H](CCOC)[C@H]2C[C@@H]2C(=O)N[C@H]2CC(C)(C)Oc3ccc(Cl)cc32)C(=N)N1] | 505.3 |
| 125A | 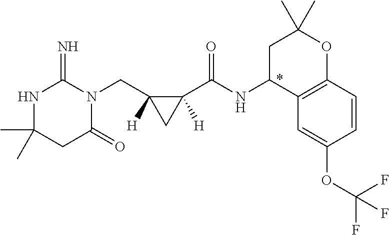 [SMILES: CC1(C)CC(=O)N(C[C@H]2C[C@@H]2C(=O)NC2CC(C)(C)Oc3ccc(OC(F)(F)F)cc32)C(=N)N1] | 483.1 |
| 125B | 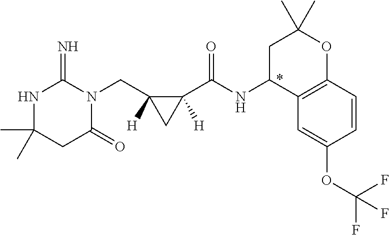 [SMILES: CC1(C)CC(=O)N(C[C@H]2C[C@@H]2C(=O)NC2CC(C)(C)Oc3ccc(OC(F)(F)F)cc32)C(=N)N1] | 483.1 |
| 126A | 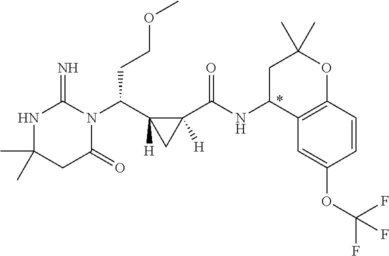 [SMILES: COCC[C@H]([C@H]1C[C@@H]1C(=O)NC1CC(C)(C)Oc2ccc(OC(F)(F)F)cc21)N1C(=N)NC(C)(C)CC1=O] | 541.3 |
| 126B | 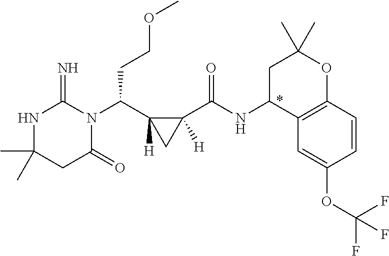 [SMILES: COCC[C@H]([C@H]1C[C@@H]1C(=O)NC1CC(C)(C)Oc2ccc(OC(F)(F)F)cc21)N1C(=N)NC(C)(C)CC1=O] | 541.3 |
| 127A | 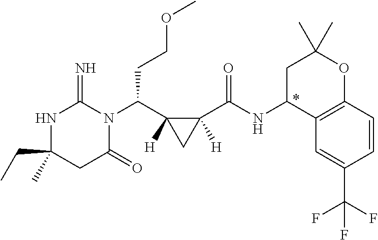 [SMILES: CC[C@]1(C)CC(=O)N([C@H](CCOC)[C@H]2C[C@@H]2C(=O)NC2CC(C)(C)Oc3ccc(C(F)(F)F)cc32)C(=N)N1] | 539.3 |
| 127B | 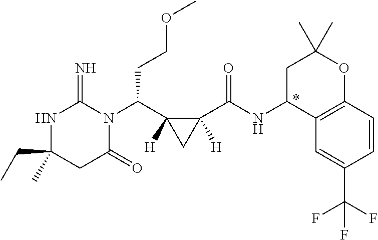 [SMILES: CC[C@]1(C)CC(=O)N([C@H](CCOC)[C@H]2C[C@@H]2C(=O)NC2CC(C)(C)Oc3ccc(C(F)(F)F)cc32)C(=N)N1] | 539.3 |
| 128 | 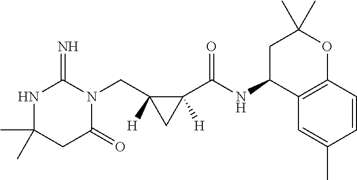 [SMILES: Cc1ccc2c(c1)[C@@H](NC(=O)[C@H]1C[C@@H]1CN1C(=N)NC(C)(C)CC1=O)CC(C)(C)O2] | 413.3 |
| 129A | 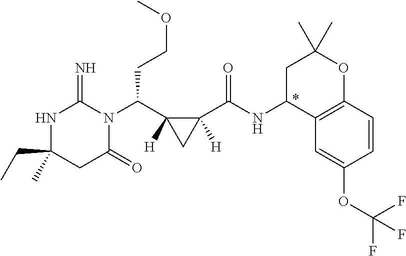 [SMILES: CC[C@]1(C)CC(=O)N([C@H](CCOC)[C@H]2C[C@@H]2C(=O)NC2CC(C)(C)Oc3ccc(OC(F)(F)F)cc32)C(=N)N1] | 555.3 |
| 129B | 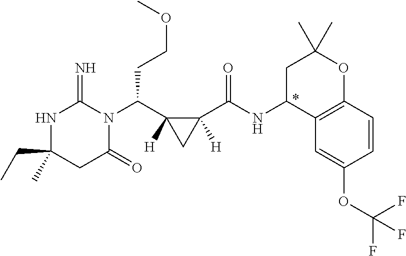 [SMILES: CC[C@]1(C)CC(=O)N([C@H](CCOC)[C@H]2C[C@@H]2C(=O)NC2CC(C)(C)Oc3ccc(OC(F)(F)F)cc32)C(=N)N1] | 555.2 |
| 130A | 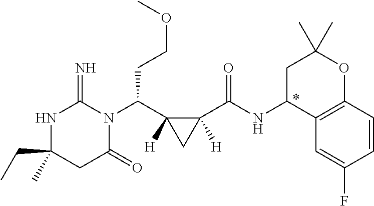 [SMILES: CC[C@]1(C)CC(=O)N([C@H](CCOC)[C@H]2C[C@@H]2C(=O)NC2CC(C)(C)Oc3ccc(F)cc32)C(=N)N1] | 489.3 |
| 130B | 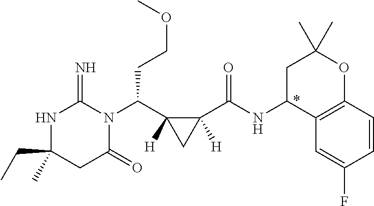 [SMILES: CC[C@]1(C)CC(=O)N([C@H](CCOC)[C@H]2C[C@@H]2C(=O)NC2CC(C)(C)Oc3ccc(F)cc32)C(=N)N1] | 489.2 |
| 131A | 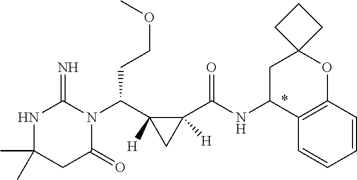 [SMILES: COCC[C@H]([C@H]1C[C@@H]1C(=O)NC1CC2(CCC2)Oc2ccccc21)N1C(=N)NC(C)(C)CC1=O] | 469.2 |
| 131B | 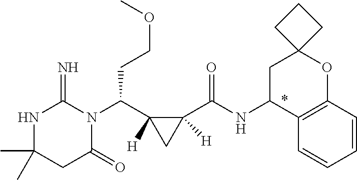 [SMILES: COCC[C@H]([C@H]1C[C@@H]1C(=O)NC1CC2(CCC2)Oc2ccccc21)N1C(=N)NC(C)(C)CC1=O] | 469.3 |
| 132 | 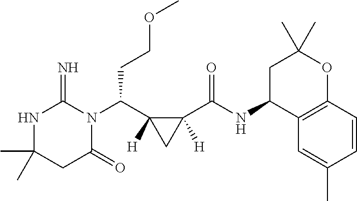 [SMILES: COCC[C@H]([C@H]1C[C@@H]1C(=O)N[C@H]1CC(C)(C)Oc2ccc(C)cc21)N1C(=N)NC(C)(C)CC1=O] | 471.3 |
| 133A | 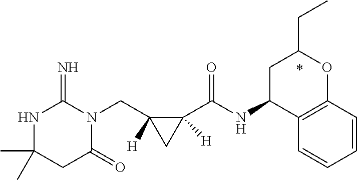 [SMILES: CCC1C[C@H](NC(=O)[C@H]2C[C@@H]2CN2C(=N)NC(C)(C)CC2=O)c2ccccc2O1] | 399.3 |
| 133B | 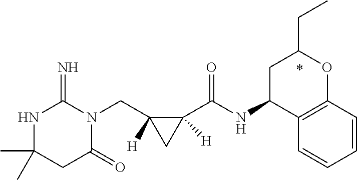 [SMILES: CCC1C[C@H](NC(=O)[C@H]2C[C@@H]2CN2C(=N)NC(C)(C)CC2=O)c2ccccc2O1] | 399.2 |
| 134A | 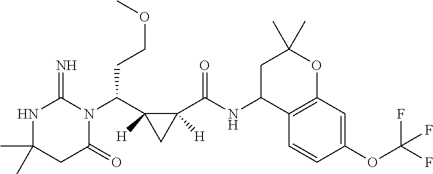 [SMILES: COCC[C@H]([C@H]1C[C@@H]1C(=O)NC1CC(C)(C)Oc2cc(OC(F)(F)F)ccc21)N1C(=N)NC(C)(C)CC1=O] | 541.1 |
| 134B | 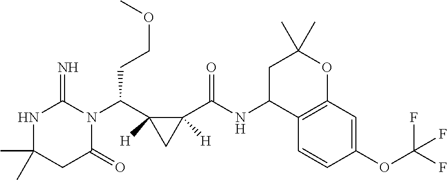 [SMILES: COCC[C@H]([C@H]1C[C@@H]1C(=O)NC1CC(C)(C)Oc2cc(OC(F)(F)F)ccc21)N1C(=N)NC(C)(C)CC1=O] | 541.1 |
| 135 | 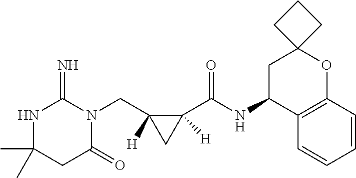 [SMILES: CC1(C)CC(=O)N(C[C@H]2C[C@@H]2C(=O)N[C@H]2CC3(CCC3)Oc3ccccc32)C(=N)N1] | 411.2 |
| 136 | 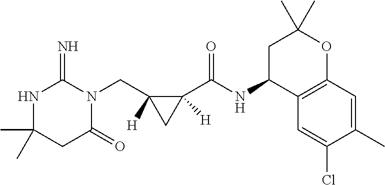 | 447.3 |
| 137A | 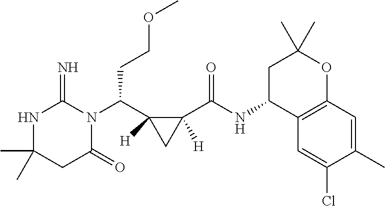 | 505.3 |
| 137B | 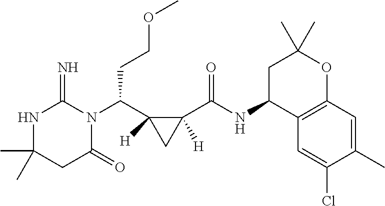 | 505.3 |
| 138A | 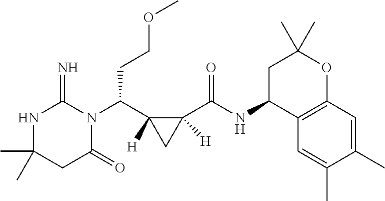 | 485.2 |
| 138B | 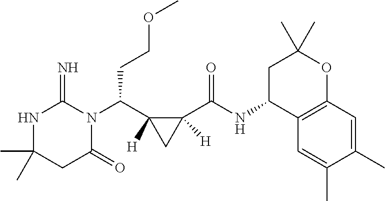 | 485.2 |
| 139A | 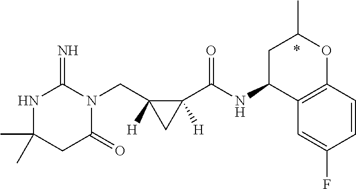 [SMILES: CC1C[C@H](NC(=O)[C@H]2C[C@@H]2CN2C(=N)NC(C)(C)CC2=O)c2cc(F)ccc2O1] | 403.2 |
| 139B | 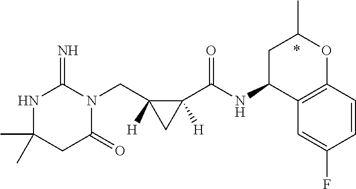 [SMILES: CC1C[C@H](NC(=O)[C@H]2C[C@@H]2CN2C(=N)NC(C)(C)CC2=O)c2cc(F)ccc2O1] | 403.1 |
| 140A | 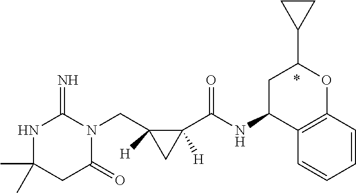 [SMILES: CC1(C)CC(=O)N(C[C@H]2C[C@@H]2C(=O)N[C@H]2CC(C3CC3)Oc3ccccc32)C(=N)N1] | 411.1 |
| 140B | 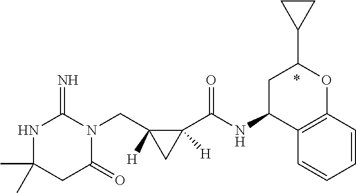 [SMILES: CC1(C)CC(=O)N(C[C@H]2C[C@@H]2C(=O)N[C@H]2CC(C3CC3)Oc3ccccc32)C(=N)N1] | 411.2 |

TABLE 2

| | | LC/ |
|---|---|---|
| | | MS |
| Ex. | Structure | (M + 1)+ |
| 141 | 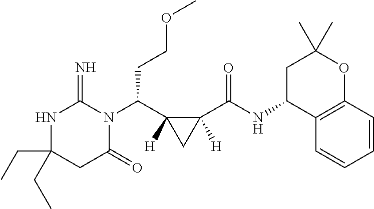 [SMILES: CCC1(CC)CC(=O)N([C@H](CCOC)[C@H]2C[C@@H]2C(=O)N[C@@H]2CC(C)(C)Oc3ccccc32)C(=N)N1] | 485.3 |
| 142A | 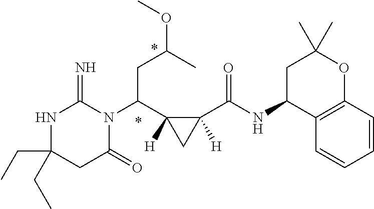 [SMILES: CCC1(CC)CC(=O)N(C(CC(C)OC)[C@H]2C[C@@H]2C(=O)N[C@H]2CC(C)(C)Oc3ccccc32)C(=N)N1] | 499.4 |
| 142B | 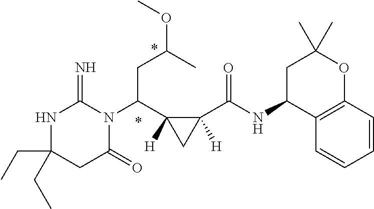 [SMILES: CCC1(CC)CC(=O)N(C(CC(C)OC)[C@H]2C[C@@H]2C(=O)N[C@H]2CC(C)(C)Oc3ccccc32)C(=N)N1] | 499.4 |
| 142C | 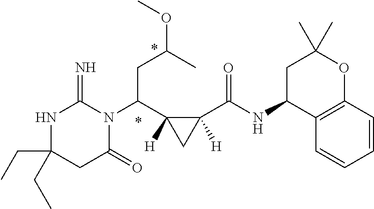 [SMILES: CCC1(CC)CC(=O)N(C(CC(C)OC)[C@H]2C[C@@H]2C(=O)N[C@H]2CC(C)(C)Oc3ccccc32)C(=N)N1] | 499.4 |
| 142D | 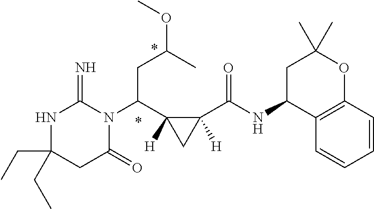 [SMILES: CCC1(CC)CC(=O)N(C(CC(C)OC)[C@H]2C[C@@H]2C(=O)N[C@H]2CC(C)(C)Oc3ccccc32)C(=N)N1] | 499.3 |
| 143A | 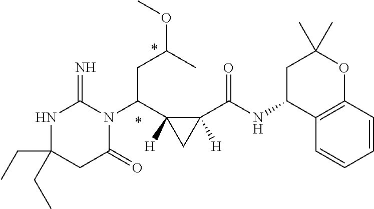 [SMILES: CCC1(CC)CC(=O)N(C(CC(C)OC)[C@H]2C[C@@H]2C(=O)N[C@@H]2CC(C)(C)Oc3ccccc32)C(=N)N1] | 499.4 |
| 143B | 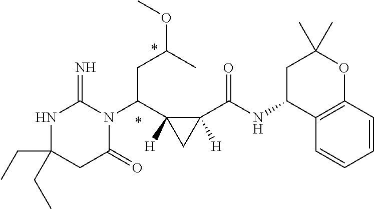 [SMILES: CCC1(CC)CC(=O)N(C(CC(C)OC)[C@H]2C[C@@H]2C(=O)N[C@@H]2CC(C)(C)Oc3ccccc32)C(=N)N1] | 499.4 |
| 143C | 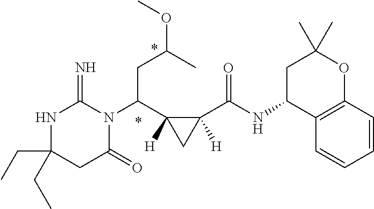 [SMILES: CCC1(CC)CC(=O)N(C(CC(C)OC)[C@H]2C[C@@H]2C(=O)N[C@@H]2CC(C)(C)Oc3ccccc32)C(=N)N1] | 499.4 |
| 144 | 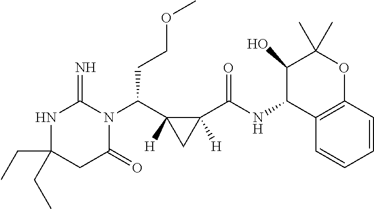 [SMILES: CCC1(CC)CC(=O)N([C@H](CCOC)[C@H]2C[C@@H]2C(=O)N[C@H]2c3ccccc3OC(C)(C)[C@@H]2O)C(=N)N1] | 501.3 |
| 145 | 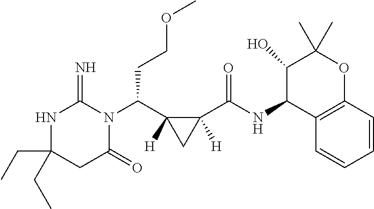 [SMILES: CCC1(CC)CC(=O)N([C@H](CCOC)[C@H]2C[C@@H]2C(=O)N[C@@H]2c3ccccc3OC(C)(C)[C@H]2O)C(=N)N1] | 501.1 |
| 146 | 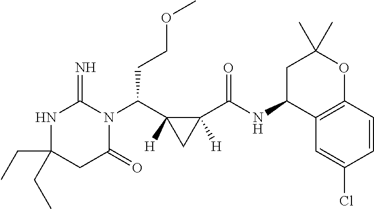 [SMILES: CCC1(CC)CC(=O)N([C@H](CCOC)[C@H]2C[C@@H]2C(=O)N[C@H]2CC(C)(C)Oc3ccc(Cl)cc32)C(=N)N1] | 519.3 |
| 147A | 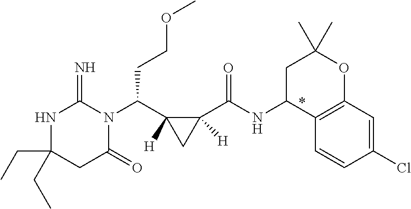 [SMILES: CCC1(CC)CC(=O)N([C@H](CCOC)[C@H]2C[C@@H]2C(=O)NC2CC(C)(C)Oc3cc(Cl)ccc32)C(=N)N1] | 519.1 |
| 147B | 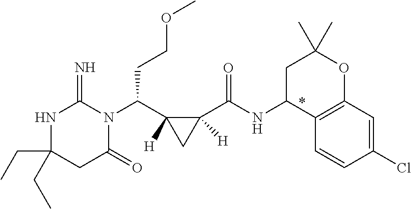 [SMILES: CCC1(CC)CC(=O)N([C@H](CCOC)[C@H]2C[C@@H]2C(=O)NC2CC(C)(C)Oc3cc(Cl)ccc32)C(=N)N1] | 519.1 |
| 148A | 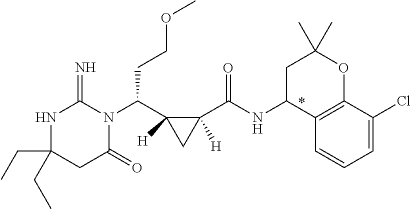 [SMILES: CCC1(CC)CC(=O)N([C@H](CCOC)[C@H]2C[C@@H]2C(=O)NC2CC(C)(C)Oc3c(Cl)cccc32)C(=N)N1] | 519.1 |
| 148B | 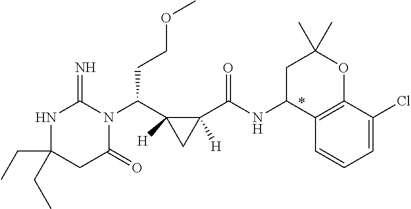 [SMILES: CCC1(CC)CC(=O)N([C@H](CCOC)[C@H]2C[C@@H]2C(=O)NC2CC(C)(C)Oc3c(Cl)cccc32)C(=N)N1] | 519.1 |
| 149A | 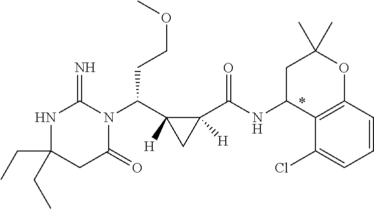 [SMILES: CCC1(CC)CC(=O)N([C@H](CCOC)[C@H]2C[C@@H]2C(=O)NC2CC(C)(C)Oc3cccc(Cl)c32)C(=N)N1] | 519.3 |
| 149B | 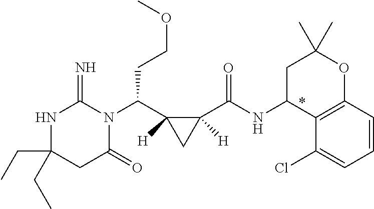 [SMILES: CCC1(CC)CC(=O)N([C@H](CCOC)[C@H]2C[C@@H]2C(=O)NC2CC(C)(C)Oc3cccc(Cl)c32)C(=N)N1] | 519.3 |
| 150 | 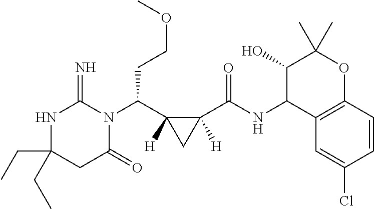 [SMILES: CCC1(CC)CC(=O)N([C@H](CCOC)[C@H]2C[C@@H]2C(=O)NC2c3cc(Cl)ccc3OC(C)(C)[C@H]2O)C(=N)N1] | 535.2 |

### Assessing Antiparasite Potency in a Parasite Lactate Dehydrogenase (LDH) Growth Assay (Parasite Assay)

The parasite stock was maintained at 4% haematocrit in RPMI-Hepes media buffered with sodium bicarbonate and supplemented with 5% heat inactivated human serum and 0.5% albumax.

Approximately 42 hours prior to the potency assay being set up, parasites were synchronized with 5% sorbitol to select for ring stage parasites. On the day of assay set up, a blood smear of the parasite culture was Giemsa stained and counted. The parasitemia was adjusted to 0.7% rings and the haematocrit was diluted to 2% in RPMI-Hepes media buffered with sodium bicarbonate and supplemented with 5% heat inactivated human serum and 0.5% albumax. 30 μl of diluted parasites are then added into 10 μl of media+compound in pre-prepared Greiner TC assay plates. Parasite assay plates were placed in gassed humidified boxes in single layer and allowed to incubate at 37° C. for 72 hours. After 72 hours growth, assay plates were sealed with parafilm and frozen flat, in single file at −80° C. overnight. On the following day, assay plates were allowed to thaw at room temperature for 4 hours to which an LDH assay was performed to measure parasite growth.

Assay EC₅₀ results are shown in Table 3 and Table 4.

TABLE 3

| Example | EC₅₀ (nM) |
|---|---|
| 1A | 1 |
| 1B | 250 |
| 2 | 0.9 |
| 3A | 234 |
| 3B | 151 |
| 4A | 94 |
| 4B | 26 |
| 4C | 85 |
| 4D | 91 |
| 5 | 49 |
| 6A | 188 |
| 6B | 269 |
| 7A | 135 |
| 7B | 29 |
| 8A | 37 |
| 8B | 5.3 |
| 9 | 6.9 |
| 10 | 27 |
| 11A | 4.1 |
| 11B | 24 |
| 12 | 0.9 |
| 13 | 24 |
| 14 | 0.9 |
| 15A | 240 |
| 15B | 230 |
| 16A | 33 |
| 16B | 119 |
| 17A | 47 |
| 17B | 20 |
| 18A | 39 |
| 18B | 180 |
| 19A | 28 |
| 19B | 4.2 |
| 20 | 5.3 |
| 21A | 260 |
| 21B | 1 |
| 22 | 44 |
| 23A | 16 |
| 23B | 15 |
| 24A | 3.4 |
| 24B | 2 |
| 24C | 120 |
| 25A | 84 |
| 25B | 25 |
| 26 | 4.7 |
| 27 | 78 |
| 28A | 12 |
| 28B | 14 |
| 29A | 203 |
| 29B | 351 |
| 29C | 113 |
| 30A | 45 |
| 30B | 7.7 |
| 31A | 215 |
| 31B | 213 |
| 32 | 1.8 |
| 33 | 76 |
| 34 | 286 |
| 35A | 85 |
| 35B | 220 |
| 36A | 247 |
| 36B | 301 |
| 37 | 1.2 |
| 38 | 6.5 |
| 39 | 10.1 |
| 40 | 3.1 |
| 41 | 1.1 |
| 42 | 18.4 |
| 43A | 4 |
| 43B | 37 |
| 44A | 8.3 |
| 44B | 0.9 |
| 45A | 2.8 |
| 45B | 220 |
| 46 | 2.5 |
| 47A | 155 |
| 47B | 0.9 |
| 48A | 2.7 |
| 48B | 7.4 |
| 49A | 20 |
| 49B | 14 |
| 50 | 3 |
| 51 | 5.3 |
| 52 | 1 |
| 53A | 3.1 |
| 53B | 2.1 |
| 54A | 1 |
| 54B | 130 |
| 55A | 0.8 |
| 55B | 180 |
| 56A | 65 |
| 56B | 0.7 |
| 57A | 3.2 |
| 57B | 100 |
| 58A | 210 |
| 58B | 1 |
| 59 | 3.8 |
| 60A | 0.5 |
| 60B | 100 |
| 61 | 6.9 |
| 62A | 31 |
| 62B | 0.8 |
| 63 | 1.2 |
| 64A | 2.3 |
| 64B | 195 |
| 65A | 2.8 |
| 65B | 8 |
| 66 | 2.6 |
| 67A | 0.9 |
| 67B | 0.6 |
| 68 | 0.3 |
| 69A | 1 |
| 69B | 1.9 |
| 69C | 0.8 |
| 69D | 0.3 |
| 69E | 300 |
| 70A | 77 |
| 70B | 19 |
| 71 | 125 |
| 72A | 11 |
| 72B | 2.5 |
| 73 | 192 |
| 74A | 33 |
| 74B | 3.1 |
| 75A | 8.2 |
| 75B | 7.5 |
| 75C | 7 |
| 75D | 7 |
| 76A | 17 |
| 76B | 15 |
| 77A | 1.7 |
| 77B | 2.3 |
| 78 | 300 |
| 79A | 255 |
| 79B | 275 |
| 79C | 289 |
| 80A | 9.4 |
| 80B | 6.6 |
| 80C | 14 |
| 80D | 21 |
| 81A | 2.7 |
| 81B | 1 |
| 81C | 0.9 |
| 81D | 1.3 |
| 82A | 24.4 |
| 82B | 57 |
| 82C | 53 |
| 82D | 27 |
| 83A | 6.6 |
| 83B | 26 |
| 84 | 289 |
| 85A | 2.4 |
| 85B | 0.9 |
| 86A | 2.2 |
| 86B | 2.8 |
| 87 | 160 |
| 88A | 5.2 |
| 88B | 10 |
| 89A | 5.6 |
| 89B | 22 |
| 90 | 225 |
| 91A | 0.8 |
| 91B | 2.7 |
| 92A | 7.6 |
| 92B | 7.2 |
| 93A | 3.3 |
| 93B | 2.4 |
| 94A | 0.9 |
| 94B | 0.9 |
| 95 | 110 |
| 96A | 2.7 |
| 96B | 3.3 |
| 97A | 180 |
| 97B | 220 |
| 98A | 0.9 |
| 98B | 2.2 |
| 99 | 130 |
| 100 | 170 |
| 101 | 2.9 |
| 102A | 0.8 |
| 102B | 2.5 |
| 103A | 29 |
| 103B | 2.1 |
| 104A | 4 |
| 104B | 22 |
| 105A | 0.9 |
| 105B | 1.8 |
| 105C | 0.8 |
| 105D | 0.9 |
| 106A | 7.4 |
| 106B | 10 |
| 107A | 2.1 |
| 107B | 11 |
| 108A | 70 |
| 108B | 3.7 |
| 108C | 6 |
| 108D | 8.5 |
| 109A | 2.3 |
| 109B | 1 |
| 110A | 21 |
| 110B | 7.2 |
| 111A | 24 |
| 111B | 15 |
| 112A | 18 |
| 112B | 9.4 |
| 113A | 19 |
| 113B | 7.5 |
| 114A | 89 |
| 114B | 5.1 |
| 115A | 6.6 |
| 115B | 1 |
| 116A | 0.8 |
| 116B | 18 |
| 117A | 0.8 |
| 117B | 2.7 |
| 118 | 63 |
| 119 | 26 |
| 120 | 1 |
| 121A | 2.5 |
| 121B | 2 |
| 122A | 2.3 |
| 122B | 2.2 |
| 123 | 2 |
| 124 | 0.8 |
| 125A | 231 |
| 125B | 160 |
| 126A | 4.7 |
| 126B | 8.4 |
| 127A | 1.1 |
| 127B | 2.6 |
| 128 | 280 |
| 129A | 1.3 |
| 129B | 2.5 |
| 130A | 0.9 |
| 130B | 2.4 |
| 131A | 6.8 |
| 131B | 6.1 |
| 132 | 6.7 |
| 133A | 210 |
| 133B | 100 |
| 134A | 71 |
| 134B | 25 |
| 135 | 190 |
| 136 | 160 |
| 137A | 12 |
| 137B | 6.4 |
| 138A | 7.4 |
| 138B | 18 |
| 139A | 99 |
| 139B | 130 |
| 140A | 220 |
| 140B | 280 |

TABLE 4

| | |
|---|---|
| 141 | 4.7 |
| 142A | 1.9 |
| 142B | 0.7 |
| 142C | 204.9 |
| 142D | 114.9 |
| 143A | 4.6 |
| 143B | 2.4 |
| 143C | 57.9 |
| 144 | 164.9 |
| 145 | 8.4 |
| 146 | 1.1 |
| 147A | 2.8 |
| 147B | 8.2 |
| 148A | 2.7 |
| 148B | 1.9 |
| 149A | 1.0 |
| 149B | 1.2 |
| 150 | 3.9 |

### FRET Based Assay for Plasmepsin IX and Plasmepsin X Cleavage of Peptide Substrates

### General Protocol:

For cleavage assays, 10-point dilution series of the compounds (Compound EC₅₀ determined by LDH assay) were prepared in 1536 well black low volume assay plates (Corning #3728) using an Acoustic Dispenser Echo555 (Labcyte). Appropriate volumes of 10 mM compound stocks were transferred into the assay plates starting at a top dose of 0.9 μM for PfPMX and 90 μM for PfPMIX in a 1:3 fold dilution series with 1% DMSO final concentration. IC₅₀ values were analysed in Graphpad Prism software using a nonlinear regression four-parameter fit analysis. The equation used was sigmoidal dose response (variable slope), Y=bottom+(top_bottom)/(1+10((log EC₅₀_X) 3 Hill Slope)).

All compound potency assays were conducted in 5 μl total volume. For each assay, 2.5 μL of recombinant PMIX or PMX in respective assay buffers, shown in Table 5, were dispensed into compound containing assay plates using a Multidrop Combi dispenser and allowed to incubate for 15 min. The reactions were started with a further 2.5 μL addition of FRET peptide substrates and reactions incubated at 37° C. for various time, as shown in Table 5. Samples were excited at 340 nm and fluorescence emission measured at 490 nm using PHERAstar FSX plate reader (BMG). The 0% inhibition control contained DMSO (1% final) and the 100% inhibition control was minus enzyme.

TABLE 5
Peptide Cleavage Assays Parameters

| | PMIX | PMX |
|---|---|---|
| Enzyme quantity | 8 nM enzyme | 0.2 nM enzyme |
| Buffer | 25 mM Sodium | 0.005% Tween-20, |
| | Acetate, 100 mM | pH 5.5 |
| | NaCl, 0.005% | 25 mM Sodium |
| | Tween-20, pH 5.5 | Acetate, |
| Substrate peptide | RON3 | Rh2 |
| Peptide final concentration | 7 μM | 1.8 μM |
| Incubation time at 37° C. | 2 hr | 2 hr |

To confirm that Example 2 selectively inhibits PMX function, the ability to inhibit cleavage of a known substrate for this protease was tested using a FRET (Fluorescence Resonance Energy Transfer) based assay, recombinantly expressed PMX and synthetic fluorogenic peptides corresponding to P. falciparum sequences of the PMX-specific substrates Rh2. Rh2 is a protein required for merozoite invasion and is processed by PMX (Triglia et al., 2011, Favuzza et al. 2020).

To confirm whether Example 2 inhibits PMIX function, the ability to inhibit cleavage of a known substrate for this protease was tested using the already established FRET based assay, recombinantly expressed PMIX, and RON3 fluorogenic peptide. RON3 is a protein required for ring-stage parasite development after invasion and is processed by PMIX (Low et al., 2019, Favuzza et al. 2020).

For cleavage assays, 10-point dilution series of the compounds, recombinant PMIX or PMX, and FRET peptide substrates were prepared in 1536 well plates. Samples were excited at 340 nm and fluorescence emission measured at 492 nm using an Envision fluorescence plate reader (Perkin-Elmer).

IC₅₀ values were calculated by Dotmatics 5.3 and Spotfire 7.11.1 software using a nonlinear regression four-parameter fit analysis. The equation used is sigmoidal dose response (variable slope). GraphPad Prism software was used to plot the kinetic data.

The results of assays described above are shown in Table 6. As shown in Table 6, Example 2 is a selective PMX inhibitor with over 200-fold selectivity for PMX over PMIX.

TABLE 6

| | PfLDH | | | Fold |
|---|---|---|---|---|
| | Assay | | | Selectivity |
| Example | (EC50, | PfPMX | PfPMIX | PMX over |
| No. | nM) | (Ki, nM) | (Ki, nM) | PMIX |
| 2 | 0.86 (AVG) | 0.026 (AVG) | 5.95 (AVG) | 228.8 |
| | 0.33 (SD) | 0.004 (SD) | 0.14 (SD) | |
| | 3 (N) | 2 (N) | 2 (N) | |

### Chemovaccination of Mice

### General Experimental Procedure

The indicated number of Plasmodium berghei sporozoites expressing mCherry and luciferase reporters (PbmCherryLuci) were inoculated intravenously, and compounds were administered to mice by oral gavage 36 and 48 hours after injection during liver infection. Plasmodium berghei liver infection levels and egress of parasites from the liver were measured by bioluminescence signal from the parasites using the luciferase reporter and an In Vivo Imaging System (IVIS, Perkin Elmer) at the peak of liver infection (52 hours post infection, hpi), during liver egress (55 hpi) as well as during the first round of blood infection (65 hpi). Initiation of blood infection was also measured by flow cytometry at 65 hpi using the mCherry reporter to determine the parasitemia of this first round of blood infection. These analyses allowed quantification of the efficacy of drug killing of parasites in the liver (52 hpi), preventing their egress from the liver (55 hpi) or preventing their successful infection of the blood (65 hpi). Mice were monitored by giemsa stained thin blood smears for the presence of parasites in the blood for 30 days post infection. If no blood infection was seen during the subsequent 30 days, the mice were declared cured of malaria infection thus indicating the chemoprophylactic activity of these compounds.

The chemoprophylactic activity of administered compounds is not to kill liver Plasmodium berghei parasites nor to prevent their egress from the liver, but rather that liver-derived merozoites were unable to initiate a blood infection in these mice. The parasites are thus attenuated by the administered compounds. Such parasite attenuation by genetic means is a powerful vaccination strategy, and as such cure of liver parasites by the compounds could simultaneously be considered as chemoprophylaxis as well as a chemovaccination strategy. Mice previously cured of liver infection with the administered compounds (Chemovaccinated) were challenged with bites from 10 PbmCherryLuci infected mosquitoes at the indicated times after cure of liver infection with the administered compounds to determine the level of immunity engendered by this chemovaccination approach.

Chemovaccination of Mice with Example 2

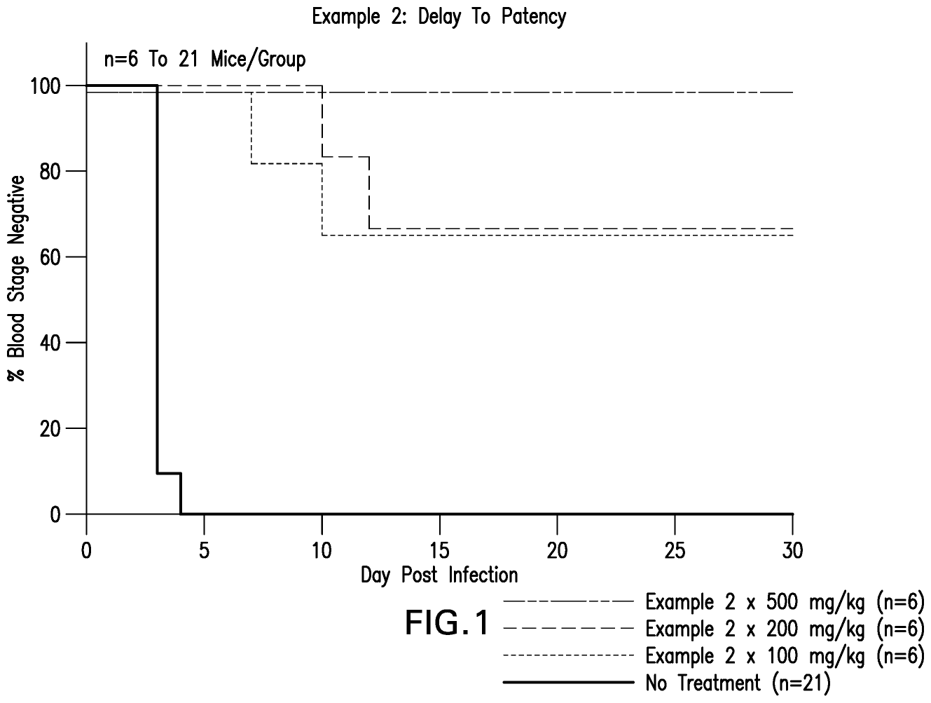

C57BL/6 mice were administered Example 2, as described in the general procedure. Thirty nine mice were infected with 40,000 sporozoites. Twenty-one mice were untreated. Six C57BL/6 mice were administered 2×100 mg/kg of Example 2. Six C57BL/6 mice were administered 2×200 mg/kg. Six C57BL/6 mice were administered 2×500 mg/kg. Example 2 dosed at 2×100 mg/kg cured four of six mice of their infection, as did mice dosed with 2×200 mg/kg of Example 2. As shown in the graph of FIG. 1, Example 2 dosed at 2×500 mg/kg cured all mice of their infection, demonstrating complete chemoprophylaxis. These infections and treatments can be considered immunizations or chemovaccinations, since the parasite is attenuated by Example 2 and is unable to initiate the blood infection.

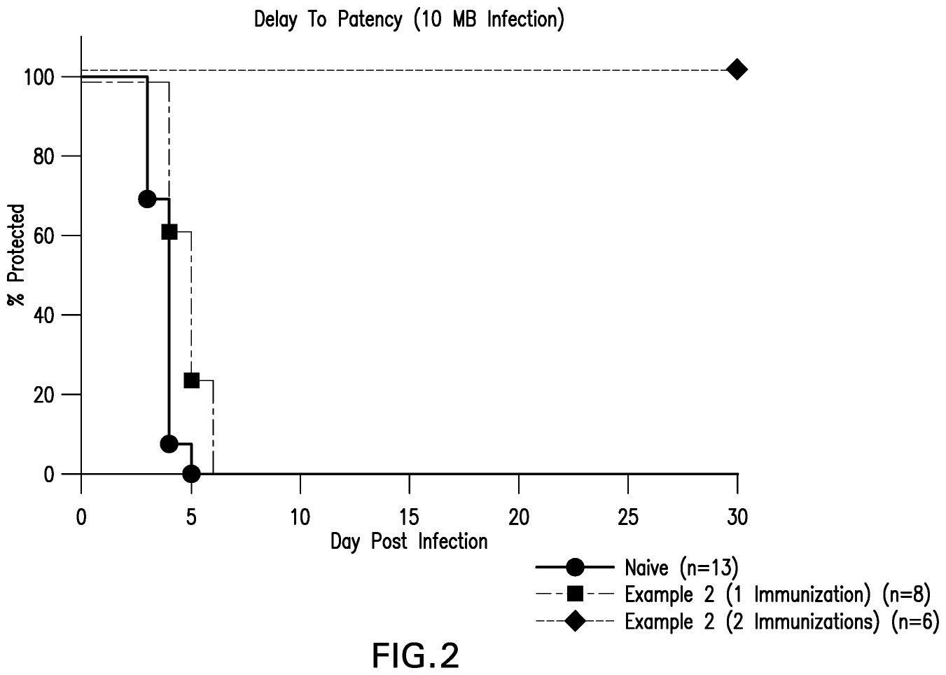

Additionally, the eight mice previously chemovaccinated with 2×100 mg/kg and 2×200 mg/kg of Example 2 were challenged three months later with bites from 10 mosquitoes infected with PbmCherryLuci sporozoites. These mice that were immunized once showed a reduction in infection upon challenge, demonstrating partial immunity, but all mice ultimately developed malaria disease. The six mice cured with 2×500 mg/kg Example 2 were six weeks later immunized again with 20,000 sporozoites and treated with 2×500 mg/kg of Example 2 that cured them of this second infection. This second infection and treatment is considered a secondary ‘boost’ immunization, and when these mice were challenged 3 months later with the bites from 10 mosquitoes infected with PbmCherryLuci they were completely protected from infection. Therefore, as shown in FIG. 2, two immunizations were sufficient for 100% protection in the C57BL/6 mice when challenged 3 months after the initial infection by mosquito bite challenge.

1. A method of chemovaccination against Plasmodium infection comprising administration to a patient of an effective amount of a selective inhibitor of plasmepsin X.

2. The method of claim 1, wherein the selective inhibitor of plasmepsin X is a compound of structural Formula (I): 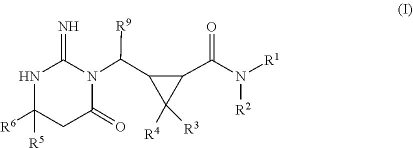
or a pharmaceutically acceptable salt thereof, wherein:
R¹ is a heterocycloalkyl, C₃-C₁₂cycloalkyl, aryl or C₁-C₆alkylaryl, wherein the heterocycloalkyl, C₃-C₁₂cycloalkyl, aryl, or C₁-C₆alkylaryl is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), N(R⁷)(R⁸) and C₁-C₆alkylN(R⁷)(R⁸);
R² is hydrogen, C₁-C₆alkylCOOH, —COOH, C₃-C₆cycloalkyl, C₁-C₆alkyl, haloC₁-C₆alkyl or C₁-C₆alkylOH;
R³ is hydrogen, halogen, —CN, —OH, C₃-C₆cycloalkyl or C₁-C₆alkyl;
R⁴ is hydrogen, halogen, —CN, —OH, C₃-C₆cycloalkyl or C₁-C₆alkyl;
R⁵ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, —COOH, C₃-C₆cycloalkyl, C₁-C₆alkyl, haloC₁-C₆alkyl or C₁-C₆alkylOH;
R⁸ is hydrogen, C₁-C₆alkylCOOH, —COOH, C₃-C₆cycloalkyl, C₁-C₆alkyl, haloC₁-C₆alkyl or C₁-C₆alkylOH; and
R is hydrogen, halogen, —CN, —OH, alkoxy, C₁-C₆alkyl, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and N(R⁷)(R⁸), wherein the C₁-C₆alkyl is unsubstituted or substituted with one, two or three substituents independently selected from the group consisting of halogen, —CN, —OH, alkoxy, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and —N(R⁷)(R⁸), and wherein the heterocycloalkyl, heteroaryl, C₃-C₁₂cycloalkyl and aryl are unsubstituted or substituted with one, two or three substituents independently selected from the group consisting of halogen, —CN, —OH, alkoxy, C₁-C₆alkyl, —COOH, oxo, —COOC₁-C₆alkyl, haloC₁-C₆alkyl, —CON(R⁷)(R⁸) and —N(R⁷)(R⁸).

3. The method of claim 2, wherein in the compound of structural formula (I), or pharmaceutically acceptable salt thereof, R¹ is selected from the group consisting of: 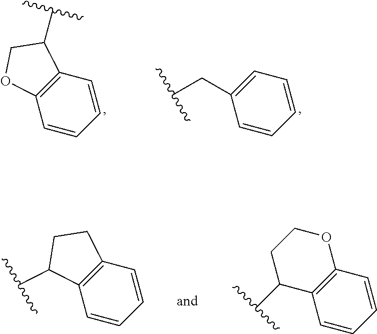
wherein R¹ is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), N(R⁷)(R⁸) and C₁-C₆alkylN(R⁷)(R⁸);
R⁷ is hydrogen, C₁-C₆alkylCOOH, —COOH, C₃-C₆cycloalkyl, C₁-C₆alkyl, haloC₁-C₆alkyl or C₁-C₆alkylOH; and
R⁸ is hydrogen, C₁-C₆alkylCOOH, —COOH, C₃-C₆cycloalkyl, C₁-C₆alkyl, haloC₁-C₆alkyl or C₁-C₆alkylOH.

4. The method of claim 2, wherein in the compound of structural formula (I), or pharmaceutically acceptable salt thereof, R¹ is: 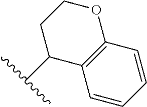
wherein R¹ is unsubstituted or substituted with 1 to 5 substituents independently selected from the group consisting of halogen, —CN, —OH, alkoxy, haloalkoxy, C₁-C₆alkylOC₁-C₆alkyl, oxo, —COOC₁-C₆alkyl, C₁-C₆alkyl, haloC₁-C₆alkyl and C₁-C₆alkylOH.

5. The method of claim 2, wherein in the compound of structural formula (I), or pharmaceutically acceptable salt thereof, R¹ is: 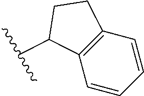
wherein R¹ is unsubstituted or substituted with 1 to 5 substituents independently selected from the group consisting of halogen, —CN, —OH, alkoxy, haloalkoxy, C₁-C₆alkylOC₁-C₆alkyl, oxo, —COOC₁-C₆alkyl, C₁-C₆alkyl, haloC₁-C₆alkyl C₃-C₆cycloalkyl, spiroC₃-C₆cycloalkyl, and C₁-C₆alkylOH.

6. The method of claim 2, wherein in the compound of structural formula (I), or pharmaceutically acceptable salt thereof, R² is hydrogen.

7. The method of claim 2, wherein in the compound of structural formula (I), or pharmaceutically acceptable salt thereof, R¹ is a chromane or indane.

8. The method of claim 7, wherein in the compound of structural formula (I), or pharmaceutically acceptable salt thereof, R¹ is:

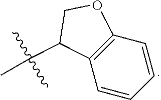 [SMILES: *C1COc2ccccc21]

9. The method of claim 2, wherein in the compound of structural formula (I), or pharmaceutically acceptable salt thereof, R³ is hydrogen, halogen or C₁-C₆alkyl.

10. The method of claim 2, wherein in the compound of structural formula (I), or pharmaceutically acceptable salt thereof, R⁴ is hydrogen, halogen or C₁-C₆alkyl.

11. The method of claim 2, wherein in the compound of structural formula (I), or pharmaceutically acceptable salt thereof, R⁵ is hydrogen or C₁-C₆alkyl.

12. The method of claim 2, wherein in the compound of structural formula (I), or pharmaceutically acceptable salt thereof, R⁶ is hydrogen or C₁-C₆alkyl.

13. (canceled)

14. (canceled)

15. The method of claim 2, wherein in the compound of structural formula (I), or pharmaceutically acceptable salt thereof, R⁹ is hydrogen, C₁-C₆alkyl, heteroaryl, or aryl, wherein the C₁-C₆alkyl, heteroaryl or aryl is unsubstituted or substituted with one, two or three substituents independently selected from the group consisting of halogen, —CN, —OH, alkoxy.

16. The method of claim 2, wherein the selective inhibitor of plasmepsin X is a compound having a structure selected from the group consisting of: 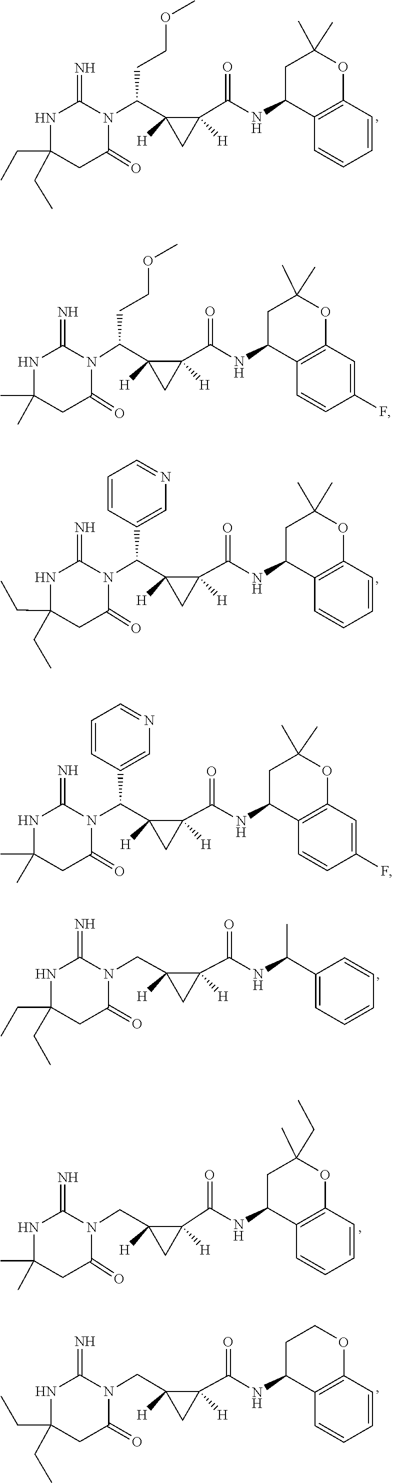 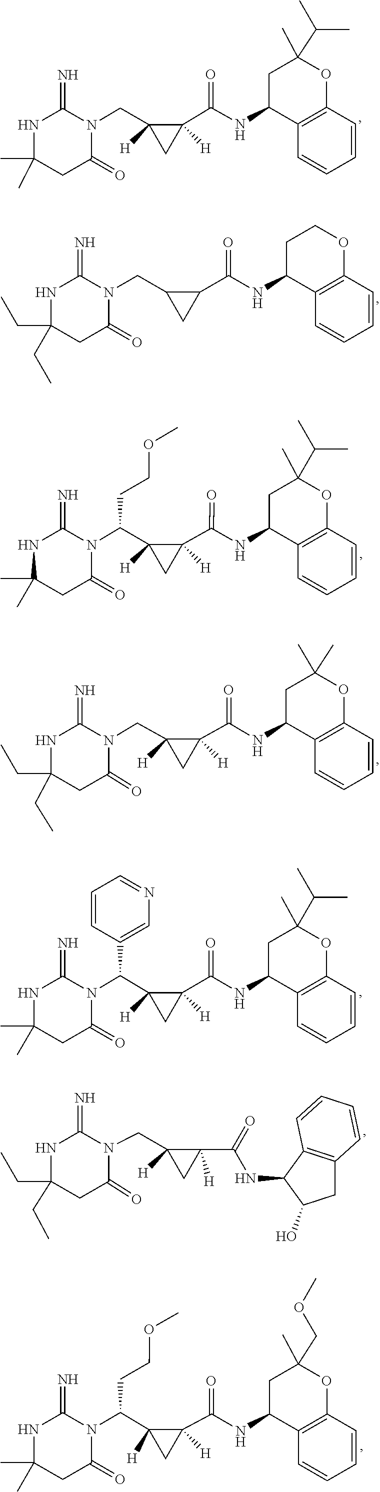 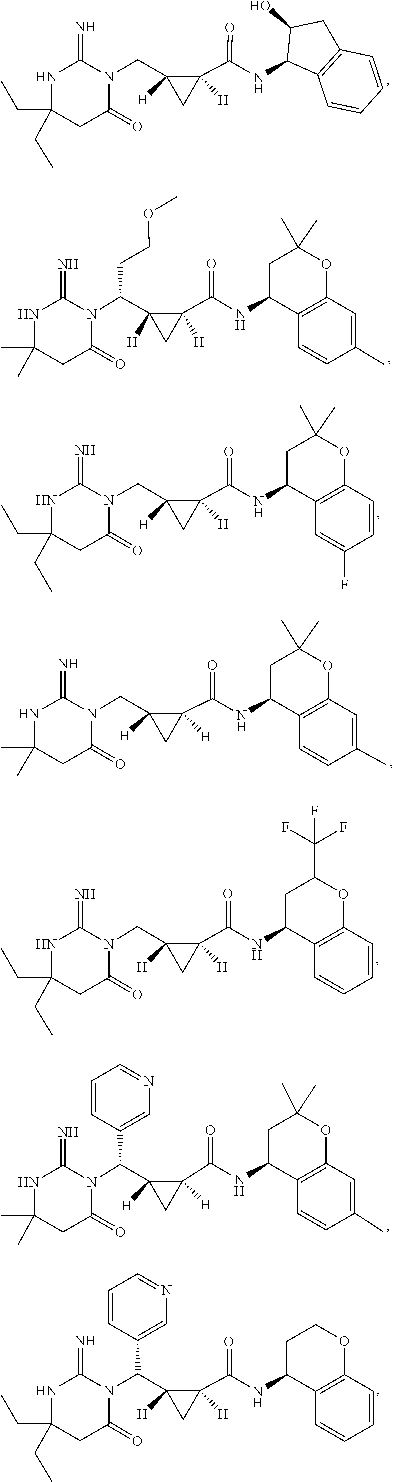 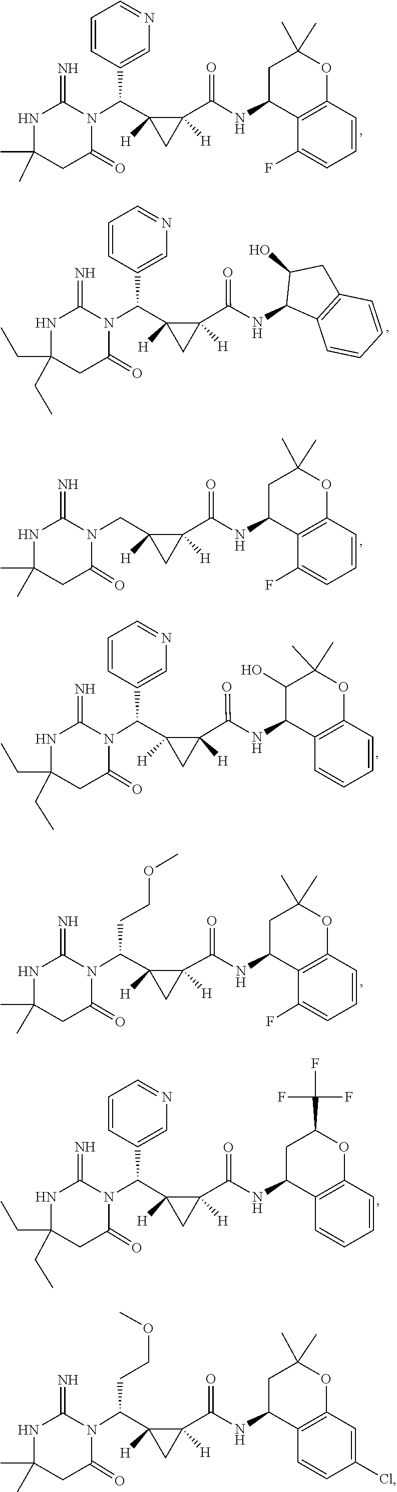 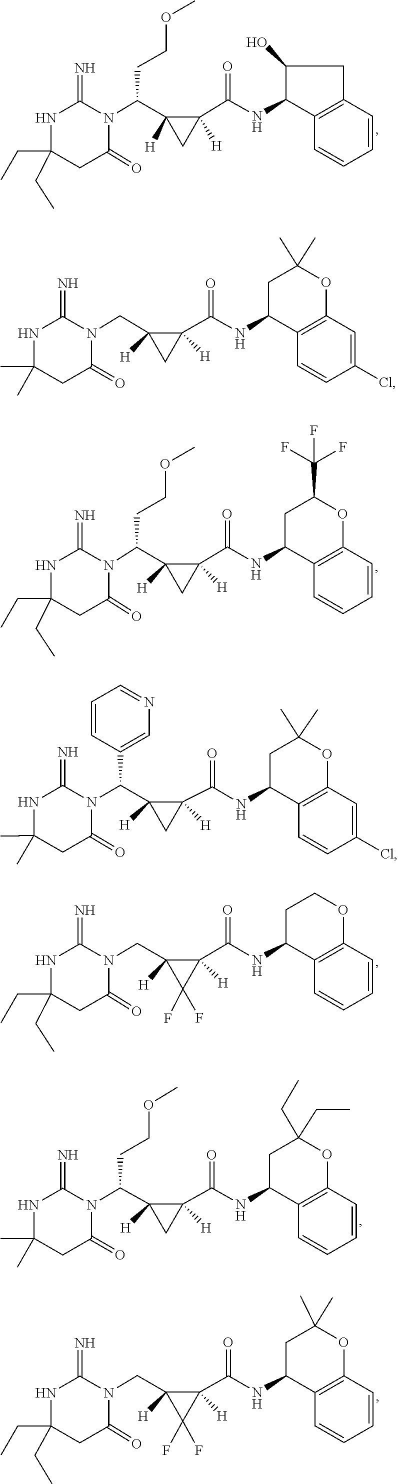 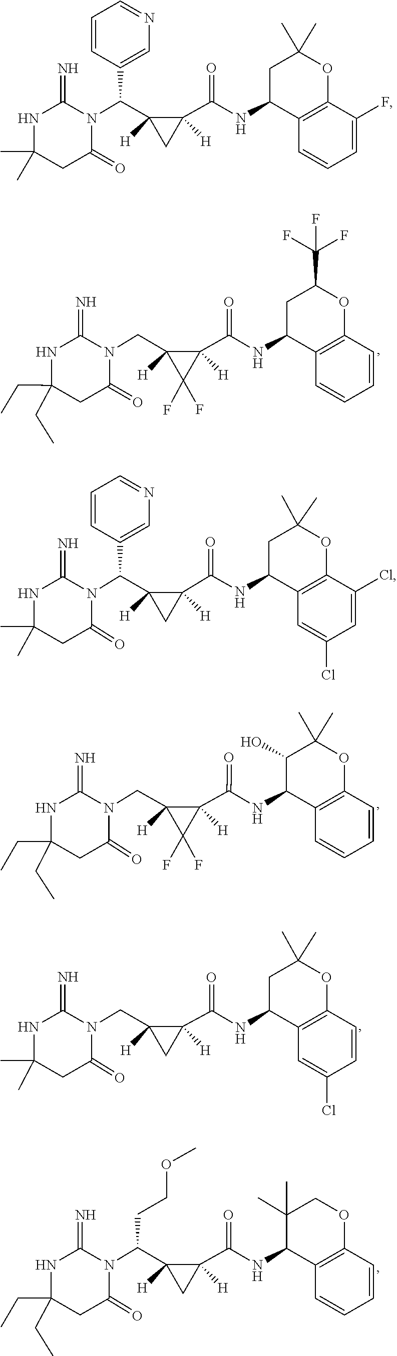 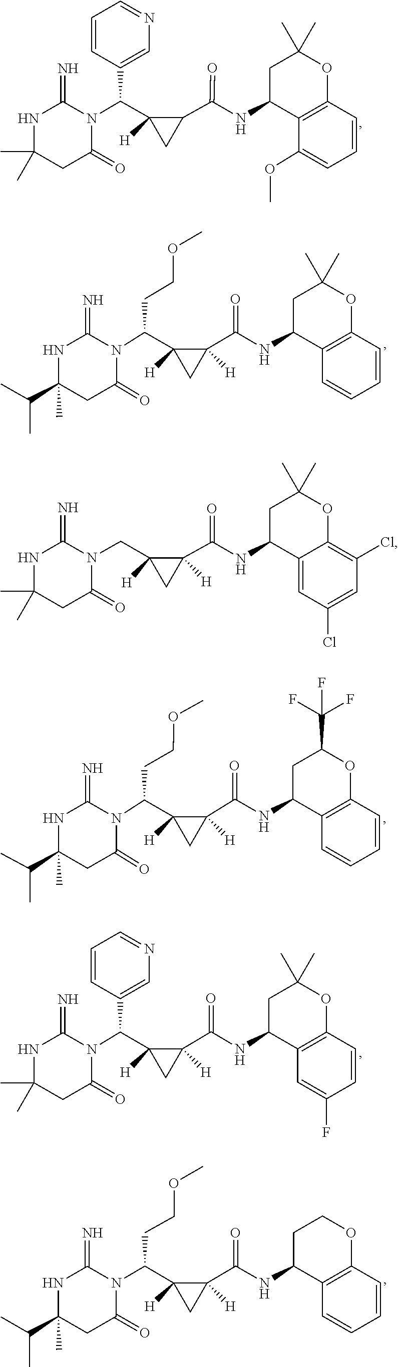 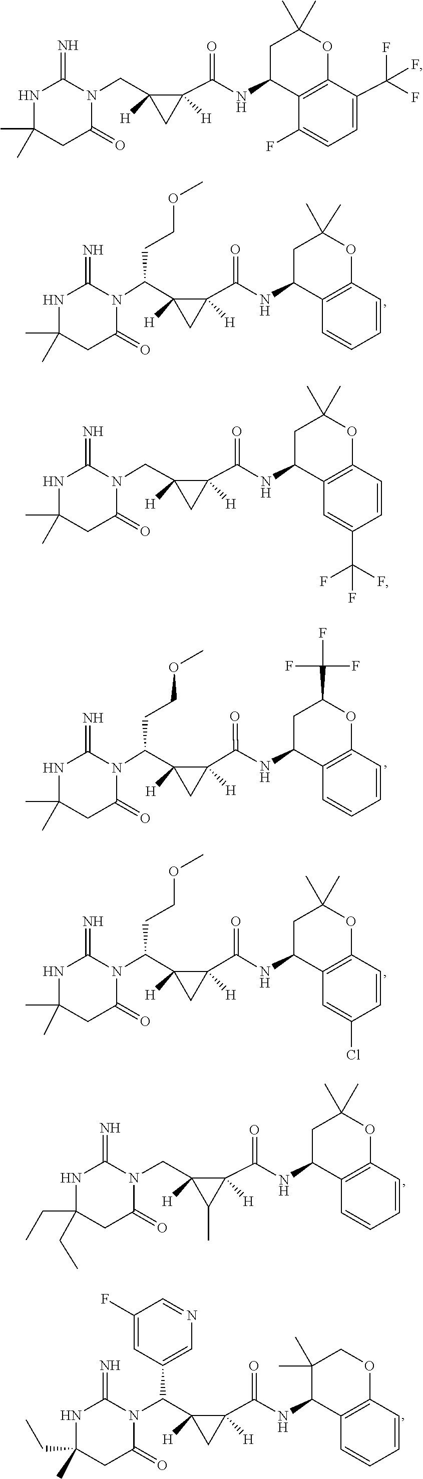 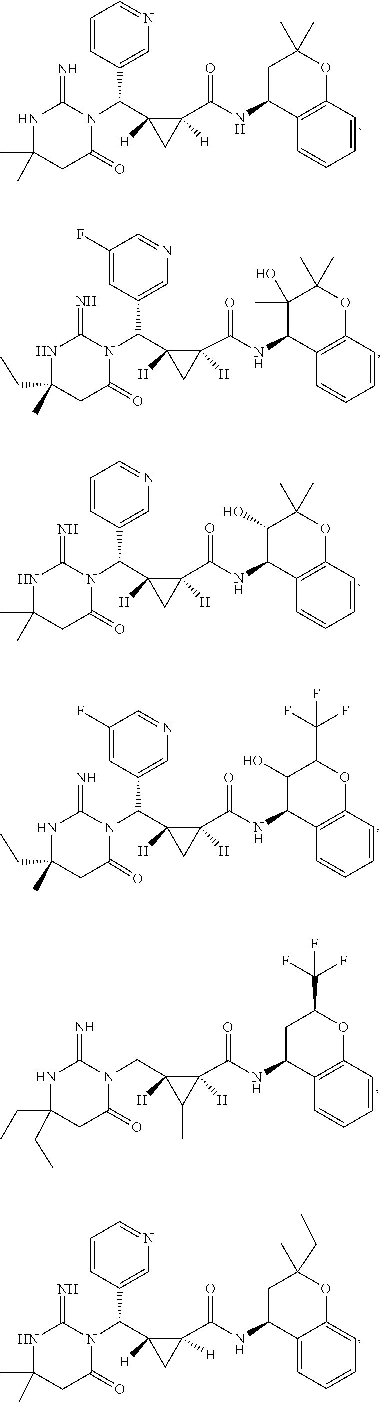 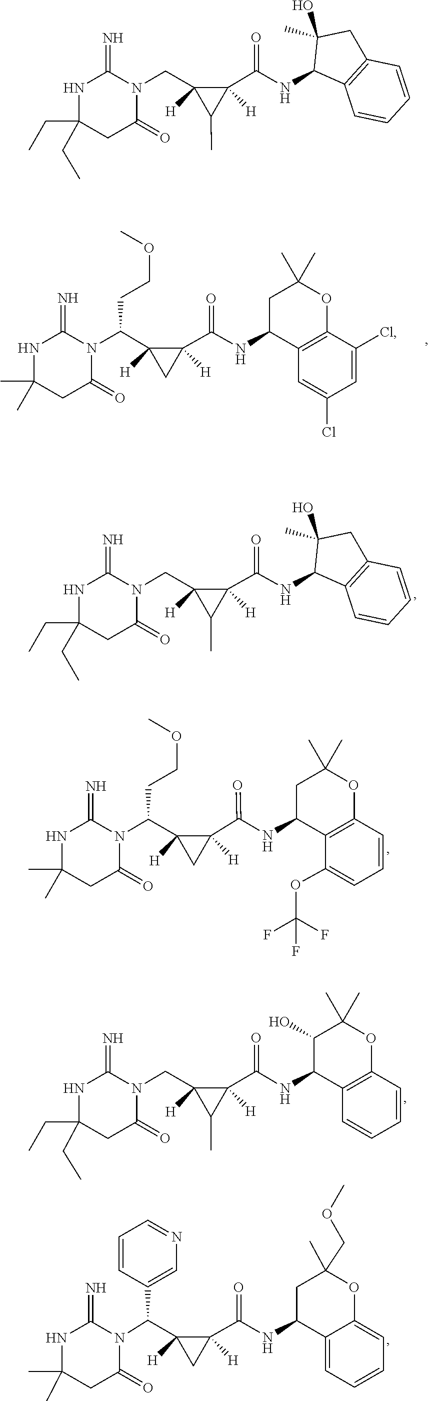 [SMILES: CCC1(CC)CC(=O)N(C[C@H]2C(C)[C@@H]2C(=O)N[C@@H]2c3ccccc3C[C@]2(C)O)C(=N)N1 ; COCC[C@H]([C@H]1C[C@@H]1C(=O)N[C@H]1CC(C)(C)Oc2c(Cl)cc(Cl)cc21)N1C(=N)NC(C)(C)CC1=O ; CCC1(CC)CC(=O)N(C[C@H]2C(C)[C@@H]2C(=O)N[C@@H]2c3ccccc3C[C@]2(C)O)C(=N)N1 ; COCC[C@H]([C@H]1C[C@@H]1C(=O)N[C@H]1CC(C)(C)Oc2cccc(OC(F)(F)F)c21)N1C(=N)NC(C)(C)CC1=O ; CCC1(CC)CC(=O)N(C[C@H]2C(C)[C@@H]2C(=O)N[C@@H]2c3ccccc3OC(C)(C)[C@H]2O)C(=N)N1 ; COCC1(C)C[C@H](NC(=O)[C@H]2C[C@@H]2[C@@H](c2cccnc2)N2C(=N)NC(C)(C)CC2=O)c2ccccc2O1] 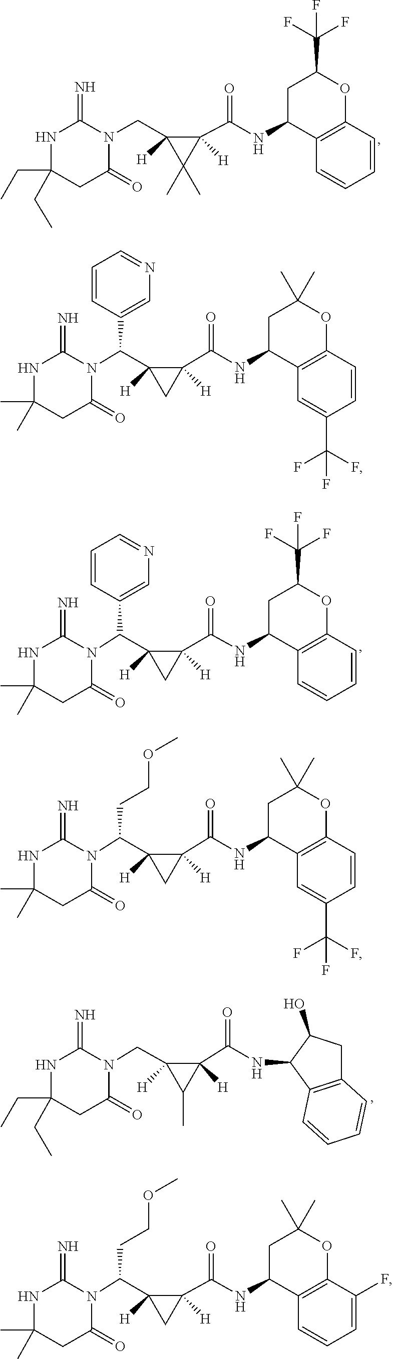 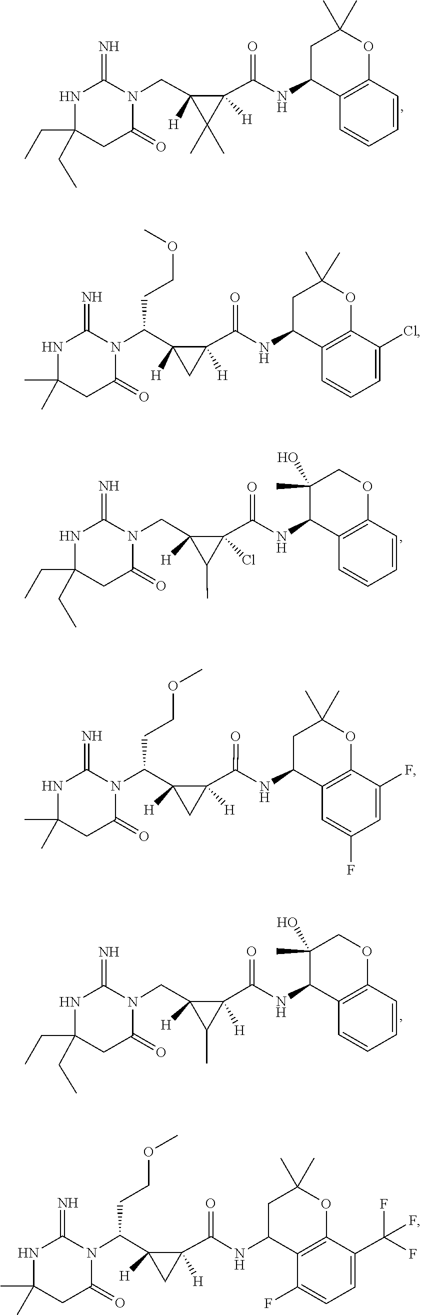 [SMILES: CCC1(CC)CC(=O)N(C[C@@H]2[C@@H](C(=O)N[C@H]3CC(C)(C)Oc4ccccc43)C2(C)C)C(=N)N1 ; COCC[C@H]([C@H]1C[C@@H]1C(=O)N[C@H]1CC(C)(C)Oc2c(Cl)cccc21)N1C(=N)NC(C)(C)CC1=O ; CCC1(CC)CC(=O)N(C[C@H]2C(C)[C@]2(Cl)C(=O)N[C@@H]2c3ccccc3OC[C@@]2(C)O)C(=N)N1 ; COCC[C@H]([C@H]1C[C@@H]1C(=O)N[C@H]1CC(C)(C)Oc2c(F)cc(F)cc21)N1C(=N)NC(C)(C)CC1=O ; CCC1(CC)CC(=O)N(C[C@H]2C(C)[C@@H]2C(=O)N[C@@H]2c3ccccc3OC[C@@]2(C)O)C(=N)N1 ; COCC[C@H]([C@H]1C[C@@H]1C(=O)NC1CC(C)(C)Oc2c(C(F)(F)F)ccc(F)c21)N1C(=N)NC(C)(C)CC1=O] 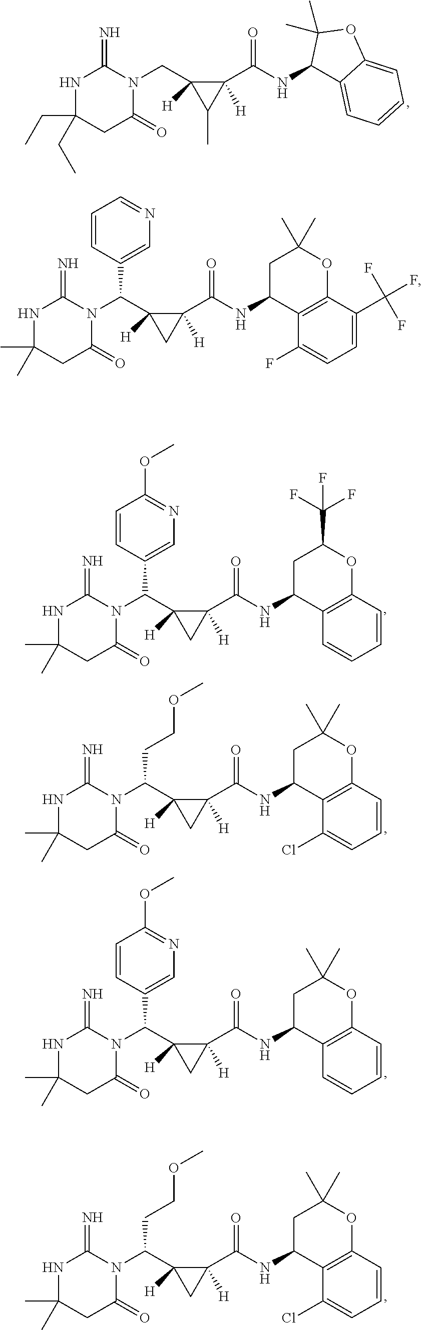 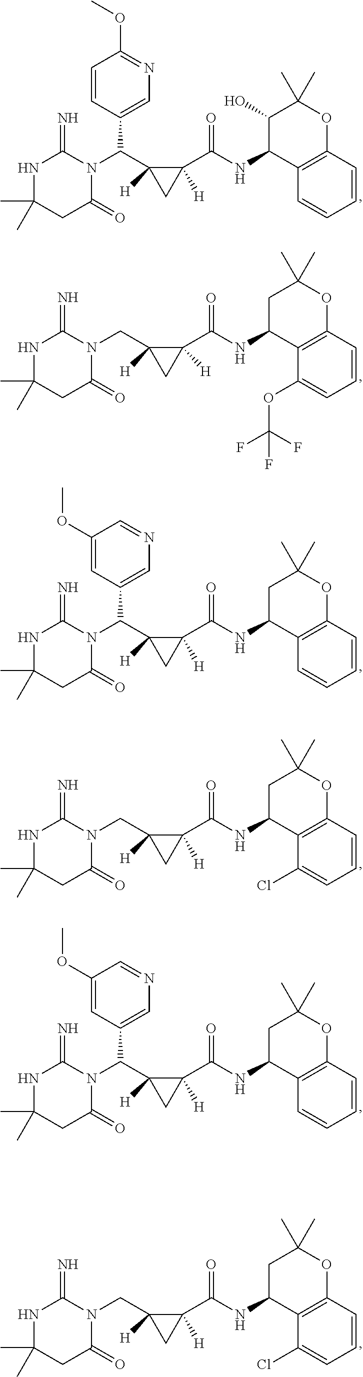 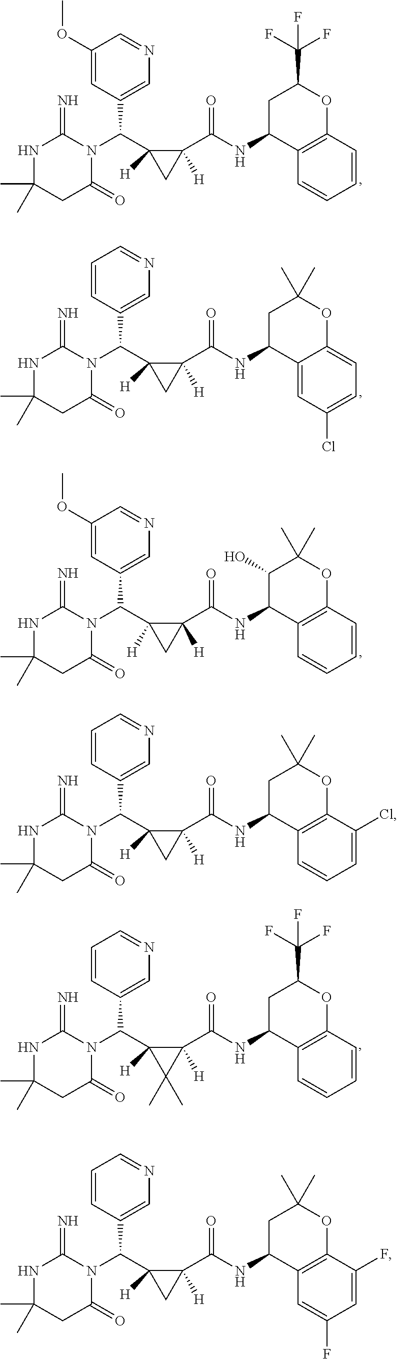 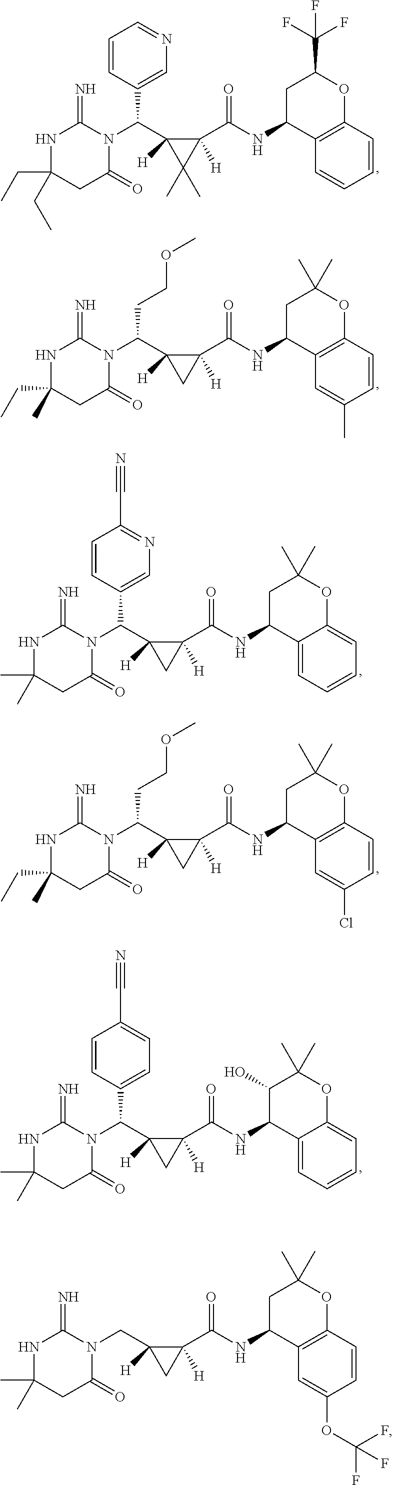 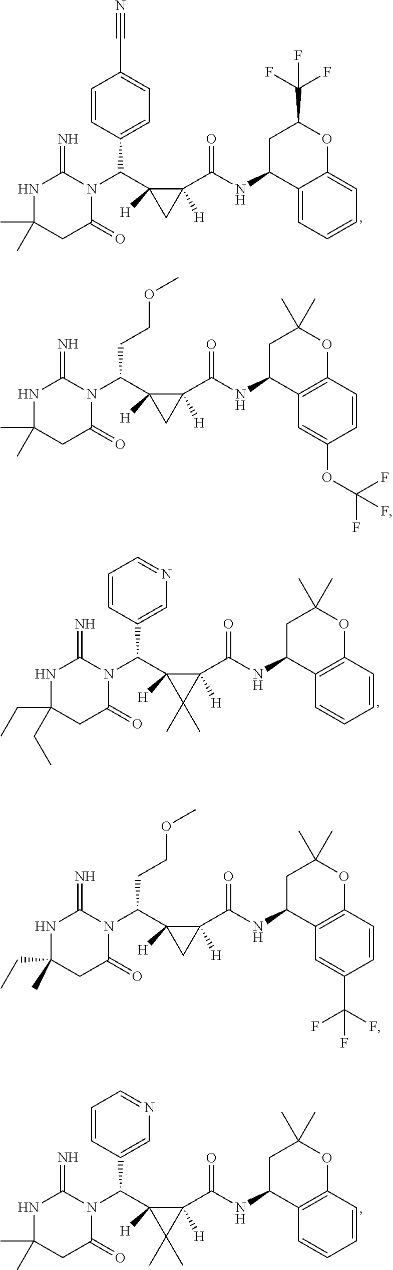 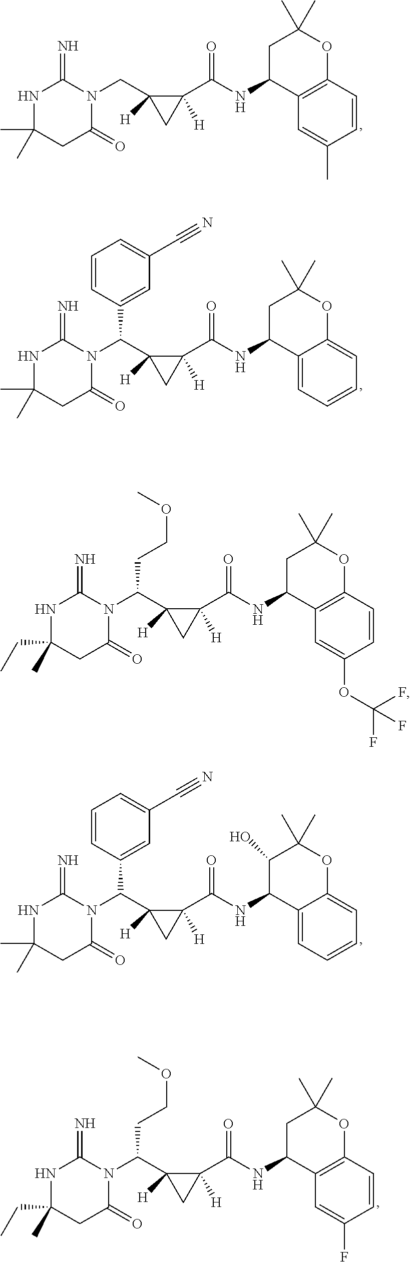 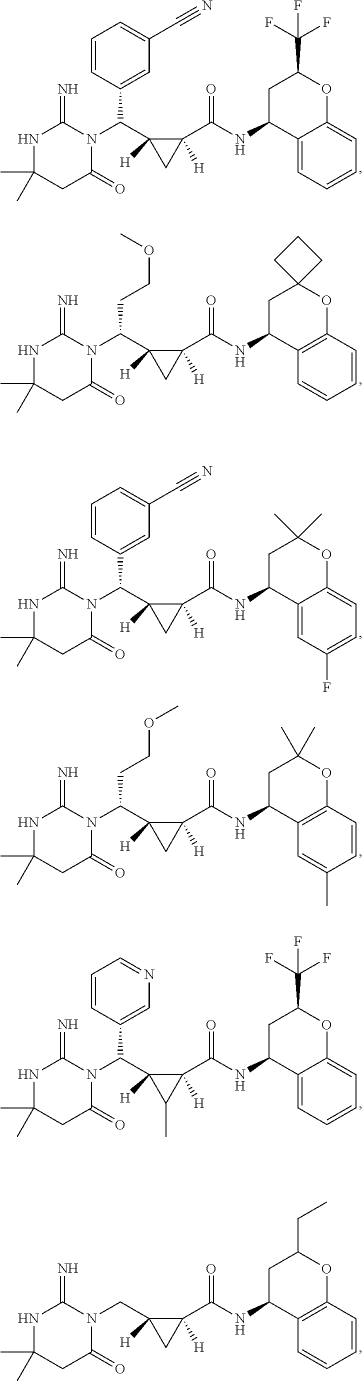 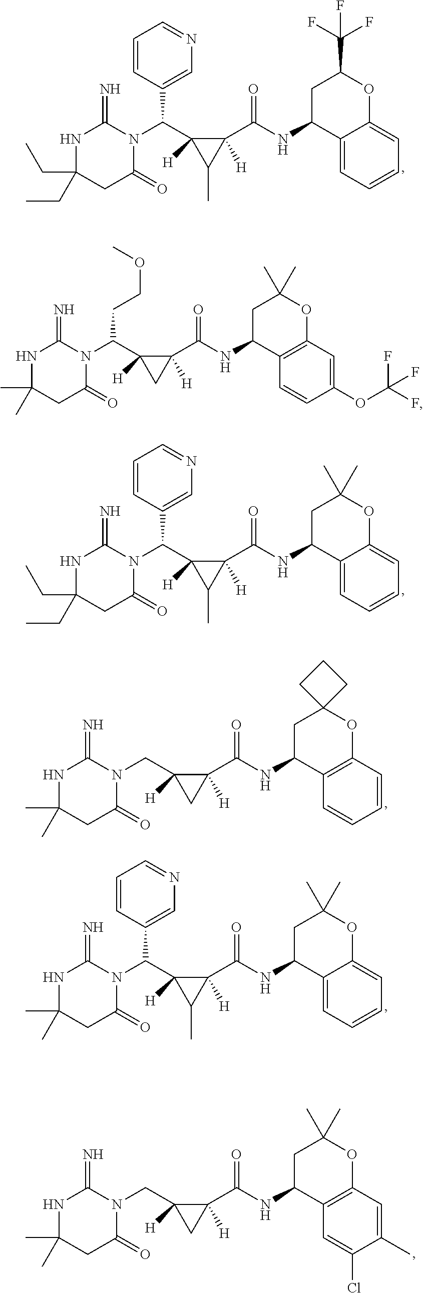 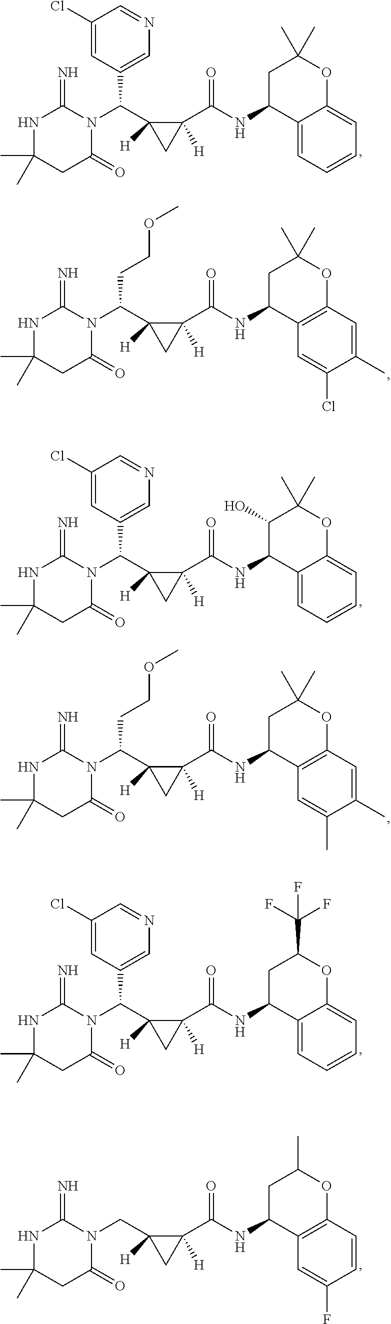 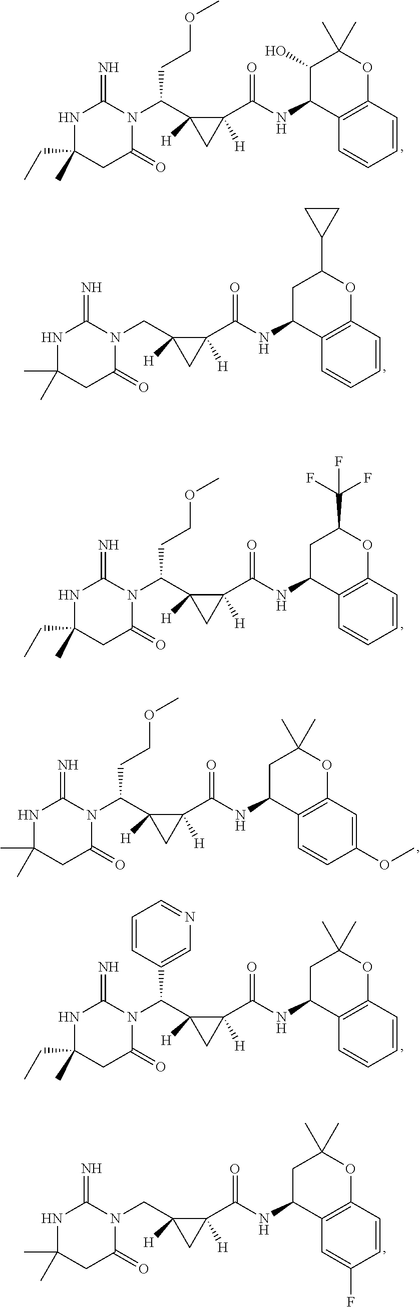 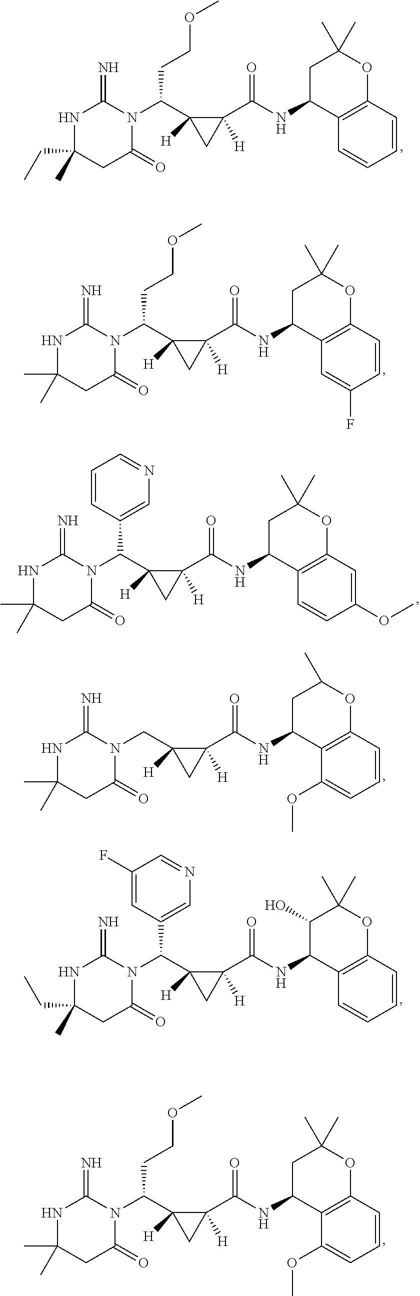 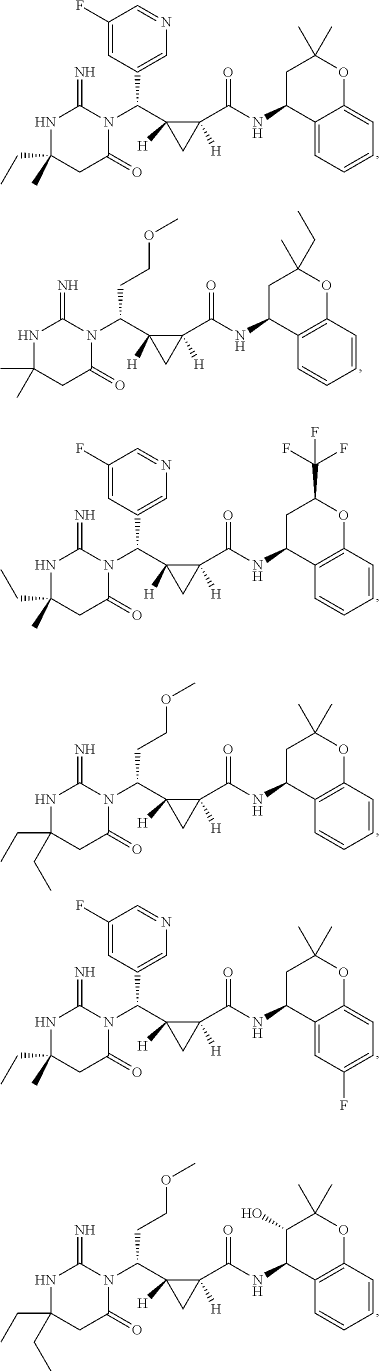 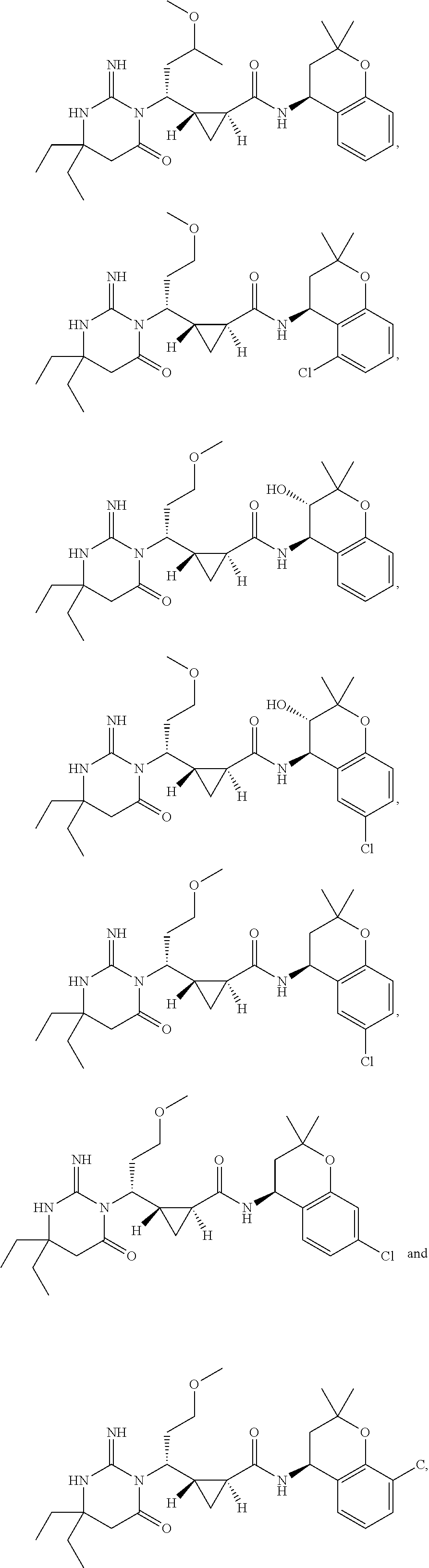
or a pharmaceutically acceptable salt thereof.

17. A method of chemovaccination against Plasmodium infection comprising administering to a patient an effective amount of a compound having the formula 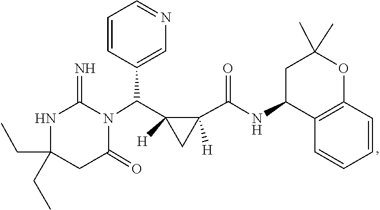
or a pharmaceutically acceptable salt thereof.

18. A method of chemovaccination against Plasmodium infection in a patient comprising administering to the patient 0.1-10 mg of a selective inhibitor of plasmepsin X, or a pharmaceutically acceptable salt thereof.

19. (canceled)

20. (canceled)

21. The method of chemovaccination of claim 20, wherein the Plasmodium parasite infection is a P. falciparum or P. vivax infection.

22. (canceled)

23. The method of chemovaccination of claim 1, wherein the patient does not have a Plasmodium parasite infection, and wherein the patient is simultaneously or sequentially administered a wild-type Plasmodium parasite, or patient is later exposed to a wild-type Plasmodium parasite.

24. (canceled)

25. (canceled)

26. (canceled)

27. (canceled)

28. (canceled)

29. A method of chemovaccination against Plasmodium infection in a patient comprising administering to the patient an effective amount of a selective inhibitor of plasmepsin X, or a pharmaceutically acceptable salt thereof, and an effective amount of one or more additional anti-malarial agents.

30. A method of inducing an immune response to a Plasmodium parasite infection, comprising administering to a patient an effective amount of a compound of Formula (I): 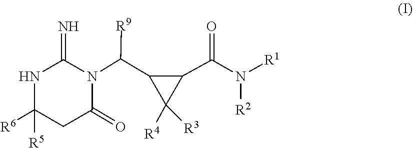
or a pharmaceutically acceptable salt thereof, wherein:
R¹ is a heterocycloalkyl, C₃-C₁₂cycloalkyl, aryl or C₁-C₆alkylaryl, wherein the heterocycloalkyl, C₃-C₁₂cycloalkyl, aryl, or C₁-C₆alkylaryl is unsubstituted or substituted with 1 to 5 substituents independently selected from the group consisting of halogen, —CN, —OH, alkoxy, haloalkoxy, C₁-C₆alkylOC₁-C₆alkyl, C₁-C₆alkylCOOH, —COOH, oxo, —COOC₁-C₆alkyl, C₃-C₆cycloalkyl, spiroC₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
R² is hydrogen, C₁-C₆alkylCOOH, —COOH, C₃-C₆cycloalkyl, C₁-C₆alkyl, haloC₁-C₆alkyl or C₁-C₆alkylOH;
R³ is hydrogen, halogen, —CN, —OH, C₃-C₆cycloalkyl or C₁-C₆alkyl;
R⁴ is hydrogen, halogen, —CN, —OH, C₃-C₆cycloalkyl or C₁-C₆alkyl;
R⁵ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R⁶ forms a C₃-C₆cycloalkyl or C₃-C₆heterocycloalkyl;
R⁶ is hydrogen, halogen, —CN, —OH, alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R⁵ forms a C₃-C₆cycloalkyl or C₃-C₆heterocycloalkyl;
R⁷ is hydrogen, C₁-C₆alkylCOOH, —COOH, C₃-C₆cycloalkyl, C₁-C₆alkyl, haloC₁-C₆alkyl or C₁-C₆alkylOH;
R⁸ is hydrogen, C₁-C₆alkylCOOH, —COOH, C₃-C₆cycloalkyl, C₁-C₆alkyl, haloC₁-C₆alkyl or C₁-C₆alkylOH; and
R⁹ is hydrogen, halogen, —CN, —OH, alkoxy, C₁-C₆alkyl, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and N(R⁷)(R⁸), wherein the C₁-C₆alkyl is unsubstituted or substituted with one, two or three substituents independently selected from the group consisting of halogen, —CN, —OH, alkoxy, heterocycloalkyl, heteroaryl, C₃-C₁₂cycloalkyl, aryl, —COOH, oxo, —COOC₁-C₆alkyl, haloC₁-C₆alkyl, —CON(R⁷)(R⁸) and —N(R⁷)(R⁸), and wherein the heterocycloalkyl, heteroaryl, C₃-C₁₂cycloalkyl and aryl are unsubstituted or substituted with one, two or three substituents independently selected from the group consisting of halogen, —CN, —OH, alkoxy, C₁-C₆alkyl, —COOH, oxo, —COOC₁-C₆alkyl, haloC₁-C₆alkyl, —CON(R⁷)(R⁸) and —N(R⁷)(R⁸).
